(19) United States
(12) Patent Application Publication
(10) Pub. No.: US 2005/0118586 A1
(43) Pub. Date: Jun. 2, 2005

(54) Human cdnas and proteins and uses thereof

(76) Inventors: Stephane Bejanin, Saint Jacques (FR); Hiroaki Tanaka, Antony (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/495,300

(22) PCT Filed: Nov. 25, 2002
(86) PCT No.: PCT/EP02/13210

Related U.S. Application Data
(60) Provisional application No. 60/334,147, filed on Nov. 28, 2001.
(60) Provisional application No. 60/340,465, filed on Dec. 14, 2001.
(60) Provisional application No. 60/373,947, filed on Apr. 18, 2002.

(51) Int. Cl. C12Q 1/68; A01K 67/00; C07H 21/04; C07K 14/705

(52) U.S. Cl. 435/6; 435/69.1; 435/320.1; 435/325; 530/350; 530/388.22; 536/23.5; 800/8

(57) ABSTRACT

The invention concerns NOVEL polynucleotides and polypeptides. Such NOVEL products may be used as reagents in forensic analyses, as chromosome markers, as tissue/cell/organelle-specific markers, in the production of expression vectors. In addition, they may be used in screening and diagnosis assays for abnormal NOVEL expression and/or biological activity and for screening compounds that may be used in the treatment of NOVEL-related disorders.

### RELATED APPLICATION INFORMATION

This application claims priority on U.S. provisional patent application Ser. No. 60/334,147, filed Nov. 28, 2001, entitled “Human cDNAs and proteins and uses thereof”; U.S. provisional patent application Ser. No. 60/340,465, filed Dec. 14, 2001, entitled Human cDNAs and proteins and uses thereof”; and U.S. provisional patent application Ser. No. 60/373,947, filed Apr. 18, 2002, entitled Human cDNAs and proteins and uses thereof”.

### FIELD OF THE INVENTION

The present invention is directed to NOVEL polynucleotides and polypeptides, and fragments, derivatives, and variants thereof. The present invention also relates to recombinant vectors including the polynucleotides of the present invention, particularly recombinant vectors comprising a NOVEL gene regulatory region or a sequence encoding a NOVEL polypeptide, and to host cells containing the polynucleotides of the invention. The invention further relates to antibodies that specifically bind to the polypeptides of the invention and to methods for producing such antibodies and fragments thereof. The invention also provides for methods of detecting the presence of the polynucleotides and polypeptides of the present invention in a sample, methods of diagnosis and screening of abnormal NOVEL polypeptide expression and/or biological activity, methods of screening compounds for their ability to modulate the activity or expression of the NOVEL polypeptides, and uses of such compounds.

### BACKGROUND OF THE INVENTION

cDNAs encoding secreted proteins or fragments thereof represent a particularly valuable source of therapeutic agents. Thus, there is a need for the identification and characterization of secreted proteins and the nucleic acids encoding them.

In addition to being therapeutically useful themselves, secretory proteins include short peptides, called signal peptides, at their amino termini which direct their secretion. These signal peptides are encoded by the signal sequences located at the 5′ ends of the coding sequences of genes encoding secreted proteins. Because these signal peptides will direct the extracellular secretion of any protein to which they are operably linked, the signal sequences may be exploited to direct the efficient secretion of any protein by operably linking the signal sequences to a gene encoding the protein for which secretion is desired. In addition, fragments of the signal peptides called membrane-translocating sequences may also be used to direct the intracellular import of a peptide or protein of interest. This may prove beneficial in gene therapy strategies in which it is desired to deliver a particular gene product to cells other than the cells in which it is produced. Signal sequences encoding signal peptides also find application in simplifying protein purification techniques. In such applications, the extracellular secretion of the desired protein greatly facilitates purification by reducing the number of undesired proteins from which the desired protein must be selected. Thus, there exists a need to identify and characterize the 5′ fragments of the genes for secretory proteins which encode signal peptides.

Sequences coding for secreted proteins may also find application as therapeutics or diagnostics. In particular, such sequences may be used to determine whether an individual is likely to express a detectable phenotype, such as a disease, as a consequence of a mutation in the coding sequence for a secreted protein. In instances where the individual is at risk of suffering from a disease or other undesirable phenotype as a result of a mutation in such a coding sequence, the undesirable phenotype may be corrected by introducing a normal coding sequence using gene therapy. Alternatively, if the undesirable phenotype results from overexpression of the protein encoded by the coding sequence, expression of the protein may be reduced using antisense or triple helix based strategies.

The secreted human polypeptides encoded by the coding sequences may also be used as therapeutics by administering them directly to an individual having a condition, such as a disease, resulting from a mutation in the sequence encoding the polypeptide. In such an instance, the condition can be cured or ameliorated by administering the polypeptide to the individual.

In addition, the secreted human polypeptides or fragments thereof may be used to generate antibodies useful in determining the tissue type or species of origin of a biological sample. The antibodies may also be used to determine the cellular localization of the secreted human polypeptides or the cellular localization of polypeptides which have been fused to the human polypeptides. In addition, the antibodies may also be used in immunoaffinity chromatography techniques to isolate, purify, or enrich the human polypeptide or a target polypeptide which has been fused to the human polypeptide.

### SUMMARY OF THE INVENTION

The present invention provides a purified or isolated polynucleotide comprising, consisting of, or consisting essentially of a nucleotide sequence selected from the group consisting of: (a) the sequences of the odd SEQ ID NOs:1-39; (b) the coding sequences of the odd SEQ ID NOs:1-39; (c) the sequences encoding one of the polypeptides of the even SEQ ID NOs:2-40; (d) the genomic sequences coding for the NOVEL polypeptides; (e) the 5′transcriptional regulatory regions of NOVEL genes; (f) the 3′ transcriptional regulatory regions of NOVEL genes; (g) the polynucleotides comprising the nucleotide sequence of any combination of (d)-(f); (h) the variant polynucleotides of any of the polynucleotides of (a)-(g); (i) the polynucleotides comprising a nucleotide sequence of (a)-(h), wherein the polynucleotide is single stranded, double stranded, or a portion is single stranded and a portion is double stranded; (j) the polynucleotides comprising a nucleotide sequence complementary to any of the single stranded polynucleotides of (i). The invention further provides for fragments of the nucleic acids and polypeptides of (a)-(j) described above.

Further embodiments of the invention include purified or isolated polynucleotides that comprise, consist of, or consist essentially of a nucleotide sequence at least 70% identical, more preferably at least 75%, and even more preferably at least 80%, 85%, 90%, 95%, 96%, 97%, 98%, or 99% identical, to any of the nucleotide sequences in (a)-(j) above, e.g. over a region of contiguous nucleotides at least about any one integer between 10 and the last integer representing the last integer representing the last nucleotide of a specified sequence of the sequence listing, or a polynucleotide which hybridizes under stringent hybridization conditions to a polynucleotide of the present invention including (a) through (j) above.

The present invention also relates to recombinant vectors, which include the purified or isolated polynucleotides of the present invention, and to host cells recombinant for the polynucleotides of the present invention, as well as to methods of making such vectors and host cells. The present invention further relates to the use of these recombinant vectors and recombinant host cells in the production of NOVEL polypeptides. The present invention further relates to a polynucleotide of the present invention operably linked to a regulatory sequence including promoters, enhancers, etc.

The invention further provides a purified or isolated polypeptide comprising, consisting of, or consisting essentially of an amino acid sequence selected from the group consisting of: (a) the full length polypeptides of even SEQ ID NOs:2-40; (b) the epitope-bearing fragments of the polypeptides of even SEQ ID NOs:2-40; (c) the domains of the polypeptides of even SEQ ID NOs:2-40; (d) the signal peptides of the polypeptides of even SEQ ID NOs:2-40; (e) the mature polypeptides of even SEQ ID NOs:2-40; and (f) the allelic variant polypeptides of any of the polypeptides of (a)-(e). The invention further provides for fragments of the polypeptides of (a)-(f) above, such as those having biological activity or comprising biologically functional domain(s).

The present invention further includes polypeptides with an amino acid sequence with at least 70% similarity, and more preferably at least 75%, 80%, 85%, 90%, 95%, 96%, 97%, 98%, or 99% similarity to those polypeptides described in (a)-(f), or fragments thereof, as well as polypeptides having an amino acid sequence at least 70% identical, more preferably at least 75% identical, and still more preferably 80% 85%, 90%, 95%, 96%, 97%, 98%, or 99% identical to those polypeptides described in (a)-(i), or fragments thereof, e.g. over a region of amino acids at least any one integer between 6 and the last integer representing the last amino acid of a specified polypeptide sequence of the sequence listing. The invention further relates to methods of maling the polypeptides of the present invention.

The present invention further relates to transgenic-plants or animals, wherein said transgenic plant or animal is transgenic for a polynucleotide of the present invention and expresses a polypeptide of the present invention.

The invention further relates to antibodies that specifically bind to NOVEL polypeptides of the present invention and fragments thereof as well as to methods for producing such antibodies and fragments thereof.

The invention also provides kits, uses and methods for detecting NOVEL gene expression and/or biological activity in a biological sample. One such method involves assaying for the expression of a NOVEL polynucleotide in a biological sample using the polymerase chain reaction (PCR) to amplify and detect NOVEL polynucleotides or Southern and Northern blot hybridization to detect NOVEL genomic DNA, cDNA or mRNA. Alternatively, a method of detecting NOVEL gene expression in a test sample can be accomplished using a compound which binds to a NOVEL polypeptide of the present invention or a portion of a NOVEL polypeptide.

The present invention also relates to diagnostic methods and uses of NOVEL polynucleotides and polypeptides for identifying individuals or non-human animals having elevated or reduced levels of NOVEL gene products, which individuals are likely to benefit from therapies to suppress or enhance NOVEL gene expression, respectively, and to methods of identifying individuals or non-human animals at increased risk for developing, or at present having, certain diseases/disorders associated with NOVEL polypeptide expression or biological activity.

The present invention also relates to kits, uses and methods of screening compounds for their ability to modulate (e.g. increase or inhibit) the activity or expression of NOVEL polypeptides including compounds that interact with NOVEL gene regulatory sequences and compounds that interact directly or indirectly with a NOVEL polypeptide. Uses of such compounds are also within the scope of the present invention.

The present invention also relates to pharmaceutical or physiologically acceptable compositions comprising, an active agent, the polypeptides, polynucleotides or antibodies of the present invention, as well as, typically, a physiologically acceptable carrier.

The present invention also relates to computer systems containing cDNA codes and polypeptide codes of sequences of the invention and to computer-related methods of comparing sequences, identifying homology or features using NOVEL polypeptides or NOVEL polynucleotide sequences of the invention.

In another aspect, the present invention provides an isolated polynucleotide, the polynucleotide comprising a nucleic acid-sequence encoding a polypeptide of the present invention including the polypeptide of (a) through (f) above.

In another aspect, the present invention provides a non-human transgenic animal comprising the host cell.

In another aspect, the present invention provides a method of making a NOVEL polypeptide, the method comprising a) providing a population of host cells comprising a herein-described polynucleotide and b) culturing the population of host cells under conditions conducive to the production of the polypeptide within said host cells.

In one embodiment, the method further comprises purifying the polypeptide from the population of host cells.

In another aspect, the present invention provides a method of making a NOVEL polypeptide, the method comprising a) providing a population of cells comprising a polynucleotide encoding a herein-described polypeptide; b) culturing the population of cells under conditions conducive to the production of the polypeptide within the cells; and c) purifying the polypeptide from the population of cells.

In another aspect, the present invention provides a biologically active polypeptide encoded by any of the herein-described polynucleotides.

In one embodiment, the polypeptide is selectively recognized by an antibody raised against an antigenic polypeptide, or an antigenic fragment thereof, the antigenic polypeptide comprising any one of the sequences shown as even SEQ ID NOs:2-40.

In another aspect, the present invention provides an antibody that specifically binds to any of the herein-described polypeptides and methods of binding antibody to said polypeptide.

In another aspect, the present invention provides a method of determining whether a NOVEL gene is expressed within a mammal, the method comprising the steps of: a) providing a biological sample from said mammal; b) contacting said biological sample with either of: (i) a polynucleotide that hybridizes under stringent conditions to any of the herein-described polynucleotides; or (ii) a polypeptide that specifically binds to any of the herein-described polypeptides; and c) detecting the presence or absence of hybridization between the polynucleotide and an RNA species within the sample, or the presence or absence of binding of the polypeptide to a protein within the sample; wherein a detection of the hybridization or of the binding indicates that the NOVEL gene is expressed within the mammal.

In one embodiment, the polynucleotide is a primer, and the hybridization is detected by detecting the presence of an amplification product comprising the sequence of the primer. In another embodiment, the polypeptide is an antibody.

In another aspect, the present invention provides a method of determining whether a mammal has an elevated or reduced level of NOVEL gene expression, the method comprising the steps of: a) providing a biological sample from the mammal; and b) comparing the amount of any of the herein-described polypeptides, or of an RNA species encoding the polypeptide, within the biological sample with a level detected in or expected from a control sample; wherein an increased amount of the polypeptide or the RNA species within the biological sample compared to the level detected in or expected from the control sample indicates that the mammal has an elevated level of the NOVEL gene expression, and wherein a decreased amount of the polypeptide or the RNA species within the biological sample compared to the level detected in or expected from the control sample indicates that the mammal has a reduced level of the NOVEL gene expression.

In another aspect, the present invention provides a method of identifying a candidate modulator of a NOVEL polypeptide, the method comprising: a) contacting any of the herein-described polypeptides with a test compound; and b) determining whether the compound specifically binds to the polypeptide; wherein a detection that the compound specifically binds to the polypeptide indicates or inhibits or activates of a specified biological activity that the compound is a candidate modulator of the NOVEL polypeptide.

### BRIEF DESCRIPTION OF TABLES

Table I provides the Applicants' internal designation number (Clone ID_Clone Name) which corresponds to each sequence identification number (SEQ ID NO) of the Sequence Listing, and indicates whether the sequence is a nucleic acid sequence (DNA) or a polypeptide sequence (PRT). Further provided is information regarding the name of the corresponding nucleic acid or polypeptide sequence.

Table II provides the positions of the nucleotides of the corresponding SEQ ID NOs of the Sequence Listing which comprise the open reading frame (ORF), signal peptide, mature peptide, polyadenylation signal, and the polyA tail of the polynucleotides of the invention.

Table III provides the positions of the anino acid of the corresponding SEQ ID NOs. of the Sequence Listing which comprise the positions of immunogenic epitopes of the polypeptides of the invention, which are useful in antibody generation.

Table IV provides the positions of the amino acid of the corresponding SEQ ID NOs. of the Sequence Listing which comprise a domain, a signature or a motif.

### BRIEF DESCRIPTION OF SEQUENCES

Sequences are presented in the accompanying Sequence Listing.

Odd SEQ ID NOs:1-39 are the nucleotide sequences of cDNAs, with open reading frames as indicated. When appropriate, the potential polyadenylation site and polyadenylation signal are also indicated.

Even SEQ ID NOs:2-40 are the amino acid sequences of proteins encoded by the cDNAs of odd SEQ ID NOs:1-39.

In accordance with the regulations relating to Sequence Listings, the following codes have been used in the Sequence Listing to describes nucleotide sequences. The code “r” in the sequences indicates that the nucleotide may be a guanine or an adenine. The code “y” in the sequences indicates that the nucleotide may be a thymine or a cytosine. The code “m” in the sequences indicates that the nucleotide may be an adenine or a cytosine. The code “k” in the sequences indicates that the nucleotide may be a guanine or a thymine. The code “s” in the sequences indicates that the nucleotide may be a guanine or a cytosine. The code “w” in the sequences indicates that the nucleotide may be an adenine or a thymine. In addition, all instances of the symbol “n” in the nucleic acid sequences mean that the nucleotide can be adenine, guanine, cytosine or thymine.

In some instances, the polypeptide sequences in the Sequence Listing contain the symbol “Xaa.” These “Xaa” symbols indicate either (1) a residue which cannot be identified because of nucleotide sequence ambiguity or (2) a stop codon in the determined sequence where applicants believe one should not exist (if the sequence were determined more accurately). In some instances, several possible identities of the unknown amino acids may be suggested by the genetic code.

In the case of secreted proteins, it should be noted that, in accordance with the regulations governing Sequence Listings, in the appended Sequence Listing the encoded protein (i.e. the protein containing the signal peptide and the mature protein or fragment thereof) extends from an amino acid residue having a negative number through a positively numbered amino acid residue. Thus, the first anmino acid of the mature protein resulting from cleavage of the signal peptide is designated as amino acid number 1, and the first amino acid of the signal peptide is designated with the appropriate negative number.

In the case that a polynucleotide or polypeptide sequence described in the specification for SEQ ID NOs:1-40 is in conflict with the corresponding sequence provided in the Sequence listing, the sequences provided in the Sequence listing controls.

It should be appreciated that the polynucleotide and polypeptide sequences of SEQ ID NO:1-40 of the Sequence Listing are hereby incorporated by reference in their entireties.

### DETAILED DESCRIPTION OF THE INVENTION AND PREFERRED EMBODIMENTS

Definitions.

Before describing the invention in greater detail, the following definitions are set forth to illustrate and define the meaning and scope of the terms used to describe the invention herein.

The term “NOVEL gene,” when used herein, encompasses genomic, mRNA and cDNA sequences encoding a NOVEL polypeptide, including the 5′ and 3′ untranslated regions of said sequences.

The term “NOVEL polypeptide biological activity” or “NOVEL biological activity” is intended for polypeptides exhibiting any activity similar, but not necessarily identical, to an activity of a NOVEL polypeptide of the invention. The NOVEL polypeptide biological activity of a given polypeptide may be assessed using any suitable biological assay, a number of which are known to those skilled in the art. In contrast, the term “biological activity” refers to any activity that any polypeptide may have.

The term “corresponding mRNA” refers to mRNA which was or can be a template for cDNA synthesis for producing a cDNA of the present invention.

The term “corresponding genomic DNA” refers to genomic DNA which encodes an mRNA of interest, e.g. corresponding to a cDNA of the invention, which genomic DNA includes the sequence of one of the strands of the mRNA, in which thymidine residues in the sequence of the genomic DNA (or cDNA) are replaced by uracil residues in the mRNA.

The term “heterologous”, when used herein in reference to a polypeptide or polynucleotide, is intended to designate any polynucleotide or polypeptide other than a particular NOVEL polynucleotide or NOVEL polypeptide of the invention, respectively.

“Providing” with respect to, e.g. a biological sample, population of cells, etc. indicates that the sample, population of cells, etc. is somehow used in a method or procedure. Significantly, “providing” a biological sample or population of cells does not require that the sample or cells are specifically isolated or obtained for the purposes of the invention, but can instead refer, for example, to the use of a biological sample obtained by another individual, for another purpose.

An “amplification product” refers to a product of any amplification reaction, e.g. PCR, RT-PCR, LCR, etc.

A “modulator” of a protein or other compound refers to any agent that has a functional effect on the protein, including physical binding to the protein, alterations of the quantity or quality of expression of the protein, altering any measurable or detectable activity, property, or behavior of the protein, or in any way interacts with the protein or compound.

“A test compound” can be any molecule that is evaluated for its ability to modulate a protein or other compound.

An antibody or other compound that specifically binds to a polypeptide or polynucleotide of the invention is also said to “selectively recognize” the polypeptide or polynucleotide.

The term “isolated” with respect to a molecule requires that the molecule be removed from its original environment (e. g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment. Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified polynucleotide makes up less than 5% (may also be specified as 10%, 25%, 50%, or 75%) of the number of nucleic acid inserts in the vector molecules. Further specifically excluded are whole cell genomic DNA or whole cell RNA preparations (including said whole cell preparations which are mechanically sheared or enzymatically digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

The term “purified” does not require absolute purity; rather, it is intended as a relative definition. Purification of starting material or natural material to at least one order of magnitude, preferably two or three orders, and more preferably four or five orders of magnitude is expressly contemplated. The term “purified” is further used herein-to describe a polypeptide or polynucleotide of the invention which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero- dimers, trmers, etc. The term “purified” may also be used to specify the separation of covalently closed (i.e. circular) polynucleotides from linear polynucleotides. A substantially pure polypeptide or polynucleotide typically comprises about 50%, preferably 60 to 90% weightlweight of a polypeptide or polynucleotide sample, respectively, more usually about 95%, and preferably is over about 99% pure but, may be specificed as any integer of percent between 50 and 100. Polypeptide and polynucleotide purity, or homogeneity, is indicated by a number of means well known in the art, such as agarose or polyacrylarnide gel electrophoresis of a sample, or using HPLC. As an alternative embodiment, purification of the polypeptides and polynucleotides of the present invention may be expressed as “at least” a percent purity relative to heterologous polypeptides and polynucleotides (DNA, RNA or both). As a preferred embodiment, the polypeptides and polynucleotides of the present invention are at least; 10%, 20%, 30%, 40%, 50%, 60%, 70%, 80%, 90%, 95%, 96%, 96%, 98%, 99%, or 100% pure relative to heterologous polypeptides and polynucleotides, respectively. As a further preferred embodiment the polypeptides and polynucleotides have a purity ranging from any number, to the thousandth position, between 90% and 100% (e.g., a polypeptide or polynucleotide at least 99.995% pure) relative to either heterologous polypeptides or polynucleotides, respectively, or as a weight/weight ratio relative to all compounds and molecules other than those existing in the carrier. Each number representing a percent purity, to the thousandth position, may be claimed as individual species of purity.

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (i.e. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modification such as (a) an alternative linking group, (b) an analogous form of purine, (c) an analogous form of pyruimdine, or (d) an analogous sugar (see, e.g., WO 95/04064). Preferred modifications of the present invention include, but are not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthioN6-isopentenyladenine, uracil-5-oxyacetic acid (v) ybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6diaminopurine. The polynucleotide sequences of the invention may be prepared by any known method, including synthetic, recombinant, ex vivo generation, or a combination thereof, as well as utilizing any purification methods known in the art (for teachings regarding the preparation of modified oligos and nucleotides, see, e.g., U.S. Pat. Nos. 5,378,825; 5,386,023; 5,489,677; 5,602,240; and 5,610,289, U.S. Pat. Nos. 5,264,562 and 5,264,564, U.S. Pat. No. 5,223,618, U.S. Pat. No. 5,508,270, U.S. Pat. No. 4,469,863, U.S. Pat. Nos. 5,610,289 or 5,625,050, U.S. Pat. No. 5,256,775 or U.S. Pat. No. 5,366,878, PCT applications WO 94/17093 and WO 94/02499, U.S. Pat. No. 5,476,925, U.S. Pat. No. 5,023,243, U.S. Pat. Nos. 5,130,302 and 5,177,198).

The term “upstream” is used herein to refer to a location which is toward the 5′ end of the polynucleotide from a specific reference point.

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini, and may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-liking, cyclization, disulfide bond formation, demethylation, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, for instance Creighton, (1993), Posttranslational Covalent Modification of Proteins, W.H. Freeman and Company, New York B. C. Johnson, Ed., Academic Press, New York 1-12; Seifter, et al., (1990) Meth Enzymol 182:626-646; Rattan et al., (1992) Ann NY Acad Sci 663:48-62). Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

The term “domain” refers to an amino acid fragment with specific biological properties. This term encompasses all known structural and linear biological motifs. Examples of such motifs include but are not limited to leucine zippers, helix-turn-helix motifs, glycosylation sites, ubiquitination sites, alpha helices, and beta sheets, signal peptides which direct the secretion of proteins, sites for post-translational modification, enzymatic active sites, substrate binding sites, and enzymatic cleavage sites.

Although each of these terms has a distinct meaning, the terms “comprising”, “consisting of” and “consisting essentially of” may be interchanged for one another throughout the instant application. The term “having” has the same meaning as “comprising” and may be replaced with either the term “consisting of” or “consisting essentially of”.

Unless otherwise specified in the application, nucleotides and amino acids of polynucleotides and polypeptides, respectively, of the present invention are contiguous and not interrupted by heterologous sequences.

As used herein, the term “tumor” refers to an abnormal mass or population of cells that result from excessive cell division, whether malignant or benign, and all pre-cancerous and cancerous cells and tissues. A “tumor” is further defined as two or more physically associated neoplastic cells. The term “transformed cells,” “malignant cells” or “cancer” are interchangeable and refer to cells that have undergone malignant transformation, and also includes lymphocytes that have undergone blast transformation. Transformed cells have a greater ability to cause tumors when injected into animals, can typically proliferate without requiring adhesion to a substratum, and also lack contact inhibition. The term “cancer” or “neoplastic disease” encompasses any type of cancer, in any tissue, and includes, but is not limited to, carcinomas, lymphomas, blastomas, sarcomas, and leukemias.

The terms “inducing” or “inducing” with respect to a cellular process, e.g., apoptosis, refers to increasing or decreasing the number of cells that undergo the process, or the rate by which cells undergo the process, in a given cell population. Preferably the increase or decrease is at least 1.25, 1.5, 2, 5, 10, 50, 100, 500 or 1000 fold increase or decrease as compared to normal, untreated or negative control cells.

A “therapeutically effective amount”, in reference to the treatment of a disease or condition, refers to an amount of a compound that is capable of having any detectable, positive effect on any symptom, aspect, or characteristics of the disease or condition.

The terms “killing” or “inducing cytotoxicity” as used herein refer to inducing cell death by either apoptosis andlor necrosis, whereby embodiments of the invention include only apoptosis, only necrosis and both apoptosis and necrosis.

The terms “preventing” and “suppressing” as used herein refer to administering a compound prior to the onset of clinical symptoms of a disease or condition so as to prevent a physical maanifestation of the disease or condition. The term “prophylaxis” is distinct from “treatment” and encompasses “preventing” and “suppressing”. Herein, “protection” includes “prophylaxis”. Protection need not be absolute to be useful.

The term “treating” as used herein refers to administering a compound after the onset of clinical symptoms. The term “in need of treatment” as used herein refers to a judgment made by a caregiver that an individual or animal requires or will benefit from treatment. This judgment is made based on a variety of factors that are in the realm of a caregiver's expertise, but that include the knowledge that the individual or animal is ill, or will be ill, as the result of a condition that is treatable by a compound of the invention.

The term “individual” or “patient” as used herein refers to any animal, including mammals, preferably mice, rats, other rodents, rabbits, dogs, cats, swine, cattle, sheep, horses, or primates, and most preferably humans. The term may specify male or female or both, or exclude male or female.

As used herein, the term “non-human animal” refers to any non-human animal, including insects, birds, rodents and more usually mammals. Preferred non-human animals include: primates; farm animals such as swine, goats, sheep, donkeys, cattle, horses, chickens, rabbits; and rodents, preferably rats or mice. As used herein, the term “animal” is used to refer to any species in the animal kingdom, preferably vertebrates, including birds and fish, and more preferably a mammal. Both the terms “animal” and “mammal” expressly embrace human subjects unless preceded with the term “non-human”.

As used herein, the terms “physiologically acceptable,” “pharmaceutically acceptable,” and “pharmaceutical” are interchangeable.

Identity Between Nucleic Acids or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity or similarity is evaluated using any of the variety of sequence comparison algorithms and programs known in the art. Such algorithms and programs include, but are by no means limited to, TBLASTN, BLASTP, FASTA, TFASTA, CLUSTALW, FASTDB (Pearson and Lipman, (1988), PNAS 85(8):2444-2448; Altschul et al., (1990), J. Mol. Biol. 215(3):403-410; Thompson et al. (1994), Nucleic Acids Res. 22(2):4673-4680; Higgins et al., (1996), Meth. Enzymol. 266:383-402; Altschul et al., (1993), Nature Genetics 3:266-272; Brutlag et al. (1990) Comp. App. Biosci. 6:237-24).

In a particularly preferred embodiment, protein and nucleic acid sequence identities are evaluated using the Basic Local Alignment Search Tool (“BLAST”) which is well known in the art (e.g., Karlin and Altschul, (1990), PNAS 87:2267-2268; Altschul et al., (1997), Nuc. Acids Res. 25:3389-3402). In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) BLASTN compares a query protein sequence against a nucleotide sequence database translated in all six reading frames (both strands); and
(S) BLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., (1992), Science 256:1443-1445; Henikoff and Henikoff, (1993), Proteins 17:49-61). Less preferably, the PAM or PAM250 matrices may also be used (see, e.g., Schwartz and Dayhoff, (1978), eds., Matrices for Detecting Distance Relationships: Atlas of Protein Sequence and Structure, Washington: National Biomedical Research Foundation). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990). The BLAST programs may be used with the default parameters or with modified parameters provided by the user.

Another preferred method for determining the best overall match between a query nucleotide or amino acid sequence (a sequence of the present invention). and a subject sequence, also referred to as a global sequence alignment, can be determined using the FASTDB computer program based on the algorithm of Brutlag et al. (1990). In a sequence alignment the query and subject sequences are both DNA or amino acid sequences. An RNA sequence can also be compared by first converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. If the subject sequence is shorter than the query sequence because of 5′ or 3′ deletions (for nucleotide sequences) or N- or C-terminal deletions (for amino acid sequences), not because of internal deletions, a manual correction must be made to the results, because the PASTDB program does not account for terminal truncations of a subject sequence when calculating percent identity. The manual correction involves determining the percent of the total query sequence that is not aligned because of a truncation in the subject sequence, and subtracting this percentage from the percent identity calculated by the FASTDB program. This corrected score is what is used for the purposes of the present invention. No other manual corrections are made for the purposes of the present invention.

Polynucleotides of the Invention

Many of the methods described in the present specification rely on the use of common molecular biological techniques. A number of such techniques are taught, generally, in “Molecular Cloning; A Laboratory Manual”, 2d ed., Cole Spring Harbor Laboratory Press, Sambrook, et al., eds., 1989, and “Methods in Enzymology; Guide to Molecular Cloning Techniques”, Academic Press, Berger and Kimmel eds., 1987.

The present invention concerns NOVEL genomic and cDNA sequences. The present invention encompasses NOVEL genes, polynucleotides comprising NOVEL genomic and cDNA sequences, as well as fragments and variants thereof. These polynucleotides may be purified, isolated, or recombinant.

Also encompassed by the present invention are allelic variants, orthologs, splice variants, and/or species homologues of the NOVEL genes. Procedures known in the art can be used to obtain full-length genes and cDNAs, allelic variants, splice variants, full-length coding portions, orthologs, and/or species homologues of genes and cDNAs corresponding to a nucleotide sequence selected from the group consisting of sequences of odd SEQ ID NOs:1-39. For example, allelic variants, orthologs and/or species homologues may be isolated and identified by making suitable probes or primers from the sequences provided herein and screening a suitable nucleic acid source for allelic variants and/or the desired homologue using any technique known to those skilled in the art including those described into the section entitled “To find similar sequences”.

In a specific embodiment, the polynucleotides of the invention are at least 15, 30, 50, 100, 125, 500, or 1000 continuous nucleotides. In another embodiment, the polynucleotides are less than or equal to 300 kb, 200 kb, 100 kb, 50 kb, 10 kb, 7.5 kb, 5 kb, 2.5 kb, 2 kb, 1.5 kb, or 1 kb in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 75, 50, 25, 20, 15, 10, 5, 4, 3, 2, or 1 naturally occurring genomic flanking gene(s).

cDNA Sequences of the Invention

Structural parameters of each of the cDNAs of the present invention are presented in the appended Sequence Listing. Accordingly, the coding sequence (CDS) or open reading frame (ORF) of each cDNA of the invention refers to the nucleotide sequence beginning with the first nucleotide of the start codon and ending with the nucleotide immediately 5′ to the first nucleotide of the stop codon. Similarly, the 5′ untranslated region (or 5′UTR) of each cDNA of the invention refers to the nucleotide sequence starting at nucleotide 1 and ending at the nucleotide immediately 5′ to the first nucleotide of the start codon. The 3′ untranslated region (or 3′UTR) of each cDNA of the invention refers to the nucleotide sequence starting at the first nucleotide of the stop codon and ending at the last nucleotide of the cDNA.

Coding Sequences

Another object of the invention is an isolated, purified or recombinant polynucleotide comprising the coding sequence of a sequence selected from the group consisting of the polynucleotide sequences of the appended Sequence Listing and variants thereof.

It will be appreciated that should the extent of the coding sequence differ from that indicated in the appended sequence listing as a result of a sequencing error, reverse transcription or amplification error, tRNA splicing, post-translational modification of the encoded protein, enzymatic cleavage of the encoded protein, or other biological factors, one skilled in the art would be readily able to identify the extent of the coding sequences in the polynucleotide sequences of the Sequence Listing and allelic variants thereof. Accordingly, the scope of any claims herein relating to nucleic acids containing the coding sequence of one of the polynucleotide sequences of the Sequence Listing is not to be construed as excluding any readily identifiable variations from or equivalents to the coding sequences described in the appended sequence listing. Equivalents include any alterations in a nucleotide coding sequence that does not result in an amino acid change, or that results in a conservative amino acid substitution, as defined below, in the polypeptide encoded by the nucleotide sequence. Similarly, should the extent of the polypeptides differ from those indicated in the appended Sequence Listing as a result of any of the preceding factors, the scope of claims relating to polypeptides comprising the amino acid sequence of the polypeptide sequences of the appended Sequence Listing is not to be construed as excluding any readily identifiable variations from or equivalents to the sequences described in the appended sequence listing.

The above disclosed polynucleotides that contain the coding sequence of the NOVEL genes may be expressed in a desired host cell or a desired host organism, when this polynucleotide is placed under the control of suitable expression signals. The expression signals may be either the expression signals contained in the regulatory regions in the NOVEL genes of the invention or, in contrast, the signals may be exogenous regulatory nucleic sequences. Such a polynucleotide, when placed under the suitable expression signals, may also be inserted in a vector for its expression andlor amplification.

Further included in the present invention are polynucleotides encoding the polypeptides of the present invention that are fused in frame to the coding sequences for additional heterologous amino acid sequences. Also included in the present invention are nucleic acids encoding polypeptides of the present invention together with additional, non-coding sequences, including, but not limited to, non-coding 5′ and 3′ sequences, vector sequence, sequences used for purification, probing, or priming. For example, heterologous sequences include transcribed, untranslated sequences that may play a role in transcription and mRNA processing, such as ribosome binding and stability of mRNA. The heterologous sequences may alternatively comprise additional coding sequences that provide additional functionalities, e.g. a hexahistidine or HA tag).

Regulatory Sequences of the Invention

As mentioned, the genomic sequence of NOVEL genes contain regulatory sequences in the non-coding 5′-flanking region and possibly in the non-coding 3′-flanking region that border the NOVEL polypeptide coding regions containing the exons of these genes.

Polynucleotides derived from NOVEL polynucleotide 5′ and 3′ regulatory regions are useful in order to detect the presence of at least a copy of a genomic nucleotide sequence of the NOVEL gene or a fragment thereof in a test sample.

Preferred Regulatory Sequences

Polynucleotides carrying the regulatory elements located at the 5′ end and at the 3′ end of NOVEL polypeptide coding regions may be advantageously used to control the processing, localization, stability, maturation and transcriptional and translational activity of a heterologous polynucleotide of interest, e.g; the regulatory polynucleotides may be part of a recombinant expression vector that may be used to express a coding sequence in a desired host cell or host organism (for a review on UTRs see Decker and Parker, (1995) Curr. Opin. Cell. Biol. 7(3) :368-92, Derrigo et al., (2000) Int. J. Mol. Med. 5(2): 111-23). In particular, 3′UTRs may be used in order to control the stability of heterologous mRNAs in recombinant vectors using any methods known to those skilled in the art including Makrides (1999) Protein Expr Purif 1999 November; 17(2):183-202), U.S. Pat. Nos. 5,925,564; 5,807,707 and 5,756,264.

The present invention also concerns a purified or isolated nucleic acid comprising a polynucleotide which is selected from the group consisting of the 5′ and 3′ NOVEL polynucleotide regulatory regions, sequences complementary thereto, regulatory active fragments and variants thereof, and those that hybridize under stringent hybridization conditions therewith. Further included are nucleic acids comprising a nucleotide sequence having at least 95% identity with any of the herein-described NOVEL 5′ or 3′ regulatory sequences, as well as 5′- or 3′-UTRs of the polynucleotide sequences of the appended Sequence Listing, sequences complementary thereto, regulatory active fragments and allelic variants thereof. Fragments of 5′ and 3′ regulatory regions may have a length corresponding to any one integer between 20 and 20,000 nucleotides in length.

For the purpose of the invention, a nucleic acid or polynucleotide is “functional” as a “regulatory region” for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide.

The invention also comprises a nucleic acid molecule encoding a desired polypeptide or a nucleic acid molecule of interest, wherein said nucleic acid molecule is operably linked to any of the herein-described regulatory sequences. The desired polypeptide may be of various nature or origin, encompassing proteins of prokaryotic viral or eukaryotic origin. Also encompassed are eukaryotic proteins such as intracellular proteins, such as “house keeping” proteins, membrane-bound proteins, such as mitochondrial membrane-bound proteins and cell surface receptors, and secreted proteins such as endogenous mediators such as cytokines. The desired polypeptide may be a heterologous polypeptide or a NOVEL polypeptide.

Polynucleotide Variants

The invention also relates to variants of the polynucleotides described herein and fragments thereof. “Variants” of polynucleotides, as the term is used herein, are polynucleotides that differ from a reference polynucleotide. Generally, differences are limited so that the nucleotide sequences of the reference and the variant are closely similar overall and, in many regions, identical. The present invention encompasses both allelic variants and degenerate variants.

Allelic Variant

A variant of a polynucleotide may be a naturally occurring variant such as a naturally occurring allelic variant, or it may be a variant that is not known to occur naturally. By an “allelic variant” is intended one of several alternate forms of a gene occupying a given locus on a chromosome of an organism (see Lewin, (1989), PNAS 86:9832-8935). Diploid organisms may be homozygous or heterozygous for an allelic form. Non-naturally occurring variants of the polynucleotide may be made by art-known mutagenesis techniques, including those applied to polynucleotides, cells or organisms.

Degenerate Variant

In addition to the isolated polynucleotides of the present invention, and fragments thereof, the invention further includes polynucleotides which comprise a sequence substantially different from those described above but which, due to the degeneracy of the genetic code, still encode a NOVEL polypeptide of the present invention. These polynucleotide variants are referred to as “degenerate variants” throughout the instant application. That is, all possible polynucleotide sequences that encode the NOVEL polypeptides of the present invention are contemplated. This includes the genetic code and species-specific codon preferences known in the art.

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions. In the context of the present invention, preferred embodiments are those in which the polynucleotide variants encode polypeptides which retain substantially the same biological properties or activities as the NOVEL protein. More preferred polynucleotide variants are those containing conservative substitutions.

Similar Polynucleotides

Other embodiments of the present invention provide a purified, isolated or recombinant polynucleotide which is at least 80%, 85%, 90%, 95%, 96%, 97%, 98% or 99% identical to a polynucleotide of the present invention. Although similar polynucleotides encoding polypeptides with NOVEL biological activity are preferred, the presence of NOVEL biological activity in an encoded protein is not necessary because even where a particular nucleic acid molecule does not encode a polypeptide having activity, one of skill in the art would still know how to use the nucleic acid molecule, for instance, as a hybridization probe or primer. Uses of the nucleic acid molecules of the present invention that do not encode a polypeptide having NOVEL activity include, inter alia, isolating a NOVEL gene or allelic variants thereof from a DNA library, and detecting NOVEL mRNA expression in biological samples suspected of containing NOVEL mRNA or DNA, e.g., by Northern Blot or PCR analysis.

Hybridizing Polvnucleotides

In another aspect, the invention provides an isolated or purified nucleic acid molecule comprising a polynucleotide which hybridizes under stringent hybridization conditions to any polynucleotide of the present. Such hybridizing polynucleotides may be of at least any one integer between 10 and 10,000 nucleotides in length.

Of course, a polynucleotide which hybridizes only to polyA+sequences (such as any 3′terminal polyA+tract of a cDNA shown in the sequence listing), or to a 5′ complementary stretch of T (or U) residues, would not be included in the definition of “polynucleotide,” since such a polynucleotide would hybridize to any nucleic acid molecule containing a poly(A) stretch or the complement thereof (e.g., practically any double-stranded cDNA clone generated using oligo dT as a primer).

Complementary Polynucleotides

The invention further provides isolated nucleic acid molecules having a nucleotide sequence fully complementary to any polynucleotide of the invention.

Polynucleotide Fragments

The present invention is further directed to portions or fragments of the polynucleotides of the present invention. Uses for the polynucleotide fragments of the present invention include probes, primers, molecular weight markers and for expressing the polypeptide fragments of the present invention. Fragments include portions of polynucleotides selected from the group consisting of a) polynucleotide sequences of the Sequence Listing, b) genomic NOVEL sequences, and c) polynucleotides encoding a polypeptide of the present invention. Particularly included in the present invention is a purified or isolated polynucleotide comprising at least 8, 10, 12, 15, 18, 20, 25, 28, 30, 35, 40, 50, 75, 100, 150, 200, 300, 400, 500, 800, 1000, 1500, or 2000 consecutive nucleotides of a polynucleotide of the present invention. Further, polynucleotides are provided comprising at least X nucleotides, wherein “X” is defined as any integer between 8 and the integer representing the 3′ most nucleotide position as set forth in the sequence listing or elsewhere herein.

Further included as preferred polynucleotides of the present invention are polynucleotide fragments that are specified in terms of their 5′ and 3′ position. Where the 5′ and 3′ positions are represented by the position numbers set forth in the appended sequence listing wherein the 5′ most nucleotide is 1 and the 3′ most nucleotide is the last nucleotide for a particular SEQ ID No., all polynucleotide fragments corresponding to every combination of a 5′ and 3′ nucleotide position that a polynucleotide fragment of the present invention, at least 8 contiguous nucleotides in length, could occupy on a polynucleotide of the invention are specifically considered. These species of polynucleotide fragments may alternatively be described by the formula “a to b”; where “a” equals the 5′ most nucleotide position and “b” equals the 3′ most nucleotide position of the polynucleotide; and further where “a” equals an integer between 1 and the number of nucleotides of the polynucleotide sequence of the present invention minus 8, and where “b” equals an integer between 9 and the number of nucleotides of the polynucleotide sequence of the present invention; and where “a” is an integer smaller then “b” by at least 8. All of the polynucleotide fragments described in either of these ways can be immediately envisaged and are therefore not individually listed solely for the purpose of not unnecessarily lengthening the specification. Any of these polynucleotide fragments may also be specifically excluded from the present invention. Any number of fragments specified by 5′ and 3′ positions or by size in nucleotides, as described above, may be excluded. Preferred excluded fragments include those having substantial homology to repeated sequences including Alu, L1, THE and MER repeats, SSTR sequences or satellite, micro-sateffite, and telomeric repeats.

Other preferred fragments of the invention are polynucleotides comprising polynucleotide sequences encoding domains of polypeptides. Such fragments may be used to obtain other polynucleotides encoding polypeptides having similar domains using hybridization or RT-PCR techniques. Alternatively, these fragments may be used to express a polypeptide domain which may have a specific biological property.

Another object of the invention is an isolated, purified or recombinant polynucleotide encoding a polypeptide consisting of, consisting essentially of, or comprising a contiguous span of at least (any integer between 5 and 1,000 consecutive amino acids in length more preferably at least) 5, 6, 8, 10, 12, 15, 20, 25, 30, 35, 40, 50, 60, 75, 100, 150 or 200 consecutive amino acids.

[The present invention further encompasses any combination of the polynucleotide fragments listed in this section.

Oligonucleotide Primers and Probes

The present invention also encompasses fragments of NOVEL polynucleotides for use as primers and probes. Polynucleotides derived from the NOVEL genomic and cDNA sequences are useful in order to detect the presence of at least a copy of a NOVEL polynucleotide or fragment, complement, or variant thereof in a test sample.

Structural Definition

Any polynucleotide of the invention may be used as a primer or probe. Particularly preferred probes and primers of the invention include isolated, purified, or recombinant polynucleotides comprising a contiguous span of at least 12, 15, 18, 20, 25, 30, 35,40, 50, 60, 70, 80, 90, 100, 150, 200, 500, or 1000 nucleotides of a polynucleotide of the present invention.

For amplification purposes, pairs of primers with approximately the same Tm are preferable. Primers may be designed using methods known in the art. Amplification techniques that can be used in the context of the present invention include, but are not limited to, the ligase chain reaction (LCR) described in EP-A-320 308, WO 9320227 and EP-A-439 182, the polymerase chain reaction CPCR, RT-PCR) and techniques such as the nucleic acid sequence based amplification (NASBA) described in Guatelli et al., (1990) PNAS 35:273-286 and in Compton (1991) Nature 350(6313):91-92, Q-beta amplification as described in European Patent Application No 4544610, strand displacement amplification as described in Walker, et al. (1996), Clin. Chem. 42:9-13 and EP A 684 315 and, target mediated amplification as described in WO 9322461.

The probes of the present invention are useful for a number of purposes, including for Southern hybridization to genomic DNA, to detect PCR amplification products, to detect mismatches in a NOVEL gene or mRNA, and in in situ hybridization. Any of the polynucleotides, primers and probes of the present invention can be conveniently immobilized on any type of solid support, such as latex particles, microparticles, magnetic beads, non-mnagnetic beads (including polystyrene beads), membranes (including nitrocellulose strips), plastic tubes, walls of microtiter wells, glass or silicon chips, sheep (or other suitable animal's) red blood cells and duracytes. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent, or alternatively may retain an additional receptor which has the ability to attract and immobilize the capture reagent. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Oligonucleotide Array

A substrate comprising a plurality of oligonucleotide primers or probes of the invention may be used either for detecting or amplifying targeted sequences in NOVEL genes, may be used for detecting mutations in the coding or in the non-coding sequences of NOVEL genes, and may also be used to determine NOVEL gene expression in different contexts such as in different tissues, at different stages of a process (embryo development, disease treatment), and in patients versus healthy individuals as described elsewhere in the application.

As used herein, the term “array” means a one dimensional, two dimensional, or multidimensional arrangement of nucleic acids of sufficient length to permit specific detection of gene expression. For example, the array may contain a plurality of nucleic acids derived from genes whose expression levels are to be assessed. The array may include any of the herein-described NOVEL genomic DNA, a NOVEL cDNA, sequences complementary thereto or fragments thereof. Preferably, the fragments are at least 12, 15, 18, 20, 25, 30, 35, 40, 50, or 100 nucleotides in length.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention, including Genechips™ (see, e.g., U.S. Pat. No. 5,143,854; PCr publications WO 90/15070 and 92/10092, Fodor et al., (1991) Science 251:767-777). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip (see, e.g., U.S. Pat. Nos. 5,143,854 and 5,412,087, and in PCT Publications WO 90/15070, WO 92/10092 and WO 95/11995). Further presentation strategies known in the art may be used, such as those disclosed in WO 94/12305, WO 94/11530, WO 97/29212 and WO 97/31256.

Consequently, the invention concerns an array of nucleic acid molecules comprising at least one, two, or five polynucleotides of the invention, particularly probes or primers as described herein.

Methods of Making the Polynucleotides of the Invention

The present invention also comprises methods of making the polynucleotides of the invention. Polynucleotides of the invention may be synthesized either enzymatically using techniques well known to those skilled in the art including amplification or hybridization-based methods as described herein, or chemically.

A variety of chemical methods of synthesizing nucleic acids are known to those skilled in the art. In many of these methods, synthesis is conducted on a solid support. Alternatively, polynucleotides may be prepared as described in U.S. Pat. No. 5,049. In some embodiments, several polynucleotides prepared as described above are ligated together to generate longer polynucleotides having a desired sequence.

Polypeptides of the Invention

The term “NOVEL polypeptides” is used herein to embrace all of the proteins and polypeptides of the present invention. The present invention encompasses NOVEL polypeptides, including recombinant, isolated or purified NOVEL polypeptides consisting of: (a) the full length polypeptides of even SEQ ID NOs:2-40; (b) the epitope-bearing fragments of the polypeptides of even SEQ ID NOs:2-40; (c) the domains of the polypeptides of even SEQ ID NOs:2-40; (d) the signal peptides of the polypeptides of even SEQ ID NOs:2-40; (e) the mature polypeptides of even SEQ ID NOs:2-40; and (f) the allelic variant polypeptides of any of the polypeptides of (a)-(e). Other objects of the invention are polypeptides encoded by the polynucleotides of the invention as well as fusion polypeptides comprising such polypeptides.

Polypeptide Variants

The present invention further provides for NOVEL polypeptides encoded by allelic and splice variants, orthoiogs, and/or species homologues. Procedures known in the art can be used to obtain, allelic variants, splice variants, orthologs, and/or species homologues of polynucleotides encoding polypeptides of the Sequence Listing.

The polypeptides of the present invention also include polypeptides having an amino acid sequence at least 50% identical, more preferably at least 60% identical, and still more preferably 70%, 80%, 90%, 95%, 96%, 97%, 98% or 99% identical to a polypeptide of the present invention. By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, (indels) or substituted with another amino acid.

Further polypeptides of the present invention include polypeptides which have at least 90% similarity, more preferably at least 95% similarity, and still more preferably at least 96%, 97%, 98% or 99% similarity to those described above. “Similarity” is calculated exactly as described above for “identity”, except that for the purposes of the calculation a matching amino acid can be either identical or an amino acid representing an “equivalent” change, as defmed below.

These alterations of the reference sequence may occur at the amino or carboxy terminal positions of the reference amino acid sequence or anywhere between those terminal positions, interspersed either individually among residues in the reference sequence or in one or more contiguous groups within the reference sequence. The variant polypeptides described herein are included in the present invention regardless of whether they have their normal biological activity, as one of skill in the art would recognize that variant polypeptides lacking biological activity would still be useful, for instance, as a vaccine, to generate antibodies, as epitope tags, in epitope mapping, as molecular weight markers on SDS-PAGE gels or on molecular sieve gel filtration columns.

Preparation of the Polypeptides of the Invention

The polypeptides of the present invention can be prepared in any suitable manner known in the art. Such polypeptides include isolated naturally occurring polypeptides, recombinantly produced polypeptides, synthetically produced polypeptides, or polypeptides produced by a combination of these methods. The polypeptides of the present invention are preferably provided in an isolated form, and may be partially or preferably substantially purified.

Isolation

From Natural Sources

The NOVEL proteins of the invention may be isolated from natural sources, including bodily fluids, tissues and cells, whether directly isolated or cultured cells, of humans or non-human animals. Methods for extracting and purifying natural proteins are known in the art, and include the use of detergents or chaotropic agents to disrupt particles followed by differential extraction and separation of the polypeptides by ion exchange chromatography, affinity chromatography, sedimentation according to density, and gel electrophoresis. See, e.g., “Methods in Enzymology, Academic Press, 1993” for a variety of methods for purifying proteins. Polypeptides of the invention also can be purified from natural sources using antibodies directed against the polypeptides of the invention, using standard methods.

From Recombinant Sources Preferably, the NOVEL polypeptides of the invention are recombinantly produced using routine expression methods known in the art. The polynucleotide encoding the desired polypeptide is operably linked to a promoter into an expression vector suitable for any convenient host. Both eukaryotic and prokaryotic host systems are used in forming recombinant polypeptides. The polypeptide is then isolated from lysed cells or from the culture medium and purified to the extent needed for its intended use. Any polynucleotide of the present invention may be used to express NOVEL polypeptides.

Consequently, a further embodiment of the present invention is a method of making a polypeptide of the present invention, said method comprising the steps of providing a NOVEL polynucleotide (e.g. a polynucleotide encoding a NOVEL polypeptide), inserting the polynucleotide in an expression vector such that the cDNA is operably linked to a promoter; and introducing the expression vector into a host cell whereby said host cell produces said polypeptide.

In one aspect of this embodiment, the method further comprises the step of isolating the polypeptide. Any suitable expression vector and system (e.g. cell-based system such as 3T3 cells) may be used, according to methods well known in the art.

In one embodiment, the entire coding sequence of a NOVEL cDNA and the 3′UTR through the polyA signal of the cDNA is operably linked to a promoter in the expression vector.

In another embodiment, an additional nucleotide sequence is included which codes for secretory or leader sequences, pro-sequences, sequences which aid in purification, such as multiple histidine residues, or an additional sequence for stability during recombinant production.

Alternatively, the NOVEL polypeptide to be expressed may also be a product of transgenic animals, i.e., as a component of the milk of transgenic cows, goats, pigs or sheep which are characterized by somatic or germ cells containing a nucleotide sequence encoding the protein of interest.

Any standard method may be used to recover a NOVEL polypeptide expressed using these methods, including differential extraction, ammonium sulfate or ethanol precipitation, acid extraction, anion or cation exchange chromatography, phosphocellulose chromatography, hydrophobic interaction chromatography, antibody-based methods, affinity chromatography, hydroxylapatite chromatography, HPLC, immunochromatography, and lectin chromatography.

Depending upon the host employed in a recombinant production procedure, the polypeptides of the present invention may be glycosylated or may be non-glycosylated, and may also include an initial modified methionine residue, in some cases as a result of host-mediated processes. In addition, polypeptides of the present invention may or may not contain the amino terminal methionine.

From Chemical Synthesis

In addition, polypeptides of the invention, especially short protein fragments, can be chemically synthesized using techniques known in the art (See, e.g., Creighton (1983), Proteins: Structures and Molecular Principles, W.H. Freeman & Co. 2nd Ed., T. B., New York; and Hunkapiller et al., (1984) Nature. 310(5973):105-11). For example, a polypeptide corresponding to a fragment of a polypeptide sequence of the invention can be synthesized by use of a peptide synthesizer. Alternatively, the methods described in U.S. Pat. No. 5,049,656, may be used.

Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Modifications

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including, but not limited to, specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein.

Also provided by the invention are chemically modified derivatives of the polypeptides of the invention which may provide additional advantages such as increased solubility, stability and circulating time of the polypeptide, or decreased immunogenicity (see, e.g., U.S. Pat. No. 4,179,337). The chemical moieties for derivatization may be selected from water soluble polymers (branched or unbranched) such as polyethylene glycol (preferably between 1 and 100 kD), ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties. The chemical moiety may be attached using any method, e.g. via a free amino, carboxyl, or sulfhydryl group (see, e.g., EP 0 401 384, or Malik et al., (1992), Exp. Hematol. 20:1028-1035).

Multimerization

The polypeptides of the invention may be in monomers or multimers (i.e., dimers, trimers, tetramers and higher multimers). Accordingly, the present invention relates to monomers and multirers of the polypeptides of the invention, their preparation, and compositions containing them.

Multimers encompassed by the invention may be homomers or heteromers. As used herein, the term “homomer” refers to a multimer containing only polypeptides with the same amino acid sequence (although a small amount of variation is allowed), and “heteromer” refers to a multimer containing one or more heterologous polypeptides.

Multimers of the invention may be the result of hydrophobic, hydrophilic, ionic and/or covalent associations and/or may be indirectly linked. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by cross-linking between cysteine residues located within the polypeptide sequences. Any of these associations may involve one or more amino acid residues contained in an amino acid provided in the sequence listing or in a heterologous polypeptide sequence of a fusion protein, e.g., in an Fc fusion protein, osteoprotegerin fusion protein, peptide linker fusion protein, Flag® fusion protein, or leucine zipper fusion protein of the invention (see, e.g., U.S. Pat. No. 5,478,925, WO 98/49305, or U.S. Pat. No. 5,073,627, Landschulz et al., (1988), Science. 240:1759, WO 94/10308, Hoppe et al., (1994), FEBS Letters. 344:191 and in U.S. patent application Ser. No. 08/446,922). Other methods of making multimers include the addition of cysteine or biotin to the C-terminus or N-terminus of the polypeptide using techniques known in the art, or by generating liposomes containing the polypeptide components desired to be contained in the multiner of the invention (see, e.g., U.S. Pat. No. 5,478,925 for numerous methods of multimerization).

Multimers of the invention may be generated using chemical or genetic engineering techniques known in the art.

Mutated Polypeptides

To improve or alter the characteristics of NOVEL polypeptides of the present invention, recombinant DNA technology can be used to create NOVEL mutant proteins or muteins including single or multiple amino acid substitutions, deletions, additions, or fusion proteins. Such modified polypeptides can show, e.g., increased/decreased biological activity or increased/decreased stability. In addition, they may be purified in higher yields and show better solubility than the corresponding natural polypeptide, at least under certain purification and storage conditions.

N- and C-terminal Deletions

It is known in the art that one or more amino acids may be deleted from the N-terminus or C-terminus without substantial loss of biological function. (See, e.g., Ron et al., (1993), Biol Chem., 268 2984-2988 ; Dobeli, et al. 1988.) Accordingly, the present invention provides polypeptides having one or more residues deleted from the amino and/or carboxy terminus.

Other Mutations

The invention includes numerous variations of the NOVEL polypeptides which show substantial NOVEL polypeptide activity. Such mutants include deletions, insertions, inversions, repeats, and substitutions selected according to general rules known in the art so as to have little effect on activity.

There are two main approaches for studying the tolerance of an amino acid sequence to change (see, Bowie et al., (1994), Science. 247:1306-1310). The first method relies on the process of evolution, in which mutations are either accepted or rejected by natural selection. The second approach uses genetic engineering to introduce amino acid changes at specific positions of a cloned gene and selections or screens to identify sequences that maintain functionality. Examples of this include site-directed mutagenesis or alanine-scanning mutagenesis (see, e.g., Cunningham et al. (1989), Science 244:1081-1085). These studies have revealed that proteins are surprisingly tolerant of amino acid substitutions.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Thus, the polypeptide of the present invention may be, for example, one in which one or more of the amino acid residues are substituted with a conserved or non-conserved, amino acid residue (preferably a conserved amino acid residue). Such substituted amino acid residue may or may not be one encoded by the genetic code, and may include a substituent group.

As indicated, changes are preferably of a minor nature, such as conservative amino acid substitutions that do not significantly affect the folding or activity of the protein. The following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

Furthermore, NOVEL polypeptides of the present invention may include one or more amino acid substitutions that mimic modified amino acids. An example of this type of substitution includes replacing amino acids that are capable of being phosphorylated (e.g., serine, threonine, or tyrosine) with a negatively charged amino acid that resembles the negative charge of the phosphorylated amino acid (e.g., aspartic acid or glutamic acid). Also included is substitution of amino acids that are capable of being modified by hydrophobic groups (e.g., arginine) with amino acids carrying bulky hydrophobic side chains, such as tryptophan or phenylalanine. Therefore, a specific embodiment of the invention includes NOVEL polypeptides that include one or more amino acid substitutions that mimic modified amino acids at positions where amino acids that are capable of being modified are normally positioned. Furthermore, any NOVEL polypeptide amino acid capable of being modified may be excluded from substitution with a modification-mimicking amino acid.

A specific embodiment of a modified NOVEL peptide molecule of interest according to the present invention, includes, but is not limited to, a peptide molecule which is resistant to proteolysis, is a peptide in which the —CONH— peptide bond is modified and replaced by a (CH2NH) reduced bond, a (NHCO) retro inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (see, e.g., Pinckard et al., (1967), Clin. Exp. Immunol 2:331-340; Robbins et al., (1987), Diabetes. 36:838-845; and Cleland et al., (1993) Crit. Rev. Ther. Drug Carr. Syst. 10:307-377).

A further embodiment of the invention relates to a polypeptide which comprises the amino acid sequence of a NOVEL polypeptide having an amino acid sequence which contains at least any one integer from 1 to 50 of conservative amino acid substitutions. Any conservative substitution or combination of substitutions may also be excluded.

Polypeptide Fragments

Structural Definition

The present invention is further directed to fragments of the polypeptides of the present invention. More specifically, the present invention embodies purified, isolated, and recombinant polypeptides comprising at least any one integer between 6 and 1000 (or the length of the polypeptides amino acid residues minus 1 if the length is less than 1000) of consecutive amino acid residues. Preferably, the fragments are at least 6, preferably at least 8 to 10, more preferably 12, 15, 20, 25, 30, 35,40, 50, 60, 75, 100, 125, 150, 175, 200, 225, 250, 275, or 300 consecutive amino acids of a polypeptide of the present invention.

In addition to the above polypeptide fragments, further preferred sub-genuses of polypeptides comprise at least X amino acids, wherein “X” is defined as any integer between 6 and the integer representing the C-terminal amino acid of the polypeptide of the present invention including the polypeptide sequences of the sequence listing below. Further included are species of polypeptide fragments at least 6 amino acids in length, as described above, that are further specified in terms of their N-terminal and C-terminal positions. However, included in the present invention as individual species are all polypeptide fragments, at least 6 amino acids in length, as described above, and may be particularly specified by a N-terminal and C-terminal position. That is, every combination of a N-terminal and C-terminal position that a fragment at least 6 contiguous amino acid residues in length could occupy, on any given amino acid sequence of the sequence listing or of the present invention is included in the present invention

Further preferred polypeptide fragments comprising amino acids of the sequences of the EVEN numbered SEQ ID NOs. of the Sequence listing, and polynucleotides encoding the same, are selected from the group consisting of amino acids starting at position one and continuing to any position selected from the group consisting of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485,486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, and 786, wherein the numbering of amino acids comprising any one fragment is consistent with the polypeptide sequence of any one EVEN numbered SEQ ID of the Sequence listing.

Further preferred polypeptide fragments comprising amino acids of the sequences of the EVEN numbered SEQ ID NOs. of the Sequence listing, and polynucleotides encoding the same, are selected from the group consisting of amino acids ending at the terminal amino acid of the protein (e.g. position 787) and beginning at any position selected from the group consisting of 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, and 782, wherein the numbering of amino acids comprising any one fragment is consistent with the polypeptide sequence of any one EVEN numbered SEQ ID of the Sequence listing.

Further preferred polypeptide fragments of the EVEN numbered SEQ ID NOs. of the Sequence listing, and polynucleotides encoding the same, are selected from the group consisting of fragments comprising any 50 or 100 consecutive amino acids starting from an amino acid position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, and 738, wherein the numbering of amino acids comprising any one fragment is consistent with the polypeptide sequence of any one EVEN numbered SEQ ID of the Sequence listing.

These specific embodiments, and other polypeptide and polynucleotide fragment embodiments described herein may be modified as being “at least”, “equal to”, “equal to or less than”, “less than”, “at least ______ but not greater than ______” or “from ______ to ______”. a specified size or specified N-texminal and/or C-terminal positions. It is noted that all ranges used to describe any embodiment of the present invention are inclusive unless specifically set forth otherwise.

The above species of polypeptide fragments of the present invention may alternatively be described by the formula “a to b”; where “a” equals the N-terminal most amino acid position and “b” equals the C-terminal most amino acid position of the polynucleotide; and further where “a” equals an integer between 1 and the number of amino acids of the polypeptide sequence of the present invention minus 6, and where “b” equals an integer between 7 and the number of amino acids of the polypeptide sequence of the present invention; and where “a” is an integer smaller then “b” by at least 6.

The above polypeptide fragments of the present invention can be immediately envisaged using the above description and are therefore not individually listed solely for the purpose of not unnecessarily lengthening the specification. Moreover, the above fragments need not have a NOVEL biological activity, although polypeptides having these activities are preferred embodiments of the invention, since even inactive fragments are useful, for example, in immunoassays, in epitope mapping, epitope tagging, as vaccines, as molecular weight markers, and to generate antibodies to a particular portion of the polypeptide.

The present invention also provides for the exclusion in any polypeptide of any one or more of the above-described fragments, e.g., one or more individual fragments specified by N-terminal and C-terminal positions or of any fragments specified by size in amino acid residues as described above.

Functional Definition

Domains

Preferred polynucleotide fragments of the invention comprise domains of polypeptides of the invention. Such domains may eventually comprise linear or structural motifs and signatures including, but not limited to, leucine zippers, helix-turn-helix motifs, post-translational modification sites such as glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites. Such domains may present a particular biological activity such as DNA or RNA-binding, secretion of proteins, transcription regulation, enzymatic activity, substrate binding activity, etc.

In a preferred embodiment, domains comprise a number of amino acids that is any integer between 6 and 1000. Domains may be synthesized using any methods known to those skilled in the art, including those disclosed herein. Methods for determining the amino acids which make up a domain with a particular biological activity include mutagenesis studies and assays to determine the biological activity to be tested, as well as bioinformatic methods for recognizing domains, motifs, or signatures, e.g., in a database such as Prosite (Hofmann et al., (1999) Nucl. Acids Res. 27:215-219; Bucher and Bairoch (1994) Proceedings 2nd International Conference on Intelligent Systems for Molecular Biology. Altman et al, Eds., pp53-61, AAAIPress, Menlo Park), Pfam (Sonnhammer, et al, (1997) Proteins. 28(3):405-20; Henikoff et al., (2000) Electrophoresis 21(9): 1700-6; Bateman et al., (2000) Nucleic Acids Res. 28(1):263-6), Blocks, Print, Prodom, Sbase, Smart, Dali/FSSP, HSSP, CATH, SCOP, COG. For a review on available databases, see issue 1 of volume 28 of Nucleic Acid Research (2000).

Epitopes and Antibody Fusions:

A preferred embodiment of the present invention is directed to epitope-bearing polypeptides and epitope-bearing polypeptide fragments. These epitopes may be “antigenic epitopes” or both an “antigenic epitope” and an “immunogenic epitope”. An “immunogenic epitope” is defined as a part of a protein that elicits an antibody response in vivo when the polypeptide is the immunogen. On the other hand, a region of polypeptide to which an antibody binds is defined as an “antigenic determinant” or “antigenic epitope.” The number of immunogenic epitopes of a protein generally is less than the number of antigenic epitopes (see, e.g., Geysen et al., (1984), PNAS 81:3998-4002). It is particularly noted that although a particular epitope may not be immunogenic, it is nonetheless useful since antibodies can be made to both immunogenic and antigenic epitopes. The present epitopes may be linear (i.e., composed of a contiguous sequence of amino acids repeated along the polypeptide chain) or nonlinear (also called “conformational”, i.e., composed of amino acids brought into proximity as a result of the folding of the polypeptide chain).

An epitope can comprise as few as 3 amino acids in a spatial conformation, which is unique to the epitope. Generally an epitope consists of at least 6 such amino acids, and more often at least 8-10 such amino acids. In preferred embodiment, antigenic epitopes comprise a number of amino acids that is any integer between 3 and 50. Fragments which function as epitopes may be produced by any conventional means (see, e.g., Houghten (1985), PNAS 82:5131-5135), also further described in U.S. Pat. No. 4,631,21. Methods for determining the amino acids which make up an epitope include x-ray crystallography, 2-dimensional nuclear magnetic resonance, and epitope mapping, e.g., the Pepscan method described by Geysen, et al. (1984); WO 84/03564; and WO 84/03506. Nonlinear epitopes are determined by methods such as protein footprinting (U.S. Pat. No. 5,691,448). Another example is the algorithm of Jameson and Wolf, (1988), Comp. Appl. Biosci. 4:181-186. The Jameson-Wolf antigenic analysis, for example, may be performed using the computer program PROTEAN, using default parameters (Version 4.0 Windows, DNASTAR, Inc., 1228 South Park Street Madison, Wis.). Epitopes may also be identified in vivo by testing for an antigenic response using standard methods.

All fragments of the polypeptides of the present invention, at least 6, 8, 10, 12, 15, 20, 25, 30, 35, 40, 50, 60, 75, 100, 125, 150, 175, 200, 225, 250, 275, or 300 amino acids residues in length, are included in the present invention as being useful as antigenic linear epitopes. Polypeptides of the present invention that are not specifically described as immunogenic are not considered non-antigenic as they may be antigenic in vivo. The epitope-bearing fragments of the present invention preferably comprise 6 to 50 amino acids (i.e. any integer between 6 and 50, inclusive) of a polypeptide of the present invention. Any number of epitope-bearing fragments of the present invention may also be excluded.

Nonlinear epitopes comprise more than one noncontiguous polypeptide sequence of at least one amino acid each. Such epitopes result from noncontiguous polypeptides brought into proximity by secondary, tertiary, or quaternary structural features. Preferred polypeptides providing nonlinear epitopes are formed by a contiguous surface of natively folded protein and are thus at least 10 amino acids in length, further preferably 12, 15, 20, 25, 30, 35, 40, 50, 60, 75, 100, 125, 150, 175, 200, 225, 250, 275, or 300 amino acids of a polypeptide of the present invention. Additionally, nonlinear epitopes may be formed by synthetic peptides that mimic an antigenic site or contiguous surface normally presented on a protein in the native conformation.

Immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention are used to induce antibodies according to methods well known in the art including, but not limited to, in vivo immunization, in vitro immunization, and phage display methods (see, e.g., Sutcliffe, et al., supra; Wilson, et al., supra, and Bittle, et al., supra). If in vivo immunization is used, animals may be immunized with free peptide; however, anti-peptide antibody titer may be boosted by coupling of the peptide to a macromolecular carrier using standard methods, such as keyhole limpet hemacyanin (KLH) or tetanus toxoid. Animals such as rabbits, rats and mice are immunized with either free or carrier-coupled peptides, for instance, by intraperitoneal and/or intradermal injection of emulsions containing about 100 μgs of peptide or carrier protein and Freund's adjuvant. Several booster injections may be needed, for instance, at intervals of about two weeks, to provide a useful titer of anti-peptide antibody, which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and discussed above, the polypeptides of the present invention comprising an immunogenic or antigenic epitope can be fused to heterologous polypeptide sequences. For example, the polypeptides of the present invention may be fused with the constant domain of immunoglobulins (IgA, IgE, IgG, IgM), or portions thereof (CH1, CH2, CH3, any combination thereof including both entire domains and portions thereof) resulting in chimeric polypeptides. These fusion proteins facilitate purification, and show an increased half-life in vivo (see, e.g., EPA 0,394,827; and Traunecker et al., (1988), Nature. 331:84-86; Fountoulakis et al., (1995) Biochem. 270:3958-3964). Additional fusion proteins of the invention may be generated through the techniques of gene-shuffling, motif-shuffling, exon-shuffling, or codon-shuffling (collectively referred to as “DNA shuffling”; see, for example, U.S. Pat. Nos. 5,605,793; 5,811,238; 5,834,252; 5,837,458; and Patten, et al. (1997), Curr Opinion Biotechnol. 8:724-733; Harayama (1998), Trends Biotechnol. 16(2): 76-82; Hansson et al., (1999), J. Mol. Biol. 287:265-276; and Lorenzo and Blasco (1998) Biotechniques. 24(2):308-313). The present invention further encompasses any combination of the polypeptide fragments listed in this section.

Antibodies

Definitions

The present invention further relates to antibodies and T-cell antigen receptors (TCR), which specifically bind the polypeptides, and more specifically, the epitopes of the polypeptides of the present invention. The antibodies of the present invention include IgG (including IgG1, IgG2, IgG3, and IgG4), IgA (including IgA1 and IgA2), IgD, IgE, or IgM, and IgY. The term “antibody” (Ab) refers to a polypeptide or group of polypeptides which are comprised of at least one binding domain, where a binding domain is formed from the folding of variable domains of an antibody molecule to form three-dimensional binding spaces with an internal surface shape and charge distribution complementary to the features of an antigenic determinant of an antigen, which allows an immunological reaction with the antigen. As used herein, the term “antibody” is meant to include whole antibodies, including single-chain whole antibodies, and antigen binding fragments thereof. In a preferred embodiment the antibodies are human antigen binding antibody fragments of the present invention include, but are not limited to, Fab, Fab′F(ab)2 and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes chimeric, humanized, and human monoclonal and polyclonal antibodies, which specifically bind the polypeptides of the present invention. The present invention further includes antibodies that are anti-idiotypic to the antibodies of the present invention.

The antibodies of the present invention may be monospecific, bispecific, and trispecific or have greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, et al., (1991), J. Immunol. 147:60-69; U.S. Pat. Nos. 5,573,920, 4,474,893, 5,601,819, 4,714,681, 4,925,648; Kostelny et al., (1992), J. Immunol. 148:1547-1553.

Antibodies of the present invention may be described or specified in terms of the epitope(s) or epitope-bearing portion(s) of a polypeptide of the present invention, which are recognized or specifically bound by the antibody. The antibodies may specifically bind a complete protein encoded by a nucleic acid of the present invention, or a fragment thereof. Antibodies which specifically bind any epitope or polypeptide of the present invention may also be excluded as individual species.

Thus, another embodiment of the present invention is a purified or isolated antibody capable of specifically binding to any of the polypeptides of the present invention. In one aspect of this embodiment, the antibody is capable of binding to a linear epitope-containing polypeptide comprising at least 6 consecutive amino acids, preferably at least 8 to 10 consecutive amino acids, more preferably at least 12, 15, 20, 25, 30,40, 50, or 100 consecutive amino acids of a polypeptides of the present invention. In another aspect of this embodiment, the antibody is capable of binding to a nonlinear epitope-containing polypeptide comprising 10 amino acids in length, further preferably 12, 15, 20, 25, 30, 35, 40, 50, 60, 75, or 100 amino acids, further preferably, a contiguous surface of the native conformation of a polypeptide of the present application.

Antibodies of the present invention may also be described or specified in terms of their cross-reactivity. Antibodies that do not specifically bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein, e.g., using FASTDB and the parameters set forth herein) to a polypeptide of the present invention are also included in the present invention. Further included in the present invention are antibodies, which only bind polypeptides encoded by polynucleotides, which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

The invention also concerns a purified or isolated antibody capable of specifically binding to a mutated NOVEL protein or to a fragment or variant thereof comprising an epitope of the mutated NOVEL protein.

Preparation of Antibodies

The antibodies of the present invention may be prepared by any suitable method known in the art. For example, a polypeptide of the present invention or an antigenic fragment thereof-can be administered to an animal in order to induce the production of sera containing “polyclonal antibodies”. As used herein, the term “monoclonal antibody” is not limited to antibodies produced through hybridoma technology but it rather refers to an antibody that is derived from a single clone, including eukaryotic, prokaryotic, or phage clone, and not the method by which it is produced. Monoclonal antibodies can be prepared using a wide variety of techniques known in the art including the use of hybridoma, recombinant, and phage display technology.

Hybridoma techniques include those known in the art (see, e.g., Harlow and Lane, (1988) Antibodies A Laboratory Manual. Cold Spring Harbor Laboratory. pp. 53-242; Hammerling (1981), Monoclonal Antibodies and T-Cell Hybridomas, Elsevier, N.Y. 563-681; Kohler and Milstein, (1975) Nature 256:495; Davis, et al., Basic Methods in Molecular Biology, ed., Elsevier Press, NY (1986), Section 21-2).).

Briefly, a mouse is repetitively inoculated with a few micrograms of the NOVEL protein, or a portion thereof, over a period of a few weeks. The mouse is then sacrificed, and the antibody producing cells of the spleen isolated. The spleen cells are fused by means of polyethylene glycol with mouse myeloma cells, and antibody-producing clones are identified (see, e.g., Engvall, (1980) Meth. Enzymol. 70:419). Selected positive clones can be expanded and their monoclonal antibody product harvested for use.

Further, Fab and F(ab′)2 fragments may be produced, for example, from hybridoma-produced antibodies by proteolytic cleavage, using enzymes such as papain (to produce Fab fragments) or pepsin (to produce F(ab′)2 fragments

Polyclonal antiserum containing antibodies to heterogeneous epitopes in the NOVEL protein or a portion thereof can be prepared by immunizing suitable non-human animals (e.g., mouse, rat, rabbit, goat, or horse) with the NOVEL protein or a portion thereof, which can be unmodified or modified to enhance immunogenicity (see, e.g., Vaitukaitis et al., (1971) J. Clin. Endocrinol. Metab. 33:988-991; Ouchterlony et al., (1973) Chap. 19 in: Handbook of Experimental Immunology D. Wier (ed) Blackwell). The protein or fragment is typically introduced into the non-human mammal in the presence of an appropriate adjuvant (e.g. aluminum hydroxide, RIBL etc.). Serum from the immunized animal is collected, treated and tested according to known procedures. If the serum contains polyclonal antibodies to undesired epitopes, the polyclonal antibodies can be purified by immunoaffinity chromatography. Affinity of the antisera for the antigen is determined using standard methods.

Alternatively, antibodies of the present invention can be produced through the application of recombinant DNA technology or through synthetic chemistry using methods known in the art. In phage display methods, for example, functional antibody domains are displayed on the surface of a phage particle, which carries polynucleotide sequences encoding them, and phage with a desired binding property are selected from a repertoire or combinatorial antibody library (e.g. human or murine) by selecting directly with antigen, typically antigen bound or captured to a solid surface or bead (see, e.g., Brinkman et al., (1995) J. Immunol Methods, 182:41-50; Ames et al., (1995), J. Immunol. Meth., 184:177-186.; Kettleborough et al., (1994), Eur. L Immunol., 24:952-958; Persic et al., (1997), Gene, 1879-81; Burton et al. (1994), Adv. Immunol., 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743). After phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria (see, e.g., WO 92/22324; Mullinax et al., (1992), BioTechniques. 12(6):864-869; and Sawai et al., (1995), AJRI 34:26-34; and Better et al., (1988), Science. 240:1041-1043).

Further teaching regarding the preparation of singlechain Fvs and antibodies is provided in U.S. Pat. Nos. 4,946,778 and 5,258,498; Huston et al., (1991), Meth. Enymol. 203:46_{—}88; Shu, et al., (1993), PNAS 90:7995-7999; and Skerra, et al., (1988), and Science 240:1038-1040. For some uses, including in vivo use of antibodies in humans and in vitro detection assays, it may be preferable to use chimeric, shuffled, humanized, or human antibodies (see e.g., Morrison, (1985); Oi et al., (1986), BioTechniques 4:214; Gillies et al., (1989), J. Immunol Methods. 125:191-202; and U.S. Pat. No. 5,807,715; EP 0 239 400; WO 91/09967; U.S. Pat. Nos. 5,530,101; and 5,585,089, EP 0 592 106; EP 0 519 596; Padlan (1991), Molec. Immunol. 28(415):489498; Studnicka et al., (1994), Protein Engineering. 7(6):805-814; Roguska et al., (1994), PNAS 91:969-973, U.S. Pat. No. 5,565,332, U.S. Pat. Nos. 4,444,887, 4,716,111, 5,545,806, and 5,814,318; WO 98/46645; WO 98/50433; WO 98/24893; WO 96/34096; WO 96/33735; and WO 91/10741).

Further included in the present invention are antibodies recombinantly fused or chemically conjugated (including both covalent and non-covalent conjugations) to any heterologous molecule, such as a polypeptide of the present invention, another polypeptide, a label useful for detection assays, or an effector molecule such as a drug or toxin (see, e.g., WO 92/08495; WO 91/14438; WO 89/12624; U.S. Pat. No. 5,314,995; and EP 0 396 387). Fused antibodies may also be used to target the polypeptides of the present invention to particular cell types, either in vitro or in vivo, by fusing or conjugating the polypeptides of the present invention to antibodies specific for particular cell surface receptors, and may also be used in vitro immunoassays and purification methods using methods known in the art (see e.g., Harbor, et al. supra; WO 93/21232; EP 0 439 095; Naramura et al., (1994), Immunol. Lett. 39:91-99; U.S. Pat. No. 5,474,981; Gillies et al., (1992), PNAS 89:1428-1432; Fell et al., (1991), J. Immunol. 146:2446-2452).

The present invention further includes compositions comprising the polypeptides of the present invention fused or conjugated to antibody domains other than the variable regions. For example, the polypeptides of the present invention may be fused or conjugated to an antibody Fc region, or portion thereof, and/or the hinge region, CH1 domain, CH2 domain, or CH3 domain or portions thereof, as well as to portions of IgA or IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi et al., (1991), PNAS 88:10535-10539; Zheng, et al. (1995), J. Immunol. 154:5590-5600; and Vil, et al. (1992), PNAS 89:11337-11341.

Non-human animals or mammals, whether wild-type or transgenic, which express a different species of NOVEL polypeptide than the one to which antibody binding is desired, and animals which do not express a NOVEL polypeptide (i.e. a NOVEL knock out animal as described herein) are particularly useful for preparing antibodies, as such animals will recognize all or most of the exposed regions of a NOVEL protein as foreign antigens, and therefore produce antibodies with a wider array of NOVEL epitopes. Moreover, smaller polypeptides with only 10 to 30 amino acids may be useful in obtaining specific binding to any one of the NOVEL proteins. In addition, the humoral immune system of animals which produce a species of NOVEL that resembles the antigenic sequence will preferentially recognize the differences between the animal's native NOVEL species and the antigen sequence, and produce antibodies to these unique sites in the antigen sequence. Such a technique will be particularly useful in obtaining antibodies that specifically bind to any one of the NOVEL proteins.

A preferred embodiment of the invention is a method of specifically binding an antibody or antibody fragment to a NOVEL polypeptide. This method comprises the step of contacting a NOVEL polypeptide-specific antibody or fragment thereof with a NOVEL polypeptide under antibody-binding conditions. Further included is a method of specifically binding an antibody or antibody fragment to an epitope, domain, or fragment of a NOVEL polypeptide. This method may be used to, for example, detect, purify, or modify the activity of NOVEL polypeptides, as discussed herein.

Antibodies of the invention can be used to assay protein levels in a test sample or biological sample using methods known to those of skill in the art. Antibody-based methods useful for detecting protein include inmmunoassays, such as the enzyme linked immunosorbent assay (ELISA) and radioimmunoassay (RIA). Suitable antibody assay labels are known in the art and include enzyme labels, such as glucose oxidase, horseradish peroxidase, and alkaline phosphatase; radioisotopes, such as iodine (125L, 121I), carbon (14C), sulfur (35S), tritium (3H), indium (121In), and technetium (99Tc); luminescent labels, such luminol, isoluinino, theromatic acridinium ester, irnidazole, acridinium salt, oxalate ester, luciferin, luciferase, and aequorin; and fluorescent labels, such as fluorescein isothiocyanate, rhodamine, phycoerythrin, phycocyanin, allophycocyanin, o-phthaldehyde, and fluorescarine.

Uses of Polynucleotides

Uses of Polynucleotides as Reagents

The polynucleotides of the present invention may be used as reagents in isolation procedures, diagnostic assays, and forensic procedures. For example, sequences from the NOVEL polynucleotides of the invention may be detectably labeled and used as probes to isolate other sequences capable of hybridizing to them. In addition, sequences from the NOVEL polynucleotides of the invention may be used to design PCR primers to be used in isolation, diagnostic, or forensic procedures.

To Find Corresponding Genomic DNA Sequences

The NOVEL cDNAs of the invention may also be used to clone sequences located in the vicinity, preferably upstream of the cDNAs of the invention on the corresponding genomic DNA. Such sequences may be capable of regulating gene expression, including promoter sequences, enhancer sequences, and other sequences which influence transcription or translation levels.

Use of cDNAs or Fragments Thereof to Clone Upstream Sequences from Genomic DNA

Sequences derived from polynucleotides of the inventions may be used to isolate the promoters of the corresponding genes using chromosome walking techniques (e.g., using the GenomeWalker™ kit from Clontech). Once the upstream genomic sequences have been cloned and sequenced, prospective promoters and transcription start sites within the upstream sequences may be identified by comparing the sequences upstream of the polynucleotides of the inventions with databases containing known transcription start sites, transcription factor binding sites, or promoter sequences.

In addition, promoters in the upstream sequences may be identified using promoter reporter vectors, e.g., by placing a reporter gene (e.g., secreted alkaline phosphatase, luciferase, beta-galactosidase, or green fluorescent protein) under the control of regulatory active polynucleotide fragments or variants of the NOVEL promoter region located upstream of the first exon of the NOVEL gene. A large number of suitable promoter reporter vectors are known in the art. The promoters and other regulatory sequences located upstream of the polynucleotides of the inventions may be used to design expression vectors capable of directing the expression of an inserted gene in a desired spatial, temporal, developmental, or quantitative manner.

To Find Similar Sequences

Polynucleotides of the invention may be used to isolate and/or purify nucleic acids similar thereto using any methods well known to those skilled in the art including the techniques based on hybridization or on amplification described in this section. These methods may be used to obtain the genomic DNAs which encode the mRNAs from which the NOVEL cDNAs are derived, mRNAs corresponding to NOVEL cDNAs, or nucleic acids which are homologous to NOVEL cDNAs or fragments thereof, such as variants, species homologues or orthologs.

Hybridization-based Methods

Techniques for identifying cDNA clones in a cDNA library which hybridize to a given probe sequence are disclosed in Sambrook et al., (1989) Molecular Cloning: A Laboratory Manual. (2ed., Cold Spring Harbor Laboratory, Cold Spring Harbor, N.Y.), and in Hames and Higgins (1985) Nucleic Acid Hybridization: A Practical Approach (Hames and Higgins Ed., IRL Press, Oxford). The same techniques may be used to isolate genomic DNAs.

A probe comprising at least 10 consecutive nucleotides from a NOVEL cDNA or fragment thereof, is labeled using standard methods with a detectable label such as a radioisotope or a fluorescent molecule. The cDNAs or genomic DNAs in the library are transferred to a nitrocellulose or nylon filter and denatured. After blocking of nonspecific sites, the filter is incubated with the labeled probe for an amount of time sufficient to allow binding of the probe to cDNAs or genomic DNAs containing a sequence capable of hybridizing thereto.

By varying the stringency of the hybridization conditions used to identify cDNAs or genomic DNAs which hybridize to the detectable probe, cDNAs or genomic DNAs having different levels of identity to the probe can be identified and isolated as described below.

Stringent Conditions

“Stringent hybridization conditions” are defined as conditions in which only nucleic acids having a high level of identity to the probe are able to hybridize to said probe. These conditions may be calculated as follows:

For probes between 14 and 70 nucleotides in length the melting temperature (Tm) is calculated using the formula: Tm=81.5+16.6(log (Na+))+0.41(fraction G+C)−(600/N) where N is the length of the probe.

If the hybridization is carried out in a solution containing formamide, the melting temperature may be calculated using the equation: Tm=81.5+16.6(log (Na+))+0.41(fraction G+C)−(0.63% formamide)−(600/N) where N is the length of the probe.

Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% foxmamide (see, e.g., Sambrook et al., 1986).

Hybridization is conducted according to standard methods. For probes over 200 nucleotides in length, the hybridization may be carried out at 15-25° C. below the Tm. For shorter probes, such as oligonucleotide probes, the hybridization may be conducted at 15-25° C. below the Tm. Preferably, for hybridizations in 6×SSC, the hybridization is conducted at approximately 68° C. Preferably, for hybridizations in 50% formnamide containing solutions, the hybridization is conducted at approximately 42° C.

Following hybridization, the filter is washed in 2×SSC, 0.1% SDS at room temperature for 15 minutes. The filter is then washed with 0.1×SSC, 0.5% SDS at room temperature for 30 minutes to 1 hour. Thereafter, the solution is washed at the hybridization temperature in 0.1×SSC, 0.5% SDS. A final wash is conducted in 0.1×SSC at room temperature.

Nucleic acids which have hybridized to the probe are identified by autoradiography or other conventional techniques.

Low and Moderate Conditions

Changes in the stringency of hybridization and signal detection are primarily accomplished through the manipulation of formamide concentration (lower percentages of formamide result in lowered stringency); salt conditions, or temperature. The above procedure may thus be modified to identify nucleic acids having decreasing levels of identity to the probe sequence. For example, the hybridization temperature may be decreased in increments of 5° C. from 68° C. to 42° C. in a hybridization buffer having a sodium concentration of approximately 1M. Following hybridization, the filter may be washed with 2×SSC, 0.5% SDS at the temperature of hybridization. These conditions are considered to be “moderate” conditions above 50° C. and “low” conditions below 50° C. Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of identity to the probe. Following hybridization, the filter may be washed with 6×SSC, 0.5% SDS at 50° C. These conditions are considered to be “moderate” conditions above 25% formamide and “low” conditions below 25% formamide. cDNAs or genomic DNAs which have hybridized to the probe are identified by autoradiography or other conventional techniques.

Note that variations in the above conditions may be accomplished through the inclusion and/or substitution of alternate blocking reagents (e.g. Denhardt's reagent, BLOTTO, heparin, denatured salmon sperm DNA, etc.) used to suppress background in hybridization experiments.

PCR-based Methods

In addition to the above described methods, other protocols are available to obtain homologous cDNAs using NOVEL cDNA of the present invention or fragment thereof, as described below. cDNAs may be prepared by obtaining mRNA from the tissue, cell, or organism of interest, e.g., using mRNA preparation procedures utilizing polyA selection procedures or other techniques known to those skilled in the art. A first primer capable of hybridizing to the polyA tail of the mRNA (e.g. an oligo(T) primer) is hybridized to the mRNA, and a reverse transcription reaction is performed to generate a first cDNA strand (see, e.g., Current Protocols in Molecular Biology, John Wiley and Sons, Inc. 1997 and Sambrook, et al., 1989). Typically, such oligo(T) primers comprise an additional sequence upstream of the poly(dT) stretch which facilitates subsequent manipulation of the DNA, such as a restriction site-containing sequence.

The first cDNA strand is then hybridized to a second primer containing at least 10 consecutive nucleotides of a polynucleotide of the invention. Often, the second primer used contains sequences located upstream of the translation initiation site. The second primer is extended to generate a second cDNA strand complementary to the first cDNA strand. Alternatively, RT-PCR may be performed as described above using primers from both ends of the cDNA to be obtained. See, e.g., Current Protocols in Molecular Biology, John Wiley & Sons, Inc. 1997 and Sambrook, et al., 1989.

Other Protocols

Alternatively, other procedures may be used for obtaining homologous cDNAs. In one approach, cDNAs are prepared from mRNA and cloned into double stranded phagemids using standard methods. The cDNA library in the double stranded phagemids is then rendered single stranded, a biotinylated oligonucleotide comprising the sequence of a fragment of a known NOVEL cDNA, genomic DNA or fragment thereof is hybridized to the single stranded phagemids. Hybrids between the biotinylated oligonucleotide and phagemids are isolated and the corresponding phagemids are released from the beads and converted into double stranded DNA using a primer specific for the NOVEL cDNA or fragment used to design the biotinylated oligonucleotide. Alternatively, protocols such as the Gene Trapper kit (Gibco BRL), may be used.

As a Chromosome Marker

NOVEL polynucleotides may be mapped to their chromosomal locations using any methods or techniques known to those skilled in the art including radiation hybrid (RH) mapping (See, e.g., Benham et al. (1989) Genomics 4:509-517 and Cox et al., (1990) Science 250:245-250; and Schuler et al, (1996) Science 274:540-546), PCR-based mapping, and Fluorescence in situ hybridization (FISH) mapping, as described below.

Mapping of cDNAs to Human Chromosomes using PCR Techniques

NOVEL cDNAs and genomic DNAs may be assigned to human chromosomes using PCR based methodologies. In such approaches, oligonucleotide primer pairs are designed from the cDNA sequence, and PCR is used to screen a series of somatic cell hybrid cell lines containing defined sets of human chromosomes. Only those somatic cell hybrids with chromosomes containing the human gene corresponding to the NOVEL cDNA or genomic DNA will yield an amplified fragment, and the single human chromosome present in all cell hybrids that give rise to an amplified fragment is the chromosome containing that NOVEL cDNA or genomic DNA (see, e.g., Ledbetter et al., (1990) Genomics 6:475481).

Mapping of cDNAs to Chromosomes Using Fluorescence In Situ Hybridization

Fluorescence in situ hybridization (FISH) allows the NOVEL cDNA or genomic DNA to be mapped to a particular location on a given chromosome. The chromosomes to be used for fluorescence in situ hybridization techniques may be obtained from a variety of sources including cell cultures, tissues, or whole blood (see, e.g., Cherif et al., (1990) PNAS 87:6639-6643).

Use of cDNAs to Construct or Expand Chromosome Maps

Once the NOVEL cDNAs or genomic DNAs have been assigned to particular chromosomes, they may be utilized to construct a high resolution map of the chromosomes on which they are located or to identify the chromosomes in a sample.

Chromosome mapping involves assigning a given unique sequence to a particular chromosome as described above. Once the unique sequence has been mapped to a given chromosome, it is ordered relative to other unique sequences located on the same chromosome (see, e.g., Nagaraja et al., (1997) Genome Res. 1997 March;7(3):210-22).

Identification of Genes Associated with Hereditary Diseases or Drug Response

In another embodiment, any particular NOVEL cDNA or genomic DNA may be used as a test probe to associate that NOVEL cDNA or genomic DNA with a particular phenotypic characteristic.

In one embodiment, NOVEL cDNAs or genomic DNAs are mapped to a particular location on a human chromosome using standard techniques and the location is searched in Mendelian Inheritance in Man (V. McKusick, Mendelian Inheritance in Man; available on line through Johns Hopkins University Welch Medical Library). Often, this search reveals the region of the human chromosome which contains the NOVEL cDNA or genoric DNA to be a very gene rich region containing several known genes and several diseases or phenotypes for which genes have not been identified. The gene corresponding to this NOVEL cDNA or genomic DNA thus becomes an immediate candidate for each of these genetic diseases.

Genomic DNA, mRNA or cDNA from patients with these diseases or phenotypes may then be screened, e.g. using PCR primers from the NOVEL cDNA or genomic DNA, or by sequencing, for differences in the expression, size, or sequence of the NOVEL cDNA or genomic DNAs in patients relative to in disease-free individuals. Any detected difference indicates a role for the NOVEL gene in the disease or phenotype.

Uses of Polynucleotides in Recombinant Vectors

The present invention also relates to recombinant vectors including the isolated polynucleotides of the present invention, and to host cells recombinant for a polynucleotide of the invention, such as the above vectors, as well as to methods of making such vectors and host cells and for using them for production of NOVEL polypeptides by recombinant techniques.

Recombinant Vectors

The term “vector” is used herein to designate either a circular or a linear DNA or RNA molecule, which is either double-stranded or single-stranded, and which comprises at least one polynucleotide of interest that is sought to be transferred in a cell host or in a unicellular or multicellular host organism.

In one preferred embodiment, the present invention provides expression vectors comprising either a regulatory polynucleotide or a coding nucleic acid of the invention, or both. Expression vectors may be used to express a NOVEL polypeptide for purification, as well as for constructing transgenic animals and also for gene therapy (including in vivo and ex vivo methods). The encoded protein may be transiently expressed in a host organism or cell or stably expressed in the host organism or cell. The encoded protein may have any of the activities described herein. The encoded protein may be a protein which the host organism lacks or, alternatively, the encoded protein may augment the existing levels of the protein in the host organism.

Some of the elements which can be found in the vectors of the present invention are described in further detail in the following sections.

General Features of the Expression Vectors of the Invention

Typical expression vectors of the present invention comprise a transcriptional unit comprising an assembly of a genetic element or elements having a regulatory role in gene expression, for example a promoter and a structural or coding sequence which is transcribed into mRNA and eventually translated into a polypeptide, said structural or coding sequence being operably linked to the regulatory elements described above, and appropriate transcription initiation and termination sequences. Additional features may include enhancers, a leader sequence enabling extracellular secretion of translated protein by a host cell, origins of replication, selectable markers permitting transformation of the host cell, ribosome binding sites, polyadenylation signals, splice donor and acceptor sites, transcriptional termination sequences, and 5′-flanking non-transcribed sequences.

Regulatory Elements

The suitable promoter regions used in the expression vectors according to the present invention are chosen taking into account the cell host in which the heterologous gene has to be expressed.

A suitable promoter may be heterologous with respect to the nucleic acid for which it controls the expression or alternatively can be endogenous to the native polynucleotide containing the coding sequence to be expressed. Promoter regions can be selected from any desired gene using, for example, CAT (chloramphenicol transferase) vectors and more preferably pKK232-8 and pCM7 vectors. Preferred bacterial promoters are the LacL LacZ, the T3 or T7 bacteriophage RNA polymerase promoters, the gpt, lambda PR, PL and trp promoters (EP 0036776), the polyhedrin promoter, or the p10 protein promoter from baculovirus (Kit Novagen) (Smith et al., (1983) Mol. Cell. Biol. 3:2156-2165; O'Reilly et al. (1992), “Baculovirus Expression Vectors: A Laboratory Manual”, W. H. Freeman and Co., New York), the lambda PR promoter or also the trc promoter.

Eukaryotic promoters include CMV immediate early, HSV thymidine kinase, early and late SV40, LTRs from retrovirus, and mouse metallothionein-L. Selection of a convenient vector and promoter is well within the level of ordinary skill in the art.

Selectable Markers

Selectable markers confer an identifiable change to the cell permitting easy identification of cells containing the expression construct. The selectable marker genes for selection of transformed host cells are preferably dihydrofolate reductase or neomycin resistance for eukaryotic cell culture, TRP1 for S. cerevisiae or tetracycline, rifampicin or ampicillin resistance in E. Coli, or levan saccharase for mycobacteria, this latter marker being a negative selection marker.

Preferred Vectors

The present recombinant vectors may be any sort of vector, including, but not limited to, YACs (Yeast Artificial Chromosome), BACs (Bacterial Artificial Chromosome), phage, phagemids, cosmids, plasmids, and linear DNA.

Bacterial Vectors

As a representative but non-limiting example, useful expression vectors for bacterial use can comprise a selectable marker and a bacterial origin of replication derived from commercially available plasmids comprising genetic elements of pBR322 (ATCC 37017). Such commercial vectors include, for example, pKK223-3 (Pharmacia, Uppsala, Sweden), pGEM1 (Promega Biotec, Madison, Wis., USA), pQE70, pQE60, pQE-9 (Qiagen), pbs, pD10, phagescript, psiX174, pbluescript SK, pbsks, pNH8A, pNH16A, pNH18A, pNH46A (Stratagene); ptrc99a, pKK223-3, pKK233-3, pDR540, pRIT⁵ (Pharmacia); pWLNEO, pSV2CAT, pOG44, pXT1, pSG (Stratagene); pSVK3, pBPV, pMSG, pSVL (Pharmacia); pQE-30 (QIAexpress).

Bacteriophage Vectors

The P1 bacteriophage vector may contain large inserts ranging from about 80 to about 100 kb. The construction of P1 bacteriophage vectors is well known in the art (see, e.g., Sternberg (1992) Trends Genet. 8:1-16, Sternberg (1994) Mamm. Genome. 5:397404, Linton et al., (1993) J. Clin. Invest. 92:3029-3037, McCormick et al., (1994) Genet. Anal. Tech. Appl. 11: 158-164.

Viral Vectors

Any viral vector can be used to carry out the herein-described methods. In one specific embodiment, the vector is derived from an adenovirus, e.g., human adenovirus type 2 or 5 (see, e.g., Feldman et al. (1996) Medecine/Sciences, 12:47-55, Ohno et al., (1994) Science 265:781-784, French patent application No. FR-93.05954).

Adeno-associated viral vectors are also preferred for the herein-described methods.

Particularly preferred retroviruses for the preparation or construction of retroviral in vitro or in vitro gene delivery vehicles of the present invention include retroviruses selected from the group consisting of Mink-Cell Focus Inducing Virus, Murine Sarcoma Virus, Reticuloendotheliosis virus and Rous Sarcoma virus. Particularly preferred Murine Leukemia Viruses include the 4070A and the 1504A viruses, Abelson (ATCC No VR-999), Friend (ATCC No VR-245), Gross (ATCC No VR-590), Rauscher (ATCC No VR-998) and Moloney Murine Leukemia Virus (ATCC No VR-190; PCT Application No WO 94/24298). Particularly preferred Rous Sarcoma Viruses include Bryan high titer (ATCC Nos VR-334, VR-657, VR-726, VR-659 and VR-728). Other preferred retroviral vectors are those described in Roth et al, (1996) Nature Medicine, 2(9):985-991, WO 93/25234, WO 94/ 06920, Roux et al. (1989) PNAS 86:9079-9083, Julan et al. (1992), J. Gen. Virol. 73:3251-3255, and Neda et al. (1991), J. Biol. Chem. 266:14143-14146.

BAC Vectors

The bacterial artificial chromosome (BAC) cloning system (Shizuya et al. (1992), PNAS 89:8794-8797), has been developed to stably maintain large fragments of genomic DNA (100-300 kb) in E. coli. A preferred BAC vector comprises a pBeloBAC11 vector that has been described by Kim U-J. et al. (1996), Genomics 34:213-218. BAC vectors, and the construction thereof, are well known in the art, and any suitable

Baculovirus

Another specific suitable host vector system is the pVL1392/1393 baculovirus transfer vector (Pharmingen) that is used to transfect the SF9 cell line (ATCC No. CRL 1711) which is derived from Spodoptera frugiperda. Other suitable vectors for the expression of the NOVEL polypeptide of the present invention in a baculovirus expression system include those described by Chai et al. (1993), Biotechnol. Appl. Biochem. 18:259-273; Vlasak, et al. (1983), Eur. J. Biochem. 135:123-126, and Lenhard et al., (1996) Gene. 169:187-190.

Delivery of the Recombinant Vectors

To effect expression of the polynucleotides and polynucleotide constructs of the invention, the constructs must be delivered into a cell. This delivery may be accomplished in vitro, as in laboratory procedures for transforming cell lines, or in vivo or ex vivo, as in the treatment of certain diseases states.

One mechanism is viral infection, where the expression construct is encapsulated in an infectious viral particle (see, U.S. Pat. No. 5,968,821). The expression construct, preferably a recombinant viral vector as discussed herein, is used to transduce packaging cells, which then produce infectious viral particles including the expression construct. The particles are then used to transduce eukaryotic cells (see, Miller, A. D. (1990) Blood 76:271; U.S. Pat. No. 6,228,844).

Replication defective retrovirus comprising a NOVEL polynucleotide may be packaged into virions, which can then be used to infect a target cell through the use of a helper virus by standard techniques (see, e.g., Current Protocols in Molecular Biology, Ausubel, F. M. et al. (eds.) Greene Publishing Associates, (1989)). Any of a large number of retroviruses can be used and are well known in the art (see, e.g., Eglitis, et al. (1985) Science 230:1395-1398; Danos and Mulligan (1988) PNAS 85:6460-6464; Wilson, et al. (1988) PNAS 85:3014-3018; Armentano, et al. (1990) PNAS 87:6141-6145; Huber, et al. (1991) PNAS 88:8039-8043; Ferry, et al. (1991) PNAS 88:8377-8381; Chowdhury, et al. (1991) Science 254:1802-1805; van Beusechem, et al. (1992) PNAS 89:7640-7644; Kay, et al. (1992) Human Gene Therapy 3:641-647; Dai, et al. (1992) PNAS 89:10892-10895; Hwu, et al. (1993) J. Immunol. 150:4104-4115).

Another viral gene delivery system useful in the present invention utilizes adenovirus-derived vectors. The genome of an adenovirus can be manipulated such that it encodes a gene product of interest, but is inactivated in terms of its ability to replicate in a normal lytic viral life cycle (see, e.g., Berkner, et al. (1988) BioTechniques 6:616; Rosenfeld, et al. (1991) Science 252:431-434; and Rosenfeld, et al. (1992) Cell 68:143-155). Any standard adenoviral vector may be used in the present invention (see, e.g., Jones, et al. (1979) Cell 16:683; Graham, et al. in Methods in Molecular Biology, E. J. Murray, Ed. (Hurmana, Clifton, N.J., 1991) vol. 7. pp.109-127).

Yet another viral vector system useful for delivery of polynucleotides is the adeno-associated virus (AAV). Adeno-associated virus is a naturally occurring defective virus that requires another virus, such as an adenovirus or a herpes virus, as a helper virus for efficient replication and a productive life cycle (see Muzyczka, et al., Curr. Top. Micro. Immunol. (1992) 158:97-129). Any standard AAV vector may be used in the present invention (see, e.g., Flotte et al., (1992) Am J. Respir. Cell Mol. Biol. 7:349-356; Am. J. Respir. Cell. Mol. Biol. 7:349-356; Samulski et al. (1989) J. Virol. 63:3822-3828; and McLaughlin et al. (1989) J. Virol. 62:1963-1973, Tratschin, et al. (1985) Mol. Cell. Biol. 5:3251-3260, Hermonat, et al. (1984) PNAS 81:6466-6470; Tratschin, et al. (1985) Mol. Cell. Biol. 4:2072-2081; Wondisford, et al. (1988) Mol. Endocrinol. 2:32-39; Tratschin, et al. (1984) J. Virol. 51:611-619; and Flotte, et al. (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of inserted gene expression in cells of the central nervous system and ocular tissue (Pepose, et al. (1994) Invest Ophthalmol Vis Sci 35:2662-2666).

Several non-viral methods for the transfer of polynucleotides into cells, e.g., mammalian cells, in vivo, in vitro, or ex vivo, are also contemplated by the present invention, and include, without being limited to, calcium phosphate precipitation (Graham et al., (1973) Virol. 52:456-457; Chen et al. (1987) Mol. Cell. Biol. 7:2745-2752); DEAE-dextran (Gopal (1985) Mol. Cell. Biol., 5:1188-1190); electroporation (Tur-Kaspa et al. (1986) Mol. Cell. Biol. 6:716-718; Potter et al., (1984) PNAS 81(22):7161-7165); direct microinjection (Harland et al., (1985) J. Cell. Biol. 101: 1094-1095); DNA-loaded liposomes (Nicolau et al., (1982) Biochim. Biophys. Acta. 721:185-190; Fraley et al., (1979) PNAS 76:3348-3352); and receptor-mediated transfection. (Wu and Wu (1987), J. Biol. Chem. 262:4429-4432; and Wu and Wu (1988), Biochemistry 27:887-892).

One specific embodiment for a method for delivering a protein or peptide to the interior of a cell of a vertebrate in vivo or in vitro comprises the step of introducing a preparation comprising a physiologically acceptable carrier and a naked polynucleotide operatively coding for the polypeptide of interest into the interstitial space of a tissue comprising the cell, whereby the naked polynucleotide is taken up into the interior of the cell and has a physiological effect (see, e.g., WO 90/11092, WO 95/11307, Tascon et al. (1996), Nature Medicine 2(8):888-892, and Huygen et al., (1996) Nature Medicine 2(8):893-898). Naked polynucleotides of the invention may also be introduced into cells using particle bombardment (biolistic), e.g., DNA-coated microprojectiles accelerated to a high velocity allowing them to pierce cell membranes and enter cells without killing them (Klein et al., (1987) Nature 327:70-73).

Liposomal preparations for use in the present invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner, et al., PNAS (1987) 84:7413-7416); mRNA (Malone, et al., PNAS (1989) 86:6077-6081); and purified transcription factors (Debs et al., J. Biol. Chem. (1990) 265:10189-10192), in functional form. Any of a large number of cationic liposomes can be used. N[1-2,3-dioleyloxy)propyll-N,N,N-triethylammonium (DOTMA) liposomes are particularly useful and are available under the trademark Lipofectin, from GIBCO BRL, Grand Island,N.Y. Other commercially available liposomes include transfectace (DDAB/DOPE) and DOTAP/DOPE (Boehringer).

Similarly, anionic and neutral liposomes are readily available, such as from AvantiPolar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolarnine (DOPE), among others. These materials can also be mixed with the DOTMA and DOTAP starting materials in appropriate ratios. Methods for making liposomes using these materials are well known in the art, as are various combinations of liposomes.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art (Straubinger, et al., Methods of Immunology (1983), 101:512-527; U.S. Pat. No. 5,965,421). Generally, the ratio of DNA to liposomes will be from about 10:1 to about 1:10 (with about 5:1 to about 1:5 or about 3:1 to about 1:3 being preferred). Additionally, liposomes may be targeted to specific cell types by embedding a targeting moiety such as a member of a receptor-receptor ligand pair into the lipid envelope of the vesicle (see, e.g., U.S. Pat. No. 6,177,433, U.S. Pat. No. 6,110,490, and P.C.T No. WO9704748).

The amount of vector to be injected to the desired host organism varies according to the site of injection. As an indicative dose, it will be injected between 0.1 and 100 μg of the vector in an animal body, preferably a mammal body, for example a mouse body.

Secretion Vectors

Some of the NOVEL cDNAs or genomic DNAs of the invention may also be used to construct secretion vectors capable of directing the secretion of the proteins encoded by genes inserted in the vectors. Such secretion vectors may facilitate the purification or enrichment of the proteins encoded by genes inserted therein by reducing the number of background proteins from which the desired protein must be purified or enriched. Exemplary secretion vectors are described below.

The secretion vectors of the present invention comprise a promoter capable of directing gene expression in the host cell, tissue, or organism of interest, a cloning site for inserting a coding sequence, and a signal sequence from a polynucleotide of the invention is operably linked to the promoter such that the mRNA transcribed from the promoter will direct the translation of the signal peptide. The host cell, tissue, or organism may be any cell, tissue, or organism which recognizes the signal peptide encoded by the signal sequence in the NOVEL cDNA or genomic DNA. Suitable hosts include mammalian cells, tissues or organisms, avian cells, tissues, or organisms, insect cells, tissues or organisms, or yeast. The signal sequences may also be inserted into vectors designed for gene therapy.

The secretion vector may be DNA or RNA and may integrate into the chromosome of the host, be stably maintained as an extrachromosomal replicon in the host, be an artificial chromosome, or be transiently present in the host. Preferably, the secretion vector is maintained in multiple copies in each host cell.

Cell Hosts

Another object of the invention comprises a host cell that has been transformed or transfected with one of the polynucleotides described herein, and in particular a polynucleotide either comprising a NOVEL polypeptide-encoding polynucleotide regulatory sequence or the polynucleotide coding for a NOVEL polypeptide. Also included are host cells that are transformed (prokaryotic cells), transfected (eukaryotic cells), or transduced with a recombinant vector such as one of those described above. Preferred host cells used as recipients for the expression vectors of the invention include prokaryotic host cells such as Escherichia coli strains (I.E.DH5-α strain), Bacillus subtilis, Salmonella typhimurium, and strains from species like Pseudomonas, Streptomyces and Staphylococcus, as well as eukaryotic host cells such as HeLa cells, Cv 1 cells, COS cells, Sf-9 cells, C127 cells, 3T3, CHO, human kidney 293, and BHK cells.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides (see, e.g., U.S. Pat. No. 5,641,670; WO 96/29411; WO 94/12650; Koller, et al., (1989); and Zijlstra, et al. (1989); U.S. Pat. Nos. 6,054,288; 6,048,729; 6,048,724; 6,048,524; 5,994,127; 5,968,502; 5,965,125; 5,869,239; 5,817,789; 5,783,385; 5,733,761; 5,641,670; 5,580,734; and in W096/29411, WO 94/12650; and Koller, et al., (1994).

NOVEL gene expression in mammnlian cells, preferably human cells, may be rendered defective, or alternatively may be altered by replacing endogenous NOVEL polypeptide-encoding genes in the genome of an animal cell by a NOVEL polypeptide-encoding polynucleotide according to the invention. These genetic alterations may be generated by homologous recombination using previously described specific polynucleotide constructs.

Mammal zygotes, such as murine zygotes may be used as cell hosts. For example, murine zygotes may undergo microinjection with a purified DNA molecule of interest. In addition, any one of the polynucleotides of the invention may be introduced in an embryonic stem (ES) cell line, preferably a mouse ES cell line. ES cells, and methods of their isolation and maintenance, are well known in the art, and are described, inter alia, in Abbondanzo et al., (1993), Meth. Enzymol., Academic Press, New York, pp 803-823, Robertson, (1987), Embryo-derived stem cell lines; In: E. J. Robertson Ed. Teratocarcinomas and embrionic stem cells: a practical approach. IRL Press, Oxford, pp. 71, and Pease and William, (1990), Exp. Cell. Res. 190: 209-211.

Transgenic Animals

The terms “transgenic animals” or “host animals” are used herein to designate animals that have their genome genetically and artificially manipulated so as to include one of the nucleic acids according to the invention. The cells affected may be somatic, germ cells, or both. Preferred animals are non-human mammals and include those belonging to a genus selected from Mus (e.g. mice), Rattus (e.g. rats) and Oryctogalus (e.g. rabbits). In one embodiment, the invention encompasses non-human host mammals and animals comprising a recombinant vector of the invention or a NOVEL gene disrupted by homologous recombination with a knock out vector. Thus, the present invention also concerns a transgenic animal containing a nucleic acid, a recombinant expression vector, or a recombinant host cell according to the invention. A further object of the invention comprises recombinant host cells obtained from a transgenic animal described herein.

Such transgenic animals may be good experimental models in order to study the diverse pathologies related to the increase or decrease of the expression of a given NOVEL gene. The transgenic amnimals may also be used to express a desired polypeptide of interest under the control of the regulatory polynucleotides of the NOVEL gene, leading to high yields in the synthesis of this protein of interest, or to tissue-specific expression of the gene.

In one embodiment, transgenic animals of the present invention are produced by inserting a recombinant polynucleotide of the invention into an embryonic or ES stem cell line (see, e.g., Thomas, et al. (1987) Cell 51:503-512; Mansour et al., (1988) Nature 336:348-352), and isolating, cloning and injecting positive cells into blastocysts, which are then inserted into a female host animal and allowed to grow to term. For more details regarding the production of transgenic animals, and specifically transgenic mice, see U.S. Pat. Nos. 4,873,191; 5,464,764; 5,789,215, Bradley (1987; In: E. J. Robertson (Ed.), Teratocarcinomas and embryonic stem cells: A practical approach. IRL Press, Oxford, pp.113, Wood, et al. (1993), PNAS, 90: 4582-4585, Nagy et al., (1993), PNAS 90: 8424-8428.

In another embodiment, transgenic animals are produced by microinjecting polynucleotides into a fertilized oocyte. Methods for culturing fertilized oocytes to the pre-implantation stage are described, e.g., by Gordon, et al. ((1984) Methods in Enzymology, 101, 414); Hogan, et al. ((1986) in Manipulating the mouse embryo, A Laboratory Manual. Cold Spring Harbor Laboratory Press, Cold Spring Harbor, N.Y (for the mouse embryo)); Hammer, et al. ((1985) Nature, 315, 680 (for rabbit and porcine embryos)); Gandolfi, et al. ((1987) J. Reprod. Fert. 81, 23-28); Rexroad, et al. ((1988) J. Anim. Sci. 66, 947-953) (for ovine embryos)); and Eyestone, et al. ((1989) J. Reprod. Fert. 85, 715-720); Camous et al. ((1984) J. Reprod. Fert. 72, 779-785); and Heyman, et al. ((1987) Theriogenology 27, 5968 (for bovine embryos)). Pre-implantation embryos are then transferred to an appropriate female by standard methods to permit the birth of a transgenic or chimeric animal, depending upon the stage of development when the transgene is introduced.

In a preferred embodiment of the present invention, transgenic mammals are generated that secrete recombinant NOVEL polypeptides in their milk. Preferably, expression in the mammary gland is accomplished by operably linking the polynucleotide encoding the NOVEL polypeptide to a mammary gland specific promoter (e.g., from a casein or lactoglobulin gene) and, optionally, other regulatory elements. Promoter and other regulatory sequences, vectors, and other relevant teachings are provided, e.g., by Clark (1998) J Mammary Gland Biol Neoplasia 3:337-50; Jost, et al. (1999) Nat. Biotechnol 17:1604; U.S. Pat. Nos. 5,994,616; 6,140,552; 6,013,857; Sohn, et al. (1999) DNA Cell Biol. 18:845-52; Kim, et al. (1999) J. Biochem. (Japan) 126:320-5; Soulier, et al. (1999) Euro. J. Biochem. 260:533-9; Zhang, et al. (1997) Chin. J. Biotech. 13:271-6; Rijnkels, et al. (1998) Transgen. Res. 7:5-14; Korhonen, et al. (1997) Euro. J. Biochem. 245:482-9; Uusi-Oukari, et al. (1997) Transgen. Res. 6:75-84; Hitchin, et al. (1996) Prot. Expr. Purif. 7:247-52; Platenburg, et al. (1994) Transgen. Res. 3:99-108; Heng-Cherl, et al. (1993) Animal Biotech. 4:89-107; and Christa, et al. (2000) Euro. J. Biochem. 267:1665-71.

In another embodiment, the polypeptides of the invention can be produced in milk by introducing polynucleotides encoding the polypeptides into somatic cells of the mammary gland in vivo, e.g. mammary secreting epithelial cells. For example, plasmid DNA can be infused through the nipple canal, e.g. in association with DEAE-dextran (see, e.g., Hens, et al. (2000) Biochim Biophys. Acta 1523:161-171), in association with a ligand that can lead to receptor-mediated endocytosis of the construct (see, e.g., Sobolev, et al. (1998) 273:7928-33), or in a viral vector such as a retroviral vector, e.g. the Gibbon ape leukemia virus (see, e.g., Archer, et al. (1994) PNAS 91:6840-6844). In any of these embodiments, the polynucleotide may be operably linked to a mammary gland specific promoter, as described above, or, alternatively, any strongly expressing promoter such as CMV or MoMLV LTR.

The polynucleotides used in such embodiments can either encode a full-length NOVEL protein or a NOVEL fragment. Typically, the encoded polypeptide will include a signal sequence to ensure the secretion of the protein into the mil.

Uses of Polypeptides of the Invention

Protein of SEQ ID NO:2 (Internal Designation Clone 500695767.cFS_{—}188-227-2-0-G1-F)

The cDNA of Clone 500695767.cFS_{—}188-227-2-0-G1-F (SEQ ID NO:1) encodes nemeglya of SEQ ID NO:2, comprising the amino acid sequence: MARTAGDTLDVFTKYVIYGIAAAFYGIILLMVEGFFTTGAIKDLYGDFKITTCGRCVSA WFIMLTYMLAWLGVTAFSLPVYMYFNLWTICRNTLlVEGANLCLDLRQFGIVTIGEEKKICT VSENFLRMCESTELNMTFBLFIVALAGAGAAVIAMVHYSANWAYVKDACRMQKYEDIK SKEEQELHDIHSTRSKERLNAYT. Accordingly, it will be appreciated that all characteristics and uses of the polypeptides of SEQ ID NO:2 described throughout the present application also pertain to the polypeptides encoded by the nucleic acids comprising the human cDNA in Clone 500695767.cFS_{—}188-227-2-0-G1-F. In addition, it will be appreciated that all characteristics and uses of the polynucleotides of SEQ ID NOs:1 described throughout the present application also pertain to the nucleic acids included in Clone 500695767.cFS_{—}188-227-2-0-G1-F. A preferred embodiment of the invention is directed toward the compositions of SEQ ID NO:2, SEQ ID NO:1, and Clone 500695767.cFS_{—}188-227-2-0-G1-F. Also preferred are polypeptide fragments having a biological activity as described herein and the polynucleotides encoding the fragments.

The protein of the invention, nemeglya, is a splice variant of the neuronal membrane glycoprotein M6a (GenBank accession number P51674). The first exon of nemeglya cDNA is different from the first exon of M6a cDNA. Thus nemeglya is 63 amino-acids shorter than M6a at its amino-terminal extremity. The 215 carboxyl-terminal amino-acids are identical between the two proteins. Nemeglya displays one signal anchor and two transmembrane domains. Furthermore, nemeglya displays a Myelin proteolipid protein domain (PF01275).

The nemeglya gene is specifically expressed in neurons. Nemeglya is located at the presynaptic membranes and at the synaptic vesicle membranes of neurons. It is expressed at particularly high levels during neural development. Nemeglya plays a major role in vesicle docking and fusing of a presynaptic vesicle to the presynaptic membrane. Furthermore, nemeglya is essential for synapse matching during neural development. Thus nemeglya is a key protein both for neurotransmitter function in the terminals of neurons and for neurite outgrowth.

An embodiment of the invention is directed to a composition comprising a nemeglya polypeptide consisting of a polypeptide sequence of SEQ ID NO:2.

A further embodiment of the invention is directed to a composition comprising a nemeglya polypeptide fragment having biological activity of promoting neurite outgrowth or neurotransmission.

As used herein, a nemeglya polypeptide refers either to a nemeglya polypeptide sequence of SEQ ID NO:2 or to a nemeglya polypeptide fragment having biological activity of promoting neurite outgrowth or neurotransmission.

An embodiment of the invention is directed to a composition comprising a polynucleotide sequence of SEQ ID NO:1 encoding a nemeglya polypeptide.

A further embodiment of the invention is directed to a composition comprising a polynucleotide sequence encoding a nemeglya polypeptide fragment having biological activity of promoting neurite outgrowth or neurotransmission.

As further used herein, a nenmeglya polynucleotide refers either to a nemeglya polynucleotide sequence of SEQ ID NO:1 or to a polynucleotide fragment of SEQ ID NO:1.A further embodiment of the invention is directed to an antibody recognizing a nemeglya polypeptide sequence of SEQ ID NO:2 or a nemeglya polypeptide fragment. Preferably, the antibody recognizes one or more aniino-acids located within the amnino-terminal extremity of nemeglya, wherein said one or more amino-acids are required for binding of the antibody to a nemeglya polypeptide. Also preferably, the antibody recognizes a non linear epitope. Most preferably, the antibody binds to nemeglya but not to M6a. As used herein, an anti-nemeglya antibody refers to an antibody or antigen-binding fragment thereof that specifically binds to a nemeglya polypeptide.

An embodiment of the present invention relates to a method of binding such an anti-nemeglya antibody to a nemeglya polypeptide comprising the step of: contacting a nemeglya polypeptide with said antibody or an antigen-binding fragment thereof under conditions that allow binding to take place. Such conditions are well known to those skilled in the art. Such methods are useful for detecting nemeglya polypeptides as further described herein.

An embodiment of the present invention is directed to a method of detecting nemeglya polypeptides in a biological sample, said method comprising the steps of: i) contacting a biological sample with an anti-nemeglya antibody; and ii) detecting the antigen-antibody complex formed. The antibody or antibody fragment may be monoclonal or polyclonal. In addition, the antibody or antibody fragment may be primarily or secondarily labeled by any detectable compound (e.g., radioactive, fluorescent, luminescent, or enzymatic) common in the art. Such a method may be applied in a diagnostic kit as further described below.

An embodiment of the present invention is directed to methods of detecting nemeglya expression by detecting the levels of nemeglya mRNAs or polypeptides in a biological sample, said methods comprising the steps of: i) providing a biological test sample from an individual; ii) detecting the level of nemeglya expression; and iii) comparing the level of nemeglya expression in said test sample to that of control sample(s). Such methods of detecting nemeglya expression may be used for diagnosing neurological and mental diseases associated with impaired neurotransmission such as, e.g., Alzheimer's disease, neuroparalytic syndromes of tetanus and botulism, Huntington's disease, Lambert-Eaton myasthenic syndrome, progressive cerebellar ataxia, and the velocardiofacial syndrome-like phenotype associated with a mutation at the 4q34 locus (VCFS4q34). Well-known techniques such as, e.g., western bot or immunochemistry may be used to detect nemeglya expression. Preferably, anti-nemeglya antibodies are used to detect the level of nemeglya expression. Said antibody may be monoclonal or polyclonal. In addition, said antibody may be primarily or secondarily labeled by any detectable compound (e.g., radioactive, fluorescent, luminescent and enzymatic compounds) common in the art. Also preferably, polynucleotides probes comprising a polynucleotide sequence of SEQ ID NO:1 or part thereof, or a polynucleotide sequence complementary of SEQ ID NO:1 are used to detect the level of nemeglya expression by, e.g., northern blot or RTPCR techniques.

An embodiment of the present invention relates to methods of using a nemeglya polynucleotide for DNA genotyping. Such a method comprises the steps of: i) providing a nucleotide test sample from an individual; and ii) detecting the presence of the nemeglya genomic sequence. The presence of nemeglya genomic sequence is preferably detected using standard molecular biological techniques (e.g., Southern blotting) and employing a nemeglya nucleotide or fragment thereof as a probe. Alternatively, the method comprises the steps of i) providing a nucleotide test sample from an individual; and ii) detecting the presence of a mutation in the nemeglya genomic sequence. Such mutations include deletions, translocations, inversions, and punctual mutations. These mutations are preferably detected using standard molecular biological techniques (e.g., sequencing or PCR) and employing a nemeglya nucleotide or fragment thereof as a probe or primer. Preferably, the nucleotide test sample in these methods contains DNA. Identification of either less than two functional copies of the genomic region in which nemeglya is located or a mutation in said region is indicative of a likelihood that said individual has a genetic mutation associated with VCFS associated with a mutation at the 4q34 locus. Such methods of using a nemeglya polynucleotide for DNA genotyping are useful to diagnose VCFS-4q34. Preferably, said nemeglya polynucleotide is an oligonucleotide primer of less than 30 nucleotides. Also preferably, a nemeglya polynucleotide of SEQ ID NO:1 is used as a probe in such a method. Genotyping of an individual can be performed by any technique well-known to those skilled in the art such as, e.g., sequencing, microsequencing or hybridization. Such methods can be accomplished as described in U.S. Pat. No. 5,935,783 and U.S. Pat. No. 6,303,294, which disclosures are incorporated by reference in their entireties. Said nemeglya polynucleotide may be used alone or in combination with other polynucleotidic sequences indicative of a likelihood that an individual has a genetic mutation associated with VCFS such as, e.g., those described in U.S. Pat. No. 5,935,783 and U.S. Pat. No. 6,303,294. An additional aspect of this embodiment relates to an array of oligonucleotides probes comprising a nemeglya polynucleotide. Preferably, said array of oligonucleotides probes are directed to conduct efficient screening of mutations associated with VCFS. Preferably, such microarrays also comprise several oligonucleotides indicative of different genetic mutations associated with VCFS such as, e.g., those described in U.S. Pat. No. 5,935,783 and U.S. Pat. No. 6,303,294. These microarrays can for example be used to perform epidemiological studies in families predisposed to VCFS, or to diagnose VCFS.

Another embodiment of the present the invention is directed to a diagnostic kit for detecting in vitro the presence of a nemeglya polypeptide. Such kit comprises: i) an anti-nemeglya antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-nemeglya antibodies is used for diagnosing VCFS4q34 or various diseases associated with impaired neurotransmission such as those listed above. Optionally, said diagnostic kit comprises a sample of a normal individual. Optionally, said diagnostic kit comprises a sample of an individual suffering from VCFS4q34 or a disease associated with impaired neurotransmission.

Still another embodiment is directed to methods of using anti-nemeglya antibodies for detecting nemeglya polypeptides in vivo, for example by immunoelectron microscopy or by immunohistochemical staining. Such methods are described in Roussel et al. (J Neurocytol. 27:695-703 (1998)) and in Shetty et al. (J Neurobiol. 38:391-413 (1999)), which disclosures are both incorporated by reference in their entireties. Detecting nemeglya polypeptides iii vivo is useful for following neurite outgrowth in model animals when studying, e.g., neurotrophic factors, brain development and neuron grafts.

Another embodiment relates to a method of producing a nemeglya polypeptide comprising the steps of: i) culturing a cell expressing a nemeglya polypeptide, and ii) purifying the produced protein. The purification of the protein can be done following any technique well-known to those skilled in the art. Preferably, an anti-nemeglya antibody may be bound to a chromatographic support to form an affinity chromatography column. Preferably, the cell expressing a nemeglya polypeptide is a recombinant host cell as described below. Producing nemeglya polypeptides may be useful in methods and compositions as further described in the present invention.

A preferred aspect of the invention is a host cell recombinant for polynucleotides encoding a nemeglya polypeptide. An additional preferred aspect is a host cell recombinant for polynucleotides capable of directing nemeglya expression. An embodiment is directed to a method of constructing a host cell recombinant for polynucleotides encoding a nemeglya polypeptide comprising the steps of: i) constructing a recombinant molecule that comprises a nucleic acid sequence encoding a nemeglya polypeptide and that allows expression of said nemeglya polypeptide under given physiological conditions, and ii) introducing said recombinant molecules into a cell. In preferred embodiments, the recombinant molecule is introduced into an Escherichia coli cell or into a human neuronal cell. Alternatively, a host cell recombinant for polynucleotides capable of directing nemeglya expression may be constructed by a method comprising the steps of: i) constructing a recombinant molecule comprising a polynucleotide capable of directing nemeglya expression, and ii) introducing said recombinant molecules into a cell. Preferably, the polynucleotides capable of directing nemeglya expression are located in the 5′ regulatory region of the nemeglya gene. Further preferably, these polynucleotides are located within 500 base pairs of the nemeglya coding region. These polynucleotides preferably comprise a promoter sequence. Techniques known in the art for introducing polynucleotide sequences to endogenous sequences are described in U.S. Pat. No. 5641670 and PCT WO9629411, which disclosures are hereby incorporated by reference in their entireties. Such recombinant host cell producing nemeglya polypeptides may be used to produce nemeglya polypeptides.

Another preferred embodiment relates to test substances for modulators of nemeglya expression and to a method of screening for such substances comprising the steps of: i) contacting a cell with a test substance; and ii) comparing nemeglya expression in the cell after exposure to the test substance to that of an unexposed control cell. Nemeglya expression is determined by methods common to the art or included herein. Methods of determining nemeglya expression include but are not limited to methods of quantifying nemeglya polynucleotides (e.g., detection of nemeglya mRNA by northern blot or RTPCR) or to methods of quantifying nemeglya polypeptides (e.g., detection of nemeglya proteins by western blot or immunochemistry). Preferably, the test substance modifies the expression of nemeglya in a specific cell type while not in others. Most preferably, the test substance modifies nemeglya expression specifically in neural cells.

A further embodiment of the present invention is directed to test substances for modulators of nemeglya activity and to a method of screening for such test substances comprising the steps of: i) contacting a cell with a test substance, ii) determining nemeglya activity, and iii) comparing nemeglya activity in the cell after exposure to that of an unexposed control cell. Nemeglya activity can, for example, be monitored by studying neurotransmission at synapses in slices of rat brain. Several methods for studying the level of neurotransmitter presynaptic release are common to the art. One assay for measuring electrophysiologically the postsynaptic depolarization caused by the presynaptic release is described in U.S. Pat. No. 6,051,610, which disclosure is hereby incorporated by reference in its entirety. Nemeglya activity may also be monitored by studying neurite outgrowth in cultures of neural cells. Neurite outgrowth can for example be quantificated as described in Lagenaur et al.. (J Neurobiol. 23:71-88 (1992)), disclosure of which is incorporated by reference in its entirety.

Test substances that decrease nemeglya expression or activity are defined as nemeglya antagonists. Test substances that increase nemeglya expression or activity are defined as nemeglya agonists. Tests substances that modulate the expression or activity of nemeglya include but are not limited to chemical compounds, oligonucleotides, antisense polynucleotides, ribozymes and anti-nemeglya antibodies. These substances may be made and used according to methods well known in the art.

Another embodiment of the present invention provides a method for promoting neurite outgrowth comprising the step of: delivering a nemeglya polypeptide or a nemeglya agonist to a neuron. As used herein, the term neurite outgrowth comprises dendritic and axonal outgrowth from neuronal cells. Preferably, a nemeglya agonist is used in such a method. Also preferably, nemeglya polypeptides are delivered to the cell by introducing a polynucleotide construct comprising an expression control element operably linked to a polynucleotide encoding a nemeglya polypeptide to the cell.

In a preferred embodiment, said method for promoting neurite outgrowth is performed in vitro by delivering a nemeglya polypeptide or a nemeglya agonist to a in vitro tissue culture of neuronal cells. Use of such a method is useful for the survival and growth of neurons that will be grafted into the central nervous system. Intracerebral grafting of neurons to the CNS is a commomly used method of treating a subject with a neuronal disorder. Such methods for grafting will be known to those skilled in the art and are described in Neural Grafting in the Mammalian CNS, Bjorklund and Stenevi, eds., (1985), and U.S. Pat. No. 5,082,670, which are incorporated by reference herein in their entireties.

In another preferred embodiment, said method for promoting neurite outgrowth is applied to an individual in vivo. Preferably, a composition comprising a nemeglya agonist and a physiologically acceptable carrier is administered to an individual. Also preferably, nemeglya polypeptides are delivered to the neuron by introducing in vitro a polynucleotide construct comprising an expression control element operably linked to a polynucleotide encoding a nemeglya polypeptide to said neuron, and by grafting said neuron. Said method for promoting neurite outgrowth using nemeglya polypeptides or nemeglya agonists may be useful to treat or reduce in severity damages consecutive to brain injuries. Such brain injuries may be caused by a traumatism or by infections. Such infections comprise, but are not limited to, Aspergillus CNS infections, meningitis, streptococcal infections associated with neuropsychiatric disorders and influenza virus-associated encephalopathies. Said method for promoting neurite outgrowth using nemeglya polypeptides or nemeglya agonists may also be useful to treat or reduce in severity neurodegenerative disorders such as, e.g., Parkinson's disease, Alzheimer's disease, cerebellum-type Creutzfeldt-Jakob disease, Huntington's disease and amyotrophic lateral sclerosis. Preferably, said method is applied to promote neurite outgrowth of the cholinergic neurons of the basal forebrain when treating Alzheimer's disease. Also preferably, said method is applied to promote neurite outgrowth of dopamine neurons in the substantia-nigra of the midbrain when treating Parkinson's disease.

A preferred embodiment of the present invention provides a method for restoring vesicle docking and fusion abilities to a cell comprising the step of. delivering a nemeglya polypeptide or a nemeglya agonist to a cell. Preferred cells are neurons. Preferably, a composition comprising a nemeglya agonist and a physiologically acceptable carrier is used in this method. Also preferably, nemeglya polypeptides are introduced to the cell by introducing a polynucleotide construct comprising an expression control element operably linked to a polynucleotide encoding a nemeglya polypeptide to the cell. Said method can be performed in vitro on cultures of neural cells. Said method can also be performed in vivo to treat an individual suffering from diseases associated with impaired vesicle docking and neurotransmission. Such diseases comprise various neurological, mental and paralytic disorders. For example, this method may be applied to, e.g., increase vesicular transmission of endocannabinoids to treat or prevent Huntington's disease, increase vesicular transmission of synaptotagrnin to treat or prevent Lambert-Eaton myasthenic syndrome, or increase vesicular transmission of GABA to treat or prevent progressive cerebellar ataxia. This method may also be applied to overcome impaired synaptic transmission caused by generation of aggregated beta-amyloid in Alzheimer's disease, or to overcome the blockage of neurotransmitter release caused by neurotoxins in the neuroparalytic syndromes of tetanus and botulism.

Still another preferred embodiment of the present invention provides a method for treating the velocardiofacial syndrome-like phenotype associated with a deletion at the 4q34 locus (VCFS-4q34), described by Tsai et al.. (Am J Med Genet. 82(4):336-9 (1999)). VCFS4q34 is characterized by, among other symptoms, learning difficulties. Said method comprises the step of: delivering a nemeglya polypeptide or a nemeglya agonist to an individual presenting the VCFS4q34 phenotype. Preferably, a composition comprising a nemeglya agonist and a physiologically acceptable carrier is used in this method. Also preferably, nemeglya polypeptides are introduced to the cell by introducing a polynucleotide construct comprising an expression control element operably linked to a polynucleotide encoding a nemeglya polypeptide to the cell.

Another embodiment relates to methods of using nemeglya polypeptides for inhibiting the release of neurotransmitters by preventing the docking and/or fusing of a presynaptic vesicle to the presynaptic membrane. These nemeglya polypeptides may be referred to as excitation-secretion uncoupling peptides (ESUPs). Fragments of nemeglya having this blocking activity can be identified using methods known in the art, for example by monitoring the inhibitory effect of nemeglya test fragments on catecholarnine release from detergent-permeabilized chromaffin cells as described in U.S. Pat. No. 6,169,074, which is incorporated by reference in its entirety. For optimal activity, ESUPs of the invention have a rninimum length of about 20 amino acids and a maximal length of about 100 amino acids, although they may be larger or smaller. Nemeglya ESUPs are preferably covalently linked to a targeting moiety. Preferred targeting moieties comprise a ligand to a cell-surface binding site present on a specific cell type (e.g., neuron) that is capable of functionally interacting with the binding site. Preferred targeting moieties are nerve growth factor and functional derivatives thereof. Alternatively, nemeglya ESUPs may be expressed in a cell by introducing polynucleotides encoding nemeglya ESUPs to the cell as described above. Nemeglya ESUPs may be particularly useful for alleviating chronic muscle spasm that occurs in, e.g., dystonia, cerebral palsy and muscular dystrophy.

An additional embodiment relates to methods of using nemeglya antagonists to inhibit or treat pain. Such methods comprise the step of administering a composition comprising an effective amount of a nemeglya antagonist and a physiologically acceptable carrier to an individual in need of treatment. For example, nemeglya antagonists may be used according to U.S. Pat. No. 5,989,545 (disclosure of which is hereby incorporated by reference in its entirety) by substituting the polypeptides of the present invention for neurotoxin. In addition, nemeglya antagonists of the invention may be used as anticonvulsants. Anticonvulsants effectively reduce infarct size in cases of stroke and are effective therapies for seizures, such as those resulting from epilepsy or exposure to neurotoxins.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising a nemeglya agonist and a physiologically acceptable carrier can be for, e.g., intravenous, topical, rectal, local, inhalant or subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a nemeglya polypeptide or a nucleic acid sequence that encodes a cDNA that is complementary to nemeglya polynucleotides. Methods of constructing transgenic animals are well-known to those skilled in the art. For example, a transgenic mouse may be constructed according to U.S. Pat. No. 5,723,719, which disclosure is hereby incorporated by reference in its entirety, by substituting the polynucleotides of the present invention for the Ren-2d renin gene. Such transgenic animals may be useful for obtaining animal models for VCFS-4q34 and diseases associated with impaired neurotransmission, or for screening for drugs treating neurological diseases and chronic muscle spasm.

Protein of SEQ ID NO:4 (Internal Designation Clone 1000772225_{—}208-30-4-0-H4-F)

The cDNA of Clone 1000772225_{—}208-304-0-H4-F (SEQ ID NO:3) encodes sica19 of SEQ ID NO:4, comprising the amino acid sequence:

MALLLALSLLVLWTSPAPTLSGTNDAEDCCLSVTQKPIPGYIVRNFHYLLIKDGCRVPAVV.

Accordingly, it will be appreciated that all characteristics and uses of the polypeptides of SEQ ID NO:4 described throughout the present application also pertain to the polypeptides encoded by the nucleic acids comprising the human cDNA in Clone 1000772225_{—}208-304-0-H4-F. In addition, it will be appreciated that all characteristics and uses of the polynucleotides of SEQ ID NOs:3 described throughout the present application also pertain to the nucleic acids comprising the human cDNA in Clone 1000772225_{—}208-30-4-0-H4-F. A preferred embodiment of the invention is directed toward the compositions of SEQ ID NO:4, SEQ ID NO:3, and Clone 1000772225_{—}208-30-4-0-H4-F. Preferred sica19 polypeptide fragments for uses in the methods described below include the sica19 polypeptide comprising the amino sequence of: GTNDAEDCCLSVTQKPIPGYIVRNIKDGCRVPAVV. Also preferred are polypeptide fragments having a biological activity as described herein and the polynucleotides encoding the fragments.

The protein of the invention, sica19, is a NOVEL splice variant of the small inducible Wytokine A19 (MIP-3-beta, Genbank accession number Q99731). The first exon of sica19 cDNA is longer than the first exon of MIP-3-beta cDNA. The second exon is identical between the two proteins. Sica19 cDNA lacks the third exon of MIP-3-beta cDNA, and the third exon of sica19 cDNA is identical to the fourth exon of MIP-3-beta cDNA. Thus the protein of the invention, the sica19 precursor, is 61 amino-acids long whereas the MIP-3-beta precursor is 98 amino-acids long. Sica19 belongs to the CC chemokine family. Sica19 is a secreted protein that displays a signal peptide (MALLLALSLLVLWTSPAPTLS), and the mature sica19 protein is 40 amino-acids long. While most of CC chemokines, including MIP-3-beta, are thought to posses two distinct receptor binding sites (Clark-Lewis et al. J Leukoc Biol. 57:703-11 (1995)), sica19 displays only one binding site for CCR7. This unique binding site is located at the amino-terminal extremity of the processed protein.

Sica19 exerts chemotactic activities for B, T and activated NK cells, and plays a fundamental role in their trafficking to targets such as mucosal surfaces, thymus, secondary lymphoid organs or infection or inflammation sites. Sica19 is expressed in thymus, lymph node, appendix, tonsil as well as in mature dendritic cells, monocytes, lymphocytes and neutrophils. It is also expressed at lower levels in colon, trachea, spleen, small intestine, lung, kidney and stomach. Sica19 binds to the CCR7 receptor (Genbank accession number P32248), which is expressed in normal lymphoid tissues and in several B and T lymphocyte cell lines. Sica19 plays a major role in normal lymphocyte recirculation and homing. Sica19 also plays a major role in inflammatory and immunological responses, both by attracting lymphocytes to inflammatory sites and by stimulating lymphocyte response. Notably, sica19 enhances interleukin-10 (lL-10) production by monocytes and T cells. Sica19 also regulates interactions between T cells and NK cells that are crucial for the generation of optimal immune responses (Robertson et al. Cell Immunol 199:8-14 (2000)).

An embodiment of the invention is directed to a composition comprising a sica19 polypeptide consisting of a polypeptide sequence of SEQ ID NO:4.

A further embodiment of the invention is directed to a composition comprising a sica19 polypeptide fragment having biological activity of regulating normal lymphocyte trafficking and of regulating lymphocyte inflammatory and immunological response.

As used herein, a sica19 polypeptide refers either to a sica19 polypeptide sequence of SEQ ID NO:4 or at least 70%, 80%; 90%, 95%, or more identical to SEQ ID NO:4, or to a sica19 polypeptide fragment having biological activity of regulating normal lymphocyte trafficking and of regulating lymphocyte inflammatory and immunological response.

An embodiment of the invention is directed to a composition comprising a polynucleotide sequence of SEQ ID NO:3 encoding a sica19 polypeptide.

A further embodiment of the invention is directed to a composition comprising a polynucleotide sequence encoding a sica19 polypeptide fragment.

As further used herein, a sica19 polynucleotide refers either to a sica19 polynucleotide sequence of SEQ ID NO:3 or having at least 70%, 80%, 90%, 95%, or more identity to SEQ ID NO:3, to a nucleotide sequence encoding a sica19 polypeptide, or to a polynucleotide fragment of SEQ ID NO:3.

A further embodiment of the invention is directed to an antibody recognizing a sica19 polypeptide sequence of SEQ ID NO:4 or a sica19 polypeptide fragment. Preferably, the antibody recognizes one or more amio-acids located within the carboxyl-terminal extremity of sica19, wherein said one or more amino-acids are required for binding of the antibody to a sica19 polypeptide. Also preferably, the antibody recognizes a non linear epitope. Most preferably, the antibody binds to sica19 but not to MIP-3-beta. As used herein, an anti-sica19 antibody refers to an antibody or antigen-binding fragment thereof that specifically binds to a sica19 polypeptide.

An embodiment of the present invention relates to a method of binding an anti-sica19 antibody to a sica19 polypeptide comprising the step of: contacting a sica19 polypeptide with said antibody under conditions that allow binding to occur. Such conditions are well known to those skilled in the art. Such methods are useful for detecting sica19 polypeptides as further described herein.

An embodiment of the present invention is directed to a method of detecting sica19 polypeptides in a biological sample, said method comprising the steps of: i) contacting a biological sample with an anti-sica19 antibody; and ii) detecting the antigen-antibody complex formed. The antibody or antibody fragment may be monoclonal or polyclonal. In addition, the antibody or antibody fragment may be primarily or secondarily labeled by any detectable compound (e.g., radioactive, fluorescent, luminescent, or enzymatic) common in the art. Such a method may be applied to, e.g., diagnosis of graft rejection reactions as further described below.

An embodiment of the present invention is directed to methods of detecting sica19 expression in a biological sample, said methods comprising the steps of: i) providing a biological sample from an individual; ii) detecting the level of sica19 expression in the sample; and iii) comparing the level of sica19 expression in said sample to that of a control sample. Detecting the level of sica19 expression is useful to diagnose, e.g., various abnormal inflammatory and immunological responses. For example, a lower level of sica19 expression in said biological sample in comparison to the level in a control sample representative of the level in a normal individual is indicative of a graft rejection reaction. The level of sica19 expression in the sample can be assessed using any method, such as by detecting the level of sica19 mRNA or protein in the sample. For example, well-known techniques such as, e.g., western bot or immunochemistry may be used to detect sica19 expression. Preferably, anti-sica19 antibodies are used to detect the level of sica19 expression. Said antibody may be monoclonal or polyclonal. In addition, said antibody may be primarily or secondarily labeled by any detectable compound (e.g., radioactive, fluorescent, luminescent and enzymatic compounds) common in the art. Also preferably, polynucleotide probes comprising a polynucleotide sequence of SEQ ID NO:3 or part thereof, or a polynucleotide sequence complementary of SEQ ID NO:3, are used to detect the level of sica19 expression by, e.g., northern blot or RTPCR techniques. A preferred method of detecting sica19 expression is described by Akalin et al. (Transplantation 72:948-53 (2001)), which disclosure is hereby incorporated by reference in its entirety.

Another embodiment of the present invention is directed to a diagnostic kit for detecting in vitro the presence of a sica19 polypeptide. Such kit comprises: i) an anti-sica19 antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-sica19 antibodies is used for diagnosing graft rejection reactions or various abnormal inflammatory and immunological responses. Optionally, said diagnostic kit comprises a negative control sample representative of the level expected from a normal individual. Optionally, said diagnostic kit comprises a positive control sample representative of the level expected from an individual suffering from a graft rejection reaction or from a given abnormal inflammatory or immunological response.

Another embodiment relates to a method of producing a sica19 polypeptide comprising the steps of: i) culturing a cell expressing a sica19 polypeptide, and ii) purifying the produced protein. The purification of the protein can be done following any technique well-known to those skilled in the art. Preferably, an anti-sica19 antibody may be bound to a chromatographic support to form an affinity chromatography column. Preferably, the cell expressing a sica19 polypeptide is a recombinant host cell as described below. Producing sica19 polypeptides may be useful in methods and compositions as further described in the present invention.

A preferred aspect of the invention is a host cell recombinant for polynucleotides encoding a sica19 polypeptide. An additional preferred aspect is a host cell recombinant for polynucleotides capable of directing sica19 expression. An embodiment is directed to a method of constructing a host cell recombinant for polynucleotides encoding a sica19 polypeptide comprising the steps of: i) constructing a recombinant vector that comprises a nucleic acid sequence encoding a sica19 polypeptide and that allows expression of said sica19 polypeptide under given physiological conditions, and ii) introducing said recombinant vector into a cell. In preferred embodiments, the recombinant vector is introduced into an Escherichia coli cell or into a human cell. Alternatively, a host cell recombinant for polynucleotides capable of directing sica19 expression may be constructed by a method comprising the steps of: i) constructing a recombinant vector comprising a polynucleotide capable of directing sica19 expression, and ii) introducing said recombinant vector into a cell. Preferably, the polynucleotides capable of directing sica19 expression are located in the 5′ regulatory region of the sica19 gene. Further preferably, these polynucleotides are located within 500 base pairs of the sica19 coding region. These polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing sica19 polypeptides may be used to produce sica19 polypeptides.

A preferred embodiment of the invention is a method comprising the step of: contacting a sica19 polypeptide with CCR7 under conditions that allow binding of said sica19 polypeptide to CCR7. In a preferred embodiment, binding of a sica19 polypeptide to CCR7 is used to purify cells expressing CCR7. In one such embodiment, a method for purifying cells expressing CCR7 comprises the steps of: i) contact a biological sample comprising CCR7-expressing cells with a labeled sica19 polypeptide; and ii) introducing said biological sample into a sorting apparatus, e.g. a fluorescence-activated cell sorter or a magnetic activated cell-sorting apparatus, wherein CCR7 cells within said saenple are sorted away from non-expressing cells by virtue of their bound, labeled sica19 polypeptide. . In peculiar, CCR7 is expressed by subsets of memory T cells that efficiently stimulate dendritic cells and that differentiate into cytotoxic effector cells upon secondary stimulation. Purifying CCR7+memory cells may thus be useful for purifying subsets of memory T cells that can be used to prevent various infections caused by, e.g., cytomegalovirus, HIV, T cell lymphotropic virus type I and Epstein-Barr virus.

Additional aspects of this embodiment include methods of using a sica19 polypeptide to detect and quantify cells expressing CCR7 using techniques common in the art. This method comprises the steps of: i) providing a biological sample suspected of containing cells expressing CCR7; ii) contacting said sample with a sica19 polypeptide under conditions suitable for binding of sica19; and iii) detecting the binding of sica19 to cells in the sample. Preferably, said sica19 polypeptide is covalently attached to a detectable compound. Alternatively, a detectable anti-sica19 antibody may be used to detect sica19. This method may be used as a diagnostic tool. For example, CCR7 is expressed differentially between T lymphocytes from normal individuals and T lymphocytes from individuals suffering from T cell leukemia or asthma.

Another preferred embodiment relates to methods of screening test substances for the ability to modulate sica19 expression comprising the steps of: i) contacting a cell with a test substance; and ii) comparing sica19 expression in the cell after exposure to the test substance to that of an unexposed control cell, wherein an observed difference in sica19 expression between the exposed cell and the unexposed control cell indicates that the test substance modulates sica19 expression. Sica19 expression may be determined by methods common to the art or included herein. Methods of determining sica19 expression include but are not limited to methods of quantifying sica19 polynucleotides (e.g., detection of sica19 mRNA by northern blot or RTPCR) or to methods of quantifying sica19 polypeptides (e.g., detection of sica19 polypeptides by western blot or immunochemistry). Preferably, the test substance modifies the expression of sica19 in a specific cell type while not in others. Most preferably, the test substance modifies sica19 expression specifically in dendritic cells, monocytes, lymphocytes, neutrophils or tumoral cells. Modulators identified using such methods are also encompassed by the present invention.

A further embodiment of the present invention is directed to methods of screening test substances for the ability to modulate sica19 activity comprising the steps of: i) contacting a cell with a test substance, and ii) comparing sica19 activity in the cell after exposure to the test substance that of an unexposed control cell, wherein an observed difference in sica19 activity between the exposed and unexposed cells indicates that the test substance modulates sica19 activity. Sica19 activity can, for example, be monitored by studying its chemotactic activity. The chemotactic activity of sica19 can be studied by a wide variety of techniques well-known to those skilled in the art. These techniques comprise the chemotaxis assay using a FACScan cell analyzer described in Braun et al. (J Immunol. 164:4025-31 (2000)) and the migration assay using a chemotaxis chamber described in Scapini et al. (Bur J Immunol. 31:1981-8 (2001)), disclosure of which are both incorporated by reference in their entireties. Modulators identified using such methods are also encompassed by the present invention.

A further embodiment of the present invention is directed to methods of screening test substances for the ability to modulate binding of sica19 to CCR7. For example, test substances for modulators of binding of sica19 to CCR7 can be screened according to U.S. Pat. No. 6,153,441, which disclosure is hereby incorporated by reference in its entirety, by substituting sica19 for the CK>9 ligand. Modulators identified using such methods are also encompassed by the present invention.

Test substances that decrease sica19 expression, activity or binding to CCR7 are defined as sica19 antagonists. Test substances that increase sica19 expression, activity or binding to CCR7 are defined as sica19 agonists. Test substances that modulate the expression or activity of sica19 include, but are not limited to, chemical compounds (e.g. small-molecule inhibitors or activators), oligonucleotides, antisense polynucleotides, polypeptides, ribozymes, dominant negative forms of sica19, and anti-sica19 antibodies. These substances may be made and used according to methods well known in the art.

A preferred embodiment of the present invention provides a method for enhancing an immunological response in a lymphocyte, the method comprising the step of: administering a sica19 polypeptide or a sica19 agonist to the lymphocyte, wherein an immunological response in the lymphocyte is enhanced. Such methods may be carried out in vitro, e.g., by delivering a sica19 polypeptide or a sica19 agonist to an in vitro culture of lymphocytes. Stimulating lymphocytes in vitro is for example useful when measuring the reactivity of T cells from a patient prior to transplantation, or when screening for immunosuppressive drugs. Such a method for enhancing lymphocyte immunological response may be applied in vivo or ex vivo by delivering a physiologically acceptable carrier and an effective amount of a sica19 polypeptide or a sica19 agonist to an individual or to a population of lymphocytes to be administered to the individual. As used herein, an effective amount of a sica19 polypeptide or a sica19 agonist refers to an amount of sica19 polypeptide or sica19 agonist that is sufficient to enhance the lymphocyte immunological response. For example, such a method may be applied to, e.g., attracting T lymphocytes into atherosclerotic lesions for inhibiting development of atherosclerotic plaques, enhancing antitumor responses for treating cancer, inhibiting proliferation of normal human marrow progenitors for treating chronic myelogenous leukemia, and attracting and stimulating T lymphocytes for treating infections by HIV and other pathogens. The sica19 polypeptide or sica19 agonist may be administered systemically, for example by intravenous injection, for enhancing antitumor responses, for treating chronic myelogenous leukemia or for treating infections by HIV and other pathogens. Alternatively, the sica19 polypeptide or sica19 agonist may be administered locally, e.g., at a tumor site or at an atherosclerotic plaque. Said sica19 polypeptide or sica19 agonist may be administered alone or in combination with other molecules, for example in combination with interleukin-2 and granulocyte-macrophage colony-stimulating factor for enhancing antitumor immunity.

An additional embodiment relates to methods of using sica19 antagonists to inhibit or reduce Iymphocyte immunological response. Such methods comprise the step of: administering a composition comprising an effective amount of a sica19 antagonist and a physiologically acceptable carrier to an individual. Preferably, such methods are directed to treat allergy and asthma, and to prevent on-going immune responses. More particularly, such methods can be used to prevent or reduce the severity of graft rejections, graft versus host diseases and autoimmune diseases (e.g., rheumatoid arthritis, systemic lupus, multiple sclerosis, insulin-dependent diabetes, hepatitis, rheumatoid arthritis, Graves disease), and to induce tolerance to graft transplantation (e.g., transplantation of cells, bone marrow, tissue, solid-organ, bone).

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising a sica19 agonist and a physiologically acceptable carrier can be for, e.g., intravenous, topical, rectal, local, inhalant, subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a sica19 polypeptide or a nucleic acid sequence that encodes a cDNA that is complementary to sica19 polynucleotides. Methods of constructing transgenic animals are well-known to those skilled in the art. For example, a transgenic mouse may be constructed according to U.S. Pat. No. 5,434,340, which disclosure is hereby incorporated by reference in its entirety. Such transgenic animals may be useful for obtaining animal models for patients suffering from diseases associated with impaired immunological response, and for screening for sica19 agonists and antagonists.

Protein of SEQ ID NO:6 (Internal Designation Clone 500737075_cFS_{—}205-46-1-0-D7-F)

The cDNA of Clone 500737075_cFS_{—}20546-1-0-D7-F (SEQ ID NO:5) encodes Plap11 of SEQ ID NO:6, comprising the amino acid sequence: MRACISLVLAVLCGLAWAGKIESCASRCNEKFNRDAACQCDRRCLWHGNCCEDYEHLCTEDHK ESEPLPQLEEETEEALASNLYSAPTSCQGRCYEAFDKHHQCHCNARCQEFGNCCKDFESLCSDHE VSHSSDAITKEEIQSISEKYIATNKAQKEDIVLNSQNCISPSETRNQVDRCPKPLFTYVNEKLFS KPTYAAINLLNNYQRATGHGEHFSAQELAEQDAFLREIMKTAVMKELYSFLHHQNRYGSEQIEF VDDLKNMWFGLYSRGNEEGDSSGFEHVFSGEVKKGKVTGHNWIRFYLEEKEGLVDYYSHIYD GPWDSYPDVLAMQFNWDGYYKEVGSAFIGSIPEFEFALYSLCFIARPGKVCQLSLGGYPLAVRT YTWDKSTYGNGKKYIATAYIVSST. Accordingly, it will be appreciated that all characteristics and uses of the polypeptides of SEQ ID NO:6 described throughout the present application also pertain to the polypeptides encoded by the nucleic acids comprising the human cDNA in Clone 500737075_cFS_{—}205-46-1-0-D7-F. In addition, it will be appreciated that all characteristics and uses of the polynucleotides of SEQ ID NOs:5 described throughout the present application also pertain to the nucleic acids included in Clone 500737075_cFS_{—}20546-1-0-D7-F. A preferred embodiment of the invention is directed toward the compositions of SEQ ID NO:6, SEQ ID NO:5, and Clone 500737075_cFS_{—}20546-1-0-D7-F. Preferred plap11 polypeptide fragments for uses in the methods described below include the plap11 polypeptide comprising the amino sequence of: GKIESCASRCNEKFNRDAACQCDRRCLWHGNCCEDYEHLCTEDHKESEPLPQLEEETlEALASN LYSAPTSCQGRCYEAFDKHHQCHCNARCQEFGNCCKDFESLCSDHEVSHSSDAITKEEIQSISEKI YRADTNKAQKEDWWSQNCISPSETRNQVDRCPKPLFTYVNEKLFSKPTYAAFINLLNNYQRAT GHGEHFSAQELAEQDAFLREIMKTAVMKELYSFLHHQNRYGSEQEFVDDLKNMWFGLYSRGNE EGDSSGFEHVFSGEVKKGKVTGFHNWIRFYLEEKEGLVDYYSHBMIGPWDSYPDVLAMQFNWD GYYKEVGSAPIGSIPEFEFALYSLCIARPGKVCQLSLGGYPLAVRTYTWDKSTYGNGKKYIATA YIVSST. Another preferred plap11 polypeptide fragments for uses in the methods described below include the plap11 polypeptide comprising the amino sequence of: KIESCASRCNEKFNRDAACQCDRRCLWHGNCCEDYEHLCTEDHKSEPLPQLEEETEEALASNL YSAPTSCQGRCYEAFDKHHQCHCNARCQElFGNCCKDFESLCSDHEV. Also preferred are polypeptide fragments having a biological activity as described herein and the polynucleotides encoding the fragments.

The protein of the invention, plap11, is a NOVEL splice variant of the placental protein 11 (PP11, GenBank accession number P21128). Exon I of plap11 is identical to exon I of PP11. Exon 2 is unique to plap11. Exons 3 to 10 of plap 1 are identical to exons 2 to 9 of PP11. Thus, after cleavage of the signal peptide (MRACISLVLAVLCGLAWA), plap11 displays 41 more amino-acids than PP11 at its amino-terminal extremity. While PP11 displays only one somatomedin B domain, plap11 displays two somatomedin B domains, the first one being unique to plap11 (KIESCASRCNEKFNRDAACQDRRCLWHGNCCEDYEHLCTEDHK). The second domain (APTSCQGRCYEAFDKHHOCHCNARCQEFGNCCKDFESLCSDHEV) is common to both PP11 and plap11. Somatomedin B domains permit binding to serine protease inhibitors (Seiffert et al. J Biol Chem. 266:2824-30 (1991)).

Proteases involved in the matrix degradation play a major role during implantation, placentation and trophoblast invasion (Salamonsen, Rev Reprod 4:11-22 (1999)). Furthermore, many changes in the coagulation-fibrinolysis system take place during pregnancy. Trophoblasts produce fibrinolytic proteins that can promote normal implantation and regulate blood flow to the fetus and placenta throughout pregnancy. The coagulation-fibrinolysis system is regulated by the balance between proteases and protease inhibitors. Normal pregnancy is associated with a hypofibrinolytic state that is fundamentally caused by an increase in protease activator inhibitors (Gilabert et al. Hum Reprod 10 Suppl 2:121-31 (1995)). Plap11 is a secreted serine protease that is specifically expressed in placental tissues and in various malignant tumors. Plap11 is involved both in degradation of the extracellular matrix and in regulation of the coagulation-fibrinolysis system. Its activity it down-regulated by serine protease inhibitors that bind to its somatomedin domains.

An embodiment of the invention is directed to a composition comprising a plap 11 polypeptide sequence of SEQ ID NO:6.

A further embodiment of the invention is directed to a composition comprising a plap11 polypeptide fragment having serine protease activity or serine protease inhibitor binding activity.

As further used herein, a “plap11 polypeptide” refers either to a plap11 polypeptide sequence of SEQ ID NO:6 or to a plap 11 polypeptide having serine protease activity or serine protease inhibitor binding activity.

A further embodiment of the invention is directed to a composition comprising a polynucleotide sequence of SEQ ID NO:5 encoding a plap11 polypeptide.

A further embodiment of the invention is directed to a composition comprising a polynucleotide sequence encoding a plap 11 polypeptide fragment having serine protease activity or serine protease inhibitor binding activity.

A further embodiment of the invention is directed to an antibody recognizing a plap11 polypeptide. Preferably, the antibody recognizes one or more amino-acids located within the 41 amino-terminal amino-acids of plap11, wherein said one or more amino-acids are required for binding of the antibody to a plap11 polypeptide. Most preferably, the antibody binds to plap11 but not to PP11. Preferred amino-acid sequences that are recognized by said antibody comprise the ASRCNEKFNRDAA, the CQCDRRCLW and the GNCCEDYE epitopes. As used herein, an “anti-plap11 antibody” refers to an antibody or to an antigen binding fragment that specifically binds to a plap11 polypeptide.

An embodiment of the present invention relates to a method of binding such an anti-plap11 antibody to a plap11 polypeptide comprising the step of: contacting a plap11 polypeptide with said antibody or an antigen-binding fragment thereof under conditions that allow binding to take place. Such conditions are well known to those skilled in the art. Such methods are useful for detecting plap11 polypeptides as further described herein.

An embodiment of the present invention is directed to a method of detecting plap11 polypeptides in a biological sample, said method comprising the steps of: i) contacting a biological sample with an anti-plap 11 antibody; and ii) detecting the antigen-antibody complex formed. The antibody or antibody fragment may be monoclonal or polyclonal. In addition, the antibody or antibody fragment may be primarily or secondarily labeled by any detectable compound (e.g., radioactive, fluorescent, luminescent, or enzymatic) common in the art. Such a method may be applied in a diagnostic kit as further described below.

Another embodiment of the present invention is directed to methods of detecting plap11 expression in a biological sample, said methods comprising the steps of: i) providing a biological test sample from an individual; ii) detecting the level of plap11 expression; and iii) comparing the level of plap11 expression in said test sample to that of control sample(s). Plap11 may be used as a tumor marker, and the methods of detecting plap 11 expression may be used for diagnosing or for monitoring patients suffering from malignant tumors, carcinomas, sarcomas or cancers such as, e.g., breast cancers, testicular malignant tumors, ovarian adenocarcinomas, choriocarcinomas and gastric cancers. In one embodiment, the level of plap 11 expression is determined by examining the level of plap11 mRNA or polypeptides in the sample. Well-known techniques such as, e.g., western bot or immunochemistry may be used to detect plap11 expression. Preferably, anti-plap11 antibodies are used to detect the level of plap11 expression. Said anti-plap11 antibody may be monoclonal or polyclonal. Said anti-plap11 antibody may be primarily or secondarily labeled by any detectable compound (e.g., radioactive, fluorescent, luminescent and enzymatic compounds) common in the art. Most preferably, detection of plap11 polypeptides is performed using enzyme-linked immunoabsorbent assays (ELISAs), radioimmunoassays (RLAs), or immunoperoxidase (PAP) stains. Said biological test sample comprises samples such as, e.g., serum, plasma, tissue sections, effusions, ascites, urine, cerebrospinal fluid, semen, breast aspirates and fluids of ovarian origin. Alternatively, polynucleotide probes comprising a polynucleotide sequence of SEQ ID NO:5 or part thereof, or a polynucleotide sequence complementary of SEQ ID NO:5 are used to detect the level of plap11 expression by, e.g., northern blot or RTPCR techniques.

Another embodiment of the present invention is directed to a diagnostic kit for detecting in vitro the presence of a plap11 polypeptide. Such kit comprises: i) an anti-plap11 antibody; and optionally, ii) a reagent allowing the detection of the antigen-antibody complexes formed. Preferably, said antibody is detectably labeled as described above Alternatively, the optional reagent may provide a detectable signal. The optional reagent may either bind to said antibody or react with the label on said antibody. Preferably, the kit comprising anti-plap11 antibodies is used for diagnosing malignant tumors, carcinomas, sarcomas or cancers such as those listed above. Optionally, said diagnostic kit comprises a negative control sample that is representative of a level expected of a normal, disease-free individual. Optionally, said diagnostic kit comprises a positive control sample that is representative of a level expected of an individual suffering from given malignant tumors, carcinomas, sarcomas or cancers. Optionally, said kit comprises other markers used for diagnosis of malignant tumors such as, e.g., antibodies specific for the Hpr protein as described in U.S. Pat. No. 5,864,011, or antibodies that react with the CA-242 antigen as described in U.S. Pat. No. 5,552,293. In another embodiment, the kit provides reagents for detecting plap11 polynucleotides, e.g. plap11-specific probes or primers.

Another embodiment relates to a method of producing a plap11 polypeptide comprising the steps of: i) culturing a cell expressing a plap11 polypeptide, and ii) purifying the produced protein. The purification of the protein can be done following any technique well-known to those skilled in the art. Preferably, an anti-plap11 antibody may be bound to a chromatographic support to form an affinity chromatography column. Preferably, the cell expressing a plap11 polypeptide is a recombinant host cell as described below. Producing plap11 polypeptides may be useful in methods and compositions as further described in the present invention.

Another embodiment is directed to a recombinant host cell producing a plap11 polypeptide, and to a method of using plap11 polynucleotides for constructing a recombinant host cell producing a plap11 polypeptide comprising the steps of: i) constructing a recombinant vector that comprises a nucleic acid sequence encoding a plap11 polypeptide, operably linked to a promoter, and that allows expression of said plap11 polypeptide under given physiological conditions, and ii) introducing said recombinant molecules into a cell. In preferred embodiments, the recombinant vector is introduced into an Escherichia coli cell or into a human cell. Alternatively, recombinant host cells producing a plap11 polypeptide may be constructed by a method comprising the steps of: i) constructing a recombinant vector comprising a polynucleotide capable of directing plap11 expression, and ii) introducing said recombinant vector into a cell. Preferably, the polynucleotides capable of directing plap11 expression are located in the 5′ regulatory region of the plap11 gene. Further preferably, these polynucleotides are located within 500 base pairs of the plap11 coding region. These polynucleotides preferably comprise a promoter sequence. Techniques known in the art for introducing polynucleotide sequences to endogenous sequences are described in U.S. Pat. No. 5,6416,70 and PCT WO9629411, which disclosures are hereby incorporated by reference in their entireties. Such recombinant host cell producing plap11 polypeptides may be used to produce plap11 polypeptides.

Another preferred embodiment relates to the screening of test substances for modulation of plap11 expression comprising the steps of: i) contacting a cell with a test substance; and ii) comparing plap11 expression in the cell after exposure to the test substance to that of an unexposed control cell, wherein an observed difference in plap11 expression between the exposed an unexposed cells indicates that the test substance modulates plap11 expression. Plap11 expression is determined by methods common to the art or included herein.. Methods of determining plap11 expression include but are not limited to methods of quantifying plap11 polynucleotides (e.g., detection of plap11 mRNA by northern blot or RTPCR) or to methods of quantifying plap11 polypeptides (e.g., detection of plap11 proteins by western bot or immunochemistry). Preferably, the test substance modifies the expression of plap11 in a specific cell type while not in others.

A further embodiment of the present invention is directed to the screening of test substances for modulation of plap11 activity comprising the steps of: i) contacting a cell with a test substance, ii) determining plap11 activity, and iii) comparing plap11 activity in the cell after exposure to the test substance to that of an unexposed control cell, wherein an observed difference in plap11 activity between the exposed and unexposed cells indicates that the test substance modulates plap11 activity. Plap11 activity can be assessed in any of a large number of ways, for example by examining its amidolytic activity as described by Grundmann et al. (DNA Cell Biol. 9:243-50 (1990)), the disclosure of which is hereby incorporated by reference in its entirety.

Test substances that decrease plap11 expression or activity are defined as plap11 antagonists. Test substances that increase plap11 expression or activity are defined as plap11 agonists. Test substances that modulate the expression or activity of plap11 include but are not limited to chemical compounds, oligonucleotides, antisense polynucleotides, polypeptides, ribozymes, dominant negative forms of plap11 and anti-plap11 antibodies. These substances may be made and used according to methods well known in the art.

A preferred embodiment of the invention is a method comprising the step of: contacting a plap11 polypeptide with a serine protease inhibitor under conditions that allow binding of said plap11 polypeptide to said serine protease inhibitor. Preferably, plap11 polypeptides for uses in this method comprise both somatomedin domains. Even more preferably, plap11 polypeptides for uses in this method have serine protease inhibitor binding activity and not serine protease activity.

One aspect of this embodiment include a method of using a plap11 polypeptide to purify serine protease inhibitors. Such a method for purifying serine protease inhibitors comprises the steps of: i) covalently or non-covalently attaching a plap11 polypeptide to a solid matrix; ii) adding a fluid sample comprising serine protease inhibitors; iii) washing the solid matrix to remove associated contaminants; and iv) eluting said serine protease inhibitors from said solid matrix. As protease inhibitors can inhibit proteases introduced into the body of an individual by infective and parasitic agents, protease inhibitors are a useful pharmaceutical tool. Purifying serine protease inhibitors using a plap11 polypeptide can be useful for, e.g., purifying a given serine protease inhibitor from a culture or for isolating NOVEL serine protease inhibitors.

Another aspect of this embodiment include a method of using a plap11 polypeptide to remove serine protease inhibitors from a fluid sample of interest. Such a method for removing serine protease inhibitors from a fluid sample comprises the step of: adding a plap11 polypeptide to a fluid sample of interest comprising or potentially comprising serine protease inhibitors. The protease inhibitor may be removed by immunoprecipitation with said plap11 polypeptide. Alternatively, said plap11 polypeptide may be covalently or non-covalently attached to a solid matrix, and the sample of interest may be removed after contact with the plap11 polypeptide. Such a method is useful for treating samples before adding a protease to a protein-containing sample.

Additional aspects of this embodiment include methods of using a plap11 polypeptide to detect and/or quantify serine protease inhibitors in a sample using techniques common in the art. This method comprises the steps of: i) obtaining a biological sample suspected of containing serine protease inhibitors; ii) contacting said sample with a plap11 polypeptide under conditions suitable for binding of plap11; and iii) detecting complexes formed between plap11 and a serine protease inhibitor.. Preferably, said plap11 polypeptide is covalently attached to a detectable compound. Alternatively, a detectable anti-plap11 antibody may be used to detect plap11. This embodiment is useful, for example, as a diagnostic tool for detecting levels of serine protease inhibitors in a sample, e.g. in serum. For example, bloodstream levels of serine protease inhibitors correlate with the risk for thrombotic deposition, as discussed in Fareed et al. (Clin Chem 29:1641-58 (1983)), and levels of protease inhibitors in a tumor sample correlate with the malignancy grade of the tumor, as discussed in Maass et al. (Clin Biochem. 34:303-7 (2001)) and in Rao et al. (Clin Cancer Res. 7:570-6 (2001)).

Alternatively, such a method for inhibiting plap11 directed to reduce blood coagulation. Such a method to reduce blood coagulation comprises the step of: introducing a plap11 antagonist in a physiologically acceptable composition to the bloodstream of an individual. Delivery to the bloodstream can be direct (e.g., injection to a vein or artery) or indirect (e.g., inhalant or oral delivery), as further discussed herein. Preferably, such a method for reducing blood coagulation is directed to treat a patient suffering from thrombotic diseases and/or hypercoagulable syndromes, e.g., artherosclerosis, acute ischemia, stroke, myocardial infarction, coronary artery disease, and thrombotic complications associated with transplantation. Even more preferably, such a method is directed to treat pregnancy disorders associated with coagulation abnormalities such as, e.g., preeclampsia, toxemia of pregnancy, placental abruption and intrauterine fetal death associated.

Another embodiment is directed to a composition comprising a plap11 polypeptide having protease activity. Said composition can be used in a method comprising the step of: adding said composition to a biological sample under conditions that allow plap11 protease activity. Preferably, said compostion is a “protease cocktail” comprising several different proteases, wherein said protease cocktail is able to digest a wide range of proteins. Such protease cocktails are useful in laboratory assays to degrade undesirable proteins in a sample, for example for removing proteins in a DNA preparation or for removing enzymes after any enzymatic reaction.

A further embodiment relates to methods for decreasing plap11 activity comprising the step of: delivering a plap11 antagonist to a cell, a tissue sample or an individual. Such methods may be performed in vitro (e.g., in vitro culture of cells) or in vivo (e.g., administration of a plap11 antagonist to an individual). Preferred cells are tumoral cells.

In a preferred embodiment, such a method for decreasing plap11 activity in an individual is directed to inhibit ECM degradation. Preferably, such a method for preventing or reducing ECM degradation is directed to protect the ECM from degradation by malignant cells, thus blocking the invasion and spread of malignant tumors. Most preferably, a composition comprising a plap11 antagonist and a physiologically acceptable carrier is administered to an individual suffering from abnormal or undesirable cell proliferation, such as, e.g., tumor growth, endothelial cell proliferation, and angiogenesis related to tumor growth. Tumors that can be treated with the compositions of the present invention include, but are not limited to, breast cancers, testicular malignant tumors, ovarian adenocarcinomas, choriocarcinomas and gastric cancers.

In another preferred embodiment, a plap11 antagonist is fused to a targeting molecule specific for the tissue of interest. Preferably, the targeted plap11 antagonist is comprised in a pharmaceutical composition for use in prevention of ECM degradation associated with tumor growth. Most preferably, the plap11 antagonist is linked to a ligand or an antibody that recognizes a receptor or an antigen specifically expressed on tumor cells by any technique well-known to those skilled in the art. A large number of tumor-associated antigens have been described in the scientific litterature. For example, antigens and techniques described by Wikstrand et al (Cancer Metastasis Rev 18:451-64 (1999)), which disclosure is hereby incorporated by reference in its entirety, may be used. Alternatively, the plap11 antagonist is linked to a ligand that recognizes the vasculature of a tumor. A preferred method for targeting the plap11 antagonist to the vasulature of solid tumors is described in U.S. Pat. No. 6,004,554, which disclosure is incorporated by reference in its entirety.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising physiologically acceptable carrier and a plap11 polypeptide, a plap11 agonist or a plap11 antagonist can be for, e.g., intravenous, topical, rectal, local, inhalant or subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a plap11 polypeptide or a nucleic acid sequence that encodes a cDNA that is complementary to plap11 polynucleotides. Methods of constructing transgenic animals are well-known to those skilled in the art. For example, a transgenic mouse may be constructed according to U.S. Pat. No. 4,736,866, or U.S. Pat. No. 5,614,396, which disclosures are hereby incorporated by reference in their entireties. Such transgenic animals may be useful for obtaining animal models presenting a risk for thrombotic deposition or being predisposed to invasion and spread of malignant tumors, and are useful for screening for modulators of plap11 expression or activity.

Protein of SEQ ID NO:8 (Internal designation Clone 500720716_{—}204-33-1-0-E6-F_{—}1)

The cDNA of Clone 500720716_{—}204-33-1-0-E6-F_{—}1 (SEQ ID NO:7) encodes Nupre of SEQ ID NO:8, comprising the amino acid sequence: MCPRAARAPATLLLALGAVLWPAAGASELTILHTNDVHSRLEQTSEDSSKCVNASRCMGGVAR LFTKVQQIRRAEPNVLLLDAGDQYQGTIWFTVYKGAEVAHFMNALRYDAMALGNHEFYNGVE GLIEPLLKEAKFPILSANIKAKGPLASQISGLYLPYKVLPVGDEVVGIVGYTSKETPFLSNPGTNLV FEDEITALQPEVDKLKTLNVNKIIALGHSGFEMDKLIAQKVRGVDVVVGGHSNTFLYTGNCFKRI AWARMSR. Accordingly, it will be appreciated that all characteristics and uses of the polypeptides of SEQ ID NO:8 described throughout the present application also pertain to the polypeptides encoded by the nucleic acids comprising the human cDNA in Clone 500720716_{—}204-33-1-0-E6-F_{—}1. In addition, it will be appreciated that all characteristics and uses of the polynucleotides of SEQ ID NOs:7 described throughout the present application also pertain to the nucleic acids comprising the human cDNA in Clone 500720716_{—}204-33-1-0-E6-F_{—}1. A preferred embodiment of the invention is directed toward the compositions of SEQ ID NO:8, SEQ ID NO:7, and Clone 500720716_{—}204-33-1-0-E6-F_{—}1. Preferred Nupre polypeptide fragments for uses in the methods described below include the Nupre polypeptide comprising the amino sequence of: SELTILHTNDVHSRLEQTSEDSSKCVNASRCMGGVARLFKVQQIRRAEPNVLLLDAGDQYQGTI WFTVYKGAEVAHFMNALRYDAMALGNHEFYNGVEVGLIEPLLKEAKFPILSANIKAKGPLASQIS GLYLPYKVLPVGDEVVGIVGYTSKETPFLSNPGTNLVFEDEITALQPEVDKLKTLNVNKIIALGHS GFEMDKLIAQKVRGVDVVVGGHSNTFLYTGNCFKRIAWARMSR. Also preferred are polypeptide fragments having a biological activity as described herein and the polynucleotides encoding the fragments.

The protein of the invention, Nupre, is a NOVEL, secreted splice variant of the 5′-nucleotidase precursor (5NTD, Genbank accession number P21589). Exons 1 and 2 are identical between Nupre cDNA and 5NTD cDNA. The third exon of Nupre cDNA is longer than the third exon of 5NTD cDNA. Thus both mature proteins are identical on their 226 amino-terminal amino-acids, and the twelve carboxy-terminal-amino-acids of Nupre are unique to this splice variant. Nupre is 264 amino-acids long whereas 5NTD is 574 amino-acids long. Nupre displays a signal peptide (MCPRAARAPATLLLALGAVLWPAAGAS) and a 5′-nucleotidase signature_{—}1 consensus (LTILHTNDVHSRL). Nupre also displays a 5′-nucleotidase catalytic domain. While 5NTD is a GPI-anchored ectoenzyme, Nupre is a secreted protein that is not attached to the cell wall.

Nupre is a soluble extracellular 5′-nucleotidase, which catalyzes the dephosphorylation of 5′-ribonucleotides to the corresponding ribonucleosides. Notably, Nupre produces adenosine and thereby promotes adenosine receptor-mediated signalling. Adenosine receptors mediate many important physiological responses, including cardiac rate and contractility, neurotransmission, renal function, smooth muscle vasodilation, platelet aggregation, lipolysis and mast cell activation. In particular, adenosine mediates inhibition of lipolysis in adipocytes (Ramkumar et al. Jpn J Pharmacol 86:265-74 (2001)), and also has several functions within the CNS such as inhibition of neurotransmission and neuroprotective actions in pathological conditions (see, e.g., Latini et al., J Neurochem 79:463-84 (2001), the disclosure of which is incorporated herein in its entirety). In contrast to 5NTD, Nupre does not act as a co-stimulatory molecule for the CD3/T-cell receptor complex, and is not involved in cell adhesion. Instead, while 5NTD has several different functions, some of which do not require 5′-nucleotidase activity, Nupre is specifically involved in the control of extracellular adenosine levels. Nupre is widely expressed by various cell types, including epithelial cells, glial cells, adipocytes and lymphocytes. Its expression correlates positively with lymphocyte differentiation.

An embodiment of the invention is directed to a composition comprising a Nupre polypeptide sequence of SEQ ID NO:8, or having at least 70%, 80%, 90%, 95%, 96%, 97%, 98%, 99%, or more identity to SEQ ID NO:8.

A further embodiment of the invention is directed to a composition comprising a Nupre polypeptide fragment having 5′-nucleotidase activity.

As used herein, a “Nupre polypeptide” refers either to a Nupre polypeptide sequence of SEQ ID NO:8 or at least 70%, 80%; 90%, 95%, 96%, 97%, 98%, 99%, or more identical to SEQ ID NO:8, or to a Nupre polypeptide fragment having 5′-nucleotidase activity.

An embodiment of the invention is directed to a composition comprising a polynucleotide sequence of SEQ ID NO:7 encoding a Nupre polypeptide.

A further embodiment of the invention is directed to a composition comprising a polynucleotide sequence encoding a Nupre polypeptide.

As further used herein, a “Nupre polynucleotide” refers either to a Nupre polynucleotide sequence of SEQ ID NO:7 or having at least 70%, 80%, 90%, 95%, or more identity to SEQ ID NO:7, to a nucleotide sequence encoding a Nupre polypeptide, or to a polynucleotide fragment of SEQ ID NO:7.

A further embodiment of the invention is directed to an antibody recognizing a Nupre polypeptide sequence of SEQ ID NO:8 or a Nupre polypeptide fragment. Preferably, the antibody recognizes one or more of the twelve carboxyl-terminal amino-acids of Nupre, wherein said one or more amino-acids are required for binding of the antibody to a Nupre polypeptide. Most preferably, the antibody binds to Nupre but not to 5NTD. As used herein, an “anti-Nupre antibody” refers to an antibody or antigen-binding fragment thereof that specifically binds to a Nupre polypeptide.

An embodiment of the present invention relates to a method of binding an anti-Nupre antibody to a Nupre polypeptide comprising the step of: contacting a Nupre polypeptide with said antibody under conditions that allow binding to occur. Such conditions are well known to those skilled in the art. Such methods are useful for detecting Nupre polypeptides as further described herein.

An embodiment of the present invention is directed to a method of detecting Nupre polypeptides in a biological sample, said method comprising the steps of: i) contacting a biological sample with an anti-Nupre antibody; and ii) detecting the antigen-antibody complex formed. The anti-Nupre antibody may be monoclonal or polyclonal. In addition, the anti-Nupre antibody may be primarily or secondarily labeled by any detectable compound (e.g., radioactive, fluorescent, luminescent, or enzymatic) common in the art. Such a method may be applied to, e.g., identification of lymphoid populations at different stages of maturation in B cell disorders and diagnosis of immunodeficiencies as further described below.

An embodiment of the present invention is directed to methods of detecting Nupre expression in a biological sample, said methods comprising the steps of: i) providing a biological sample from an individual; ii) detecting the level of Nupre expression in the sample; and iii) comparing the level of Nupre expression in said sample to that of a control sample. In one such embodiment, detecting the level of Nupre expression is used to diagnose immunodeficiencies. Preferably, said biological sample comprises circulating lymphocytes. A lower level of Nupre expression in said biological sample in comparison to the level in a control sample representative of a level in a normal individual is indicative of an immunodeficiency disease such as, e.g., severe combined immunodeficiency, virus-induced immunodeficiencies including AIDS, hypogammaglobulinemia, agammaglobulinemia and primary humoral immunodeficiencies. In another such embodiment, detecting the level of Nupre expression is directed to identify lymphoid populations at different stages of maturation in chronic B cell disorders such as, e.g., B-cell lymphocytic leukemia and chronic myelogenous leukemia. The level of Nupre expression in the sample can be assessed using any method, such as by detecting the level of Nupre mRNA or Nupre polypeptides in the sample. For example, well-known techniques such as, e.g., western blot, immunochemical techniques and cytochemical techniques may be used to detect Nupre expression. Preferably, anti-Nupre antibodies are used to detect the level of Nupre expression. Also preferably, polynucleotide probes comprising a polynucleotide sequence of SEQ ID NO:7 or part thereof, or a polynucleotide sequence complementary of SEQ ID NO:7, are used to detect the level of Nupre expression by, e.g., northern blot or RTPCR techniques.

An embodiment of the present invention relates to methods a method for identifying mutations in a Nupre gene to detect a predisposition for an immunodeficiency. Such a method comprises the steps of: i) providing a nucleotide test sample from an individual; and ii) determining the presence of the genomic DNA encoding a Nupre polypeptide in one or more cells of the sample. The presence of Nupre genomic sequence is preferably detected using standard molecular biological techniques (e.g., Southern blotting) and employing a Nupre nucleotide or fragment thereof as a probe. Alternatively, the method comprises the steps of i) providing a nucleotide test sample from an individual; and ii) detecting the presence of a mutation in a Nupre genomic sequence within said sample. Such mutations include deletions, translocations, inversions, and punctual mutations (e.g. mutations known to cause or be associated with one or more disease traits). These mutations are preferably detected using standard molecular biological techniques (e.g., sequencing or PCR) and employing a Nupre nucleotide or fragment thereof as a probe or primer. Preferably, the nucleotide test sample in these methods contains DNA. Identification of either less than two functional copies of the genomic region in which Nupre is located or a mutation in said region is indicative of a likelihood that said individual has a genetic mutation associated with immunodeficiency. Preferably, said Nupre polynucleotide is an oligonucleotide primer of less than 30 nucleotides. Also preferably, a Nupre polynucleotide of SEQ ID NO:7 is used as a probe in such a method. Genotyping of an individual can be performed by any technique well-known to those skilled in the art such as, e.g., sequencing, microsequencing or hybridization. Said Nupre polynucleotide may be used alone or in combination with other polynucleotidic sequences indicative of a likelihood that an individual has a genetic mutation associated with immunodeficiency. An additional aspect of this embodiment relates to an array of oligonucleotides probes comprising a Nupre polynucleotide. Preferably, said array of oligonucleotides probes comprise several oligonucleotides indicative of different genetic mutations associated with immunodeficiency and are used to conduct efficient screening of mutations associated with immunodeficiency. These microarrays can for example be used to perform epidemiological studies in families predisposed to immunodeficiencies, or to diagnose the presence of or predisposition for an immunodeficiency in an individual.

Another embodiment of the present invention is directed to a diagnostic kit for detecting in vitro the presence of a Nupre polypeptide. Such kit comprises: i) an anti-Nupre antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Nupre antibodies is used for diagnosing immunodeficiency disorders such as those listed above. Optionally, said diagnostic kit comprises a negative control sample representative of the level from a normal individual. Optionally, said diagnostic kit comprises a positive control sample representative of the level from an individual suffering from a given immunodeficiency disorder.

Another embodiment relates to a method of producing a Nupre polypeptide comprising the steps of: i) culturing a cell expressing a Nupre polypeptide, and ii) purifying the produced protein. The purification of the protein can be done following any technique well-known to those skilled in the art. Preferably, an anti-Nupre antibody may be bound to a chromatographic support to form an affinity chromatography column. Preferably, the cell expressing a Nupre polypeptide is a recombinant host cell as described below. Producing Nupre polypeptides may be useful in methods and compositions as further described in the present invention.

A preferred aspect of the invention is a host cell recombinant for polynucleotides encoding a Nupre polypeptide, operably linked to a promoter. An embodiment is directed to a method of constructing a host cell recombinant for polynucleotides encoding a Nupre polypeptide comprising the steps of: i) constructing a recombinant vector that comprises a nucleic acid sequence encoding a Nupre polypeptide, operably linked to a promoter, that allows expression of said Nupre polypeptide under given conditions, and ii) introducing said recombinant vector into a cell. In preferred embodiments, the cell is an Escherichia coli cell or a human cell. An additional preferred aspect is a host cell recombinant for polynucleotides that, when present in a cell, cause an alteration in Nupre expression. A host cell recombinant for such polynucleotides may be constructed by a method comprising the step of: i) providing a cell comprising the Nupre gene; and ii) introducing a recombinant vector comprising Nupre expression-altering polynucleotides into said cell, wherein the presence of said polynucleotides in said cell increases or decreases Nupre expression compared to Nupre expression in said cell before said recombinant vector is introduced. Preferably, said polynucleotides are inserted into or replace all or part of the 5′ regulatory region of the Nupre gene. Further preferably, said polynucleotides are located within 500 base pairs of the Nupre coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing Nupre polypeptides may be used to produce Nupre polypeptides.

Another preferred embodiment relates to methods of screening test substances for the ability to modulate Nupre expression comprising the steps of: i) contacting a cell with a test substance; and ii) comparing Nupre expression in the cell after exposure to the test substance to that of an unexposed control cell, wherein an observed difference in Nupre expression between the exposed cell and the unexposed control cell indicates that the test substance modulates Nupre expression. Nupre expression may be determined by methods common to the art or included herein. Methods of determining Nupre expression include but are not limited to methods of quantifying Nupre polynucleotides (e.g., detection of Nupre mRNA by northern blot or RTPCR) or to methods of quantifying Nupre polypeptides (e.g., detection of Nupre polypeptides by western blot or immunochemistry). In one embodiment, the test substance modifies the expression of Nupre in a specific cell type (preferably adipocytes, lymphocytes, glial cells or epithelial cells) but not in others. Modulators identified using such methods are also encompassed by the present invention, as are methods of using the modulators, e.g., to alter the expression of Nupre in a cell, tissue, or individual.

A further embodiment of the present invention is directed to methods of screening test substances for the ability to modulate Nupre activity comprising the steps of: i) contacting a cell with a test substance, and ii) comparing Nupre activity in the cell after exposure to the test substance to that of an unexposed control cell, wherein an observed difference in Nupre activity between the exposed and unexposed cells indicates that the test substance modulates Nupre activity. Nupre activity can be monitored, e.g., by detecting its 5′nucleotidase activity. The 5′nucleotidase activity of Nupre can be assessed using a wide variety of techniques well-known to those skilled in the art. For example, these techniques comprise the ion-exchange column chromatographic method described by Ward et al. (J Chromatogr B Biomed Sci Appl 707:295-300 (1998)) and the methods using NADH and NADPH described in U.S. Pat. No. 5,801,006, the disclosures of both of which are incorporated herein by reference in their entireties. Modulators identified using such methods are also encompassed by the present invention, as are methods of using the modulators, e.g. to alter the activity of Nupre in a cell, tissue, or individual.

Test substances that decrease Nupre expression or activity are defined as Nupre antagonists. Test substances that increase Nupre expression or activity are defined as Nupre agonists. Test substances that modulate the expression or activity of Nupre include, but are not linited to, chemical compounds (e.g. small-molecule inhibitors or activators), oligonucleotides, antisense polynucleotides, polypeptides, ribozymes, dominant negative forms of Nupre, and anti-Nupre antibodies. These substances may be made and used according to methods well known in the art.

A preferred embodiment is directed to methods of producing adenosine comprising the step of: contacting a Nupre polypeptide with-a biological sample comprising a 5′-ribonucleotide under conditions that allow catalysis to occur. A preferred 5′-ribonucleotide is adenosine-5′-monophospate (AMP). A wide variety of conditions allow dephosphorylation of 5′-ribonucleotides to the corresponding ribonucleosides. For example, the conditions described in Navarro et al. (Mol Cell Biochem 187:121-31 (1998)) may be used. Producing adenosine is useful in a number of methods, e.g., the methods further detailed below.

A preferred embodiment is directed to methods of using a Nupre polypeptide for determining the degree of freshness of food (e.g., fish and meat) by monitoring the autolytic degradation of adenosine triphosphate into inosine monophosphate, inosine and hypoxanthine in muscles. Such a method is described in U.S. Pat. No. 5,288,613, which disclosure is hereby incorporated by reference in its entirety.

Another preferred embodiment is directed to methods of using a Nupre polypeptide for increasing adenosine levels and activating adenosine receptor-mediated signalling. Such a method comprises the step of: contacting a tissue comprising cells expressing an adenosine receptor with a Nupre polypeptide or a Nupre agonist under conditions that allow catalytic dephosphorylation of AMP by Nupre polypeptides to occur. Alternatively, AMP analogues that are dephosphorylated into adenosine analogues may be used in such a method. For example, AMP analogues that are dephosphorylated into N6-cyclohexyladenosine, L-N6-phenylisopropyladenosine, D-N6-phenylisopropyladenosine and N6-methyladenosine or 2-chloroadenosine may be used.

In one such embodiment, the present invention provides a method of increasing adenosine levels and activating adenosine receptor-mediated signalling comprising the step of: administering a Nupre polypeptide or a Nupre agonist to a tissue comprising neurons. Preferably, said method is directed to inhibit neurons postsynaptically by inducing or modulating ionic currents and presynaptically by reducing transmitter release. Such a method may be carried out in vitro, e.g., by delivering a Nupre polypeptide or a Nupre agonist to in vitro brain-slice preparations or cultured neurons. Such a method for inhibiting neurons may also be carried out in vivo by delivering a physiologically acceptable carrier and an effective amount of a Nupre polypeptide or a Nupre agonist to an individual. As used herein, an “effective amount of a Nupre polypeptide or a Nupre agonist” refers to an amount of Nupre polypeptide or Nupre agonist that is sufficient to activate adenosine receptor-mediated signalling in a cell such as a neuron. Activating neuronal adenosine receptor-mediated signalling is useful for a number of applications, e.g., to treat neurological and psychiatric diseases such as epilepsy, sleep diseases and movement diseases (e.g., Parkinson's and Huntington's disease). Activating neuronal adenosine receptor-mediated signalling is also useful to treat psychiatric disorders such as schizophrenia, depression, Alzheimer's disease and addiction. Additionally, activating neuronal adenosine receptor-mediated signalling is useful to treat pain. The Nupre polypeptide or Nupre agonist may be administered systemically, for example by intravenous injection, or locally, for example by intracranial injection.

Still another preferred embodiment is directed to methods of using a Nupre polypeptide for lowering adenosine levels and inhibiting adenosine receptor-mediated signalling. Such methods comprise the step of: contacting a tissue comprising cells expressing an adenosine receptor with a Nupre antagonist, wherein said Nupre antagonist inhibits catalytic dephosphorylation of AMP by Nupre polypeptides to occur.

In one aspect, the present invention provides a method of lowering adenosine levels and inhibiting adenosine receptor-mediated signalling for increasing lipolysis in adipocytes. Said method comprises the step of: administering a Nupre antagonist to an adipocyte or adipose tissue. Such a method may be carried out in vitro, e.g., by delivering a Nupre antagonist to in vitro white adipocyte cultures. Such a method may be carried out in vivo by delivering a phamaceutical composition comprising a physiologically acceptable carrier and an effective amount of a Nupre antagonist to an individual. As used herein, an “effective amount of a Nupre antagonist” refers to an amount of Nupre antagonist that is sufficient inhibit adenosine receptor-mediated signalling in a cell such as an adipocyte. Preferably, inhibiting adipocytic adenosine receptor-mediated signalling is used to treat obesity. In another aspect, inhibiting adenosine receptor-mediated signalling is used to regulate coronary blood flow and cardiac conduction. Said method of regulating coronary blood flow and cardiac conduction comprises the step of: delivering a physiologically acceptable carrier and an effective amount of a Nupre antagonist to an individual. Preferably, said method is carried out in vivo by delivering a pharmaceutical composition comprising a physiologically acceptable carrier and an effective amount of a Nupre antagonist to an individual in order to treat or prevent disorders such as, e.g., syncope, heart failure, ischemia-reperfusion injury, or heart failure. Such pharmaceutical compositions may be administered in any way, preferably systemically such as intravenously.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising a Nupre agonist or antagonist and a physiologically acceptable carrier can be for, e.g., intravenous, topical, rectal, local, inhalant, subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a Nupre polypeptide or a nucleic acid sequence that encodes a cDNA that is complementary to Nupre polynucleotides. Methods of constructing transgenic animals are well-known to those skilled in the art. For example, a Nupre deficient mouse may be constructed according to U.S. Pat. No. 6,207,876, which disclosure is hereby incorporated by reference in its entirety. Alternatively, a transgenic mouse over-expressing Nupre may be constructed. Such transgenic animals may be useful for obtaining animal models for dysfunctions associated with low or high adenosine levels, for treating such dysfunctions and for screening compounds for pharmaceutical activity in the treatment of these dysfunctions.

Protein of SEQ ID NO:10 (internal designation Clone 599222_{—}181-13-3-0-F11-F)

The cDNA of Clone 599222_{—}181-13-3-0-F11-F (SEQ ID NO:9) encodes a NOVEL complement H-related protein precursor (NFHR) of SEQ ID NO:10, comprising the amino acid sequence: MWLLVSVILISRISSVGGEVSAEKCGPPPPIDNGDITSFLLSVYAPGSSVEYQCQNLYQLEGNNQIT CRNGQWSEPPKCLDPCVISQEIMEKYNIKLKWTNQQKLYSRTGDIVEFVCKSGYHPTKSHSFRA MCQNGKLVYPSCEEK. Accordingly, it will be appreciated that all characteristics and uses of the polypeptides of SEQ ID NO:10 described throughout the present application also pertain to the polypeptides encoded by the human cDNA included in Clone 599222_{—}181-13-3-0-F11-F. In addition, it will be appreciated that all characteristics and uses of the polynucleotides of SEQ ID NO:9 described throughout the present application also pertain to the nucleic acids comprising the human cDNA in 599222_{—}181-13-3-0-F11-F. A preferred embodiment of the invention is directed toward compositions comprising the polypeptides and polynucleotides shown as SEQ ID NO: 10, SEQ ID NO:9 and the human cDNA and encoded protein of Clone 599222_{—}181-13-3-0-F11-F. Also preferred are polypeptide fragments having a biological activity as described herein and the polynucleotides encoding the fragments.

The protein of SEQ ID:10, NFHR, is a NOVEL splice variant of the complement factor H-related protein 2 precursor (swissprot accession number P36980). NFHR displays 2 Sushi domains:

CGPPPPIDNGDITSFLLSVYAPGSSVEYQCQNLYQLEGNNQITCRNGQWSEPPKC;
and
CVISQEIMEKYNIKLKWTNQQKLYSRTGDIVEFVCKSGYHPTKSHSFRAMCQNGKLVYPSC.

Also, NFHR displays 1 transmembrane domain: GDITSFLLSVYAPGSSVEYQC. Each of these fragments corresponding to these domains is specifically encompassed by the present invention.

NFHR is a secreted plasma protein that is primarily synthesized in the liver and plays an important regulatory role in the complement reaction. Specifically, NFHR binds to C3b and prevents factor H from binding, thereby promoting the binding of factor B to C3b and thereby promoting the complement reaction. Accordingly, levels of NFHR are elevated in complement-mediated inflammatory responses, such as otitis media with effusion (see, e.g. Clin Immunol. 2001 Jul;100(1):118-26, which is hereby incorporated by reference in its entirety. In addition to its role in the complement reaction, NFHR is also associated in plasma with lipoproteins and other proteins, e.g. in a FALP particle that comprises apolipoprotein A-1, lipopolysaccharide binding protein, phospholipids, fibrinogen, and other components (see, e.g., Park et al. (2000) Blood 95(i):198-204, which is incorporated herein by reference in its entirety). Among other activities, these particles facilitate the adhesive response of neutrophils to lipopolysaccharide. Furthermore, NFHR plays a role in carrying and/or regulating the function of lipopolysaccharide binding protein, as well as the coagulation function of fibrinogen. In addition, levels of NFHR are elevated in various cancers, such as bladder cancer.

An embodiment of the invention is directed to a composition comprising an NFHR polypeptide sequence of SEQ ID NO:10, or comprising at least about 70%, 80%, 90%, 95%, 96%, 97%, 98%, 99%, or more identity to SEQ ID NO:10.

A further embodiment of the invention is directed to a composition comprising an NFHR polypeptide fragment having any of the biological activities described herein, including, but not limited to, binding to Apolipoprotein A1, lipopolysaccharide binding protein, fibrinogen, or phospholipids, promotion of the complement system and elevated expression in cancer cells.

A further embodiment of the invention is directed to a composition comprising a polynucleotide sequence of SEQ ID NO:9, or at least about 70%, 80%, 90%, 95%, 96%, 97%? 98%, 99%, or more identity to SEQ ID NO:9. In one embodiment, the present invention provides a polynucleotide sequence that encodes an NFHR polypeptide.

A further embodiment of the invention is directed to a composition comprising a polynucleotide sequence encoding an NFHR polypeptide fragment having any of the biological activities described herein.

A further embodiment of the invention is directed to an antibody recognizing an NFHR polypeptide sequence or an NFHR polypeptide fragment. Preferably, the antibody recognizes a non-linear epitope. As used herein, an “anti-NFHR antibody” refers to an antibody or antigen-binding fragment thereof that specifically binds to an NFHR polypeptide.

An embodiment of the present invention relates to a method of binding an anti-NFHR antibody to an NFHR polypeptide comprising the step of: contacting an NFHR polypeptide with said antibody under conditions that allow binding to occur. Such conditions are well known to those skilled in the art. Such anti-NFHR antibodies and methods of binding NFHR polypeptides using the antibodiescan be used for NFHR purification, detection and diagnosis as described herein.

In another embodiment, the invention relates to methods and compositions for detecting and quantifying the level of NFHR polypeptide present in a biological sample, said method comprising the steps of: i) contacting a biological sample with an antibody or antibody fragment that specifically binds NFHR polypeptide; and ii) detecting the antigen-antibody complex formed. The antibody or antibody fragment may be monoclonal or polyclonal. In addition, the antibody or antibody fragment may be primarily or secondarily labeled by a detectable compound (e.g., radioactive, fluorescent, luminescent, or enzymatic) common in the art. Diagnostic assays to detect the protein of the invention may practiced on any biological sample, including a biopsy, in situ assay of cells from organ or tissue sections, an aspirate of cells from a tumor or normal tissue, a cellular extract from organs, tissues, cells, urine, or serum or blood, or any other bodily fluid or extract. Detection and quantification of NFHR polypeptides using either specific polyclonal or monoclonal antibodies are known in the art. Examples of such techniques include enzyme-linked immunoabsorbent assays (ELISAs), radioimmunoassays (RIAs), and fluorescence activated cell sorting (FACS). Such methods may be used in the diagnosis of any disease or condition in which NFHR polypeptides are present at a higher or lower level than normal. For example, the middle ear effusion from patients with Otitis media with effusion contains elevated levels of complement factor related proteins such as NFHR. In addition, urine samples from patients with bladder cancer contain elevated levels of complement factor related proteins. Accordingly, the present methods may be used to diagnose or detect a predisposition for these diseases or conditions, or in any of a large number of inflammatory or neoplastic conditions or diseases.

In some embodiments, the invention also concerns a diagnostic kit for detecting in vitro the presence of NFHR polypeptide. This kit comprises: a polyclonal or monoclonal antibody or fragment thereof that specifically binds an NFHR polypeptide; and optionally, ii) a reagent allowing the detection of the antigen-antibody complexes formed. Preferably, the antibody or antibody fragment is detectably labeled. Such labels include fluorescent, luminescent, and radioactive compounds, as well as enzymatic substrates. The optional reagent may provide a detectable signal and either bind to the antibody or react with the label on such antibody. NFHR antibodies may be used to diagnose otitis media with effusion or cancers such as bladder cancer. In addition, the levels of NFHR can be used as a marker for lipid metabolism, and can therefore be used to detect any of a number of lipid metabolism related disorders such as, but not limited to, diabetes and atherosclerosis. To detect such disorders, an appropriate biological sample can be tested to determine the level of NFHR being produced.

Certain embodiments of the present invention relate to methods of producing NFHR polypeptides. In one such embodiment, the method comprises the step of: i) transfecting a mammalian host cell with a recombinant expression vector encoding an NFHR polypeptide; ii) culturing the cell under conditions conducive to the expression of the polypeptide; and iii) purifying the expressed polypeptide. The purification of the protein can be done following any technique well known to those skilled in the art. For example, an antibody directed against NFHR or part thereof may be bound to a chromatographic support to form an affinity chromatography column. The NFHR protein purified using the present methods has numerous uses, for example to purify or detect proteins or other molecules associated with NFHR, such as Apolipoprotein A1, lipopolysaccharide binding protein, fibrinogen, phospholipids, factor B, or C3b.

The present invention also provides host cells recombinant for polynucleotides encoding an NFHR polypeptide or a biologically fragment thereof. An additional aspect is a host cell recombinant for polynucleotides that, when present in a cell, result in an alteration in the level of NFHR expression. In one such embodiment, polynucleotides are introduced into the 5′ regulatory region of the NFHR gene, resulting in the increase or decrease of the endogenous level of expression of the NFHR gene. Further preferably, the polynucleotides are located within 50 base pairs of the NFHR coding region. The polynucleotides may comprise a promoter sequence. Techniques known in the art for introducing polynucleotides sequences to endogenous sequences are described in U.S. Pat. No. 5,641,670 and PCT WO9629411, which disclosures are hereby incorporated by reference in their entireties.

In a preferred embodiment, an NFHR polypeptide is used to detect or purify an NFHR-interacting protein or molecule, such as Apolipoprotein A1, lipopolysaccharide binding protein, fibrinogen, phospholipids, factor B, or C3b. In one such method, the NFHR polypeptide is covalently or non-covalendy attached to a solid matrix and contacted with a solution containing the interacting molecule, thereby removing the interacting molecule and allowing its purification or quantification. Typically, this method further comprises the steps of: i) washing the solid matrix to get rid of contaminants, ii) eluting the particle of interest using more stringent conditions. In another embodiment, the method comprises the steps: i) obtaining a biological sample suspected of containing an NFHR-interacting molecule; ii) contacting said sample with an NFHR polypeptide or fragment thereof under conditions suitable for binding of NFHR; and iii) detecting the presence of the interacting molecule by detecting NFHR. Preferably, the NFHR polypeptide or fragment thereof is covalently attached to a detectable compound such as a fluorescent label. Alternatively, a detectable NFHR-specific antibody or fragment thereof may be used to detect NFHR. This embodiment is useful, for example, as a diagnostic tool for detecting plasma levels of NFHR interacting molecules such as Apolipoprotein A1, lipopolysaccharide binding protein, fibrinogen, or phospholipids. Also, purified interacting molecules may be used for numerous purposes. For example, purified Apolipoprotein A1 can be used to treat atherosclerosis and cardiovascular diseases according to the methods described by Ageland et al in U.S. Pat. No. 5,990,081, which disclosure is hereby incorporated by reference in its entirety. Also, purified fibrinogen can be administered, e.g. in the treatment of forms of hemophilia (see, e.g., Di Paola et al. (2001) Haemophilia Jan;7 Suppl 1:16-22, the entire disclosure of which is incorporated herein in its entirety by reference). In addition, purified lipopolysaccharide binding protein can be used to reduce the inflammatory potential of bacteria and their endotoxin when administered to certain subjects prior to exposure to the bacterial endotoxin according to the methods described in U.S. Pat. No. 6,306,824, which disclosure is hereby incorporated by reference in its entirety.

In another embodiment of the invention, the NFHR polypeptide is used promote the complement reaction or coagulation in an individual. This method comprises the step of: introducing an effective amount of NFHR polypeptide or fragment thereof to the bloodstream of an individual. A preferred method of delivering NFHR polypeptides or biologically active fragments thereof to an individual includes direct, intravenous injection of said polypeptides or fragments in a physiologically acceptable solution (e.g., pH-buffered isotonic saline solutions, pH-buffered isotonic saline solutions modified by addition of viscous elements such as glycerol). Alternatively, NFHR polynucleotides may be introduced to express NFHR polypeptides in the bloodstream. This method comprises the step of: introducing into a cell of a patient a recombinant vector a polynucleotide encoding an NFHR polypeptide operatively linked to a promoter, wherein the NFHR polypeptide is expressed within the cell. Any vector can be used in such methods, including viral (adenovirus, adeno-associated virus, HSV, retroviruses, etc.) or non-viral vectors (e.g. plasmids, naked DNA, etc.) The vector may be introduced into the individual or into the cell using any means, e.g. liposome-mediated transfection, viral infection, biolistic particles, etc.) Such methods are useful, e.g., for promoting inflammatory responses, e.g. to aid in defense against infections.

In another embodiment, NFHR is used to determine circulating levels of an NFHR-interacting molecule such as Apolipoprotein A1, lipopolysaccharide binding protein, fibrinogen, phospholipids, factor B, or C3b in the blood of an individual, the method comprising obtaining a blood sample from the individual, using the protein of the invention to purify the interacting molecule from the blood sample (e.g. by affinity column chromatography), and measuring the level of the purified molecule. Any method may be used to assess the level of the purified molecule, such as ELISA, western blot, mass spectrometry, or radioimmunoessay (RIA). Determining, e.g., Apolipoprotein A1, lipopolysaccharide binding protein and phospholipids levels in circulating blood could be of special interest for the monitoring of patients with diseases such as, but not limited to, lipid metabolism related disorders, diabetes, and atherosclerosis.

Another embodiment of the invention relates to compositions and methods using polynucleotide sequences encoding the protein of the invention or fragment thereof to establish transgenic model animals (e.g. D. melanogaster, M. musculus), by any method familiar to those skilled in the art. By modulating in vivo the expression of the transgene with drugs or modifier genes (activator or suppressor genes), animal models can be developed that mimic lipid-metabolism related disorders. These animal models would thus allow the identification of potential therapeutic agents for treatment of the disorders described above. In addition, recombinant cell lines derived from these transgenic animals may be used for similar approaches ex vivo.

In another embodiment, the present invention provides a method of detecting the presence of, or a predisposition for, cancer such as bladder cancer. Such methods typically involve the steps of: i) providing a biological sample from a patient suspected of having cancer or being at risk of developing cancer; ii) and detecting the level of NFHR in the sample, wherein an elevated level of NFHR in the sample relative to a level expected of a normal, cancer-free individual (or an individual without an elevated risk of developing cancer) indicates that the patient has cancer or is at an elevated risk of developing cancer. Such methods may be used to detect any type of cancer, including carcinoma, lymphoma, blastoma, sarcoma, and leukemia, more specifically, breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. In a preferred embodiment, the cancer is bladder cancer.

Protein of SEQ ID NO:12 (Internal Designation Clone 238757_{—}116-105-3-0-D9-F)

The cDNA of Clone 238757_{—}116-105-3-0-D9-F (SEQ ID NO:11) encodes NARF protein of SEQ ID NO:12, comprising the amino acid sequence: MSDLRITEAFLYMDYLCFRALCCKGPPPARPEYDLVCIGLTGSGKTSLLSKLCSESPDNVVSTTGF SIKAVPFQNAILNVKELGGADNIRKYWSRYYQGSQGVIFVLDSASSEDDLEAARNELHSALQHPQ LCTLPFLILANHQDKPAARSVQEKIQD. Accordingly, it will be appreciated that all characteristics and uses of the polypeptides of SEQ ID NO:12 described throughout the present application also pertain to the polypeptides encoded by the human cDNA included in Clone 238757_{—}116-105-3-0-D9-F. In addition, it will be appreciated that all characteristics and uses of the polynucleotides of SEQ ID NO:11 described throughout the present application also pertain to the nucleic acids comprising the human cDNA in Clone 238757_{—}116-105-3-0-D9-F. A preferred embodiment of the invention is directed toward the compositions of SEQ ID NO:12, SEQ ID NO:11 and Clone 238757_{—}116-105-3-0-D9-F. Also preferred are polypeptide fragments having a biological activity as described herein and the polynucleotides encoding the fragments.

The protein of SEQ ID NO:12, NARF, is a NOVEL ADP ribosylation family related protein. NARF displays an ADP ribosylation factor family (ARF) motif:

YLCFRALCCKGPPPARPEYDLVCIGLTGSGKTSLLSKLCSESPDNVVSTTGFSIKAVPFQNAILNV
KELGGADNIRKYWSRYYQGSQGVIFVLDSASSEDDLEAARNELHSALQHPQLCTLPFLILANHQ
DKPAARSVQEKIQD.

ARF proteins are members of the ras superfamily, and become active and membrane associated when bound to GTP. The biological roles of ARFs are central to many steps in signalling, vesicular traffic, particularly those involving the Golgi and in regulation of phospholipid-modifying enzyme activities and cytoskeletons. For example, NARF is required for association of non-clathrin coat proteins with intracellular transport vesicles and is critical during an early step in endocytosis as well as in nuclear vesicle fusion. NARF plays also an important role in epithelial cells and in secretion. ARF proteins also activate various proteins and processes, such as phospholipase D and cholera toxin mediated ADP ribosylation. The activation of phospholipase D leads to multiple cellular effects. For example, following its activation by NARF, phospholipase D hydrolyzes phosphatidylcholine. The phosphatidic acid produced by this reaction facilitates formation of stable binding sites for coatomer, leading to budding of coated vesicles.

An embodiment of the invention is directed to a composition comprising a NARF polypeptide sequence of SEQ ID NO:12. In another embodiment, the polypeptide sequence is at least 70%, 80%, 90%, 95%, or more identical to SEQ ID NO:12.

A further embodiment of the invention is directed to a composition comprising a NARF polypeptide fragment having any of the biological activities described herein.

A further embodiment of the invention is directed to a composition comprising a polynucleotide sequence of SEQ ID NO:11 encoding a NARF polypeptide. In another embodiment, the polynucleotide sequence has at least 70%, 80%, 90%, 95%, or more identity to SEQ ID NO:11.

In another embodiment, the present invention provides a polynucleotide that encodes a polypeptide comprising the sequence of SEQ ID NO:12, or a biologically active fragment thereof.

A further embodiment of tne invention is directed to a composition comprising a polynucleotide sequence encoding a NARF polypeptide having any of the herein-described biological activities.

A further embodiment of the invention is directed to a method of binding an antibody to a NARF polypeptide or fragment thereof. Preferably, the antibody recognizes a non-linear epitopes, and specifically binds to the C-terminus of NARF. Such NARF-specific antibodies can be used for NARF purification, inhibition, detection and diagnosis, e.g., as described herein.

An embodiment of the present invention relates to methods of producing NARF polypeptides. The method of producing NARF polypeptides comprises the steps of: i) transfecting a mammalian host cell with a recombinant expression vector encoding a NARF polypeptide of the present invention, ii) culturing the cell under conditions conducive to the expression of said polypeptide; and iii) purifying the expressed NARF polypeptide. The purification of the protein can be done using any technique well known to those skilled in the art. Preferably, an antibody directed against NARF, such as an antibody directed against the NARF C-terminus, is bound to a chromatographic support to form an affinity chromatography column. The purified protein can be used for any of a number of applications, such as activating phospholipase D, generating anti-NARF antibodies, screening for NARF modulators, or modulating membrane trafficking in a cell.

A preferred aspect of the invention is a host cell recombinant for polynucleotides encoding a NARF polypeptide or a biologically active fragment thereof. An additional preferred aspect is a host cell recombinant for polynucleotides capable of directing NARF expression. Preferably, the polynucleotides capable of directing NARF expression are located in the 5′ regulatory region of the NARF gene. Further preferably, these polynucleotides are located within 500 base pairs of the NARF coding region. These polynucleotides preferably comprise a promoter sequence. Techniques known in the art for introducing polynucleotide sequences to endogenous sequences are described in U.S. Pat. No. 5,641,670 and PCT WO9629411, which disclosure are hereby incorporated by reference in their entireties. NARF protein produced by said host cell may be used as described herein, e.g., for in vitro detection and purification methods as well as diagnosis and in vivo applications.

ARFs mRNA is differentially expressed in some processes essential to nerve regeneration as well as neuronal differentiation and maturation (see Suzuki, I. et al. (2001) Brain Res. Mol. Brain Res. 31:124-34; which disclosure is hereby included by reference in its entirety). In another embodiment, the level of NARF mRNA is detected in a mammal, preferably a human, for the determination or diagnosis of whether someone is at risk of developing or has a neuronal disorder, ADP-ribosylation deficiency disease, epithelial disorder and/or secretory disorders. Assays for the detection and/or quantification of mRNA are well known in the art (see, for example, Maniatis, Fitsch and Sambrook, Molecular Cloning; A Laboratory Manual (1982), or Current Protocols in Molecular Biology, Ausubel, F. M. et al. (Eds), Wiley & Sons, Inc.). In one such embodiment, a nucleic acid probe is labeled by standard methods and added to a biological sample under conditions that allow the formation of hybridization complexes. After an incubation period, the sample is washed and the amount of label associated with hybridization is quantified and/or compared with a control value. If the amount of label in the sample is detectably different from the control value, then the presence of the associated condition, disease or disorder, or of a predisposition thereto, is indicated. Such assays may also be used to evaluate the efficacy of a particular therapeutic treatment regimen in animal studies, in clinical trials, or in the monitoring of a treatment regimen in an individual patient. Once the presence of a condition is established and a treatment protocol is initiated, these diagnostic assays may be repeated on a regular basis to determine if the level of expression in the patient begins to approximate that which is observed in a normal subject.

Since NARF is associated with Golgi, endoplasmic Reticulum (ER) as well as endosomes, NARF can be useful as a marker for secretion or intracellular trafficking, and therefore it can be used to diagnose diseases associated with abnormal trafficking. Such Goldi, ER or endomes-related secretory disorders can include, but are not limited to, cystic fibrosis, menkes syndrome, Niemann-Pick disease, and other conditions associated with abnormal vesicle trafficking, including, but not limited to, acquired immunodeficiency syndrome (AIDS). Also, NARF is useful to diagnose epithelial disorders including, but not limited to, eczema, dermatitis, psoriasis, acne dermatomyositis and irritable bowel syndrome.

Another embodiment of the present invention relates to methods of diagnosing epthielial and/or secretory disorders by detecting NARF polypeptides. Diagnostic assays to detect and/or quantify the level of NARF polypeptides can be performed on any biological sample, including a biopsy, in situ assay of cells from organ or tissue sections, an aspirate of cells from a tumor or normal tissue, cellular extracts from organs, tissues, cells, urine, or serum or blood, or any other body fluid or extract. Detection and quantification of NARF polypeptides using either specific polyclonal or monoclonal antibodies are known in the art. Examples of such techniques include enzyme-linked immunoabsorbent assays (ELISAs), radioimmunoassays (RIAs), and fluorescence activated cell sorting (FACS).

In another embodiment, a pharmaceutical composition comprising a substantially purified NARF polypeptide and a pharmaceutical carrier may be administered to a subject to treat or prevent a condition associated with altered expression or activity of NARF including, but not limited to, those diseases and conditions listed above.

A preferred embodiment of the invention is a method of using NARF to bind cholera toxin. This method comprises the step of contacting a NARF polypeptide or active fragment thereof with a cholera toxin under conditions that allow NARF binding, whereby binding inhibits the activity of said cholera toxin. This method may be applied to in vitro uses (e.g., detection of cholera toxin).

A preferred embodiment of the invention is a method of purifying cholera toxins. This method comprises the steps of: contacting a NARF polypeptide or a biologically active fragment thereof with a cholera toxin under conditions that allow binding; removing contaminants; and eluting the cholera toxin with more stringent conditions. Preferably, the NARF peptide is immobilized on a solid or semi-solid matrix to facilitate washing of sample to remove contaminants. These may be purified from common biological fluids such as cell culture media and body fluids. Purified cholera toxin can be useful as vaccines or in the treatment of inflammatory bowel disease (see for example PCT WO0134175A2, which disclosure is hereby included in its entirety).

A preferred embodiment of the invention provides a method of screening for modulators of NARF polypeptides comprising the steps of: contacting a cell with a test substance, detecting the level of NARF activity in the cell, and comparing the level of NARF activity in the cell to that in an unexposed control cell, wherein an increase in the level of NARF activity in the exposed cell in comparison with the level in an unexposed cell indicates that the test substance is an agonist of NARF polypeptides and wherein a decrease in the level of NARF activity in the exposed cell in comparison with the level in unexposed cell indicates that the test substance is an antagonist.

A preferred embodiment of the invention provides a method of using modulators of NARF polypeptides in the treatment of diseases associated with secretory or epithelial disorders, such as those listed above. These methods comprise the step of administering a therapeutically effective amount of a NARF modulator in a physiologically acceptable composition to an individual in need of treatment.

In an additional embodiment, an expression vector encoding a NARF polypeptide, or biologically active fragment thereof, is administered to a subject to treat or prevent any of the diseases or conditions listed above.

In one embodiment, the present invention relates to methods of administering NARF to a subject to treat an ADP-ribosylation deficiency disease, epithelial and/or secretory disorder, such as any of those listed above. NARF polypeptides may be administered alone or in combination with other agents. Another embodiment of the invention provides methods of activating phospholipase D with NARF. Such methods comprise the step of contacting phospholipase D with a NARF polypeptide under conditions suitable to activate phospholipase D. The activation of phospholipase D by NARF can be accomplished using any technique well known in the art or as described in U.S. Pat. No. 6,043,073, which disclosure is hereby included in its entirety. This method is useful, e.g., in the treatment of autoimmune or inflammatory diseases such as rheumatoid arthritis, psoriasis ulcerative colitis, wound healing, or any other disease or condition characterized by exhibition of an inflammatory response, or in the treatment of cancer.

In some embodiments, the invention also concerns a diagnostic kit for detecting in vitro the presence of NARF polypeptides or polynucleotides. In one embodiment, this kit comprises: a polyclonal or monoclonal antibody or fragment thereof that specifically binds a NARF polypeptide; and optionally, ii) a reagent allowing the detection of the antigen-antibody complexes formed. Preferably, the antibody, or antibody fragment is detectably labeled.

Such labels may include fluorescent, luminescent, and radioactive compounds, as well as enzymatic substrates.

Alternatively, the optional reagent may provide a detectable signal. The optional reagent may either bind to the antibody or react with the label on said antibody. Preferably, the kit comprising anti-NARF antibodies is used for diagnosing any of the diseases or conditions listed above. Optionally, said diagnostic kit comprises a negative control sample representative of the level expected from a normal individual. Optionally, said diagnostic kit comprises a positive control sample representative of the level expected from an individual suffering from any of the diseases or conditions listed above. In another embodiment, a kit may comprise a reagent for detecting NARF polynucleotides in a sample, such as a labeled NARF polynucleotide probe.

In another embodiment, one may use competitive drug screening assays in which neutralizing antibodies capable of binding NARF specifically compete with a test compound for binding NARF. In this manner, antibodies can be used to detect the presence of any peptide which shares one or more antigenic determinants with NARF.

Another embodiment of the invention relates to composition and methods using polynucleotide sequences encoding the protein of the invention or a fragment thereof to establish transgenic model animals (D. melanogaster, M. musculus), by any method familiar to those skilled in the art. By modulating in vivo the expression of the transgene with drugs or modifier genes (activator or suppressor genes), animal models can be developed that mimic secretory and/or epithelial disorders such as those described above cancers. These animal models would thus allow the identification of potential therapeutic agents for treatment of the disorders. In addition, recombinant cell lines derived from these transgenic animals may be used for similar approaches ex vivo.

Protein of SEQ ID NO:14 (Internal Designation Clone 106614_{—}105-031-2-0-E2-F)

The cDNA of Clone 106614_{—}105-031-2-0-E2-F (SEQ ID NO:13) encodes SgIIb of SEQ ID NO:14, comprising the amino acid sequence: MKSILFVLSLLLILEKQAAVMGQKGGSKGQLPSGSSQFPHGQKGQHYFTGQKDQQHTKSKGSFSI QHTYHVDINDHDWTRKSQQYDLNALHKATKSKQHLGGSQQLLNYKQEGRDHDKSKGHFHMIV IHHKGGQAHHGTQNPSQDQGNSPSGKGLSSQCSNTEKRLWVHGLSKEQASASGAQKGRTQGGS QSSYVLQTEELVVNKQQRETKNSHQNKGHYQNVVDVREEHSSKLQTSLHPAHQDRLQHGPKDI FITQDELLVYNKNQHQTKNLSQDQEHGRKAHKISYPSSRTEERQLHHGEKSVQKDVSKGSISIQT EEKIHGKSQNQVTIHSQDQEHGHKENKISYQSSSTEERHLNCGEKGIQKGVSKGSISIQTEEQIHG KSQIQTPNPNQDQWSGQNAKGKSGQSADSKQDLLSHEQKGRYKQESSESHNIVITEHEVAQDDH LTQQYNEDRNPIST. Accordingly, it will be appreciated that all characteristics and uses of the polypeptides of SEQ ID NO:14 described throughout the present application also pertain to the polypeptides encoded by the nucleic acids comprising the human cDNA in Clone 106614_{—}105-031-2-0-E2-F. In addition, it will be appreciated that all characteristics and uses of the polynucleotides of SEQ ID NOs:13 described throughout the present application also pertain to the nucleic acids comprising the human cDNA in Clone 106614_{—}105-031-2-0-E2-F. A preferred embodiment of the invention is directed toward the compositions of SEQ ID NO:14, SEQ ID NO:13, and Clone 106614_{—}105-031-2-0-E2-F. Preferred SgIIb polypeptide fragments for uses in the methods described below include the SgIIb polypeptide comprising the amino sequence of: QKGGSKGQLPSGSSQFPHGQKGQHYFGQKDQQHTKSKGSFSIQHTYHVDINDHDWTRKSQQYD LNALHKATKSKQHLGGSQQLLNYKQEGRDHDKSKGHFHMIVIHHKGGQAHHGTQNPSQDQGN SPSGKGLSSQCSNTEKRLWVHGLSKEQASASGAQKGRTQGGSQSSYVLQTEELVVNKQQRETK NSHQNKGHYQNVVDVREEHSSKLQTSLPAHQDRLQHGPKDIFTTQDELLVYNKNQHQTKNLS QDQEHGRKAHKISYPSSRTEERQLHHGEKSVQKDVSKGSISIQTEEKIHGKSQNQVTIHSQDQEH GHKENKISYQSSSTEERHLNCGEKGIQKGVSKGSISIQTEEQIHGKSQIQTPNPNQDQWSGQNAKG KSGQSADSKQDLLSHEQKGRYKQESSESHNIVITEHEVAQDDHLTQQYNEDRNPIST. Also preferred are the SgIIb polypeptide fragments comprising the amino sequence of: IQTPNPNQDQWSGQNAKGKSGQSADSKQDLLSHEQKGRYKQESSESHNIVITEHEVAQDDHLTQ QYNEDRNPIST. Also preferred are polypeptide fragments having a biological activity as described herein and the polynucleotides encoding the fragments.

The protein of the invention, SgIIb, is a NOVEL, secreted, splice and polymorphism variant of the semenogelin II protein (SgII, Genbank accession number M81651). The second exon of the SgII gene is split into 2 exons and 1 intron in the SgIIb mRNA. Furthermore, the amino-acid shown at position 305 of SEQ ID NO:14 is an isoleucine in SgIIb and an asparagine in SgII. The resulting full-lenght polypeptides are identical on their 387 amino-terminal amino-acids. The 75 carboxyl-terminal amino-acids of SgIIb are unique to this splice variant. SgIIb displays a signal peptide (MKSIILFVLSLLLILEKQAAVMG), and the mature SgIIb polypeptide is 439 amino-acids long.

SgIIb is a new member of the semenogelin family. SgIIb is a secretory protein that is specifically expressed in the seminal vesicles. SgIIb is a component of the sperm-entrapping gel that is formed immediately after ejaculation. SgIIb interacts non-covalently via disulfide bridges with other semenogelins such as SgI and SgII to instantly form a coagulum upon ejaculation. Limited proteolysis of SgIIb by the prostate-secreted PSA serine protease participates to progressive dissolution of the sperm-entrapping gel, which permits the release of motile spermatozoa. In addition, the peptides released by SgIIb proteolysis bind to acceptor sites on spermatozoa and enhance functions such as capacitation and egg-binding. Furthermore, SgIIb is ectopically expressed in various carcinomas.

An embodiment of the invention is directed to a composition comprising a SgIIb polypeptide sequence of SEQ ID NO:14, or having at least 70%, 80%, 90%, 95%, 96%, 97%, 98%, 99%, or more identity to SEQ ID NO:14.

A further embodiment of the invention is directed to a composition comprising a SgIIb polypeptide fragment having a structural role in the sperm-entrapping gel that is formed after ejaculation, having a role in spermatozoa capacitation and egg-binding, or being cleaved by PSA.

As used herein, a “SgIIb polypeptide” refers either to a SgIIb polypeptide sequence of SEQ ID NO:14 or at least 70%, 80%; 90%, 95%, 96%, 97%, 98%, 99%, or more identical to SEQ ID NO:14, or to a SgIIb polypeptide fragment having a structural role in the sperm-entrapping gel that is formed after ejaculation, having a role in spermatozoa capacitation and egg-binding, or being cleaved by PSA.

An embodiment of the invention is directed to a composition comprising a polynucleotide sequence of SEQ ID NO:13 encoding a SgIIb polypeptide.

A further embodiment of the invention is directed to a composition comprising a polynucleotide sequence encoding a SgIIb polypeptide.

As further used herein, a “SgIIb polynucleotide” refers either to a SgIIb polynucleotide sequence of SEQ ID NO:13 or having at least 70%, 80%, 90%, 95%, 96%, 97%, 98%, 99%, or more identity to SEQ ID NO:13, to a nucleotide sequence encoding a SgIIb polypeptide, or to a polynucleotide fragment of SEQ ID NO:13.

A further embodiment of the invention is directed to an antibody recognizing a SgIIb polypeptide sequence of SEQ ID NO:14 or a SgIIb polypeptide fragment. Preferably, the antibody recognizes one or more of the 75 carboxyl-terminal amino-acids of SgIIb, wherein said one or more amino-acids are required for binding of the antibody to a SgIIb polypeptide. More preferably, the antibody recognizes the QTEEQIHGKSQIQTPNPNQDQWSGQNAKGKSGQSADS epitope, the SHEQKGRYKQES epitope or the DHLTQQYNEDRNPIS epitope. Most preferably, the antibody binds to SgIIb but not to SgII. As used herein, an “anti-SgIIb antibody” refers to an antibody or antigen-binding fragment thereof that specifically binds to a SgIIb polypeptide.

An embodiment of the present invention relates to a method of binding an anti-SgIIb antibody to a SgIIb polypeptide comprising the step of: contacting a SgIIb polypeptide with said antibody under conditions that allow binding to occur. Such conditions are well known to those skilled in the art. Such methods are useful for detecting SgIIb polypeptides as further described herein.

An embodiment of the present invention is directed to a method of detecting SgIIb polypeptides in a biological sample, said method comprising the steps of: i) contacting a biological sample with an anti-SgIIb antibody; and ii) detecting the antigen-antibody complex formed. The anti-SgIIb antibody may be monoclonal or polyclonal. In addition, the anti-SgIIb antibody may be primarily or secondarily labeled by any detectable compound (e.g., radioactive, fluorescent, luminescent, or enzymatic) common in the art. Detecting SgIIb polypeptides is for example useful to corroborate an alleged rape in the field of forensic science. Using an anti-SgIIb antibody rather than a spermatozoon-specific antibody for corroborating a sexual-assault is particularly reliable as SgIIb is even present in the semen of vasectomized men and of men suffering from azoospermia. Such a method of using SgIIb in the field of forensic science may be performed as described in U.S. Pat. No. 5,047,508, which disclosure is hereby incorporated by reference in its entirety.

An embodiment of the present invention is directed to methods of detecting SgIIb expression in a biological sample, said methods comprising the steps of: i) providing a biological sample from an individual; ii) detecting the level of SgIIb expression in the sample; and iii) comparing the level of SgIIb expression in said sample to that of a control sample. The level of SgIIb expression in the sample can be assessed using any method, such as by detecting the level of SgIIb mRNA or SgIIb polypeptides in the sample. For example, well-known techniques such as, e.g., western blot, immunochemical techniques and cytochemical techniques may be used to detect SgIIb expression. Preferably, anti-SgIIb antibodies are used to detect the level of SgIIb expression. Also preferably, polynucleotide probes comprising a polynucleotide sequence of SEQ ID NO:13 or part thereof, or a polynucleotide sequence complementary of SEQ ID NO:13, are used to detect the level of SgIIb expression by, e.g., northern blot or RTPCR techniques.

In one such embodiment, detecting the level of SgIIb expression is directed to diagnose agenesis of seminal vesicles and vas deferens in infertile men. A lower level of SgIIb expression in said biological sample in comparison to the level in a control sample representative of a level in a normal individual is indicative of agenesis of seminal vesicles and vas deferens. Preferred biological sample is obtained from semen. Such a method may for example be performed as described in Calderon et al. (J Androl 15:603-7 (1994)), the disclosure of which is incorporated herein by reference in its entirety.

In another such embodiment, detecting the level of SgIIb expression is used for diagnosing cancers such as, e.g., small cell lung carcinoma, papillary renal cell carcinoma and colon cancer. A higher level of SgIIb expression in said biological sample in comparison to the level in a control sample representative of a level in a normal individual is indicative of malignant tumor burden. Preferred biological sample is obtained from biopsied tissue. Other preferred biological sample is obtained from peripheral blood. This method may for example be performed as described in Rodrigues et al. (Clin Cancer Res. 7:854-60 (2001)), the disclosure of which is incorporated herein by reference in its entirety.

In still another such embodiment, detecting the level of SgIIb expression is used for diagnosing prostate carcinomas. Detecting the level of SgIIb expression for diagnosing prostate carcinomas may for example be performed as described in U.S. Pat. No. 5,972,615, the disclosure of which is incorporated herein by reference in its entirety.

Another embodiment of the present the invention is directed to a diagnostic kit for detecting in vitro the presence of a SgIIb polypeptide. Such kit comprises: i) an anti-SgIIb antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. The kit comprising anti-SgIIb antibodies can used for diagnosing agenesis of seminal vesicles and vas deferens or for diagnosing various cancers such as those listed above. Optionally, said diagnostic kit comprises a negative control sample representative of a level from a normal individual. Optionally, said diagnostic kit comprises a positive control sample representative of a level from an individual suffering from a given disorder.

Another embodiment relates to a method of producing a SgIIb polypeptide comprising the steps of: i) culturing a cell expressing a SgIIb polypeptide, and ii) purifying the produced protein. The purification of the protein can be done following any technique well-known to those skilled in the art. Preferably, an anti-SgIIb antibody may be bound to a chromatographic support to form an affinity chromatography column. Preferably, the cell expressing a SgIIb polypeptide is a recombinant host cell as described below. Producing SgIIb polypeptides may be useful in methods and compositions as further described in the present invention.

A preferred aspect of the invention is a host cell recombinant for polynucleotides encoding a SgIIb polypeptide. An embodiment is directed to a method of constructing a host cell recombinant for polynucleotides encoding a SgIIb polypeptide comprising the steps of: i) constructing a recombinant vector that comprises a nucleic acid sequence encoding a SgIIb polypeptide, operably linked to a promoter, that allows expression of said SgIIb polypeptide under given physiological conditions, and ii) introducing said recombinant vector into a cell. In preferred embodiments, said cell is an Escherichia coli cell or into a human cell. An additional preferred aspect is a host cell recombinant for polynucleotides that, when present in a cell, cause an alteration in SgIIb expression. A host cell recombinant for polynucleotides modifying SgIIb expression may be constructed by a method comprising the step of: i) providing a cell comprising the SgIIb gene; and ii) introducing a recombinant vector comprising polynucleotides into said cell, wherein the presence of said polynucleotides in said cell increases or decreases SgIIb expression compared to SgIIb expression in said cell before said recombinant vector is introduced. Preferably, said polynucleotides are inserted into or replace all or part of the 5′ regulatory region of the SgIIb gene. Further preferably, said polynucleotides are located within 500 base pairs of the SgIIb coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing SgIIb polypeptides may be used to produce SgIIb polypeptides.

Another preferred embodiment relates to methods of screening test substances for the ability to modulate SgIIb expression comprising the steps of: i) contacting a cell with a test substance; and ii) comparing SgIIb expression in the cell after exposure to the test substance to that of an unexposed control cell, wherein an observed difference in SgIIb expression between the exposed cell and the unexposed control cell indicates that the test substance modulates SgIIb expression. SgIIb expression may be determined by methods common to the art or included herein. Methods of determining SgIIb expression include but are not limited to methods of quantifying SgIIb polynucleotides (e.g., detection of SgIIb mRNA by northern blot or RTPCR) or to methods of quantifying SgIIb polypeptides (e.g., detection of SgIIb polypeptides by western blot or immunochemistry). Preferably, the test substance modifies the expression of SgIIb in a specific cell type (preferably epididymal cells) while not in others. Most preferably, the test substance modifies SgIIb expression specifically in epididymal cells. Modulators identified using such methods are also encompassed by the present invention, as are methods of using the modulators, e.g., to alter the expression of SgIIb in a cell, tissue or individual.

A further embodiment of the present invention is directed to methods of screening test substances for the ability to modulate SgIIb activity comprising the steps of: i) contacting a cell with a test substance, and ii) comparing SgIIb activity in the cell after exposure to the test substance that of an unexposed control cell, wherein an observed difference in SgIIb activity between the exposed and unexposed cells indicates that the test substance modulates SgIIb activity. SgIIb activity can for example be monitored by studying gelation and liquefaction of human semen. Gelation and liquefaction of human semen can for example be assessed as described in Lilja et al. (J Clin Invest. 80:281-5 (1987)), which disclosure is incorporated herein by reference in its entirety. Alternatively, activity of a SgIIb polypeptide can be monitored by studying sperm capacitation. Sperm capacitation can for example be measured as described in Ericsson et al. (Proc Soc Exp Biol Med. 125:1115-8 (1967)), disclosure of which is incorporated by reference in its entirety. Modulators identified using such methods are also encompassed by the present invention, as are methods of using the modulators, e.g., to alter the expression of SgIIb in a cell, tissue or individual.

Test substances that decrease SgIIb expression or activity are defined as SgIIb antagonists. Test substances that increase SgIIb expression or activity are defined as SgIIb agonists. Test substances that modulate the expression or activity of SgIIb include, but are not limited to, chemical compounds (e.g. small-molecule inhibitors or activators), oligonucleotides, antisense polynucleotides, polypeptides, ribozymes, dominant negative forms of SgIIb and anti-SgIIb antibodies. These substances may be made and used according to methods well known in the art.

A further embodiment of the present invention is directed to methods of screening SgIIb polypeptides that are recognized and proteolytically cleaved by PSA. Preferred such SgIIb polypeptides are oligopolypeptides of less than 20 amino-acids. Said method comprises the steps of: i) identifying a PSA-mediated cleavage site of SgIIb; ii) preparing SgIIb oligopeptides that comprise said PSA-mediated cleavage site; and iii) assessing the recognition of said SgIIb oligopeptides by free PSA. Said method can be performed as described in U.S. Pat. No. 6,143,864, disclosure of which is incorporated by reference herein in its entirety.

A further embodiment of the present invention is directed to methods of using SgIIb polypeptides that are recognized and proteolytically cleaved by PSA.

In one aspect, said SgIIb polypeptides that are cleaved by PSA are used in assays for measuring free PSA activity. Such assays comprise the steps of i) labeling said SgIIb polypeptide so that one can measure the appearance of such a label in both the uncleaved peptide and the portion of the peptide remaining after cleavage which contains the label; ii) reacting said labeled SgIIb polypeptides with a test sample suspected of containing PSA under conditions that allow proteolytic degradation of SgIIb by PSA to occur; and iii) measuring the amount of said SgIIb polypeptide that has been cleaved. Such an assay for measuring free PSA activity can be performed as described in U.S. Pat. No. 6,143,864. The test sample may be obtained from physiological fluids and tissues. Measurement of serum PSA activity is useful for a number of applications, e.g., for monitoring the treatment of adenocarcinomas of the prostate, for following isolation and purification of free PSA or for screening inhibitors of the proteolytic activity of free PSA.

In another aspect, said SgIIb polypeptides that are cleaved by PSA are used as carriers for cytotoxic prodrugs. In such a method, SgIIb oligopolypeptides that are cleaved by PSA are covalently bonded, directly or through a chemical linker, to a cytotoxic agent. As used herein, “a SgIIb conjugate” refers to a cytotoxic agent bounded to a SgIIb oligopeptide that is cleaved by PSA. Preferably, the cytotoxic activity of the cytotoxic agent is greatly reduced or absent when the SgIIb conjugate is intact. Also preferably, the cytotoxic activity of the cytotoxic agent increases significantly or returns to the activity of the unmodified cytotoxic agent upon proteolytic cleavage by PSA of the SgIIb oligopeptide. As PSA has a highly restricted tissue distribution and is expressed in epithelial cells of the prostate gland, the active cytotoxic agent is specifically released to prostate in such a method. Cytotoxic agents that can be used in such methods comprise, but are not limited to, doxorubicin, carminomycin, daunorubicin, aminopterin, methotrexate, methopterin and vinblastine. Such SgIIb conjugates can be made and used according to U.S. Pat. No. 6,143,864. In peculiar, said SgIIb conjugates can be used in a method comprising the step of: administering a SgIIb conjugate in a pharmaceutically acceptable composition to an individual, wherein the cytotoxic agent is released into the physiological environment at the place of proteolytic cleavage by PSA. Preferably, this method is directed to the treatment of prostate cancer.

The present invention provides a method of enhancing spermatozoa functions such as capacitation and egg-binding comprising the step of: administering a SgIIb polypeptide or a SgIIb agonist to a spermatozoon. Such a method may be carried out in vitro, e.g., by delivering a SgIIb polypeptide or a SgIIb agonist in vitro to an ejaculate sample before artificial insemination. Such a method for enhancing spermatozoa capacitation or egg-binding may also be carried out in vivo by delivering a pharmaceutical composition comprising a physiologically acceptable carrier and an effective amount of a SgIIb polypeptide or a SgIIb agonist to an individual. As used herein, an “effective amount of a SgIIb polypeptide or a SgIIb agonist” refers to an amount of SgIIb polypeptide or SgIIb agonist that is sufficient to enhance spermatozoa capacitation or egg-binding. Enhancing spermatozoa capacitation or egg-binding may be useful in treatments against male infertility. Such pharmaceutical compositions may be administered in any way, preferably locally.

The present invention further provides contraceptive methods using SgIIb polypeptides or SgIIb antagonists. Such a method may comprise the step of: administering a SgIIb antagonist to a spermatozoon. Said method may be carried out in vitro, e.g., by delivering a SgIIb antagonist to an ejaculate sample before screening for fertility-enhancing drugs. Said method may be carried out in vivo by delivering a pharmaceutical composition comprising a physiologically acceptable carrier and an effective amount of a SgIIb antagonist to an individual. As used herein, an “effective amount of a SgIIb antagonist” refers to a SgIIb antagonist that either decrease spermatozoa capacitation or egg-binding or inhibit proteolytic cleavage of SgIIb polypeptides by PSA. Alternatively, SgIIb polypeptides may be used as contraceptive immunogens in a method comprising the step of: administering to an individual a SgIIb polypeptide as described in U.S. Pat. No. 6,197,940, which disclosure is hereby incorporated in its entirety, in an effective amount to reduce the fertility of that individual via generation of anti-SgIIb antibodies. Preferably, immunogenic SgIIb polypeptides are used in such compositions. The administered amount of immunogenic SgIIb polypeptides depends upon factors such as route of administration, species, and the use of booster administration. For example, a dose of about 0.1 to 100 micrograms of SgIIb polypeptides per kg of body weight may be used. Compositions of the present invention may be prepared as both human and veterinary vaccine formulations. Contraceptive vaccines can be produced by combining SgIIb polypeptides with a pharmaceutically suitable carrier and with an adjuvant that contains non-specific stimulators of the immune system such as, e.g., immunogenic fragments of Bordatella pertussis.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising a SgIIb agonist and a physiologically acceptable carrier can be for, e.g., intravenous, topical, rectal, local, inhalant, subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a SgIIb polypeptide or a nucleic acid sequence that encodes a cDNA that is complementary to SgIIb polynucleotides. Methods of constructing transgenic animals are well-known to those skilled in the art. For example, a transgenic mouse for SgIIb polunucleotides may be constructed according to U.S. Pat. No. 6,100,444, which disclosure is hereby incorporated by reference in its entirety. Such transgenic animals may for example be useful for obtaining animal models for dysfunctions associated with low spermatozoa capacitation and egg-binging, for treating such dysfunctions and for screening compounds for pharmaceutical activity in the treatment of these dysfunctions.

Protein of SEQ ID NO:16 (Internal Designation Clone 1000770389_{—}208-24-4-0-G7-F)

The cDNA of Clone 1000770389_{—}208-24-4-0-G7-F (SEQ ID NO:15) encodes Sepin of SEQ ID NO:16, comprising the amino acid sequence: MKSSGLFPFLVLLALGTLAPWAVEGSGKSFKAGVCPPKKSAQCLRYKKPECQSDWQCPGKKRC CPDTCGIKCLDPVDTPNPTRRKPGKCPVTYGQCLMLNPPNFCEMDGQCVT. Accordingly, it will be appreciated that all characteristics and uses of the polypeptides of SEQ ID NO:16 described throughout the present application also pertain to the polypeptides encoded by the nucleic acids comprising the human cDNA in Clone 1000770389_{—}208-24-4-0-G7-F. In addition, it will be appreciated that all characteristics and uses of the polynucleotides of SEQ ID NO:15 described throughout the present application also pertain to the nucleic acids comprising the human cDNA in Clone 1000770389_{—}208-24-4-0-G7-F. A preferred embodiment of the invention is directed toward the compositions of SEQ ID NO:16, SEQ ID NO:15, and Clone 1000770389_{—}208-24-4-0-G7-F. Preferred Sepin polypeptide fragments for uses in the methods described below include the Sepin polypeptide comprising the amino sequence of: SGKSFKAGVCPPKKSAQCLRYKKPECQSDWQCPGKKRCCPDTCGIKCLDPVDTPNPTRRKPGKC PVTYGQCLMLNPPNFCEMDGQCVT. Also preferred are polypeptide fragments having a biological activity as described herein and the polynucleotides encoding the fragments.

The protein of the invention, Sepin, is a NOVEL variant of the Secretory leukocyte proteinase inhibitor (SLPI, Genbank accession number P03973). Sepin is 113 amino-acids long whereas SLPI is 32 amino-acids long. The 111 amino-terminal amino-acids are identical between Sepin and SLPI and the 2 carboxyl-terminal amino-acids are unique to Sepin. Sepin is a secreted protein that displays a signal peptide (MKSSGLFPFLVLLALGTLAPWAVEG). Sepin displays one complete WAP domain (KAGVCPPKKSAQCLRYKKPECQSDWQCPGKKRCCPDTCGIKCLDP) with a WAP-type “four disulfide core” domain signature (CQSDWQCPGKKRCC). This WAP domain is specifically involved in trypsin inhibition. The WAP domain that is involved in chymotrypsin, elastase and cathepsin G inhibition in SLPI is not present in Sepin. Thus Sepin is a trypsin-specific proteinase inhibitor, contrarily to SLPI that exhibits a broad spectrum inhibitory activity. Sepin is present in various human mucous secretions such as bronchial mucus, nasal secretion, parotid secretion, seminal plasma and cervical mucus.

An embodiment of the invention is directed to a composition comprising a Sepin polypeptide sequence of SEQ ID NO:16, or having at least 70%, 80%, 90%, 95%, 96%, 97%, 98%, 99%, or more identity to SEQ ID NO:16.

A further embodiment of the invention is directed to a composition comprising a Sepin polypeptide fragment having trypsin inhibitory activity.

As used herein, a “Sepin polypeptide” refers either to a Sepin polypeptide sequence of SEQ ID NO:16 or at least 70%, 80%; 90%, 95%, 96%, 97%, 98%, 99%, or more identical to SEQ ID NO:16, or to a Sepin polypeptide fragment having trypsin inhibitory activity.

An embodiment of the invention is directed to a composition comprising a polynucleotide sequence of SEQ ID NO:15 encoding a Sepin polypeptide.

A further embodiment of the invention is directed to a composition comprising a polynucleotide sequence encoding a Sepin polypeptide.

As further used herein, a “Sepin polynucleotide” refers either to a Sepin polynucleotide sequence of SEQ ID NO:15 or having at least 70%, 80%, 90%, 95%, 96%, 97%, 98%, 99%, or more identity to SEQ ID NO:15, to a nucleotide sequence encoding a Sepin polypeptide, or to a polynucleotide fragment of SEQ ID NO: 15.

A further embodiment of the invention is directed to an antibody recognizing a Sepin polypeptide sequence of SEQ ID NO:16 or a Sepin polypeptide fragment. Preferably, the antibody recognizes one or more of the two carboxyl-terminal amino-acids of Sepin, wherein said one or more amino-acids are required for binding of the antibody to a Sepin polypeptide. Most preferably, the antibody binds to Sepin but not to SLPI. As used herein, an “anti-Sepin antibody” refers to an antibody or antigen-binding fragment thereof that specifically binds to a Sepin polypeptide.

An embodiment of the present invention relates to a method of binding an anti-Sepin antibody to a Sepin polypeptide comprising the step of: contacting a Sepin polypeptide with said antibody under conditions that allow binding to occur. Such conditions are well known to those skilled in the art. Such methods are useful for detecting Sepin polypeptides as further described herein.

An embodiment of the present invention is directed to a method of detecting Sepin polypeptides in a biological sample, said method comprising the steps of: i) contacting a biological sample with an anti-Sepin antibody; and ii) detecting the antigen-antibody complex formed. The anti-Sepin antibody may be monoclonal or polyclonal. In addition, the anti-Sepin antibody may be primarily or secondarily labeled by any detectable compound (e.g., radioactive, fluorescent, luminescent, or enzymatic) common in the art. Such a method may be applied in methods further described below.

An embodiment of the present invention is directed to methods of detecting Sepin expression in a biological sample, said methods comprising the steps of: i) providing a biological sample from an individual; ii) detecting the level of Sepin expression in the sample; and iii) comparing the level of Sepin expression in said sample to that of a control sample. In one such embodiment, detecting the level of Sepin expression is used to diagnose various chronic and acute diseases of the respiratory tract, such as acute respiratory distress syndrome (ARDS), emphysema or asthma. Said biological sample may be derived from any biological fluid or tissue, and is preferably obtained from bronchoalveolar lavage fluids. A higher level of Sepin expression in said biological sample in comparison to the level in a control sample representative of a level in a normal individual is indicative of the presence of ARDS or of an elevated risk of developing ARDS. The level of Sepin expression in the sample can be assessed using any method, such as by detecting the level of Sepin mRNA or Sepin polypeptides in the sample. For example, well-known techniques such as, e.g., western blot, immunochemical techniques and cytochemical techniques may be used to detect Sepin expression. Preferably, anti-Sepin antibodies are used to detect the level of Sepin expression. Also preferably, polynucleotide probes comprising a polynucleotide sequence of SEQ ID NO:15 or part thereof, or a polynucleotide sequence complementary of SEQ ID NO:15, are used to detect the level of Sepin expression by, e.g., northern blot or RTPCR techniques. Detecting Sepin expression in a bronchoalveolar lavage fluid may be performed as described in Sallenave et al. (Eur Respir J. 13:1029-36 (1999)), the disclosure of which is incorporated by reference herein in its entirety.

Another embodiment of the present invention is directed to a diagnostic kit for detecting in vitro the presence of a Sepin polypeptide. Such kit comprises: i) an anti-Sepin antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Sepin antibodies is used for diagnosing ARDS, e.g. by detecting the presence of a Sepin polypeptide in a biological sample taken from an individual suspected of having ARDS. Optionally, said diagnostic kit comprises a negative control sample representative of the level from a normal individual. Optionally, said diagnostic kit comprises a positive control sample representative of the level from an individual with established ARDS. Optionally, said diagnostic kit comprises a positive control sample representative of the level from an individual at risk of developing ARDS and that went on to develop ARDS.

Another embodiment relates to a method of producing a Sepin polypeptide comprising the steps of: i) culturing a cell expressing a Sepin polypeptide, and ii) purifying the produced protein. The purification of the protein can be carried out following any technique known to those skilled in the art. Preferably, an anti-Sepin antibody may be bound to a chromatographic support to form an affinity chromatography column. Preferably, the cell expressing a Sepin polypeptide is a recombinant host cell as described below. Producing Sepin polypeptides may be useful in methods and compositions as further described in the present invention.

A preferred aspect of the invention is a host cell recombinant for polynucleotides encoding a Sepin polypeptide, operably linked to a promoter. An embodiment is directed to a method of constructing a host cell recombinant for polynucleotides encoding a Sepin polypeptide comprising the steps of: i) constructing a recombinant vector that comprises a nucleic acid sequence encoding a Sepin polypeptide, operably linked to a promoter, and ii) introducing said recombinant vector into a cell. In preferred embodiments, the cell is an Escherichia coli cell, a plant cell or a human cell. In most preferred embodiments, the cell is an Aspergillus niger cell as described in Mikosch et al. (J Biotechnol. 52:97-106 (1996)), which disclosure is incorporated herein by reference in its entirety. In other preferred embodiments, the cell is a plant cell. An additional preferred aspect is a host cell recombinant for polynucleotides that, when present in a cell, cause an alteration in Sepin expression. A host cell recombinant for such polynucleotides may be constructed by a method comprising the step of: i) providing a cell comprising the Sepin gene; and ii) introducing a recombinant vector comprising Sepin expression-altering polynucleotides into said cell, wherein the presence of said polynucleotides in said cell increases or decreases the level of Sepin expression compared to the level of Sepin expression in said cell in the absence of said recombinant vector. Preferably, said polynucleotides are inserted into or replace all or part of the 5′ regulatory region of the Sepin gene. Further preferably, said polynucleotides are located within 500 base pairs of the Sepin coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cells producing Sepin polypeptides may be used to produce Sepin polypeptides, which may be used for multiple purposes as described herein.

Another preferred embodiment relates to methods of screening test substances for the ability to modulate Sepin expression comprising the steps of: i) contacting a cell with a test substance; and ii) comparing Sepin expression in the cell after exposure to the test substance to that of an unexposed control cell, wherein an observed difference in Sepin expression between the exposed cell and the unexposed control cell indicates that the test substance modulates Sepin expression. Sepin expression may be determined by methods common to the art or described herein. Methods of determining Sepin expression include, but are not limited to, methods of quantifying Sepin polynucleotides (e.g., detection of Sepin mRNA by northern blot or RTPCR) or to methods of quantifying Sepin polypeptides (e.g., detection of Sepin polypeptides by western blot or immunochemistry). In one embodiment, the test substance modifies the expression of Sepin in a specific cell type (preferably bronchial epithelial cells) but not in others. Modulators identified using such methods are also encompassed by the present invention, as are methods of using the modulators, e.g., to alter the expression of Sepin in a cell, tissue, or individual.

A further embodiment of the present invention is directed to methods of screening test substances for the ability to modulate Sepin activity comprising the steps of: i) contacting a cell with a test substance, and ii) comparing Sepin activity in the cell after exposure to the test substance to that of an unexposed control cell, wherein an observed difference in Sepin activity between the exposed and unexposed cells indicates that the test substance modulates Sepin activity. Sepin activity can be monitored, e.g., by measuring the inhibition of human pancreatic trypsin 1 or 2 as described in Belorgey et al. (Biochem J 313:555-60 (1996)), the disclosure of which is incorporated herein by reference in its entirety. Modulators identified using such methods are also encompassed by the present invention, as are methods of using the modulators, e.g. to alter the activity of Sepin in a cell, tissue, or individual.

Test substances that decrease Sepin expression or activity are defined as Sepin antagonists. Test substances that increase Sepin expression or activity are defined as Sepin agonists. Test substances that modulate the expression or activity of Sepin include, but are not limited to, chemical compounds (e.g. small-molecule inhibitors or activators), oligonucleotides, antisense polynucleotides, polypeptides, ribozymes, dominant negative forms of Sepin, and anti-Sepin antibodies. These substances may be made and used according to methods well known in the art.

A preferred embodiment is directed to a method of inhibiting trypsin comprising the step of: contacting a Sepin polypeptide with a biological sample comprising trypsin under conditions that allow binding to occur.

In one aspect, such a method of inhibiting trypsin may be used in vitro to inhibit or remove contaminating proteases in a sample. Compositions comprising Sepin polypeptides may be added to biological samples as a “cocktail” with other protease inhibitors to prevent degradation of protein samples. Such protease inhibitor cocktails are widely used in laboratories in order to inhibit undesirable proteases susceptible of degrading a protein of interest without knowing the specificity of the undesirable proteases. Alternatively, Sepin polypeptides may be bound to a chromatographic support, either alone or in combination with other protease inhibitors, using techniques well-known to those skilled in the art, to form a chromatography column. A sample susceptible of containing an undesirable protease is run through the column to remove said proteases from said sample.

In another aspect, such a method of inhibiting trypsin may be used in vivo to treat or reduce the severity of diseases associated with increased trypsin activity or with trypsin inhibitor deficiencies. Such diseases include, but are not limited to, acute pancreatitis, chronic pancreatitis, biliary atresia, biliary hypoplasia, neonatal hepatitis and pulmonary disorders induced by cigarette smoke. Such methods for inhibiting trypsin can be carried out by delivering a physiologically acceptable carrier and an effective amount of a Sepin polypeptide or a Sepin agonist to an individual. As used herein, an “effective amount of a Sepin polypeptide or a Sepin agonist” refers to an amount of Sepin polypeptide or Sepin agonist that is sufficient to reduce trypsin activity in a body fluid such as, e.g., biliary fluids, intestinal fluids and bronchial mucus. Sepin polypeptides or Sepin agonists are preferably systemically administered, for example by intravenous injection, by oral ingestion or by oral inhalation.

In still another aspect, such a method of inhibiting trypsin may be used to inhibit exogenous trypsins that are produced by various bacteria and viruses during infectious diseases. Such methods for inhibiting trypsin can be carried out by delivering a physiologically acceptable carrier and an effective amount of a Sepin polypeptide or a Sepin agonist to an individual suffering from an infectious disease. For example, HIV infections, rotavirus infections and cytomegalovirus infections may be treated by such a method. Alternatively, a Sepin polynucleotide may be introduced in a plant cell in order to decrease digestive trypsin activity of various insect pests, as described in U.S. Pat. No. 5,981,722, the disclosure of which is incorporated herein by reference in its entirety.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising a Sepin agonist or antagonist and a physiologically acceptable carrier can be for, e.g., intravenous, topical, rectal, local, inhalant, subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a Sepin polypeptide or a nucleic acid sequence that encodes a cDNA that is complementary to Sepin polynucleotides. Methods of constructing transgenic animals are well-known to those skilled in the art. For example, a Sepin overexpressing mouse can be used as an in vivo model of ARDS, and may be constructed according to U.S. Pat. No. 5,858,784, which disclosure is hereby incorporated by reference in its entirety.

Protein of SEQ ID NO:18 (Internal Designation Clone 1000888456_{—}159-16-3-0-E4-F)

The cDNA of Clone 1000888456_{—}159-16-3-0-E4-F (SEQ ID NO:17) encodes ULBP2A of SEQ ID NO:18, comprising the amino acid sequence: MAAAAATKILLCLPLLLLLSGWSRAGRADPHSLCYDITVIPKFRPGPRWCAVQGQVDEKTFLHY DCGNKTVTPVSPLGKKLNVTTAWKAQNPVLREVVDILTEQLRDIQLENYTPKEPLTLQARMSCE QKAEGHSSGSWQFSFDGQIFLLFDSEKRMWTTVHPGARKMKEKWENDKVVAMSFHYFSMGDC IGWLEDFLMGMDSTLEPSAGG. Accordingly, it will be appreciated that all characteristics and uses of the polypeptides of SEQ ID NO:18 described throughout the present application also pertain to the polypeptides encoded by the nucleic acids comprising the human cDNA in Clone 1000888456_{—}159-16-3-0-E4-F. In addition, it will be appreciated that all characteristics and uses of the polynucleotides of SEQ ID NO:17 described throughout the present application also pertain to the nucleic acids comprising the human cDNA in Clone 1000888456_{—}159-16-3-0-E4-F. A preferred embodiment of the invention is directed toward the human polynucleotide and polypeptide compositions of SEQ ID NO:18, SEQ ID NO:17, and Clone 1000888456_{—}159-16-3-0-E4-F. Preferred ULBP2A polypeptide fragments for uses in the methods described below include the ULBP2A polypeptide comprising the amino sequence of:

RADPHSLCYDITVIPKFRPGPRWCAVQGQVDEKTFLHYDCGNKTVTPVSPLGKKLNVTTAWKA
QNPVLREVVDILTEQLRDIQLENYTPKEPLTLQARMSCEQKAEGHSSGSWQFSEDGQIFLLFDSEK
RMWTTVHPGARKMKEKWENDKVVAMSFHYFSMGDCIGWLEDFLMGMDSTLEPSAGG.

Further preferred ULBP2A polypeptide fragments for uses in the methods described below include the ULBP2A polypeptide comprising the amino sequence of: MGDCIGWLEDFLMGMDSTLEPSAGG. Also preferred are polypeptide fragments having a biological activity as described herein, and the polynucleotides encoding the fragments.

ULBP2A is a NOVEL splice variant of UL16 binding protein 2 (ULBP2). The ULBP proteins (ULBP1, ULBP2, and ULBP3) are a NOVEL family of MHC Class I-related glycoproteins that were identified based on their ability to bind to the human cytomegalovirus glycoprotein, UL16. ULBP2 is a cell surface protein that is tethered to the plasma membrane through a glycosylphosphatidylinositol (GPI) linkage. The GPI linkage domain of ULBP2 corresponds to the last 20 amino acids of the protein. ULBP2 protein is expressed on a wide variety of cell lines and tissues. Expression of the ULBP2 protein is normally very low, but is highly induced in response to viral infection and other cellular stresses, such as oncogenic activation. ULBP's bind to the NKG2D/DAP10 receptor protein present on the surface of natural killer (NK) and cytotoxic T lymphocyte (CTL) cells. ULBP2 binding activates NK and CTL cells, leading to cytokine production and cytotoxicity. Thus, several types of cells may potentially deliver ULBP2-mediated signals to NK and CTL cells and be the targets of ULBP-mediated killing.

In contrast to ULBP2, ULBP2A is not associated with the cellular membrane. ULBP2A is spliced such that it lacks the C terminal 35 amino acids, which includes the GPI linkage domain. Accordingly, ULBP2A is predicted to be a soluble, secreted protein.

An embodiment of the invention is directed to a composition comprising an ULBP2A polypeptide sequence of SEQ ID NO:18, or having at least 70%, 80%, 90%, 95%, 96%, 97%, 98%, 99%, or more identity to SEQ ID NO:18.

A further embodiment of the invention is directed to a composition comprising an ULBP2A polypeptide fragment having a role in the activation of NK and CTL cells.

As used herein, a “ULBP2A polypeptide” refers either to an ULBP2A polypeptide sequence of SEQ ID NO:18 or at least 70%, 80%; 90%, 95%, 96%, 97%, 98%, 99%, or more identical to SEQ ID NO:18, or to an ULBP2A polypeptide fragment having a role in the activation of NK and CTL cells.

An embodiment of the invention is directed to a composition comprising a polynucleotide sequence of SEQ ID NO:17 encoding an ULBP2A polypeptide.

A further embodiment of the invention is directed to a composition comprising a polynucleotide sequence encoding an ULBP2A polypeptide.

As further used herein, a “ULBP2A polynucleotide” refers either to an ULBP2A polynucleotide sequence of SEQ ID NO:17 or having at least 70%, 80%, 90%, 95%, 96%, 97%, 98%, 99%, or more identity to SEQ ID NO:17, to a nucleotide sequence encoding an ULBP2A polypeptide, or to a polynucleotide fragment of SEQ ID NO:17.

A further embodiment of the invention is directed to an antibody recognizing an ULBP2A polypeptide sequence of SEQ ID NO:18 or an ULBP2A polypeptide fragment. As used herein, an “anti-ULBP2A antibody” refers to an antibody or antigen-binding fragment thereof that specifically binds to an ULBP2A polypeptide. An embodiment of the present invention relates to a method of binding an anti-ULBP2A antibody to an ULBP2A polypeptide comprising the step of: contacting an ULBP2A polypeptide with said antibody under conditions that allow binding to occur. Such conditions are well known to those skilled in the art. Such methods are useful for detecting ULBP2A polypeptides, as further described herein.

An embodiment of the present invention is directed to a method of detecting ULBP2A polypeptides in a biological sample, said method comprising the steps of: i) contacting a biological sample with an anti-ULBP2A antibody; and ii) detecting the antigen-antibody complex formed. The anti-ULBP2A antibody may be monoclonal or polyclonal. In addition, the anti-ULBP2A antibody may be primarily or secondarily labeled by any detectable compound (e.g., radioactive, fluorescent, luminescent, or enzymatic). Detecting ULBP2A polypeptides is for example useful in the field of medical oncology, e.g. as a marker for tumor metastasis. Such a method of using ULBP2A in the field of medical oncology may be performed as described in WO publication 01/53837, which disclosure is hereby incorporated by reference in its entirety.

An embodiment of the present invention is directed to methods of detecting ULBP2A expression in a biological sample, said methods comprising the steps of: i) providing a biological sample from an individual; ii) detecting the level of ULBP2A expression in the sample; and iii) comparing the level of ULBP2A expression in said sample to that of a control sample. The level of ULBP2A expression in the sample can be assessed using any method, such as by detecting the level of ULBP2A mRNA or ULBP2A polypeptides in the sample. Any of a number of well-known techniques such as, western blot, immunochemical techniques and cytochemical techniques may be used to detect ULBP2A expression. Preferably, anti-ULBP2A antibodies are used to detect the level of ULBP2A expression. Also preferably, polynucleotide probes comprising a polynucleotide sequence of SEQ ID NO:17 or part thereof, or a polynucleotide sequence complementary of SEQ ID NO:17, are used to detect the level of ULBP2A expression by, e.g., northern blot or RTPCR techniques.

In one embodiment, detecting the level of ULBP2A expression is used for diagnosing cancers such as, e.g., leukemias and lymphomas. A higher level of ULBP2A expression in said biological sample in comparison to the level in a control sample representative of a level in a normal individual is indicative of malignant tumor burden. Preferred biological sample is obtained from peripheral blood. This method may for example be performed as described in Rodrigues et al. (Clin Cancer Res. 7:854-60 (2001)), the disclosure of which is incorporated herein by reference in its entirety.

Another embodiment of the present invention is directed to a diagnostic kit for detecting in vitro the presence of an ULBP2A polypeptide. Such kit comprises: i) an anti-ULBP2A antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. The kit comprising anti-ULBP2A antibodies can used for many purposes, e.g., for diagnosing various cancers such as those listed above. Optionally, said diagnostic kit comprises a negative control sample representative of a level from a normal individual. Optionally, said diagnostic kit comprises a positive control sample representative of a level from an individual suffering from a given disorder.

Another embodiment relates to a method of producing an ULBP2A polypeptide comprising the steps of: i) culturing a cell expressing an ULBP2A polypeptide, and ii) purifying the produced protein. The purification of the protein can be carried out using any technique, a number of which are well-known to those skilled in the art. Preferably, an anti-ULBP2A antibody is bound to a chromatographic support to form an affinity chromatography column. Preferably, the cell expressing the ULBP2A polypeptide is a recombinant host cell as described below. Producing ULBP2A polypeptides may be useful in methods and compositions as further described in the present application.

A preferred aspect of the invention is a host cell recombinant for polynucleotides encoding an ULBP2A polypeptide. An embodiment is directed to a method of constructing a host cell recombinant for polynucleotides encoding an ULBP2A polypeptide comprising the steps of: i) constructing a recombinant vector that comprises a nucleic acid sequence encoding an ULBP2A polypeptide, operably linked to a promoter, and ii) introducing said recombinant vector into a cell. In preferred embodiments, said cell is an Escherichia coli cell or a human cell. An additional preferred aspect is a host cell recombinant for polynucleotides that, when present in a cell, cause an alteration in ULBP2A expression. A host cell recombinant for polynucleotides capable of modifying ULBP2A expression may be constructed by a method comprising the step of: i) providing a cell comprising the ULBP2A gene; and ii) introducing a recombinant vector comprising said polynucleotides into said cell, wherein the presence of said polynucleotides in said cell increases or decreases ULBP2A expression compared to the level of ULBP2A expression in said cell in the absence of said recombinant vector. Preferably, said polynucleotides are inserted into or replace all or part of the 5′ regulatory region of the ULBP2A gene. Further preferably, said polynucleotides are located within 500 base pairs of the ULBP2A coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing ULBP2A polypeptides may be used, e.g., to produce ULBP2A polypeptides.

Another preferred embodiment relates to methods of screening test substances for the ability to modulate ULBP2A expression comprising the steps of: i) contacting a cell with a test substance; and ii) comparing ULBP2A expression in the cell after exposure to the test substance to that of an unexposed control cell, wherein an observed difference in ULBP2A expression between the exposed cell and the unexposed control cell indicates that the test substance modulates ULBP2A expression. ULBP2A expression may be determined by methods common to the art, including but not limited to, methods of quantifying ULBP2A polynucleotides (e.g., detection of ULBP2A mRNA by northern blot or RTPCR) or to methods of quantifying ULBP2A polypeptides (e.g., detection of ULBP2A polypeptides by western blot or immunochemistry). Modulators identified using such methods are also encompassed by the present invention, as are methods of using the modulators, e.g., to alter the expression of ULBP2A in a cell, tissue or individual.

A further embodiment of the present invention is directed to methods of screening test substances for the ability to modulate ULBP2A activity. In one embodiment, the method comprises the steps of: i) contacting an ULBP2A polypeptide with a test substance, and ii) comparing the biological activity of the polypeptide in the presence of the test substance to the activity of an ULBP2A polypeptide in the absence of the test substance, wherein an observed difference in ULBP2A activity in the presence or absence of the test substance indicates that the test substance modulates ULBP2A activity. Such methods may be carried out by examining the ULBP2A activity in a cell, or by examining the activity of purified ULBP2A polypeptides. ULBP2A activity can for example be monitored by studying binding of ULBP2A to the surface of NK or CTL cells, or to the purified NKG2D/DAP10 receptor protein. ULBP2A binding can for example be assessed as described in (Ziebell et al, Chem. Biol. 8:1081-92, 2001), which disclosure is incorporated herein by reference in its entirety. Modulators identified using such methods are also encompassed by the present invention, as are methods of using the modulators, e.g., to alter the expression of ULBP2A in a cell, tissue or individual.

Test substances that decrease ULBP2A expression or activity are defined as ULBP2A antagonists. Test substances that increase ULBP2A expression or activity are defined as ULBP2A agonists. Test substances that modulate the expression or activity of ULBP2A include, but are not limited to, chemical compounds (e.g. small-molecule inhibitors or activators), oligonucleotides, antisense polynucleotides, polypeptides, ribozymes, dominant negative forms of ULBP2A and anti-ULBP2A antibodies. These substances may be made and used according to methods well known in the art.

A further embodiment of the present invention is directed to methods of utilizing ULBP2A polypeptides or polypeptide fragments to stimulate clonal expansion of naïve CTL cells ex vivo. This method comprises the steps of: i) isolation of lymphocytes either from a bone marrow donor or from a patient; ii) positive selection of CD34+ cells; and iii) ex vivo expansion of naïve CTL cells under ULBP2A -stimulated culture conditions which are well-known to those skilled in the art. Said method can be performed as described in van Stipdonk et al, Nat. Immunol., 2(5):423-9, 2001, disclosure of which is incorporated by reference herein in its entirety. Expanded populations of CTL cells are useful for immunoprophylaxic and immunotherapeutic treatment of viral infections and cancer.

The present invention provides a method of treatment of viral infection in vivo by delivering a pharmaceutical composition comprising a physiologically acceptable carrier and an effective amount of an ULBP2A polypeptide or an ULBP2A agonist to an individual. As used herein, an “effective amount of an ULBP2A polypeptide or an ULBP2A agonist” refers to an amount of ULBP2A polypeptide or ULBP2A agonist that is sufficient to enhance immune system-mediated eradication of virally infected cells. Such pharmaceutical compositions may be administered in any way, preferably locally.

The present invention further provides a method for treating an individual suffering from cancer comprising the step of: delivering a pharmaceutical composition comprising a physiologically acceptable carrier and an effective amount of ULBP2A to an individual suffering from cancer. ULBP2A may be administered either locally at the tumor site or systemically.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising an ULBP2A agonist and a physiologically acceptable carrier can be prepared for, e.g., intravenous, topical, rectal, local, inhalant, subcutaneous, intradermal, intramuscular, oral and intracerebral administration. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Protein of SEQ ID NO:20 (Internal Designation Clone 1000848040_{—}201-41-1-0-C1-F)

The cDNA of Clone 1000848040_{—}201-41-1-0-C1-F (SEQ ID NO:19) encodes TM4SF5A of SEQ ID NO:20, comprising the amino acid sequence: MCTGKCARCVGLSLITLCLVCIVANALLLVPNGETSWTNTNHLSLQVWLMGGFIGGGLMVLCP GLAAVRAGGKGCCGAGCCGNRCRMLRSVFSSAFGVLGAIYCLSVSGAGLRNGPRCLMNGEWG YHFGDTA. Accordingly, it will be appreciated that all characteristics and uses of the polypeptides of SEQ ID NO:20 described throughout the present application also pertain to the polypeptides encoded by the nucleic acids comprising the human cDNA in Clone 1000848040_{—}201-41-1-0-C1-F. In addition, it will be appreciated that all characteristics and uses of the polynucleotides of SEQ ID NO:19 described throughout the present application also pertain to the nucleic acids comprising the human cDNA in Clone 1000848040_{—}201-41-1-0-C1-F. A preferred embodiment of the invention is directed toward the human polynucleotide and polypeptide compositions of SEQ ID NO:20, SEQ ID NO:19, and Clone 1000848040_{—}201-41-1-0-C1-F. Preferred TM4SF5A polypeptide fragments for uses in the methods described below include the TM4SF5A polypeptide comprising the amino sequence of: GAGLRNGPRCLMNGEWGYHFGDTA. Also preferred are polypeptide fragments having a biological activity as described herein, and the polynucleotides encoding the fragments.

TM4SF5A is a NOVEL splice variant of Transmembrane 4 Super Family member 5 (TM4SF5) or tetraspanin 5. The tetraspanin superfamily and the related L6 membrane protein superfamily comprise cell surface proteins characterized by four highly conserved transmembrane domains. Other topological features shared by these proteins include short cytoplasmic domains at their N- and C-termini, a small extracellular domain between trasnmembrane domains 1 and 2, and a larger extracellular domain between transmembrane domains 3 and 4 that functions in ligand binding. Expression of TM4SF5 is restricted to the intestine in normal tissues, however overexpression of TM4SF5 has been correlated with cancers of the intestine, stomach and pancreas. TM4SF5 plays a role in the regulation of cellular proliferation, most likely through the activation of signal transduction pathways in response to binding of membrane proteins on neighboring cells.

In contrast to TM4SF5, TM4SF5A possesses only three transmembrane domains. TM4SF5A is spliced such that it lacks the C terminal 66 amino acids, which include the ligand binding cytoplasmic domain and the forth transmembrane domain. Accordingly, TM4SF5A is a dominant negative inhibitor of TM4SF5.

An embodiment of the invention is directed to a composition comprising an TM4SF5A polypeptide sequence of SEQ ID NO:20, or having at least 70%, 80%, 90%, 95%, 96%, 97%, 98%, 99%, or more identity to SEQ ID NO:20.

A further embodiment of the invention is directed to a composition comprising an TM4SF5A polypeptide fragment having a role in the inhibition of cellular proliferation.

As used herein, a “TM4SF5A polypeptide” refers either to an TM4SF5A polypeptide sequence of SEQ ID NO:20 or at least 70%, 80%; 90%, 95%, 96%, 97%, 98%, 99%, or more identical to SEQ ID NO:20, or to an TM4SF5A polypeptide fragment having a role in the inhibition of cellular proliferation.

An embodiment of the invention is directed to a composition comprising a polynucleotide sequence of SEQ ID NO:19 encoding an TM4SF5A polypeptide.

A further embodiment of the invention is directed to a composition comprising a polynucleotide sequence encoding an TM4SF5A polypeptide.

As further used herein, a “TM4SF5A polynucleotide” refers either to an TM4SF5A polynucleotide sequence of SEQ ID NO:19 or having at least 70%, 80%, 90%, 95%, 96%, 97%, 98%, 99%, or more identity to SEQ ID NO:19, to a nucleotide sequence encoding an TM4SF5A polypeptide, or to a polynucleotide fragment of SEQ ID NO:19.

A further embodiment of the invention is directed to an antibody recognizing an TM4SF5A polypeptide sequence of SEQ ID NO:20 or an TM4SF5A polypeptide fragment. As used herein, an “anti-TM4SF5A antibody” refers to an antibody or antigen-binding fragment thereof that specifically binds to an TM4SF5A polypeptide. An embodiment of the present invention relates to a method of binding an anti-TM4SF5A antibody to an TM4SF5A polypeptide comprising the step of: contacting an TM4SF5A polypeptide with said antibody under conditions that allow binding to occur. Such conditions are well known to those skilled in the art. Such methods are useful for detecting TM4SF5A polypeptides, as further described herein.

An embodiment of the present invention is directed to a method of detecting TM4SF5A polypeptides in a biological sample, said method comprising the steps of: i) contacting a biological sample with an anti-TM4SF5A antibody; and ii) detecting the antigen-antibody complex formed. The anti-TM4SF5A antibody may be monoclonal or polyclonal. In addition, the anti-TM4SF5A antibody may be primarily or secondarily labeled by any detectable compound (e.g., radioactive, fluorescent, luminescent, or enzymatic). Detecting TM4SF5A polypeptides is for example useful in the field of medical oncology, e.g. as a marker for tumor cells.

An embodiment of the present invention is directed to methods of detecting TM4SF5A expression in a biological sample, said methods comprising the steps of: i) providing a biological sample from an individual; ii) detecting the level of TM4SF5A expression in the sample; and iii) comparing the level of TM4SF5A expression in said sample to that of a control sample. The level of TM4SF5A expression in the sample can be assessed using any method, such as by detecting the level of TM4SF5A mRNA or TM4SF5A polypeptides in the sample. Any of a number of well-known techniques such as, western blot, immunochemical techniques and cytochemical techniques may be used to detect TM4SF5A expression. Preferably, anti-TM4SF5A antibodies are used to detect the level of TM4SF5A expression. Also preferably, polynucleotide probes comprising a polynucleotide sequence of SEQ ID NO:19 or part thereof, or a polynucleotide sequence complementary of SEQ ID NO:19, are used to detect the level of TM4SF5A expression by, e.g., northern blot or RTPCR techniques.

In one embodiment, detecting the level of TM4SF5A expression is used for diagnosing cancers such as, e.g., stomach, intestinal, and pancreatic cancers. A higher level of TM4SF5A expression in said biological sample in comparison to the level in a control sample representative of a level in a normal individual is indicative of malignant tumor burden. Preferred biological sample is obtained from tissues biopsies. This method may for example be performed as described in Rodrigues et al. (Clin Cancer Res. 7:854-60 (2001)), the disclosure of which is incorporated herein by reference in its entirety.

Another embodiment of the present invention is directed to a diagnostic kit for detecting in vitro the presence of a TM4SF5A polypeptide. Such kit comprises: i) an anti-TM4SF5A antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. The kit comprising anti-TM4SF5A antibodies can used for many purposes, e.g., for diagnosing various cancers such as those listed above. Optionally, said diagnostic kit comprises a negative control sample representative of a level from a normal individual. Optionally, said diagnostic kit comprises a positive control sample representative of a level from an individual suffering from a given disorder.

Another embodiment relates to a method of producing a TM4SF5A polypeptide comprising the steps of: i) culturing a cell expressing a TM4SF5A polypeptide, and ii) purifying the produced protein. The purification of the protein can be carried out using any technique, a number of which are well-known to those skilled in the art. Preferably, an anti-TM4SF5A antibody is bound to a chromatographic support to form an affinity chromatography column. Preferably, the cell expressing the TM4SF5A polypeptide is a recombinant host cell as described below. Producing TM4SF5A polypeptides may be useful in methods and compositions as further described in the present application.

A preferred aspect of the invention is a host cell recombinant for polynucleotides encoding a TM4SF5A polypeptide. An embodiment is directed to a method of constructing a host cell recombinant for polynucleotides encoding a TM4SF5A polypeptide comprising the steps of: i) constructing a recombinant vector that comprises a nucleic acid sequence encoding a TM4SF5A polypeptide, operably linked to a promoter, and ii) introducing said recombinant vector into a cell. In preferred embodiments, said cell is an Escherichia coli cell or a human cell. An additional preferred aspect is a host cell recombinant for polynucleotides that, when present in a cell, cause an alteration in TM4SF5A expression. A host cell recombinant for polynucleotides capable of modifying TM4SF5A expression may be constructed by a method comprising the step of: i) providing a cell comprising the TM4SF5A gene; and ii) introducing a recombinant vector comprising said polynucleotides into said cell, wherein the presence of said polynucleotides in said cell increases or decreases TM4SF5A expression compared to the level of TM4SF5A expression in said cell in the absence of said recombinant vector. Preferably, said polynucleotides are inserted into or replace all or part of the 5′ regulatory region of the TM4SF5A gene. Further preferably, said polynucleotides are located within 500 base pairs of the TM4SF5A coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing TM4SF5A polypeptides may be used, e.g., to produce TM4SF5A polypeptides.

Another preferred embodiment relates to methods of screening test substances for the ability to modulate TM4SF5A expression comprising the steps of: i) contacting a cell with a test substance; and ii) comparing TM4SF5A expression in the cell after exposure to the test substance to that of an unexposed control cell, wherein an observed difference in TM4SF5A expression between the exposed cell and the unexposed control cell indicates that the test substance modulates TM4SF5A expression. TM4SF5A expression may be determined by methods common to the art, including but not limited to, methods of quantifying TM4SP5A polynucleotides (e.g., detection of TM4SF5A mRNA by northern blot or RTPCR) or to methods of quantifying TM4SF5A polypeptides (e.g., detection of TM4SF5A polypeptides by western blot or immunochemistry). Modulators identified using such methods are also encompassed by the present invention, as are methods of using the modulators, e.g., to alter the expression of TM4SF5A in a cell, tissue or individual.

A further embodiment of the present invention is directed to methods of screening test substances for the ability to modulate TM4SF5A activity. In one embodiment, the method comprises the steps of: i) contacting a TM4SF5A polypeptide with a test substance, and ii) comparing the biological activity of the polypeptide in the presence of the test substance to the activity of a TM4SF5A polypeptide in the absence of the test substance, wherein an observed difference in TM4SF5A activity in the presence or absence of the test substance indicates that the test substance modulates TM4SF5A activity. Such methods may be carried out by examining the TM4SF5A activity in a cell, or by examining the activity of purified TM4SF5A polypeptides. TM4SF5A activity can for example be monitored by studying its ability to suppress the proliferation of cultured cells. TM4SF5A binding can for example be assessed as described in (Ziebell et al, Chem. Biol. 8:1081-92, 2001), which disclosure is incorporated herein by reference in its entirety. Modulators identified using such methods are also encompassed by the present invention, as are methods of using the modulators, e.g., to alter the expression of TM4SF5A in a cell, tissue or individual.

Test substances that decrease TM4SF5A expression or activity are defined as TM4SF5A antagonists. Test substances that increase TM4SF5A expression or activity are defined as TM4SF5A agonists. Test substances that modulate the expression or activity of TM4SF5A include, but are not limited to, chemical compounds (e.g. small-molecule inhibitors or activators), oligonucleotides, antisense polynucleotides, polypeptides, ribozymes, dominant negative forms of TM4SF5A and anti-TM4SF5A antibodies. These substances may be made and used according to methods well known in the art.

The present invention provides a method of treatment of a disorder associated with cell proliferation by delivering a pharmaceutical composition comprising a physiologically acceptable carrier and an effective amount of a TM4SF5A polypeptide or a TM4SF5A agonist to an individual. Disorders of cell proliferation include various types of cancers including, but not limited to, adenocarcinoma, leukemia, lymphoma, melanoma, myeloma, sarcoma, and teratocarcinoma. As used herein, an “effective amount of a TM4SF5A polypeptide or a TM4SF5A agonist” refers to an amount of TM4SF5A polypeptide or TM4SF5A agonist that is sufficient to enhance immune system-mediated eradication of virally infected cells. Such pharmaceutical compositions may be administered in any way, preferably locally.

The present invention provides a method of treatment of inflammation of any type and, in particular, that which results from a particular disorder, by delivering a pharmaceutical composition comprising a physiologically acceptable carrier and an effective amount of a TM4SF5A polypeptide or a TM4SF5A agonist to an individual. Such disorders with associated inflammation include, but are not limited to, Addison's disease, adult respiratory distress syndrome, allergies, anemia, asthma, atherosclerosis, bronchitis, Crohn's disease, diabetis mellitus, gout, Graves' disease, irritable bowel syndrome, lupus erythematosus, multiple sclerosis, myocardial or pericardial inflammation, rheumatoid arthritis, viral, bacterial, fungal, parasitic, protozoal, and helminthic infections, and trauma. As used herein, an “effective amount of a TM4SF5A polypeptide or a TM4SF5A agonist” refers to an amount of TM4SF5A polypeptide or TM4SF5A agonist that is sufficient to enhance immune system-mediated eradication of virally infected cells. Such pharmaceutical compositions may be administered in any way, preferably locally.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising an ULBP2A agonist and a physiologically acceptable carrier can be prepared for, e.g., intravenous, topical, rectal, local, inhalant, subcutaneous, intradermal, intramuscular, oral and intracerebral administration. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Protein of SEQ ID NO:22 (Internal Designation Clone 500762393) The cDNA of Clone 500762393 (SEQ ID NO:21) encodes NoJAM of SEQ ID NO:22, comprising the amino acid sequence: MARRSRHRLLLLLLRYLVVALGYHKAYGFSAPKDQQVVTAVEYQEAILACKTPKKTVSSRLEW KKLGRSVSFVYYQQTLQGDFKNRAEMIDFNIRIKNVTRSDAGKYRCEVSAPSEQGQNLEEDTVT LEVLVAPAVPSCEVPSSALSGTVVELRCQDKEGNPAPEYTWFKDGIRLLENPRLGSQSTNSSYTM NTKTGTLQFNTVSKLDTGEYSCEARNSVGYRRCPGKRMQMIST. Accordingly, it will be appreciated that all characteristics and uses of the polypeptides of SEQ ID NO:22 described throughout the present application also pertain to the polypeptides encoded by the nucleic acids comprising the human cDNA of Clone 500762393. In addition, it will be appreciated that all characteristics and uses of the polynucleotides of SEQ ID NO:21 described throughout the present application also pertain to the nucleic acids comprising the human cDNA included in Clone 500762393. A preferred embodiment of the invention is directed toward the polynucleotide and polypeptide compositions comprising the sequences shown as SEQ ID NO:21 and SEQ ID NO:22, and the human cDNA and encoded polypeptide of Clone 500762393. Also preferred are polypeptide fragments having a biological activity as described herein and the polynucleotides encoding the fragments.

NoJAM is a NOVEL splice variant of the Vascular Endothelial Junctional Adhesion Molecule (VEJAM). VEJAM is a member of the immunoglobulin superfamily of transmembrane molecules and the growing family of Junctional Adhesion Molecules (JAM) involved in cell-cell contact. VEJAM is highly expressed in endothelial cells of high endothelial venules and lymphatic vessels in lymphoid organs, particularly Peyer's patches and lymph nodes, and in vascular structures of the heart. Intercellular JAM interactions mediate cell adhesion. In addition, cells expressing a JAM molecule are capable of transmigrating across JAM-expressing endothelia. JAM interactions can be homotypic or heterotypic between family members, though heterotypic binding is typically stronger. For example, VEJAM binding to JAM-3 on activated peripheral blood lymphocytes allows these cells to cross endothelial borders in to or out of lymphoid organs.

In contrast to other JAM proteins, NoJAM is entirely extracellular. NoJAM is spliced such that it ends before the transmembrane domain of VEJAM and terminates with four unique amino acids. Accordingly, NoJAM acts as a dominant negative inhibitor of JAM-mediated cell-cell interactions and transmigration of cells across epithelial borders. Preferred polypeptide fragments of the invention are those that comprise the four C-terminal amino acids. These include, but are not limited to: MQMIST, DTGEYSCEARNSVGYRRCPGKRMQMIST, and TVVELRCQDKEGNPAPEYTWFKDGIRLLENPRLGSQSTNSSYTMNTKTGTLQFNTVSKLDTGEY SCEARNSVGYRRCPGKRMQMIST.

An embodiment of the invention is directed to a composition comprising a NoJAM polypeptide sequence of SEQ ID NO:22, or having at least about 70%, 80%, 90%, 95%, 96%, 97%, 98%, or 99% identity to SEQ ID NO:22.

A further embodiment of the invention is directed to a composition comprising a NoJAM polypeptide fragment having a biological activity of binding JAM family proteins.

A further embodiment of the invention is directed to a composition comprising a polynucleotide that encodes a NoJAM polypeptide. In one embodiment, the polynucleotide comprises the sequence shown as SEQ ID NO:21, or having at least about 70%, 80%, 90%, 95%, 96%, 97%, 98% or 99% identity with SEQ ID NO:21.

A further embodiment of the invention is directed to a composition comprising a polynucleotide sequence encoding a NoJAM polypeptide fragment having biological activity of binding JAM family proteins.

A preferred embodiment is a host cell capable of expressing a NoJAM polypeptide or biologically active fragment thereof under conditions suitable for expression.

A preferred aspect of the invention is a method of binding a NoJAM specific antibody to NoJAM, comprising the step of: contacting a NoJAM specific antibody with a NoJAM polypeptide or fragment thereof under conditions suitable for antibody binding. NoJAM specific antibodies are also provided and are further described below. Such antibodies may be polyclonal or monoclonal antibodies, and preferably selectively bind to NoJAM but not to VEJAM.

A preferred aspect of the invention is a method of using NoJAM to prevent cellular transmigration of epithelial and endothelial linings, comprising the step of: contacting a cell with a physiologically acceptable composition comprising a NoJAM polypeptide or a JAM-binding fragment thereof. Preferably, this method is directed to in vivo treatment of an individual, e.g. an individual suffering from a disease or condition such as rheumatoid arthritis, Crohn's disease, psoriasis, endometriosis, inflammatory bowel disease, multiple sclerosis, asthma, or metastatic invasion of the heart, kidney, lymph nodes, or Peyer's patches. Preferably, this method also includes the step of determining if an individual has or is at risk of developing any of these diseases or conditions, e.g. using the above-described methods of detecting sites of JAM expression.

A preferred embodiment is a host cell capable of expressing a NoJAM polypeptide or a biologically active fragment thereof under conditions suitable for expression. This aspect of the invention is useful, e.g.,to enable purification of NoJAM using methods common to the art and described herein. Preferred host cells are bacterial, yeast, and mammalian cells. Preferably, the host cell is recombinant for NoJAM encoding nucleotide sequences. Such nucleotide sequences are typically comprised in extrachromosomal plasmid DNA. Appropriate vectors for introduction of recombinant sequences are discussed herein. Alternatively, the host cell is recombinant for polynucleotides that, when present in a cell, cause an alteration in the expression of endogenous NoJAM. For example, as NoJAM and VEJAM are expressed from the same gene, preferred polynucleotides for altering NoJAM expression are those that increase the ratio of NoJAM splice product to VEJAM splice product. Techniques known in the art for introducing polynucleotide sequences to endogenous sequences are described in U.S. Pat. No. 5,641,670 and PCT Application WO96/29411, which disclosures are hereby incorporated by reference in their entireties. NoJAM polypeptides are useful, e.g., for in vitro assays that include cell purification and detection, as well as in vivo applications. Such applications are detailed herein.

Another embodiment of the invention is directed to an antibody specific for NoJAM, and compositions comprising said antibodies. Preferred antibodies are those that bind specifically to the preferred NoJAM fragments listed above. A further preferred aspect is a method of binding a NoJAM specific antibody to a NoJAM polypeptide or fragment thereof. This method comprises the step of contacting a NoJAM specific antibody with a solution suspected of comprising a NoJAM polypeptide or fragment thereof under conditions suitable for antibody binding. Such antibodies and methods are useful for purification and detection of NoJAM, as described elsewhere herein.

A preferred embodiment of the invention is a method of purifying a NoJAM polypeptide. This method comprises the steps of: i) binding a NoJAM specific antibody to NoJAM under conditions that allow binding to occur, and ii) removing substances that are not bound to the antibody. Preferably, the NoJAM specific antibody is directly or indirectly associated with a solid or semisolid matrix to facilitate protein purification. Further preferably, an additional step of dissociating NoJAM from the NoJAM specific antibody is included in a method of purification. Purified NoJAM is useful for example, for in vitro cell purification and detection and in vivo to prevent localized immune cell invasion of a tissue. These applications are detailed presently.

An additional embodiment of the invention is a method of detecting a NoJAM polypeptide. This method comprises the steps of: i) contacting a NoJAM specific antibody with a sample suspected of containing a NoJAM polypeptide; and ii) detecting any antibody-NoJAM complexes formed in step i). In one embodiment, said sample is a biological sample. Preferably, the additional step of removing substances that are not bound to the NoJAM specific antibody is included in this method. Further preferable is direct or indirect labeling of the NoJAM specific antibody with a detectable compound, such as a fluorescent, luminescent, or radioactive compound. Detection of NoJAM is useful for example to determine sites of JAM expression, which is indicative of high transepithelial traffic. Sites of transmigration include immune cell invasion in the case of inflammation or infection. In addition, metastatic cell migration causes vascular cell walls become less resistant to transmigration. Thus, detection of NoJAM is useful for diagnosis of rheumatoid arthritis, Crohn's disease, psoriasis, endometriosis, inflammatory bowel disease, multiple sclerosis, asthma, and sites of tumor invasion, especially in the heart, kidney, lymph nodes, and Peyer's patches.

A preferred embodiment of the invention is a method of binding NoJAM to JAM-expressing cells such as myeloid cells, monocytes, neutrophils, antigen presenting cells, platelets, and T cells. This method comprises the step of: contacting a NoJAM polypeptide or JAM-binding fragment thereof with a JAM-expressing cell under conditions suitable for binding. Optionally a second step of separating cells that are not complexed with NoJAM is included in the method of binding. This optional method is particularly useful for purification of the listed cell types. Alternatively, a second step of separating unbound NoJAM from complexed NoJAM is included in the method of binding. This alternative method is particularly useful for detection of the listed cell types.

As described above, a NoJAM-specific antibody may be used to indirectly detect sites of JAM expression. Alternatively, NoJAM polypeptide or a JAM-binding fragment thereof is useful to identify these sites directly. This method comprises the step of contacting a detectably-labeled NoJAM polypeptide or JAM-binding fragment thereof with a cell or tissue under conditions suitable for JAM binding; ii) separating unbound NoJAM from complexed NoJAM; and iii) detecting the complexed NoJAM. This method may be accomplished by techniques common in the art such as immunofluorescence. Preferably, NoJAM is detected in a tissue sample, such as a sample containing endothelial venules, lymphatic vessels, or vascular structures of the heart. Further preferable is fixation of the tissue sample so that cellular borders are at least partially intact. This method of detection is useful for diagnosis of diseases or conditions including, but not limited to, rheumatoid arthritis, Crohn's disease, psoriasis, endometriosis, inflammatory bowel disease, multiple sclerosis, asthma, and sites of tumor invasion.

NoJAM may be used to purify a cell that expresses a JAM molecule on its surface. A preferred embodiment is a method comprising the steps of: i) contacting a NoJAM polypeptide or JAM-binding fragment thereof with a plurality of cells under conditions suitable for JAM binding; and ii) removing any unbound cells. Preferably, NoJAM is directly or indirectly associated with a solid or semisolid matrix to facilitate the purification of JAM-expressing cells from a sample comprising many cell types. This method is useful for purification of cells such as myeloid cells, monocytes, neutrophils, antigen presenting cells, platelets, and T cells. This method is also useful for removing these cell types from a tissue sample, e.g. toto prevent contamination from inappropriate cell types in tissue-specific assays. Removal of reactive immune cells from a tissue is especially important for grafted tissue. NoJAM conjugated to a magnetic particle, for example, may be used to bind JAM-expressing cells in a tissue which can then be removed by applying a magnetic field sufficient to remove any bound cells. A less intense magnetic field may be used to maintain the integrity of the remaining tissue. Magnetic purification systems are available from several commercial sources including Dynal (Oslo, Norway) and Miltenyi Biotec (Bergisch Gladbach, Germany).

JAM proteins expressed on the surface of circulating cells are readily accessible to NoJAM binding. NoJAM is therefore particularly useful for blocking sites of interaction on JAM-expressing blood and lymphoid cells. Blocking these sites reduces the amount of cell transmigration through JAM-expressing epithelial and endothelial borders. Thus, increasing the expression or activity of NoJAM is useful for preventing inflammatory diseases such as rheumatoid arthritis, Crohn's disease, psoriasis, endometriosis, inflammatory bowel disease, multiple sclerosis and asthma. NoJAM activity is also particularly useful in preventing tissue invasion by circulating metastatic cells that express JAM. NoJAM is especially useful in preventing invasion of the heart, kidney, lymph nodes, and Peyer's patches. Conversely, inhibition of NoJAM increases transmigration. This is particularly useful to promote wound healing, especially in poorly vascularized tissue.

To this end, a preferred embodiment of the invention is a method of screening for substances that increase the expression of NoJAM polypeptides or polynucleotides. This method comprises the steps of: i) contacting a cell with a test substance; and ii) comparing NoJAM expression in the cell after exposure to the test substance to that of an unexposed control cell, wherein an observed difference in the expression of NoJAM in the exposed and the unexposed cells indicates that the test substance modulates the expression of NoJAM polypeptides or polynucleotides. NoJAM expression is determined by methods common to the art or included herein. Methods of determining NoJAM expression include but are not limited to methods of quantifying NoJAM polynucleotides (e.g., Northern blot or RTPCR) or to methods of quantifying cytogram polynucleotides (e.g., Western blot or immunochemistry). Preferably, the test substance increases the ratio of NoJAM to VEJAM expression. Further preferred is a test substance that increases NoJAM expression in a specific cell type while not in others. Most preferably, the test substance modifies NoJAM expression specifically in one or more of the following: epithelial cells, endothelial cells, vascular endothelial cells, high endothelial venule cells, myeloid cells, monocytes, neutrophils, antigen presenting cells, platelets, and activated T cells. The method described herein may also be used to screen for substances that decrease the expression of NoJAM polypeptides. Preferred test substances that decrease NoJAM expression are those that decrease the ratio of NoJAM to VEJAM expression.

An additional aspect of the invention is a screen for substances that modulate the activity of NoJAM polypeptides. This method comprises the steps of: i) contacting a NoJAM polypeptide or JAM-binding fragment thereof with a test substance; ii) detecting NoJAM activity; and iii) comparing NoJAM activity in the presence of the test substance to that of an unexposed control, wherein an observed difference in NoJAM activity in the presence or absence of said test substance indicates that the test substance modulates the activity of NoJAM polypeptides. Such methods may be performed using a cell expressing the NoJAM polypeptide or fragment, or using a purified NoJAM polypeptide or fragment. NoJAM activity can be assessed by virtue of any of the herein-described NoJAM activities, preferably JAM binding ability. For example, NoJAM in the presence of the test substance may be exposed to an immobilized JAM family protein under varying conditions and the binding compared to the NoJAM only control. A test substance is said to increase NoJAM activity if there is NoJAM-JAM binding in more stringent conditions than in the absence of that substance. Alternatively, this method may be used to screen for substances that decrease the activity of NoJAM polypeptides. A test substance is said to decrease NoJAM activity if there is NoJAM-JAM binding in more stringent conditions in the absence of the substance. An example of a NoJAM inhibitor is an antibody that interferes with NoJAM binding to a JAM family member.

While activators of JAM expression or activity may be used to prevent or treat inflammation and metastatic invasion, these conditions may also be addressed directly with NoJAM polypeptides. NoJAM is advantageous because it represents a naturally occurring polypeptide that is less likely to cause an unanticipated reaction. Accordingly, a preferred aspect of the invention is a method of using NoJAM to prevent cellular transmigration of epithelial and endothelial linings. This method comprises the step of: contacting a pharmaceutical composition comprising a NoJAM polypeptide or JAM-binding fragmen thereof with a cell. Preferably, this method also includes the step of determining if an individual has or is at risk of one of the listed inflammatory disorders or metastatic invasion. Preferably, this method is directed to prevention or treatment of rheumatoid arthritis, Crohn's disease, psoriasis, endometriosis, inflammatory bowel disease, multiple sclerosis, asthma, and metastatic invasion of the heart, kidney, lymph nodes, and Peyer's patches. The pharmaceutical composition may be administered in any way, preferably orally or locally to a particular site of cellular transmigration in an individual. Localized delivery is preferably accomplished by injection into an appropriate blood or lymphoid vessel or by means of an implanted stent (see, for example, U.S. Pat. Nos. 5,500,013 and 5,449,382 disclosures of which are hereby incorporated by reference in their entireties).

An inhibitor of NoJAM expression or activity may be used to promote transmigration of endothelial and epithelial borders, and thus, to promote wound healing. A preferred embodiment of the invention is a method comprising the steps of: i) determining if an individual has a wound in need of treatment; and ii) introducing a pharmaceutical composition comprising a physiologically acceptable NoJAM inhibitor to the wound in need of treatment. A wound may be deemed in need of treatment by any qualified medical caregiver, and is typically considered in need of treatment if it is healing more slowly than a comparable wound on a more vascularized site on the affected individual or on a healthy individual. Preferably, a NoJAM inhibitor is delivered locally to the site of the wound. Delivery is preferably accomplished by injection into an appropriate blood or lymphoid vessel or by means of an implanted stent, as discussed herein. In another embodiment, the composition may be formulated for topical administration and is administered directly onto said wound.

Protein of SEQ ID NO:24 (Internal Designation Clone 1000848660_{—}181-42-1-0-C11-F)

The cDNA of Clone 1000848660_{—}181-42-1-0-C11-F (SEQ ID NO:23) encodes NOVEL small inducible cytokine type 14 (NCCL14) protein of SEQ ID NO:24, comprising the amino acid sequence: MKISVAAIPFFLLITIALGTKTESSSRGPYHPSECCFTYTTYKIPRQRIMDYYETNSQCSKPGIV. Accordingly, it will be appreciated that all characteristics and uses of the polypeptides of SEQ ID NO:23 described throughout the present application also pertain to the polypeptides encoded by the human cDNA included in Clone 1000848660_{—}181-42-1-0-C11-F. In addition, it will be appreciated that all characteristics and uses of the polynucleotides of SEQ ID NO:23 described throughout the present application also pertain to the nucleic acids comprising the human cDNA in Clone 1000848660_{—}181-42-1-0-C11-F. A preferred embodiment of the invention is directed toward the compositions of SEQ ID NO:23, SEQ ID NO:24 and Clone 1000848660_{—}181-42-1-0-C11-F. Preferred NCCL14 polypeptide fragments for uses in the methods described below include the NCCL14 polypeptide comprising the amino sequence of: TKTESSSRGPYHPSECCFTYTTYKIPRQRIMDYYETNSQCSKPGIV. Also preferred are polypeptide fragments having a biological activity as described herein and the polynucleotides encoding the fragments.

The protein of the invention, NCCL14 is a NOVEL splice variant of small inducible cytokine A14 (CCL14, Genbank accession number Q16627). The fist exon of NCCL14 is identical to the first exon of CCL14. The second exon is different between the two proteins. Thus the protein of the invention, NCCL14, is 65 amino-acids long whereas CCL14 precursor is 93 amino-acids long. NCCL14 belongs to the CC chemokine family. NCCL14 is a secreted protein that displays a signal peptide (MKISVAAIPFFLLITIALG), and the mature NCCL14 protein is 46 amino-acids long. NCCL14 displays an IL8 domain, located at the amino-terminal extremity of the processed protein (SSRGPYHPSECCFTYTTYKIPRQRIMDYYETNSQCSKPGIV).

Chemokines play a major role in the recruitment, navigation and activation of different leukocytes, including for example monocytes, lymphocytes, and neutrophils. They regulate myelopoiesis, exert antiviral effects and possess angiogenetic activity. They exert their biological activity via activation of seven transmembrane domain G-protein coupled receptors. Such activity is useful, e.g., for immune enhancement or suppression, myeloprotection, stem cell mobilization, acute and chronic inflammatory control and treatment of leukemia.

NCCL14 is expressed in numerous tissues, including thespleen, liver, skeletal and heart muscle, gut, and bone marrow, and is also present in plasma. NCCL14 also acts on human monocytes, acting via the CCR1 and CCR5 receptors. In addition, NCCL14 modulates the proliferation of stem cells and myeloid progenitors, for example NCCL14 modulates colony formation of bone marrow progenitor cells. NCCL14 is involved in the trafficking of inflammatory cells. NCCL14 contributes to the development of B-cells areas of secondary lymphoid tissues. Also, NCCL14 is responsible for a basic level of circulating leukocytes by mobilizing them into the plasma and NCCL14 represent a factor for homeostasis of CCR1+ inflammatory cells in the blood stream.

An embodiment of the invention is directed to a composition comprising a NCCL14 polypeptide sequence of SEQ D NO:24. In another embodiment, the polypeptide is at least 70%, 80%, 90%, 95%, or more identical to SEQ ID NO:24.

A further embodiment of the invention is directed to a composition comprising a NCCL14 polypeptide fragment having any of the biological activities described herein, or to a composition comprising a NCCL14 polypeptide fragment that binds to CCR1 and/or CCR5.

As further used herein, a NCCL14 polypeptide refers either to a NCCL14 polypeptide sequence of SEQ ID NO:24 or having at least 70%, 80%, 90%, 95%, or more identity to SEQ ID NO:24 or to a polynucleotide fragment of SEQ ID NO:24 having biological activity.

A further embodiment of the invention is directed to a composition comprising a polynucleotide sequence of SEQ ID:23 encoding a NCCL14 polypeptide. In another embodiment, the polynucleotide sequence has at least 70%, 80%, 90 %, 95%, or more identity to SEQ ID NO:23.

In another embodiment, the present invention provides a polynucleotide that encodes a polypeptide comprising the sequence of SEQ ID NO:24 or a biologically fragment thereof.

A further embodiment of the invention is directed to a composition comprising a polynucleotide sequence encoding a NCCL14 polypeptide having any of the herein-described biological activities.

A further embodiment of the invention is directed to an antibody recognizing a NCCL14 polypeptide sequence of SEQ ID NO:24 or a NCCL14 polypeptide fragment. Preferably, the antibody recognizes one or more amino-acids located within the carboxyl-terminal extremity of NCCL14, wherein said one or more amino-acids are required for binding of the antibody to a NCCL14 polypeptide. Also preferably, the antibody recognizes a non linear epitope. Most preferably, the antibody binds to NCCL14 but not to CCL14. As used herein, an anti-NCCL14 antibody refers to an antibody or antigen-binding fragment thereof that specifically binds to a NCCL14 polypeptide. Such NCCL14-specific antibodies can be used for NCCL14 purification, inhibition, detection and diagnosis, e.g., as described herein.

A further embodiment of the invention is directed to a method of binding an antibody to a NCCL14 polypeptide or a fragment thereof comprising the step of: contacting a NCCL14 polypeptide with said antibody under conditions that allow binding to occur. Such conditions are well known to those skilled in the art.

An embodiment of the present invention is directed to a method of detecting NCCL14 polypeptides in a biological sample, said method comprising the steps of: i) contacting a biological sample with an anti-NCCL14 antibody; and ii) detecting the antigen-antibody complex formed. The antibody or antibody fragment may be monoclonal or polyclonal. In addition, the antibody or antibody fragment may be primarily or secondarily labeled by any detectable compound (e.g., radioactive, fluorescent, luminescent, or enzymatic) common in the art. Such methods may be applied to, e.g., diagnosis of chronic renal failure as further described below.

An embodiment of the present invention relates to methods of producing NCCL14 polypeptides. The method of producing NCCL14 polypeptides comprises the step of: i) transfecting a mammalian host with a recombinant expression vector encoding a NCCL14 polypeptide of the present invention, ii) culturing the cell under conditions conducive to the expression of said polypeptide; and iii) purifying the expressed NCCL14 polypeptide. The purification of the protein can be carried out using any technique known to those skilled in the art. Preferably, an antibody directed against NCCL14 is bound to a chromatographic support to form an affinity chromatography column. The purified protein can be used for any number of applications, such as those described below.

A preferred aspect of the invention is a host cell recombinant for polynucleotides encoding a NCCL14 polypeptide or a biologically active fragment thereof. An additional preferred aspect is a host cell recombinant for polynucleotides whose presence in a cell alters NCCL14 expression, e.g. causes an increase in the level of NCCL14 expression. Preferably, the polynucleotides capable of altering NCCL14 expression are inserted into the genome of the host cell, e.g. inserted into the 5′ regulatory region of the NCCL14 gene. Further preferably, these polynucleotides are located within 500 base pairs of the NCCL14 coding region. These polynucleotides preferably comprise a promoter sequence. Techniques known in the art for introducing polynucleotides sequences to endogenous sequences are described in U.S. Pat. No. 5,641,670 and PCT WO9629411, which disclosure are herein incorporated by reference in their entireties. NCCL14 protein produced by said host cells may be used as described herein, e.g., for in vitro detection and purification methods as well as diagnosis and in vivo applications.

An embodiment of the present invention is directed to methods of detecting NCCL14 expression in a biological sample, said methods comprising the steps of: i) providing a biological sample from an individual; ii) detecting the level of NCCL14 expression; and iii) comparing the level of NCCL14 expression in said sample to that of a control sample. Detecting the level of NCCL14 expression is useful to diagnose, e.g., various abnormal inflammatory and immunological responses. For example, a lower level of NCCL14 expression in said biological sample in comparison to the level of a control sample that is representative of the level in a normal individual is indicative of chronic renal failure. The level of NCCL14 expression in the sample can be assessed using any methods, such as detecting the level of NCCL14 mRNA or protein in the sample. For example, well-known techniques such as, e.g., western blot or immunochemistry may be used to detect NCCL14 expression. Preferably, anti-NCCL14 antibodies are used to detect the level of NCCL14 expression. Said antibody is detectably labeled as described above. Also preferably, polynucleotide probes comprising a polynucleotide sequence of the protein of the invention or part thereof, or a polynucleotide sequence complementary to all or part of SEQ ID NO:23, are used to detect the level of NCCL14 expression by, e.g., northern blot or RTPCR techniques.

Another embodiment of the present invention is directed to a diagnostic kit for detecting in vitro the presence of NCCL14 polypeptide. Such kit comprises: i) an anti-NCCL14 antibody; and optionally, ii) a reagent allowing the detection of the antigen-antibody complexes formed. Preferably said antibody is detectably labeled as described above. Alternatively, the optional reagent may provide a detectable signal. The optional reagent may either bind to said antibody or react with the label on said antibody. Preferably, the kit comprising anti-NCCL14 antibodies is used for diagnosing chronic renal failure or various abnormal inflammatory and immunological responses. Optionally, said diagnostic kit comprises a negative control sample representative of the level expected from a normal individual. Optionally, said diagnostic kit comprises a positive control sample representative of the level expected from an individual suffering from a chronic renal failure or from a given inflammatory or immunological response.

Another preferred embodiment relates to methods of screening tests substances for the ability to modulate NCCL14 expression comprising the steps of: i) contacting a cell with a test substance; and ii) comparing NCCL14 expression in the cell after exposure in the cell to the test substance to that of an unexposed control cell, wherein an observed difference in NCCL14 expression between the exposed cell and the unexposed control cells indicates that the test substance modulates NCCL14 expression. NCCL14 expression may be determined by methods common to the art or included herein. Methods of determining NCCL14 expression include but are not limited to methods of quantifying NCCL14 polynucleotides (e.g., detection of NCCL14 mRNA by northern blot or RTPCR) or to methods of quantifying NCCL14 polypeptides (e.g., detection of NCCL14 polypeptides by western blot or immunochemistry). Preferably, the test substance modifies the expression of NCCL14 in a specific cell type while not in others.

Modulators identified using such methods are also encompassed by the present invention. Any cell type may be used in such screens, e.g. cells of the spleen, liver, muscle, rut, or bone marrow. A further embodiment of the present invention is directed to methods of screening test substances for the ability to modulate NCCL14 activity comprising the steps of: i) contacting a cell with a test substance, and ii) comparing NCCL14 activity in the cell after exposure to the test substance that of an unexposed control cell, wherein an observed difference in NCCL14 activity between the exposed and unexposed cells indicates that the test substance modulates NCCL14 activity. NCCL14 activity can, for example, be monitored by studying its chemotactic activity as described, e.g., in Pardigol et al. (Proc Natl Acad Sci U S A. 95(11):6308-13 (1998)), the disclosure of which is incorporated by reference in its entirety. Alternatively, NCCL14 activity can be monitored by studying its ability to modulate the proliferation of stem cells and myeloid progenitors, e.g., by studying its ability to enhance the proliferation of CD34+ bone marrow cells in the presence of stem cell factor as described in Schulz-Knappe et al. (J Exp Med. 183(1):295-9 (1996)), the disclosure of which is incorporated by reference in its entirety. Modulators identified using such methods are also encompassed by the present invention.

Test substances that decrease NCCL14 expression or activity are defined as NCCL14 antagonists. Alternatively, test substances that increase NCCL14 expression or activity are defined as NCCL14 agonists. Test substances that modulate the expression or activity of NCCL14 include, but are not limited to, chemical compounds (e.g., small-molecule inhibitors or activators), oligonucleotides, antisense polynucleotides, polypeptides, ribozymes, dominant negative forms of NCCL14, and anti-NCCL14 antibodies. Theses substances may be made and used according to methods well known in the art.

A preferred embodiment of the invention is a method of binding CCR1 and/or CCR5 polypeptides with a NCCL14 polypeptide, the method comprising the step of: contacting a CCR1 and/or CCR5-binding NCCL14 polypeptide under conditions that allow binding of said NCCL14 polypeptide to CCR1 and/or CCR5. In a preferred embodiment, binding of NCCL14 polypeptide to CCR1 and/or CCR5 is used to purify cells expressing CCR1 and/or CCR5. In one such embodiment, a method of purifying cells expressing CCR1 and/or CCR5 comprises the step of: i) contacting a biological sample comprising CCR1-expressing cells and/or CCR5-expressing cells with a labeled NCCL14 polypeptide; and ii) introducing said biological sample into a sorting apparatus, e.g., a fluorescence-activated cell sorter or a magnetic activated cell-sorting apparatus, wherein CCR1 cells and/or CCR5 cells with said sample are sorted away from non-expressing cells by virtue of their bound, labeled NCCL14 polypeptide. This method is useful forthe enumeration, morphological identification or separation of CCRS-expressing lymphocyte populations. In particular, CCR5 density on CD4+ lymphocytes correlates positively with cell infectability by the HIV virus. Thus purifying CCR5 expressing cells, for example CD4+ lymphocytes, may be useful for, e.g., obtaining highly infectable cell populations when screening for anti-HIV drugs. This correlation also indicates that agents that block CCR5 accessibility, such as NCCL14 or derivatives thereof, can be used to block HIV infection in cells.

Additional aspects of this embodiment include methods of using a CCR1 and/or CCR5-binding NCCL14 polypeptide to detect and quantify cells expressing CCR1 and/or CCR5 using techniques common in the art. This method comprises the steps of: i) providing a biological sample suspected of containing cells expressing CCR1 and/or CCR5; ii) contacting said sample with a NCCL14 polypeptide under conditions suitable for binding of NCCL14; and iii) detecting the binding of CCL14 to cells in the sample. Preferably, said CCL14 polypeptide is covalently attached to a detectable compound.

Alternatively, a detectable anti-NCCL14 may be used to detect NCLL14. This method may be used as a diagnostic tool. For example, CCR1 as well as processes and cellular response mediated by CCR1 are involved in rejection of transplanted grafts (see Gao et al., J Clin Invest. 105(1):35-44 (2000)), which disclosure is herein incorporated by reference in its entirety), and CCR5 expressed by T cells is involved in diseases including, but not limited to, asthma and atopic disorders (for example, atopic dermatitis and allergies), rheumatoid arthritis, atherosclerosis, sarcoidosis, or idiopathic pulmonary fibrosis and other fibrotic diseases, psoriasis, autoimmune diseases such as multiple sclerosis, treating and/or preventing rejection of transplanted organs, and inflammatory bowel disease, in particular in mammals, preferably humans. In addition, CCR5 is a co-receptor for the entry of HIV into cells. The present invention also relates to a kit for use in detecting the presence of CCR1 and/or CCR5 or a portion thereof in a biological sample, comprising a NCCL14 polypeptide which binds to a mammalian CCR1 and/or CCR5 or a portion of said receptor, and one or more ancillary reagents suitable for detecting the presence of the complex between said NCCL14 polypeptide and CCR1 and/or CCR5 or portion thereof (see, for example U.S. Pat. No. 6,329,510, which disclosure is herein incorporated by reference in its entirety).

In another embodiment, the present invention also encompasses a method of inhibiting leukocyte trafficking in a patient, comprising administering to the patient an effective amount of CCR1 and/or CCR5 -binding NCCL14 polypeptide that binds to a mammalian CCR1 as described in U.S. Pat. No. 6,329,510, which disclosure is herein incorporated by reference in its entirety.

A further embodiment provides a method for inhibiting the rejection of transplanted grafts comprising administering an effective amount of CCR1 and/or CCR5-binding NCCL14 polypeptide to a graft recipient. In one embodiment, the graft is an allograft. In a particular embodiment, the allograft is a heart. The term “graft” as used herein, refers to organs and/or tissues which can be obtained from a first mammal or donor and transplanted into a second mammal, preferably a human. For example, see U.S. patent application Ser. No. 09/239,283, which disclosure is herein incorporated in its entirety.

A further embodiment provides a method of inhibiting (reducing or preventing) ischemia/reperfusion injury comprising administering to a subject in need thereof an effective amount of a CCR1 and/or CCR5-binding NCCL14 polypeptide. Ischemia/reperfusion injury refers to necrotic cell death that occurs when the flow of blood to an organ or tissue is restricted or stopped (ischemia), resulting in oxygen deprivation (hypoxia). The injury sustained by an organ or tissue under ischemic conditions is apparent after blood flow has been restored (reperfusion). In some embodiments, the ischemia/reperfusion injury can be a consequence of trauma or a medical procedure, for example, surgery. In other embodiments, the ischemia/reperfusion injury can be the result of a pathological condition, for example, arteriosclerosis, myocardial infection, stroke or transient ischemic attack. In a particular embodiment, the ischemia/reperfusion injury is a consequence of graft transplantation. In another particular embodiment, the graft is a kidney. In another embodiment, the method comprises administration of NCCL14 and one or more additional therapeutic agents, for example, thrombolytic agents, cell adhesion inhibitors, anti-coagulants, anti-thrombotic agents and activators or inhibitors of nitric oxide synthetase. For example, see U.S. patent application Ser. No. 09/239/283, which disclosure is herein incorporated by reference in its entirety.

In another preferred embodiment, the invention relates to a method for treating inflammatory demyelinating diseases comprising the step of administering an effective amount of a CCR1 and/or CCR5-binding NCCL14 polypeptide to a subject in need thereof. Preferably, the inflammatory demyelinating disease is multiple sclerosis. Such method is described in U.S. patent application Ser. No. 09/240,253, which disclosure is herein incorporated by reference in its entirety.

In a preferred embodiment, the present invention relates to therapeutic compositions comprising a NCCL14 polypeptide or a NCCL14 agonist that inhibits replication and/or infection of an immunodeficiency virus in vitro or in vivo, decreaes viral load, or treats and/or prevents diseases or disorders associated with human infection with an immunodeficiency virus. The immunodeficiency virus can be but is not limited to HIV, simian immunodeficiency virus, and feline immunodeficiency virus, and is most preferably HIV. See for example U.S. Pat. No. 6,214,540, and PCT application WO 00/40964, which disclosures are herein incorporated in their entirety.

Another embodiment of the invention relates to compositions and methods using polynucleotide sequences encoding A NCCL14 polypeptide to establish transgenic model animals (D. melanogaster, M. musculus), by any method familiar to those skilled in the art. By modulating in vivo the expression of the transgene with drugs or modifier genes (activator or suppressor genes), animal models can be developed that mimic diseases associated with impaired immunological response. These animal models would thus allow the identification of potential therapeutic agents for treatment of the disorders. In addition, recombinant cell lines derived from these transgenic animals may be used for similar approaches ex vivo.

Protein of SEQ ID NO:26 (Internal Designation Clone 500735264_{—}205-41-2-0-A7-F)

The cDNA of Clone 500735264_{—}205-41-2-0-A7-F (SEQ ID NO:25) encodes CS-5b of SEQ ID NO:26, comprising the amino acid sequence: MAAGSRTSLLLAFALLCLPWLQEADSIPTSSNMEETQQKSNLELLHISLLLIESRLEPVRFLRSTFT NNLVYDTSDSDDYHLLKDLEEGIQMLMGRLEDGSHLTGQTLKQTYSKFDTNSHNHDALLKNYG LLHCFRKDMDKVETFLRMVQCRSVEGSCGF. Accordingly, it will be appreciated that all characteristics and uses of the polypeptides of SEQ ID NO:26 described throughout the present application also pertain to the polypeptides encoded by the nucleic acids comprising the human cDNA in Clone 500735264_{—}205-41-2-0-A7-F. In addition, it will be appreciated that all characteristics and uses of the polynucleotides of SEQ ID NO:25 described throughout the present application also pertain to the nucleic acids comprising the human cDNA in Clone 500735264_{—}205-41-2-0-A7-F. A preferred embodiment of the invention is directed toward the human polynucleotide and polypeptide compositions of SEQ ID NO:26, SEQ ID NO:25, and Clone 500735264_{—}205-41-2-0-A7-F. Preferred CS-5b polypeptide fragments for uses in the methods described below include the CS-5b polypeptide comprising the amino sequence of: IPTSSNMEETQQKSNLELLHISLLLJESRLEPVRFLRSTFTNNLVYDTSDSDDYHILLKDLEEGIQML MGRLEDGSHLTGQTLKQTYSKFDTNSHNHDALLKNYGLLHCFRKDMDKVETFLRMVQCRSVE GSCGF. Also preferred are polypeptide fragments having a biological activity as described herein and the polynucleotides encoding the fragments.

The protein of the invention, CS-5b, is a NOVEL, secreted splice variant of the chorionic somatomammotropin 5 (CS-5, SP-TREMBL accession number Q14406). The CS-5b mRNA displays an additional exon at its 5′ extremity. The second exon of the CS-5b mRNA corresponds to the first exon of the CS-5 mRNA. In the CS-5b mRNA, this exon is shorter at its 3′ extremity than in the CS-5 mRNA. The three following exons are identical in both mRNAs. The resulting CS-5b full-length polypeptide is 160 amino-acids long whereas the CS-5 full-length polypeptide is 199 amino-acids long. After cleavage of the signal peptides, CS-5 displays 39 supplementary amino-acids at its amino-terminal extremity compared to CS-5b. The proteins are identical on their 134 carboxyl-terminal amino-acids. CS-5b displays a somatotropin_{—}2 consensus (CFRKDMDKVETFLRMVQC) and a somatotropin hormone family domain.

CS-5b is synthesized by the syncytiotrophoblast cells of the placenta and secreted in the blood. CS-5b is also present in cerebrospinal fluid of pregnant women. CS-5b plays a role in adaptation to pregnancy, and produces a variety of biological responses related to pregnancy and lactation. Notably, CS-5b stimulates adipocyte metabolism in the mother, both by stimulating adipose accumulation and by increasing the lipolysis rate, thus increasing the availability of substrates to the fetus. CS-5b also reduces insulin sensitivity. Furthermore, CS-5b increases milk production and stimulates maternal caretaking and nesting behavior during the fed state. CS-5b binds to different isoforms of the prolactin receptor (Genbank accession number P16471), which is widely expressed both in males and females, and activates prolactin receptor-mediated signaling.

An embodiment of the invention is directed to a composition comprising a CS-5b polypeptide sequence of SEQ ID NO:26, or having at least 70%, 80%, 90%, 95%, 96%, 97%, 98%, 99%, or more identity to SEQ ID NO:26.

A further embodiment of the invention is directed to a composition comprising a CS-5b polypeptide or polypeptide fragment that has a biological activity selected from the group consisting of enhancing adipose accumulation, increasing substrate availability to the fetus, increasing maternal caretaking and increasing insulin secretion.

As used herein, a “CS-5b polypeptide” refers to a polypeptide comprising, consisting of, or consisting essentially of the amino acid sequence of SEQ ID NO:26 or a amino acid sequence at least 70%, 80%; 90%, 95%, 96%, 97%, 98%, 99%, or more identical to SEQ ID NO:26, or to a polypeptide fragment thereof having a biological activity selected from the group consisting of enhancing adipose accumulation, increasing substrate availability to the fetus, increasing maternal caretaking and increasing insulin secretion. CS-5b polypeptides comprising 5 or more contiguous amino acids of the 39 amino-terminal amino acids of a mature CS-5 polypeptide are specifically excluded from the present invention.

An embodiment of the invention is directed to a composition comprising a polynucleotide sequence of SEQ ID NO:25 encoding a CS-5b polypeptide.

A further embodiment of the invention is directed to a composition comprising a polynucleotide sequence encoding a CS-5b polypeptide.

As further used herein, a “CS-5b polynucleotide” refers to a polynucleotide comprising, consisting of, or consisting essentially of the nucleotide sequence of SEQ ID NO:25 or a nucleotide sequence having at least 70%, 80%, 90%, 95%, or more identity to SEQ ID NO:25, to a nucleotide sequence encoding a CS-5b polypeptide, or to a sequence complementary to any of these sequences.

A further embodiment of the invention is directed to an antibody that recognizes a CS-5b polypeptide sequence of SEQ ID NO:26 or a CS-5b polypeptide fragment. Preferably, the antibody recognizes the amino-terminal extremity of a CS-5b polypeptide. Also preferably, the antibody recognizes non-linear epitopes. Most preferably, the antibody binds to CS-5b but not to CS-5. As used herein, an “anti-CS-5b antibody” refers to an antibody or antigen-binding fragment thereof that specifically binds to a CS-5b polypeptide.

An embodiment of the present invention relates to a method of binding an anti-CS-5b antibody to a CS-5b polypeptide comprising the step of: contacting a CS-5b polypeptide with said antibody under conditions that allow binding to occur. Such conditions are well known to those skilled in the art. Such methods are useful for detecting CS-5b polypeptides as further described herein.

An embodiment of the present invention is directed to a method of detecting CS-5b polypeptides in a biological sample, said method comprising the steps of: i) contacting a biological sample with an anti-CS-5b antibody; and ii) detecting the antigen-antibody complex formed. The anti-CS-5b antibody may be monoclonal or polyclonal. In addition, the anti-CS-5b antibody may be primarily or secondarily labeled by any detectable compound (e.g., radioactive, fluorescent, luminescent, or enzymatic) common in the art. Such a method may be applied to, e.g., diagnosis of pathologic conditions of pregnancy as further described below.

An embodiment of the present invention is directed to methods of detecting CS-5b expression in a biological sample, said methods comprising the steps of: i) providing a biological sample from an individual; ii) detecting the level of CS-5b expression in the sample; and iii) comparing the level of CS-5b expression in said sample to that of a control sample. In one such embodiment, said biological sample is obtained from a pregnant individual, and the method is used to diagnose pathologic conditions of pregnancy. A lower level of CS-5b expression in said biological sample in comparison to the level in a control sample representative of a level in a normal individual is either indicative of a risk of developing a maternal disease associated with pregnancy such as, e.g., diabetes mellitus, pre-eclampsia and hypersensitive vascular disease, or indicative of a fetal disease such as, e.g., intrauterine growth restriction, fetal growth retardation and fetal distress. Preferably, said level indicative of a pathologic condition of pregnancy corresponds to CS-5b concentration in the maternal plasma that is lower than 4 micrograms/milliliters. The level of CS-5b expression in the sample can be assessed using any method, such as by detecting the level of CS-5b mRNA or CS-5b polypeptides in the sample. Any of a number of well-known techniques such as, e.g., western blot, immunochemical techniques and cytochemical techniques may be used to detect CS-5b expression. Preferably, anti-CS-5b antibodies are used to detect the level of CS-5b expression. Also preferably, polynucleotide probes comprising a polynucleotide sequence of SEQ ID NO:25 or part thereof, or a polynucleotide sequence complementary to SEQ ID NO:25, are used to detect the level of CS-5b expression by, e.g., northern blot or RTPCR techniques.

Another embodiment of the present invention is directed to a diagnostic kit for quantifying in vitro the presence of a CS-5b polypeptide. Such kit comprises: i) an anti-CS-5b antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-CS-5b antibodies is used for diagnosing pathologic conditions of pregnancy such as those listed above. Optionally, said diagnostic kit comprises a negative control sample representative of the level from a normal pregnancy. Optionally, said diagnostic kit comprises a positive control sample representative of the level from an individual suffering from a given pathologic condition of pregnancy. Such a kit is very useful to define a population of women at risk of developing a disorder during their pregnancy and who could thus benefit from a preventive treatment.

Another embodiment relates to a method of producing a CS-5b polypeptide comprising the steps of: i) culturing a cell expressing a CS-5b polypeptide, and ii) purifying the produced protein. The purification of the protein can be carried out following any technique well-known to those skilled in the art. Preferably, an anti-CS-5b antibody may be bound to a chromatographic support to form an affinity chromatography column. Alternatively, the CS-5b polypeptide may be fused to a heterologous immunogenic peptide, and the fusion protein is purified using an antibody specific to the heterologous peptide. Preferably, the cell expressing a CS-5b polypeptide is a recombinant host cell as described below. Producing CS-5b polypeptides may be useful in methods and compositions as further described in the present invention.

A preferred aspect of the invention is a host cell recombinant for polynucleotides encoding a CS-5b polypeptide, operably linked to a promoter. An embodiment is directed to a method of constructing a host cell recombinant for polynucleotides encoding a CS-5b polypeptide comprising the steps of: i) constructing a recombinant vector that comprises a nucleic acid sequence encoding a CS-5b polypeptide, operably linked to a promoter, and ii) introducing said recombinant vector into a cell. In preferred embodiments, the cell is an Escherichia coli cell, a C127 mouse cell or a human cell. Methods for producing active CS-5b polypeptides in both glycosylated and non-glucosylated forms can be performed as described in U.S. Pat. No. 6,136,562, the disclosure of which is incorporated herein by reference in its entirety. An additional preferred aspect is a host cell recombinant for polynucleotides that, when present in a cell, cause an alteration in CS-5b expression. A host cell recombinant for polynucleotides capable of modifying CS-5b expression may be constructed by a method comprising the steps of: i) providing a cell comprising the CS-5b gene; and ii) introducing a recombinant vector comprising CS-5b expression-altering polynucleotides into said cell, wherein the presence of said polynucleotides in said cell increases or decreases CS-5b expression compared to the level of CS-5b expression in said cell before said recombinant vector is introduced. Preferably, said polynucleotides are inserted into or replace all or part of the 5′ regulatory region of the CS-5b gene. Further preferably, said polynucleotides are located within 500 base pairs of the CS-5b coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing CS-5b polypeptides may be used to produce CS-5b polypeptides.

Another preferred embodiment relates to methods of screening test substances for the ability to modulate CS-5b expression comprising the steps of: i) contacting a cell with a test substance; and ii) comparing CS-5b expression in the cell after exposure to the test substance to that of an unexposed control cell, wherein an observed difference in CS-5b expression between the exposed cell and the unexposed control cell indicates that the test substance modulates CS-5b expression. CS-5b expression may be determined by methods common to the including but not limited to methods of quantifying CS-5b polynucleotides (e.g., detection of CS-5b mRNA by northern blot or RTPCR) or to methods of quantifying CS-5b polypeptides (e.g., detection of CS-5b polypeptides by western blot or immunochemistry). In one embodiment, the test substance modifies the expression of CS-5b in a specific cell type (preferably an adipocyte or an epithelial cell) but not in others. Modulators identified using such methods are also encompassed by the present invention, as are methods of using the modulators, e.g., to alter the expression of CS-5b in a cell, tissue, or individual.

A further embodiment of the present invention is directed to methods of screening test substances for the ability to modulate CS-5b activity comprising the steps of: i) contacting a cell with a test substance, and ii) comparing CS-5b activity in the cell after exposure to the test substance to that of an unexposed control cell, wherein an observed difference in CS-5b activity between the exposed and unexposed cells indicates that the test substance modulates CS-5b activity. CS-5b activity can be determining by any of a number of techniques common to the art. CS-5b activity can be monitored, e.g., by measuring the inhibition of insulin-stimulated glucose (e.g., [¹⁴C]-glucose) incorporation into lipids in cells such as 3T3-L1 adipocytes, for example as described in U.S. Pat. No. 6,136,562, which disclosure is hereby incorporated by reference in its entirety. Alternatively, CS-5b activity can be monitored by studying its binding to prolactin receptors, e.g., using binding experiments and in vitro bioassays as described in Vashdi-Elberg et al. (J Biol Chem. 271:5558-64 (1996)), the disclosure of which is incorporated by reference herein in its entirety. Modulators identified using such methods are also encompassed by the present invention, as are methods of using the modulators, e.g. to alter the activity of CS-5b in a cell, tissue, or individual.

Test substances that decrease CS-5b expression or activity are defined as CS-5b antagonists. Test substances that increase CS-5b expression or activity are defined as CS-5b agonists. Test substances that modulate the expression or activity of CS-5b include, but are not limited to, chemical compounds (e.g. small-molecule inhibitors or activators), oligonucleotides, antisense polynucleotides, polypeptides, ribozymes, dominant negative forms of CS-5b, and anti-CS-5b antibodies. These substances may be made and used according to methods well known in the art. Such candidate modulators include but are not limited to 1,25-dihydroxyvitamin D3, interleukin-6, interleukin-1, the NF-IL transcription factor, pre-beta HDL and apoA-I, as well as derivatives, variants, and fragments of these modulators.

An embodiment of the present invention relates to a method of binding a CS-5b polypeptide to a prolactin receptor comprising the step of: contacting a CS-5b polypeptide with said prolactin receptor under conditions that allow binding to occur. This method may be applied in vitro as described by, e.g., Vashdi-Elberg et al. (J Biol Chem. 271:5558-64 (1996)), the disclosure of which is incorporated by reference herein in its entirety. A preferred embodiment is directed to methods of using a CS-5b polypeptide for activating prolactin receptor-mediated signaling. Such a method may be applied ex vivo or in vivo. A method for activating prolactin receptor-mediated signaling ex vivo comprises the step of: contacting a cell expressing a prolactin receptor with a CS-5b polypeptide under conditions that allow binding of said prolactin receptor with said CS-5b polypeptide to occur. A method for activating prolactin receptor-mediated signaling in vivo comprises the step of: delivering a physiologically acceptable carrier and an effective amount of a CS-5b polypeptide or a CS-5b agonist to an individual. As used in the context of this embodiment, an “effective amount of a CS-5b polypeptide or a CS-5b agonist” refers to an amount of CS-5b polypeptide or CS-5b agonist that is sufficient to activate prolactin receptor-mediated signaling. Preferably, such a method is directed to treat or prevent a maternal disease associated with pregnancy such as, e.g., diabetes mellitus, pre-eclampsia or hypersensitive vascular disease. Also preferably, said method is directed to treat or reduce in severity fetal disease such as, e.g., intrauterine growth restriction, fetal growth retardation and fetal distress. Alternatively, such a method may be used in breeding of mammals such as, e.g., cows and sheep, for stimulating milk production, maternal caretaking and nesting behavior during the fed state.

Another embodiment is directed to a method of stimulating lipid metabolism in adipocytes comprising the step of: contacting an adipocyte with a CS-5b polypeptide. Such a method may be applied in vitro to cultures of adipocytes. Such a method may also be applied in vivo by administering a physiologically acceptable carrier and an effective amount of a CS-5b polypeptide or a CS-5b agonist to an individual. As used in the context of this embodiment, an “effective amount of a CS-5b polypeptide or a CS-5b agonist” refers to an amount of CS-5b polypeptide or CS-5b agonist that is sufficient to stimulate lipid metabolism in an adipocyte. Preferably, such a method is directed to increase the lipolysis rate of an obese individual, thereby providing a treatment for the obesity. Preferably, this method is applied in combination with a reduced calorie diet.

In one embodiment, the present invention provides a method of inhibiting CS-5b for decreasing milk production in a mammal. Said method comprises the step of: delivering a pharmaceutical composition comprising a physiologically acceptable carrier and an effective amount of a CS-5b antagonist to an individual. As used herein, an “effective amount of a CS-5b antagonist” refers to an amount of CS-5b antagonist that is sufficient to reduce milk production. Such a method can be useful, e.g., to stop milk production when weaning a child.

Another embodiment of the present invention relates to methods of identifying CS-5b polypeptide fragments that are prolactin antagonists. Such methods comprise the steps of: i) providing a CS-5b polypeptide fragment; ii) contacting a cell with prolactin and said CS-5b polypeptide fragment, and iii) comparing prolactin receptor-mediated signaling in the cell after exposure to said CS-5b polypeptide fragment to that of a control cell that was exposed only to prolactin, wherein an observed decrease in prolactin receptor-mediated signaling between the exposed and unexposed cells indicates that said CS-5b polypeptide fragment is a prolactin antagonist. Prolactin receptor-mediated signaling may for example be assessed by determining insulin-induced leptin production in vitro as described in Ling et al. (Endocrinology 142:4880-90 (2001)), which disclosure is incorporated by reference herein in its entirety. Typically, a CS-5b polypeptide fragment that acts as a prolactin antagonist displays a functional prolactin receptor-binding domain but lacks amino-acids or domains necessary for prolactin receptor-mediated signal transduction. As further used herein, a “Pacs-5b polypeptide” refers to a CS-5b polypeptide fragment that acts as a prolactin antagonist. Prolactin antagonists are useful in methods for altering or resetting prolactin profiles, and thereby treating metabolic disorders such as, e.g., obesity, hyperlipidemia, hyperglycemia, hypercholesterolemia, insulin resistance and type II diabetes. Pacs-5b polypeptides may for example be used in place of bromocriptin in the methods described in U.S. Pat. No. 5,760,047 and in U.S. Pat. No. 4,659,715, the disclosures of which are both incorporated by reference in their entireties.

Physiologically acceptable carriers can be prepared by any method known by those skilled in the art. Physiologically acceptable carriers include but are not limited to those described in Remington's Pharmaceutical Sciences (Mack Publishing Company, Easton, USA 1985), which disclosure is hereby incorporated by reference in its entirety. Pharmaceutical compositions comprising a CS-5b agonist or antagonist and a physiologically acceptable carrier can be for, e.g., intravenous, topical, rectal, local, inhalant, subcutaneous, intradermal, intramuscular, oral and intracerebral use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

Still another embodiment relates to methods of constructing transgenic animals (e.g., mice) using recombinant molecules comprising a nucleic acid sequence encoding a CS-5b polypeptide or a nucleic acid sequence that encodes a cDNA that is complementary to CS-5b polynucleotides. Methods of constructing transgenic animals are well-known to those skilled in the art. For example, a CS-5b deficient mouse may be constructed according to U.S. Pat. No. 6,080,911, which disclosure is hereby incorporated by reference in its entirety. Alternatively, a transgenic mouse over-expressing CS-5b may be constructed. Such transgenic animals may be useful for obtaining animal models for dysfunctions associated with low or high lactogen levels (e.g., fetal growth retardation) and for testing the usefulness of, or screening for, chemical compounds in the treatment of such diseases.

Protein of SEQ ID NO:28 (Internal Designation Clone 500710542_{—}205-8-4-0-F1-F)

The cDNA of Clone 500710542_{—}205-8-4-0-F1-F (SEQ ID NO:27) encodes Placik of SEQ ID NO:28, comprising the amino-acid sequence: MAQLCGLRRSRAFLALLGSLLLSGVLAADRERSIHENATGDLATSRNAADSSVPSAPRRQDSED HSSDMFNYEEYCTANAVTGPCRASFPRWYFDVERNSCNNFIYGGCRGNKNSYRSEEACMLRCF RQQENPPLPLGSKVVVLAGLFVMVLILFLGASMVYLIRVARRNQERALRTVWSSGDDKEQLVK NTYVL. Accordingly, it will be appreciated that all characteristics and uses of the polypeptides of SEQ ID NO:28 described throughout the present application also pertain to the polypeptides encoded by the nucleic acids comprising the human cDNA in Clone 500710542_{—}205-8-4-0-F1-F. In addition, it will be appreciated that all characteristics and uses of the polynucleotides of SEQ ID NOs:## described throughout the present application also pertain to the nucleic acids comprising the human cDNA in Clone 500710542_{—}205-8-4-0-F1-F. A preferred embodiment of the invention is directed toward the human polynucleotide and polypeptide compositions of SEQ ID NO:28, SEQ ID NO:27, and Clone 500710542_{—}205-8-4-0-F1-F. Preferred Placik polypeptide fragments for uses in the methods described below include the Placik polypeptide comprising the amino sequence of: ADRERSIHENATGDLATSRNAADSSVPSAPRRQDSEDHSSDMFNYEEYCTANAVTGPCRASFPR WYFDVERNSCNNFIYGGCRGNKNSYRSEEACMLRCFRQQENPPLPLGSKVVVLAGLFVMVLILF LGASMVYLIRVARRNQERALRTVWSSGDDKEQLVKNTYVL. Also preferred are polypeptide fragments having a biological activity as described herein and the polynucleotides encoding the fragments.

The protein of the invention, Placik, is a NOVEL, secreted, splice and polymorphism variant of the Placental bikunin (SPINT2, EMBL accession number AAC02781). The first exon is identical in the Placik mRNA and in the SPINT2 mRNA. The second exon of the SPINT2 mRNA is not present in the Placik mRNA. The 3 following exons are identical in both mRNAs. Furthermore, the amino-acid shown at position −17 of SEQ ID NO:28 is an arginine in Placik and a proline in SPINT2. Placik displays a signal peptide (MAQLCGLRRSRAFLALLGSLLLSGVLA) and a Kunitz_BPTI domain (CTANAVTGPCRASFPRWYFDVERNSCNNFIYGGCRGNKNSYRSEEACMLRC) with a pancreatic trypsin inhibitor family signature. The resulting mature Placik polypeptide is 168 amino-acids long. The amino-acids shown at positions 1 to 8 of SEQ ID NO:28 are identical to the 8 amino-terminal amino-acids of the mature SPINT2 polypeptide, and the amino-acids shown at positions 9 to 168 of SEQ ID NO:28 are identical to the 160 carboxyl-terminal amino-acids of a mature SPINT2 polypeptide. Placik lacks a span of 57 amino-acids compared to SPINT2. Thus, contrarily to SPINT2 that displays two Kunitz_BPTI domains, Placik displays only one Kunitz_BPTI domain.

Placik is a broad-spectrum serine protease inhibitor. It exerts an inhibitory activity on serine proteases involved in blood coagulation and fibrinolysis such as plasmin, kallikreins and factor XIa. Kallikrein and plasmin are serine proteases that proteolitically cleave and activate fibrinolytic proenzymes such as prourokinase and plasminogen. Fibrinolysis leads to neutrophil activation, disseminated intravascular coagulation and increased vasopermeability. Furthermore, Kallikrein cleaves high molecular weight kininogen to form bradykinin, which is a potent vasodilator. Thus kallikrein and plasmin are involved in fibrin deposition and lysis, modulation of blood pressure, complement activation and support of the inflammatory system. In addition, several molecular interactions have been observed between the fibrinolytic and the matrix metalloproteinase (MMP) system, and both systems may cooperate in generating proteolytic activity and enhancing cancer cell invasiveness. Contrarily to SPINT2, Placik does not inhibit hepatocyte growth factor activators. Placik is expressed in a wide variety of tissues including, but not limited to, placenta, brain, kidney, pancreas, prostate, testis, thymus and trachea. Furthermore, Placik is overexpressed in prancreatic cancer cells, and its expression correlates inversely with the grade of human gliomas.

An embodiment of the invention is directed to a composition comprising a Placik polypeptide sequence of SEQ ID NO:28, or having at least 70%, 80%, 90%, 95%, 96%, 97%, 98%, 99%, or more identity to SEQ ID NO:28.

A further embodiment of the invention is directed to a composition comprising a Placik polypeptide fragment inhibiting serine proteases involved in blood coagulation or fibrinolysis.

As used herein, a “Placik polypeptide” refers either to a Placik polypeptide sequence of SEQ ID NO:28 or at least 70%, 80%; 90%, 95%, 96%, 97%, 98%, 99%, or more identical to SEQ ID NO:28, or to a Placik polypeptide fragment comprising the contiguous amino-acids shown as positions 8 and 9 of SEQ ID NO:28 and having biological activity of inhibiting serine proteases involved in blood coagulation or fibrinolysis.

An embodiment of the invention is directed to a composition comprising a polynucleotide sequence of SEQ ID NO:27 encoding a Placik polypeptide.

A further embodiment of the invention is directed to a composition comprising a polynucleotide sequence encoding a Placik polypeptide.

As further used herein, a “Placik polynucleotide” refers either to a Placik polynucleotide sequence of SEQ ID NO:27 or having at least 70%, 80%, 90%, 95%, or more identity to SEQ ID NO:27, to a nucleotide sequence encoding a Placik polypeptide, or to a sequence complementary to any of these sequences.

A further embodiment of the invention is directed to an antibody that recognizes a Placik polypeptide. Preferably, the antibody specifically binds to Placik but not to SPINT2. As used herein, an “anti-Placik antibody” refers to an antibody or antigen-binding fragment thereof that specifically binds to a Placik polypeptide.

An embodiment of the present invention relates to a method of binding an anti-Placik antibody to a Placik polypeptide comprising the step of: contacting a Placik polypeptide with said antibody under conditions that allow binding to occur. Such conditions are well known to those skilled in the art. Such methods are useful for detecting Placik polypeptides as further described herein.

An embodiment of the present invention is directed to a method of detecting Placik polypeptides in a biological sample, said method comprising the steps of: i) contacting a biological sample with an anti-Placik antibody; and ii) detecting the antigen-antibody complex formed. The anti-Placik antibody may be monoclonal or polyclonal. In addition, the anti-Placik antibody may be primarily or secondarily labeled by any detectable compound (e.g., radioactive, fluorescent, luminescent, or enzymatic) common in the art. Such a method may be applied to, e.g., diagnosis of prancreatic cancers or gliomas as further described below.

An embodiment of the present invention is directed to methods of detecting Placik expression in a biological sample, said methods comprising the steps of: i) providing a biological sample from an individual; ii) detecting the level of Placik expression in the sample; and iii) comparing the level of Placik expression in said sample to that of a control sample. In one such embodiment, said biological sample is obtained from an individual suspected of suffering from pancreatic cancer, and the method is used to diagnose pancreatic cancer. A higher level of Placik expression in said biological sample in comparison to the level in a control sample representative of a level in a normal individual is indicative of the presence of pancreatic cancer. In another such embodiment, said biological sample is obtained from an individual suspected of suffering from a glioma, e.g., an anaplastic astrocystoma or a glioblastoma. A lower level of Placik expression in said biological sample in comparison to the level in a control sample representative of a level in a normal individual is indicative of the presence of a glioma. The level of Placik expression in the sample can be assessed using any method, such as by detecting the level of Placik mRNA or Placik polypeptides in the sample. Any of a number of well-known techniques such as, e.g., western blot, immunochemical techniques and cytochemical techniques may be used to detect Placik expression. Preferably, anti-Placik antibodies are used to detect the level of Placik expression. Also preferably, polynucleotide probes comprising a polynucleotide sequence of SEQ ID NO:27 or part thereof, or a polynucleotide sequence complementary to SEQ ID NO:27, are used to detect the level of Placik expression by, e.g., northern blot or RTPCR techniques.

Another embodiment of the present invention is directed to a diagnostic kit for quantifying in vitro the presence of a Placik polypeptide. Such kit comprises: i) an anti-Placik antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Placik antibodies is used for diagnosing pancreatic cancer, or for detecting or determining the grade of a glioma. Optionally, said diagnostic kit comprises a negative control sample representative of the level from a normal individual. Optionally, said diagnostic kit comprises a positive control sample representative of the level from an individual suffering from a pancreatic cancer or a glioma.

Another embodiment relates to a method of producing a Placik polypeptide comprising the steps of: i) culturing a cell expressing a Placik polypeptide, and ii) purifying the produced protein. The purification of the protein can be carried out following any technique well-known to those skilled in the art. Preferably, an anti-Placik antibody may be bound to a chromatographic support to form an affinity chromatography column. Alternatively, the Placik polypeptide may be fused to a heterologous immunogenic peptide, and the fusion protein is purified using an antibody specific to the heterologous peptide. Preferably, the cell expressing a Placik polypeptide is a recombinant host cell as described below. Producing Placik polypeptides may be useful in methods and compositions as further described herein.

A preferred aspect of the invention is a host cell recombinant for polynucleotides encoding a Placik polypeptide, operably linked to a promoter. An embodiment is directed to a method of constructing a host cell recombinant for polynucleotides encoding a Placik polypeptide comprising the steps of: i) constructing a recombinant vector that comprises a nucleic acid sequence encoding a Placik polypeptide, operably linked to a promoter, and ii) introducing said recombinant vector into a cell. In preferred embodiments, the cell is an Escherichia coli cell or a human cell. An additional preferred aspect is a host cell recombinant for polynucleotides that, when present in a cell, cause an alteration in Placik expression. A host cell recombinant for polynucleotides capable of modifying Placik expression may be constructed by a method comprising the step of: i) providing a cell comprising the Placik gene; and ii) introducing a recombinant vector comprising Placik expression-altering polynucleotides into said cell, wherein the presence of said polynucleotides in said cell increases or decreases Placik expression compared to the level of Placik expression in said cell before said recombinant vector is introduced. Preferably, said polynucleotides are inserted into or replace all or part of the 5′ regulatory region of the Placik gene. Further preferably, said polynucleotides are located within 500 base pairs of the Placik coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are hereby incorporated by reference in their entireties. Such recombinant host cell producing Placik polypeptides may be used to produce Placik polypeptides.

A preferred embodiment of the invention is a method of using Placik to bind and/or inhibit serine proteases. This method comprises the step of: contacting a Placik polypeptide or active fragment thereof with a serine protease under conditions that allow Placik binding, whereby binding inhibits the activity of said serine protease. Preferred serine proteases include plasmin, kallikreins and factor XIa. This method may for example be applied to in vitro uses (e.g., protease purification and detection or prevention of protein degradation).

A preferred embodiment of the invention is a method of purifying serine proteases. This method comprises the steps of: i) contacting a Placik polypeptide that is immobilized on a support with a solution or sample comprising a serine protease under conditions that allow binding; ii) removing contaminants from said support; and iii) eluting the bound serine protease off of said support. Preferably, the Placik peptide is immobilized on a solid or semi-solid matrix to facilitate washing of sample to remove contaminants. Preferred serine proteases include plasmin, kallikreins, and factor XIa. These may be purified from common biological fluids such as cell culture media and body fluids (e.g., serum, blood or lymph). Purified proteases are useful in biological assays and techniques such as removal of adherent cells from a culture dish and site-directed peptide design.

A preferred embodiment relates to methods of screening test substances for the ability to modulate Placik expression comprising the steps of: i) contacting a cell with a test substance; and ii) comparing Placik expression in the cell after exposure to the test substance to that of an unexposed control cell, wherein an observed difference in Placik expression between the exposed cell and the unexposed control cell indicates that the test substance modulates Placik expression. Placik expression may be determined by methods common to the art including but not limited to methods of quantifying Placik polynucleotides (e.g., detection of Placik mRNA by northern blot or RTPCR) or to methods of quantifying Placik polypeptides (e.g., detection of Placik polypeptides by western blot or immunochemistry). In one embodiment, the test substance modifies the expression of Placik in a specific cell type but not in others. Modulators identified using such methods are also encompassed by the present invention, as are methods of using the modulators, e.g., to alter the expression of Placik in a cell, tissue, or individual.

A further embodiment of the present invention is directed to methods of screening test substances for the ability to modulate Placik activity comprising the steps of: i) contacting a cell with a test substance, and ii) comparing Placik activity in the cell after exposure to the test substance to that of an unexposed control cell, wherein an observed difference in Placik activity between the exposed and unexposed cells indicates that the test substance modulates Placik activity. Placik activity can be determining by any of a number of techniques common to the art. Placik activity can be monitored, e.g., by measuring in vitro its inhibitory activity against trypsin, plasma kallikrein, tissue kallikrein or plasmin as described in Delaria et al. (J Biol Chem. 272:12209-14 (1997)), which disclosure is hereby incorporated by reference in its entirety. Alternatively, Placik activity may also be monitored in cells, for example by measuring the fibrinolytic activities of gliobastoma cell lines or measuring the Matrigel invasion of U251 and YKG-1 cells as described by Hamasuna et al. (Int J Cancer 93:339-45 (2001)), the disclosure of which is incorporated by reference herein in its entirety. Modulators identified using such methods are also encompassed by the present invention, as are methods of using the modulators, e.g. to alter the activity of Placik in a cell, tissue, or individual.

Test substances that decrease Placik expression or activity are defined as Placik antagonists. Test substances that increase Placik expression or activity are defined as Placik agonists. Test substances that modulate the expression or activity of Placik include, but are not limited to, chemical compounds (e.g. small-molecule inhibitors or activators), oligonucleotides, antisense polynucleotides, polypeptides, ribozymes, dominant negative forms of Placik, and anti-Placik antibodies. These substances may be made and used according to methods well known in the art.

An embodiment of the present invention relates to a method of using a Placik polypeptide to inhibit contaminating proteases in a sample. Such a method comprises the step of: adding a protease-inhibiting amount of a Placik polypeptide to a solution under conditions that allow Placik activity. Preferably, said proteases are kallikreins, plasmin or factor XIa. Preferably, the Placik polypeptide is comprised in a “cocktail” of protease inhibitors that is able to inhibit a wide range of proteases without necessarily knowing the specificity of any of the proteases. Such protease inhibitor cocktails are widely used in laboratory assays to prevent degradation of any protein sample by contaminating proteases.

Another embodiment of the present invention relates to methods of using Placik polypeptides to inhibit kallikrein or plasmin in a cell. Such a method comprises the step of: contacting a cell with a kallikrein-inhibiting or a plasmin-inhibiting amount of a Placik polypeptide or a Placik agonist under conditions that allow Placik activity. Such methods can be performed in any cell type, most preferably neutrophils, endothelial cells or tumoral cells.

In one such embodiment, said method of using Placik polypeptides to inhibit kallikrein or plasmin in a cell is directed to decrease fibrinolysis in an individual. Such a method comprises the step of: delivering a physiologically acceptable carrier and an effective amount of a Placik polypeptide or a Placik agonist to an individual. As used in the context of this embodiment, an “effective amount of a Placik polypeptide or a Placik agonist” refers to an amount of Placik polypeptide or Placik agonist that is sufficient to inhibit kallikrein and/or plasmin proteolytic activity. Preferably, such a method is directed to treat or prevent diseases where inhibition of kallikrein and plasmin is indicated such as, e.g., sepsis or septic shock, inflammation, acute respiratory distress syndrome, disseminated intravascular coagulation, hypotension, cardiopulmonary bypass surgery and bleeding from postoperative surgery. Methods of delivering Kunitz-type protease inhibitors are for example described in U.S. Pat. No. 5,407,915 and U.S. Pat. No. 5,786,328, which disclosures are incorporated herein by reference in theirs entireties. Compared to SPINT2 and bikunin, Placik polypeptides offer the advantage of not inhibiting HGF activators and being specific for enzymes involved in the fibrinolytic system.

Another embodiment of the present invention relates to methods of using Placik polypeptides and Placik agonists to prevent or reduce ECM degradation in a mammal or in a tussue culture. Such a method comprises the step of: delivering a physiologically acceptable carrier and an effective amount of a Placik polypeptide or a Placik agonist to an individual or a tissue. As used in the context of this embodiment, an “effective amount of a Placik polypeptide or a Placik agonist” refers to an amount of Placik polypeptide or Placik agonist that is sufficient to inhibit or reduce ECM degradation. Preferably, such methods for preventing or reducing ECM degradation are directed to protect the ECM from degradation by malignant cells, thus blocking the invasion and spread of malignant tumors. Most preferably, a composition comprising a Placik polypeptide is administered to an individual suffering from abnormal or undesirable cell proliferation, such as, e.g., tumor growth, endothelial cell proliferation, and angiogenesis related to tumor growth. Tumors that can be treated with the compositions of the present invention include, but are not limited to, pancreatic cancers, gliomas, anaplastic astrocystomas and glioblastomas. In such a method, the Placik polypeptide may be fused to a targeting molecule specific for the tumor to be treated. Said targeting molecules specific for the tumor to be treated include but are not limited to ligands and antibody that recognizes a receptor or an antigen specifically expressed on tumor cells. A large number of tumor-associated antigens have been described in the scientific literature. For example, antigens and techniques described by Wikstrand et al. (Cancer Metastasis Rev 18:451-64 (1999)), which disclosure is hereby incorporated by reference in its entirety, may be used. Alternatively, the Placik polypeptide may be linked to a ligand that recognizes the vasculature of a tumor. A preferred method for targeting the Placik polypeptide to the vasulature of solid tumors is described in U.S. Pat. No. 6,004,554, which disclosure is incorporated by reference in its entirety.

Effective dosages of a Placik polypeptide or a Placik modulator can be readily determined by one of ordinary skill in the art through, e.g., routine trials establishing dose response curves. Typical dosing regimes will be analogous to treatment of the diseases listed above by the use of other analogous proteins such as, e.g., bikunin, SPINT2 and aprotinin. The frequency of administration, and the administered amount of a composition comprising a physiologically acceptable carrier and a Placik polypeptide or a Placik modulator will be in accordance with the particular disease being treated, its severity, the nature of the Placik polypeptide or Placik modulator employed, the overall condition of the individual and the judgement of the treating physician. As used herein, the term “physiologically acceptable carrier” refers to a composition that is compatible with the individual and the Placik polypeptide or Placik agonist employed, and that is approved by a regulatory agency of the Federal or a state government or listed in the U.S. Pharmacopeia or other generally recognized pharmacopeia for use in animals, and more particularly in humans. Said physiologically acceptable carrier include but are not limited to solutions and suspensions such as biological buffers (e.g., phosphate buffered saline, saline and Dulbecco's Media), starches, sugars, microcrystalline cellulose, diluents, granulating agents, lubricants, binders and disintegrating agents. The compositions comprising a physiologically acceptable carrier and the polypeptides or modulators of the present invention may be administered in any suitable manner such as, for example, intravenous, parental, topical, oral, or local (e.g., aerosol and transdermal) administration, or any combination thereof. Preferred routes of administration comprise intravenous and oral administration.

Still another embodiment relates to methods of constructing transgenic animals (e.g., mice) using recombinant molecules comprising a nucleic acid sequence encoding a Placik polypeptide or a nucleic acid sequence that encodes a cDNA that is complementary to Placik polynucleotides. Methods of constructing transgenic animals are well-known to those skilled in the art. For example, a transgenic mouse may be constructed according to U.S. Pat. No. 4,736,866, or U.S. Pat. No. 5,614,396, which disclosures are hereby incorporated by reference in their entireties. Alternatively, a transgenic mouse over-expressing Placik may be constructed. Such transgenic animals may be useful for, e.g., obtaining animal models being predisposed to invasion and spread of malignant tumors, obtaining animal models for diseases such as acute respiratory distress syndrome and disseminated intravascular coagulation, or testing the usefulness of chemical compounds in the treatment of such diseases.

Protein of SEQ ID NO:30 (Internal Designation Clone 500728413_{—}204-53-2-0-B2-F)

The cDNA of Clone 500728413_{—}204-53-2-0-B2-F (SEQ ID NO:29) encodes Codeac of SEQ ID NO:30, comprising the amino-acid sequence: MTVARPSVPAALTPLLGELPRLLLLVLLCLPAVWGDCGLPPDVPNAQPALEGRTSFPEDTVITYKC EESFVKIPGEKDSVICLKGSQWSDIEEFCNQKSCPNPGEIRNGQIDVPGGILFGATISFSCNTGYKLF GSTSSFCLISGSSVQWSDPLPECREIYCPAPPQIDNGIIQGERDHYGYRQSVTYACNKGFTMIGEHS IYCTVNNDEGEWSGPPPECRGKSLTSKVPPTVQKPTTVNVPTTEVSPTSQKTTTKTTTPNAQATR STPVSRTTKHFHETTPNKGSGTTSGTTRLLSGHTCFTLTGLLGTLVTMGLLT. Accordingly, it will be appreciated that all characteristics and uses of the polypeptides of SEQ ID NO:30 described throughout the present application also pertain to the polypeptides encoded by the nucleic acids comprising the human cDNA in Clone 500728413_{—}204-53-2-0-B2-F. In addition, it will be appreciated that all characteristics and uses of the polynucleotides of SEQ ID NOs:## described throughout the present application also pertain to the nucleic acids comprising the human cDNA in Clone 500728413_{—}204-53-2-0-B2-F. A preferred embodiment of the invention is directed toward the human polynucleotide and polypeptide compositions of SEQ ID NO:30, SEQ ID NO: 29, and Clone 500728413_{—}204-53-2-0-B2-F. Preferred Codeac polypeptide fragments for uses in the methods described below include the Codeac polypeptide comprising the amino acid sequence of: DCGLPPDVPNAQPALEGRTSFPEDTVITYKCEESFVKIPGEKDSVICLKGSQWSDIEEFCNQKSCP NPGEIRNGQIDVPGGILFGATISFSCNTGYKLFGSTSSFCLISGSSVQWSDPLPECREIYCPAPPQID NGIIQGERDHYGYRQSVTYACNKGFTMIGEHSIYCTVNNDEGEWSGPPPECRGKSLTSKVPPTVQ KPTTVNVPTTEVSPTSQKTTTKTTTPNAQATRSTPVSRTTKHFHETTPNKGSGTTSGTTRLLSGHT CFTLTGLLGTLVTMGLLT. Most preferred Codeac polypeptide fragments for uses in the methods described below include the Codeac polypeptide comprising the amino acid sequence of: DCGLPPDVPNAQPALEGRTSFPEDTVITYKCEESFVKIPGEKDSVICLKGSQWSDIEEFCNQKSCP NPGEIRNGQIDVPGGILFGATISFSCNTGYKLFGSTSSFCLISGSSVQWSDPLPECREIYCPAPPQID NGIIQGERDHYGYRQSVTYACNKGFTMIGEHSIYCTVNNDEGEWSGPPPECRGKSLTSKVPPTVQ KPTTVNVPTTEVSPTSQKTTTKTTTPNAQATRSTPVSRTTKHFHETTPNKGSGTTSG. Also preferred are polypeptide fragments having a biological activity as described herein and the polynucleotides encoding the fragments.

The protein of the invention, Codeac, is a NOVEL, secreted, splice variant of the complement decay-accelerating factor (CD55, EMBL accession number M31516). The first and second exons are identical in the Codeac mRNA and in the CD55 mRNA. The third exon of the CD55 mRNA is not present in the Codeac mRNA. The 5 following exons are identical in both mRNAs. Codeac displays a signal peptide (MTVARPSVPAALPLLGELPRLLLLVLLCLPAVWG), a propeptide at its carboxyl-terminal extremity that is removed in mature form (TTRLLSGHTCFTLTGLLGTLVTMGLLT) and three sushi domains

(CGLPPDVPNAQPALEGRTSFPEDTVITYKCEESFVKIPGEKDSVICLKGSQWSDIEEFCN,
SCPNPGEIRNGQIDVPGGILFGATISFSCNTGYKLFGSTSSFCLISGSSVQWSDPLPECR
and
CPAPPQIDNGIIQGERDHYGYRQSVTYACNKGFTMIGEHSIYCTVNNDEGEWSGPPPECRGKS).

The resulting mature Codeac polypeptide is 256 amino-acids long. The amino-acids at positions 1 to 61 of SEQ ID NO:30 are identical to the 61 amino-terminal amino-acids of the mature CD55 polypeptide. The Glutamine at position 62 of SEQ ID NO:30 is unique to Codeac. The amino-acids at positions 63 to 256 of SEQ ID NO:30 are identical to the amino-acids at positions 161 to 354 of the CD55 polypeptide shown under EMBL accession number M31516. Codeac lacks a span of 64 amino-acids compared to CD55. Thus, contrarily to CD55 that displays four sushi domains, Codeac lacks the second sushi domain of CD55 and displays only three sushi domains.

Codeac is a GPI-anchored protein that is a member of the regulators of complement activation family. It has a widespread tissue distribution and is expressed at high levels on many different cell types. In particular, Codeac is expressed on epithelial cells lining extracellular compartments and on cells that are in contact with plasma, body fluids and extracellular matrix. The cellular ligand of Codeac is CD97, a member of the EGF-TM7 family. CD97 is constitutively expressed on granulocytes and monocytes, and is rapidly up-regulated on activated T and B cells. The Codeac-CD97 interaction plays a role in adhesion and signaling within the normal inflammatory and immune responses. Furthermore, Codeac functions as a receptor for a variety of viral and bacterial pathogens. Thus Codeac contributes to maintaining a reservoir of bound pathogens in an infectious state, inhibiting membrane attack complex assembly on pathogens and mediating viral lytic cell infection. Contrarily to CD55, Codeac does not bind to the C3b and C4b convertases of the complement system, and does not protect host cells from activation of autologous complement on their surfaces.

An embodiment of the invention is directed to a composition comprising a Codeac polypeptide sequence of SEQ ID NO:30, or having at least 70%, 80%, 90%, 95%, 96%, 97%, 98%, 99%, or more identity to SEQ ID NO: 30.

A further embodiment of the invention is directed to a composition comprising a Codeac polypeptide fragment that binds to CD97 and/or to viral and bacterial pathogens.

As used herein, a “Codeac polypeptide” refers either to a Codeac polypeptide sequence of SEQ ID NO:30 or at least 70%, 80%; 90%, 95%, 96%, 97%, 98%, 99%, or more identical to SEQ ID NO: 30, or to a Codeac polypeptide fragment comprising the contiguous amino-acids shown as positions 61 to 63 of SEQ ID NO:30 and that binds to CD97 and/or to viral and bacterial pathogens.

An embodiment of the invention is directed to a composition comprising a polynucleotide sequence of SEQ ID NO:29 encoding a Codeac polypeptide.

A further embodiment of the invention is directed to a composition comprising a polynucleotide sequence encoding a Codeac polypeptide.

As further used herein, a “Codeac polynucleotide” refers either to a Codeac polynucleotide sequence of SEQ ID NO:29 or having at least 70%, 80%, 90%, 95%, or more identity to SEQ ID NO:29, to a nucleotide sequence encoding a Codeac polypeptide, or to a sequence complementary to any of these sequences.

A further embodiment of the invention is directed to an antibody that recognizes a Codeac polypeptide. Preferably, the antibody recognizes at least two of the contiguous amino-acids shown as positions 61 to 63 of SEQ ID NO:30, wherein said amino-acids are required for binding of the antibody to a Codeac polypeptide. Also preferably, the antibody recognizes the EEFCNQKSCPNPGEIRNGQID epitope. Most preferably, the antibody binds to Codeac but not to CD55. As used herein, an “anti-Codeac antibody” refers to an antibody or antigen-binding fragment thereof that specifically binds to a Codeac polypeptide.

An embodiment of the present invention relates to a method of binding an anti-Codeac antibody to a Codeac polypeptide comprising the step of: contacting a Codeac polypeptide with said antibody under conditions that allow binding to occur. Such conditions are well known to those skilled in the art. Such methods are useful for detecting Codeac polypeptides as further described herein.

An embodiment of the present invention is directed to a method of detecting Codeac polypeptides in a biological sample, said method comprising the steps of: i) contacting a biological sample with an anti-Codeac antibody; and ii) detecting the antigen-antibody complex formed. The anti-Codeac antibody may be monoclonal or polyclonal. In addition, the anti-Codeac antibody may be primarily or secondarily labeled by any detectable compound (e.g., radioactive, fluorescent, luminescent, or enzymatic) common in the art. Such a method may be applied to, e.g., diagnosis of various diseases as further described below.

An embodiment of the present invention is directed to methods of detecting Codeac expression in a biological sample, said methods comprising the steps of: i) providing a biological sample from an individual; ii) detecting the level of Codeac expression in the sample; and iii) comparing the level of Codeac expression in said sample to that of a control sample. Detecting Codeac expression is useful, for example, for diagnosis of various diseases such as, e.g., cancer, paroxysmal nocturnal hemoglobinuria, ulcerative colitis and joint diseases. The level of Codeac expression in the sample can be assessed using any method, such as by detecting the level of Codeac mRNA or Codeac polypeptides in the sample. Any of a number of well-known techniques such as, e.g., western blot, immunochemical techniques and cytochemical techniques may be used to detect Codeac expression. Most preferred well-known techniques that may be used are flow cytometric assays, immunohistochemically techniques and immunoperoxidase staining using anti-Codeac antibodies. Most preferably, anti-Codeac antibodies are used to detect the level of Codeac expression. Also preferably, polynucleotide probes comprising a polynucleotide sequence of SEQ ID NO:29 or part thereof, or a polynucleotide sequence complementary to SEQ ID NO:29, are used to detect the level of Codeac expression by, e.g., northern blot or RTPCR techniques.

In one such embodiment, said biological sample is obtained from an individual susceptible of suffering from a cancer, and the method is used to diagnose a cancer such as, e.g., a colorectal cancer, a cervical cancer, a renal cancer, a breast cancer, an ovarian cancer, a squamous cell carcinoma, a hepatoma or an osteosarcoma. For example, measuring Codeac expression for detecting a colorectal cancer in the absence of occult blood can be performed as described in U.S. Pat. No. 5,695,945, which disclosure is incorporated by reference in its entirety. In such a method, a higher level of Codeac expression in said biological sample in comparison to the level in a control sample representative of a level in a normal individual is indicative of the presence of a cancer.

In another such embodiment, said biological sample is obtained from an individual potentially suffering from an ulcerative colitis, and a higher level of Codeac expression in said biological sample in comparison to the level in a control sample representative of a level in a normal individual is indicative of an ulcerative colitis. Preferred biological samples for use in this embodiment are biological samples obtained from colonic mucosa or colonic epithelial cells. Detecting Codeac expression for diagnosis of ulcerative colitis may for example be performed as described in Uesu et al. (Lab Invest. 72:587-91 (1995)), which disclosure is incorporated herein in its entirety.

In still another such embodiment, said biological sample is obtained from an individual potentially suffering from paroxysmal nocturnal hemoglobinuria (PNH), the Inab phenotype, systemic sclerosis or various joint diseases such as, e.g., rheumatoid arthritis. In such a method, a lower level of Codeac expression in said biological sample in comparison to the level in a control sample representative of a level in a normal individual is indicative of the presence of one of these diseases. Preferred biological samples for diagnosis of systemic sclerosis are biological samples obtained from punch biopsies of skin. Detecting Codeac expression for diagnosis of systemic sclerosis may for example be performed as described in Venneker et al. (Lab Invest. 70:830-5 (1994)), which disclosure is incorporated herein in its entirety. Preferred biological samples for diagnosis of PNH are biological samples obtained from blood samples comprising erythrocytes and/or granulocytes. Detecting Codeac expression for diagnosis of PNH may for example be performed as described in Oelschlaegel et al. (Clin Lab Haernatol. 23:81-90 (2001)), which disclosure is incorporated herein in its entirety. Preferred biological samples for diagnosis of rheumatoid arthritis are biological samples obtained from blood samples comprising neutrophils. Detecting Codeac expression for diagnosis of rheumatoid arthitis may for example be performed as described in Jones et al. (Br J Rheumatol. 33:707-12 (1994)), which disclosure is incorporated herein in its entirety.

Another embodiment of the present invention is directed to a diagnostic kit for quantifying in vitro the presence of a Codeac polypeptide. Stich kit comprises: i) an anti-Codeac antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Codeac antibodies is used for diagnosing one of the diseases listed herein. Optionally, said diagnostic kit comprises a negative control sample representative of the level from a healthy, disease-free individual. Optionally, said diagnostic kit comprises a positive control sample representative of the level from an individual suffering from a given disease selected from the group consisting of cancer, ulcerative colitis, PNH, the Inab phenotype, systemic sclerosis and joint diseases.

Still another embodiment is directed to methods of using anti-Codeac antibodies for detecting Codeac polypeptides in vivo, for example by immunoelectron microscopy or by immunohistochemical staining. Such a method, which is useful for tumor imaging, is for example described in Li et al. (Br J Cancer 84:80-6 (2001)), which disclosure is incorporated by reference in its entirety.

Another embodiment relates to a method of producing a Codeac polypeptide comprising the steps of: i) culturing a cell comprising a polynucleotide encoding a Codeac polypeptide under conditions conducive to the expression of said polypeptide, and ii) purifying the produced polypeptide. The purification of the polypeptide can be carried out following any technique well-known to those skilled in the art. Preferably, an anti-Codeac antibody may be bound to a chromatographic support to form an affinity chromatography column. Alternatively, the Codeac polypeptide may be fused to a heterologous immunogenic peptide, and the fusion protein is purified using an antibody specific to the heterologous peptide. Preferably, the cell expressing a Codeac polypeptide is a recombinant host cell as described below. Producing Codeac polypeptides may be useful in methods and compositions as further described in the present invention.

A preferred aspect of the invention is a host cell recombinant for polynucleotides encoding a Codeac polypeptide, operably linked to a promoter. An embodiment is directed to a method of constructing a host cell recombinant for polynucleotides encoding a Codeac polypeptide comprising the steps of: i) constructing a recombinant vector that comprises a nucleic acid sequence encoding a Codeac polypeptide, operably linked to a promoter, and ii) introducing said recombinant vector into a cell. In preferred embodiments, the cell is an Escherichia coli cell or a human cell. An additional preferred aspect is a host cell recombinant for polynucleotides that, when present in a cell, cause an alteration in Codeac expression. A host cell recombinant for polynucleotides capable of modifying Codeac expression may be constructed by a method comprising the step of: i) providing a cell comprising the Codeac gene; and ii) introducing a recombinant vector comprising Codeac expression-altering polynucleotides into said cell, wherein the presence of said polynucleotides in said cell increases or decreases Codeac expression compared to the level of Codeac expression in said cell before said recombinant vector is introduced. Preferably, said polynucleotides are inserted into or replace all or part of the 5′ regulatory region of the Codeac gene. Further preferably, said polynucleotides are located within 500 base pairs of the Codeac coding region. Said polynucleotides preferably comprise a promoter sequence. Techniques known in the art for introducing polynucleotide sequences to endogenous sequences are described in U.S. Pat. No. 5,641,670 and PCT W0962941 1, which disclosures are herein incorporated by reference in their entireties. Such recombinant host cell producing Codeac polypeptides may be used to produce Codeac polypeptides.

In a further aspect, the present invention provides a cancer vaccine comprising a Codeac polypeptide and a physiologically acceptable carrier, and a method of using said cancer vaccine comprising the step of: delivering an effective amount of said cancer vaccine to an individual, wherein said cancer vaccine induces an immune response in said individual against said Codeac polypeptide. As used in the context of this embodiment, an “effective amount of a cancer vaccine” refers to an amount of vaccine that is sufficient to induce an immune response against Codeac polypeptides in an individual, e.g. a cancer patient. The immune response may be one or more of a T-helper cell response, a cytotoxic T-cell response, an NK cell response and/or an immune response. Such a method provides a way of selectively targeting a T-cell response to cancer cells expressing high levels of Codeac polypeptides. Such a method may be performed as described in WO application 99/43800, which is incorporated by reference in its entirety, by substituting the polypeptides of the present invention for 791Tgp72. referred Codeac polypeptides for use in such a method are Codeac polypeptides that induce an immune response against Codeac but not against CD55. Such immunogenic Codeac polypeptides comprise at least two of the contiguous amino-acids shown as positions 61 to 63 of SEQ ID NO:30. Preferred such immunogenic Codeac polypeptides comprise the EEFCNQKSCPNPGERNGQID epitope. Compared to a cancer vaccine comprising 791Tgp72, a cancer vaccine comprising such immunogenic Codeac polypeptides offers the advantage of not inducing antibodies against CD55, and thus not decreasing protection of cells from complement mediated attack.

Alternatively, an anti-Codeac antibody may be used as a targeting and delivery mechanism for bringing a pharmaceutical agent to tumor cells that express high levels of Codeac polypeptides. Such a method comprises the steps of: i) conjugating an anti-Codeac antibody to a pharmaceutical agent; and ii) administering a composition comprising said conjugate and a pharmaceutically acceptable carrier to an individual suffering from a cancer. Preferably, an anti-Codeac antibody is used in an antibody-directed enzyme-prodrug therapy comprising the steps of: i) conjugating an anti-Codeac antibody to an enzyme converting a relatively non-toxic compound into a substantially more toxic compound; and ii) administering said relatively non-toxic compound to a patient after a delay that allowed residual enzyme-antibody conjugate to be cleared from the blood. Such a method may be performed as described by Melton et al. (J. Natl. Cancer Inst. 88:153-65 (1996)), which disclosure is herein incorporated by reference in its entirety.

A preferred embodiment relates to methods of screening test substances for the ability to modulate Codeac expression comprising the steps of: i) contacting a cell with a test substance; and ii) comparing Codeac expression in the cell after exposure to the test substance to that of an unexposed control cell, wherein an observed difference in Codeac expression between the exposed cell and the unexposed control cell indicates that the test substance modulates Codeac expression. Codeac expression may be determined by methods common to the art including but not limited to methods of quantifying Codeac polynucleotides (e.g., detection of Codeac mRNA by northern blot or RTPCR) or to methods of quantifying Codeac polypeptides (e.g., detection of Codeac polypeptides by western blot or immunochemistry). In one embodiment, the test substance modifies the expression of Codeac in a specific cell type but not in others. Preferred cell types comprise, e.g., epithelial cells, fibroblasts and tumoral cells. Modulators identified using such methods are also encompassed by the present invention, as are methods of using the modulators, e.g., to alter the expression of Codeac in a cell, tissue, or individual.

A further embodiment of the present invention is directed to methods of screening test substances for the ability to modulate Codeac activity comprising the steps of: i) contacting a cell with a test substance, and ii) comparing Codeac activity in the cell after exposure to the test substance to that of an unexposed control cell, wherein an observed difference in Codeac activity between the exposed and unexposed cells indicates that the test substance modulates Codeac activity. Codeac activity can be determining by any of a number of techniques common to the art. Codeac activity can be monitored, e.g., by studying adhesion of Codeac-expressing cells to CD97-expressing cells as described in Lin et al. (3 Biol Chem. 276:24160-9 (2001)), which disclosure is herein incorporated by reference in its entirety. Alternatively, Codeac activity may be monitored by studying adhesion of Codeac-expressing cells to adhesin-expressing cells as described in Nowicki et al. (J Exp Med. 178:2115-21 (1992)), the disclosure of which is incorporated by reference herein in its entirety. Modulators identified using such methods are also encompassed by the present invention, as are methods of using the modulators, e.g. to alter the activity of Codeac in a cell, tissue, or individual.

Test substances that decrease Codeac expression or activity are defined as Codeac antagonists. Test substances that increase Codeac expression or activity are defined as Codeac agonists. Test substances that modulate the expression or activity of Codeac include, but are not limited to, chemical compounds (e.g. small-molecule inhibitors or activators), oligonucleotides, antisense polynucleotides, ribozymes, carbohydrates, lipids and polypeptides (e.g. natural ligands, transcriptional regulators, dominant negative forms of Codeac, immunogenic Codeac polypeptides and anti-Codeac antibodies). These substances may be made and used according to methods well known in the art.

A preferred embodiment of the invention is a method of using a Codeac polypeptide to bind CD97. This method comprises the step of: contacting a Codeac polypeptide with CD97 or a fragment thereof under conditions that allow Codeac binding. This method may for example be applied to in vitro uses as further described below.

In a preferred embodiment, binding of a Codeac polypeptide to CD97 is used to purify cells expressing CD97. Such a method for purifying cells expressing CD97 comprises the steps of: i) labeling by standard methods of a Codeac polypeptide with a molecule that can be used to provide a detectable signal, and ii) running a body fluid comprising cells expressing CD97 through a sorting apparatus, e.g. a fluorescence-activated cell sorter or a magnetic activated cell-sorting apparatus, which contains said labeled Codeac polypeptide. As CD97 is expressed at high levels on activated lymphocytes, such a method may for example be used for purifying human lymphocyte subpopulations. Purifying lymphocyte subpopulations is of great importance for, e.g., understanding the mechanisms and interactions of normal and pathological immune reactions, conducing a T cell therapy against viruses, or identifying T cell-stimulating antigens.

An embodiment of the present invention relates to a method of using a Codeac polypeptide or a Codeac agonist to enhance a Codeac-CD97 interaction, thereby altering or reducing an undesirable immune response. In a preferred embodiment, such a method comprises the step of: delivering a physiologically acceptable carrier and an effective amount of a Codeac polypeptide or a Codeac agonist to an individual. As used in the context of this embodiment “an effective amount of a Codeac polypeptide or a Codeac agonist” refers to an amount of Codeac polypeptide or Codeac agonist that is sufficient to reduce an immune response. Preferably, such methods for reducing an immune response are directed to treat allergy and asthma, and to prevent undesireable immune responses. More particularly, such methods can be used to prevent or reduce in severity graft rejections, graft versus host diseases and autoimmune diseases (e.g., rheumatoid arthritis, systemic lupus, multiple sclerosis, insulin-dependent diabetes, hepatitis, Graves disease), and to induce tolerance to graft transplantation (e.g., transplantation of cells, bone marrow, tissue, solid-organ, bone).

Another embodiment of the present invention relates to a method of using a Codeac antagonist to inhibit Codeac-CD97 interaction, thus enhancing an immune response. A preferred method directed to enhance an immune response comprises the step of: delivering a physiologically acceptable carrier and an effective amount of a Codeac antagonist to an individual suffering from diseases and disorders where a boost to the immune system is desirable. As used in the context of this embodiment “an effective amount of a Codeac antagonist” refers to an amount of Codeac antagonist that is sufficient to enhance an immune response. More particularly, such methods can be used in conjunction with a radiation therapy or a chemotherapy when treating a cancer, and can be used to treat neoplastic disorders (e.g., multiple myeloma, colon cancer, hepatoma), viral infections (e.g., HIV, HBV, HCV, hepatitis, measles and herpes viruses infections), and various immune deficiencies. These immune deficiencies may be genetic (e.g. severe combined immunodeficiency) or be caused by various bacterial or fungal infections (e.g. infections by mycobacteria, Leishmania spp., malaria spp. and candidiasis).

A preferred embodiment of the invention is a method of using a Codeac polypeptide to bind viral and bacterial pathogens. This method comprises the step of: contacting a Codeac polypeptide with a viral or bacterial pathogen under conditions that allow binding of said Codeac polypeptide to said pathogen. Preferred pathogens for uses in this method include, but are not limited to, pathogenic Escherichia coli, Helicobacter pylori, coxsakievirus and echovirus. Alternatively, a cell surface protein of a pathogen may be used instead of a pathogen. Preferred such surface proteins are adhesins. A wide variety of conditions that allow binding of Codeac to pathogens are available. Such conditions are for example described in Schmidtke et al. (Virology. 275(l):77-88 (2000)). Using a Codeac polypeptide to bind viral and bacterial pathogens may for example be applied to in vitro uses as further described below.

A preferred aspect of this embodiment includes a method of using a Codeac polypeptide to isolate or purify pathogens. This method comprises the steps of: i) contacting a Codeac polypeptide that is immobilized on a support with a solution or biological sample potentially comprising a pathogen under conditions that allow binding; ii) removing contaminants from said support; and iii) eluting the bound pathogen off of said support. Preferably, the Codeac polypeptide is immobilized on a solid or semi-solid matrix to facilitate washing of sample to remove contaminants. Preferred pathogens include but are not limited to E. coli, H. pylor, cocksackieviruses, picomaviruses and echoviruses. These may be isolated from common biological fluids such as cell culture media, body fluids and liquid samples obtained from body samples (e.g., seruim, blood, lymph, urine, gastric mucosa and feces). Such a method is for example useful to determine whether a disease is caused by a pathogen or to determine the pathogen that causes a chronic infection.

Another preferred embodiment of the invention relates to a method of using a Codeac antagonist to inhibit Codeac-pathogen interaction, thus reducing the reservoir of bound pathogens in an infectious state. Such a method comprises the sten of delivering a physiologically acceptable carrier and an effective amount of a Codeac antagonist to an individual suffering from a chronic infection. As used in the context of this embodiment, “an effective amount of a Codeac antagonist” refers to an amount of Codeac antagonist that is sufficient to reduce binding of pathogens to Codeac. Chronic infections that may be treated or reduced in severity by such a method comprise, but are not limited to, H. pylori-associated or E. coli-associated intestinal infections, picornavirus-associated diseases, coxsackievirus-associated diseases (such as, e.g., heart diseases, pancreatic infections, acute flaccid paralysis and neurodevelopmental delays in the newborn) and echovirus associated diseases (such as, e.g., encephalomyelitis, amyelotrophic lateral sclerosis, hepatic failure and meningitis).

Effective dosages of a Codeac polypeptide or a Codeac modulator can be readily determined by one of ordinary skill in the art through, e.g., routine trials establishing dose response curves. The frequency of administration, and the administered amount of a composition comprising a physiologically acceptable carrier and a Codeac polypeptide or a Codeac modulator will be in accordance with the particular disease being treated, its severity, the nature of the Codeac polypeptide or Codeac modulator employed, the overall condition of the individual and the judgement of the treating physician. As used herein, the term “physiologically acceptable carrier” refers to a composition that is compatible with the individual and the Codeac polypeptide or Codeac modulator employed, and that is approved by a regulatory agency of the Federal or a state government or listed in the U.S. Pharmacopeia or other generally recognized pharmacopeia for use in animals, and more particularly in humans. Said physiologically acceptable carrier include but are not limited to solutions and suspensions such as biological buffers (e.g., phosphate buffered saline, saline and Dulbecco's Media), starches, sugars, microcrystalline cellulose, diluents, granulating agents, lubricants, binders and disintegrating agents. The compositions comprising a physiologically acceptable carrier and the polypeptides or modulators of the present invention may be administered in any suitable manner such as, for example, intravenous, parental, topical, oral, or local (e.g., aerosol and transdermal) administration, or any combination thereof. Preferred routes of administration comprise intravenous and oral administration.

Still another embodiment relates to methods of constructing transgenic animals (e.g., mice) using recombinant molecules comprising a nucleic acid sequence encoding a Codeac polypeptide or a nucleic acid sequence that encodes a cDNA that is complementary to Codeac polynucleotides. Methods of constructing transgenic animals are well-known to those skilled in the art. For example, a transgenic mouse that overexpresses Codeac polypeptides and that can serve as a model for H. pylori infection of epithelial cells of the stomach and small intestine may be constructed and used according to U.S. Pat. No. 5,625,124, which disclosure is herein incorporated by reference in its entirety. Such transgenic animals may be useful for, e.g., obtaining animal models being predisposed to various chronic infections and to particular immune responses, or testing the usefulness of chemical compounds in the treatment of such diseases.

Protein of SEO ID NO:32 (Internal Designation Clone 131715_{—}105-090-4-0-D2-F)

The cDNA of Clone 131715_{—}105-090-4-D2-F (SEQ D NO:31) encodes Prospan of SEQ ID NO:32, comprising the amino-acid sequence: MWVPVVFLTLSVTWIGAAPLILSRIVGGWECEKHSQPWQVLVASRGRAVCGGVLVHPQWVLTA AHCIRNKSVILLDRSRTGPGSAAARGCRWPPYGIRLQGGRAAGMWRE. Accordingly, it will be appreciated that all characteristics and uses of the polypeptides of SEQ ID NO:32 described throughout the present application also pertain to the polypeptides encoded by the nucleic acids comprising the human cDNA in Clone 131715_{—}105-0904-0-D2-F. In addition, it will be appreciated that all characteristics and uses of the polynucleotides of SEQ ID NOs:## described throughout the present application also pertain to the nucleic acids comprising the human cDNA in Clone 131715_{—}105-0904-0-D2-F. A preferred embodiment of the invention is directed toward the human polynucleotide and polypeptide compositions of SEQ ID NO:32, SEQ ID NO:31, and Clone 131715_{—}105-0904-0-D2-F. Preferred Prospan polypeptide fragments for uses in the methods described below include the Prospan polypeptide comprising the amino-acid sequence of: PLILSRIVGGWECEKHSQPWQVLVASRGRAVCGGVLVHPQWVLTAAHCIRNKSVILLDRSRTGP GSAAARGCRWPPYGIRLQGGRAAGMWRE. Most preferred Prospan polypeptide fragments for uses in the methods described below include the Prospan polypeptide comprising the amino-acid sequence of: IVGGWECEKHSQPWQVLVASRGRAVCGGVLVHPQWVLTAAHCIRNKSVELLDRSRTGPGSAAA RGCRWPPYGIRLQGGRAAGMW E. Also preferred are polypeptide fragments having a biological activity as described herein and the polynucleotides encoding the fragments.

The protein of the invention, Prospan, is a NOVEL, secreted, splice variant of the prostate specific antigen (PSA, EMBL accession number NP_{—}001639). The two first exons are identical in the Prospan mRNA and in the PSA mRNA. The third exon of the Prospan mRNA is shorter at its 3′ extremity than the third exon of the PSA mRNA. The fourth exon of the Prospan mRNA is longer at its 3′ and its 5′extremities than the fourth exon of the PSA mRNA Prospan displays a signal peptide (MWVPVVFLTLSVTWIGAAPLILS), a propeptide that is cleaved in mature form (PLILSR) and a trypsin domain spanning a histidine active site consensus

(IVGGWECEKHSQPWQVLVASRGRAVCGGVLVHPQWVLTAAHCIRNKSVIL).

Prospan is a new member of the kallikrein family. It is secreted by normal, hyperplastic and cancerous prostatic epithelia. Prospan is upregulated by androgens, and the expression level of Prospan correlates positively with the malignancy grade of a prostate tumor. Prospan is found in semen, and at lower concentrations in blood and serum. Prospan is useful for early detection, staging, prognosis and monitoring of prostate cancer and other cancers such as breast cancer.

An embodiment of the invention is directed to a composition comprising a Prospan polypeptide sequence of SEQ ID NO:32, or having at least 70%, 80%, 90%, 95%, 96%, 97%, 98%, 99%, or more identity to SEQ ID NO:32.

A further embodiment of the invention is directed to a composition comprising a Prospan polypeptide fragment comprising one or more of the 34 carboxyl-terminal amino-acids of a Prospan polvpeptide sequence of SEQ ID NO:32.

As used herein, a “Prospan polypeptide” refers either to a Prospan polypeptide sequence of SEQ ID NO:32 or at least 70%, 80%; 90%, 95%, 96%, 97%, 98%, 99%, or more identical to SEQ ID NO: 32, or to a fragment thereof comprising one or more of the 34 carboxyl-terminal amino-acids of a Prospan polypeptide sequence of SEQ ID NO:32.

An embodiment of the invention is directed to a composition comprising a polynucleotide sequence of SEQ ID NO:31 encoding a Prospan polypeptide.

A further embodiment of the invention is directed to a composition comprising a polynucleotide sequence encoding a Prospan polypeptide.

As further used herein, a “Prospan polynucleotide” refers either to a Prospan polynucleotide sequence of SEQ ID NO:31 or having at least 70%, 80%, 90%, 95%, or more identity to SEQ ID NO:31, to a nucleotide sequence encoding a Prospan polypeptide.

A further embodiment of the invention is directed to an antibody recognizing a Prospan polypeptide sequence of SEQ ID NO:32 or a Prospan polypeptide fragment. Preferably, one or more of the 34 carboxyl-terminal amino-acids of a Prospan polypeptide sequence of SEQ ID NO:32 are necessary for binding of said antibody to said Prospan polypeptide. Preferred epitopes recognized by said antibody comprise the DRSRTGPG amino-acid sequence and the RGCRWPPY amino-acid sequence. Most preferably, the antibody binds to Prospan but not to PSA. As used herein, an “anti-Prospan antibody” refers to an antibody or antigen-binding fragment thereof that specifically binds to a Prospan polypeptide.

An embodiment of the present invention relates to a method of binding an anti-Prospan antibody to a Prospan polypeptide comprising the step of: contacting a Prospan polypeptide with said antibody under conditions that allow binding to occur. Such conditions are well known to those skilled in the art. Such methods are useful for detecting Prospan polypeptides as further described herein.

An embodiment of the present invention is directed to a method of detecting Prospan polypeptides in a biological sample, said method comprising the steps of: i) contacting a biological sample with an anti-Prospan antibody; and ii) detecting the antigen-antibody complex formed. The anti-Prospan antibody may be monoclonal or polyclonal. In addition, the anti-Prospan antibody may be primarily or secondarily labeled by any detectable compound (e.g., radioactive, fluorescent, luminescent, or enzymatic) common in the art. Detecting Prospan polypeptides is for example useful for detecting Prospan expression in a biological sample, e.g. for diagnostic purposes, as further described below.

An embodiment of the present invention is directed to methods of detecting Prospan expression in a biological sample, said methods comprising the steps of: i) providing a biological sample from an individual; ii) detecting the level of Prospan expression in the sample; and iii) comparing the level of Prospan expression in said sample to that of a control sample. The level of Prospan expression in the sample can be assessed using any method, such as by detecting the level of Prospan mRNA or Prospan polypeptides in the sample. For example, well-known techniques such as, e.g., western blot, immunochemical techniques and cytochemical techniques may be used to detect Prospan expression. Preferably, anti-Prospan antibodies are used to detect the level of Prospan expression. Also preferably, polynucleotide probes comprising a polynucleotide sequence of SEQ ID NO:31 or part thereof, or a polynucleotide sequence complementary to SEQ ID NO:31, are used to detect the level of Prospan expression by, e.g., northern blot or RTPCR techniques. Preferred biological samples include those obtained from blood, serum, urine or seminal fluid.

Detecting Prospan expression levels can serve for detection of a cancer, e.g., a prostate cancer. In such a method, a higher level of Prospan expression in said biological sample in comparison to the level in a control sample representative of a level in a normal individual is indicative of prostate cancer and/or predisposition to prostate cancer. Furthermore, the expression level of Prospan correlates positively with malignancy of the cancer and with stage of the cancer. Alternatively, the ratio between Prospan expression level and PSA expression level, or the total amount of PSA and Prospan expression level can be determined and be indicative of prostate cancer, predisposition to prostate cancer, malignancy of the prostate cancer or its stage. It is also possible to establish differential expression of Prospan and PSA in various tissues. Detecting Prospan expression levels for detecting a prostate cancer can for example be performed as described in U.S. Pat. No. 5,935,818 or PCT application WO/0049158, which disclosures are incorporated herein by reference in their entireties. Alternatively, detecting Prospan expression levels can also serve in methods for detecting ovary or breast cancers, wherein a higher level of Prospan expression in said biological sample in comparison to the level in a control sample representative of a level in a normal individual is indicative of a cancer. The methods for the diagnosis, treatment and/or monitoring of the progression or remission of breast cancer that are described in U.S. Pat. No. 6,235,486, which disclosure is incorporated herein by reference in its entirety, can for example be performed with Prospan instead of hK2.

In one aspect of this embodiment, the level of Prospan expression in a biological sample is determined using an anti-Prospan antibody. Such a method comprises the steps of: i) providing a biological sample from an individual; ii) contacting said biological sample with a detectable, anti-Prospan antibody under conditions that allow binding to occur; iii) detecting the antigen-antibody complex formed; and iv) comparing the level of antigen-antibody complex that is detected in said sample to that of a control sample. Such methods comprise immunoassays such as those described in, e.g., U.S. Pat. No. 5,614,372 and Liedtke et al. (Clin Chem 30:649-52 (1984)), which disclosures are incorporated by reference in their entireties.

In another aspect of this embodiment, the level of Prospan expression in a biological sample is determined using a Prospan polynucleotide. One such method comprises the steps of: i) providing a biological sample from an individual; ii) contacting said biological sample with a Prospan polynucleotide probe under conditions that allow hybridization of nucleic acid sequences, thereby enabling formation of hybridization complexes between said Prospan polynucleotide probe and any endogenous Prospan polynucleotides present in said sample; iii) detecting hybridization complexes; and iv) comparing the level of hybridization complexes that is detected in said sample to that of a control sample. Another such method comprises the steps of: i) providing a biological sample from an individual; ii) providing primers that specifically amplify a Prospan polynucleotide; iii) performing a nested polymerase chain reaction (PCR) on said biological sample of the subject using said primers; iv) detecting amplification products; and v) comparing the level of amplification products that is detected in said sample to that of a control sample.

Another embodiment of the present invention is directed to a diagnostic kit for detecting in vitro the presence of a Prospan polypeptide. Such kit comprises: i) an anti-Prospan antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Prospan antibodies is directed to the diagnosis of prostate cancer or breast cancer. Optionally, said diagnostic kit comprises a negative control sample representative of a level from a healthy, disease-free individual. Optionally, said diagnostic kit comprises a positive control sample representative of a level from an individual suffering from a given disorder, such as cancer, in particular prostate cancer. Optionally, said diagnostic kit comprises other antibodies such as, e.g., anti-PSA antibodies.

An additional embodiment relates to an array of polynucleotides probes comprising a Prospan polynucleotide. Preferably, said Prospan polynucleotide is part of an array of probes each present at a known location on a solid support. Preferably, said array comprises other polynucleotides that are markers for detection, staging, prognosis and monitoring of prostate cancer. These microarrays can for example be used to diagnose prostate cancer, or to perform epidemiological studies in families predisposed to prostate cancer. Other markers that may be part of said array include but are not limited to apolipoprotein E alleles, PG1 alleles, Bap28 alleles, fibronectin alleles, PurH alleles and PCTA-1 alleles.

Another embodiment relates to a method of producing a Prospan polypeptide comprising the steps of: i) culturing a cell comprising a polynucleotide encoding a Prospan polypeptide under conditions conducive to the expression of said polypeptide, and ii) purifying the produced polypeptide. The purification of the polypeptide can be done following any technique well-known to those skilled in the art. Preferably, an anti-Prospan antibody may be bound to a chromatographic support to form an affinity chromatography column. Preferably, the cell expressing a Prospan polypeptide is a recombinant host cell as described below. Producing purified Prospan polypeptides is useful to produce anti-Prospan antibodies as described in the present specification.

A preferred aspect of the invention is a host cell recombinant for polynucleotides encoding a Prospan polypeptide. An embodiment is directed to a method of constructing a host cell recombinant for polynucleotides encoding a Prospan polypeptide comprising the steps of: i) constructing a recombinant vector that comprises a nucleic acid sequence encoding a Prospan polypeptide, operably linked to a promoter, that allows expression of said Prospan polypeptide under given physiological conditions, and ii) introducing said recombinant vector into a cell. In preferred embodiments, said cell is an Eschenchia coli cell or into a human cell. An additional preferred aspect is a host cell recombinant for polynucleotides that, when present in a cell, cause an alteration in Prospan expression. A host cell recombinant for polynucleotides modifying Prospan expression may be constructed by a method comprising the step of: i) providing a cell comprising the Prospan gene; and ii) introducing a recombinant vector comprising polynucleotides into said cell, wherein the presence of said polynucleotides in said cell increases or decreases Prospan expression compared to Prospan expression in said cell before said recombinant vector is introduced. Preferably, said polynucleotides are inserted into or replace all or part of the 5′ regulatory region of the Prospan gene. Further preferably, said polynucleotides are located within 500 base pairs of the Prospan coding region. Said polynucleotides preferably comprise a promoter sequence. Techniques known in the art for introducing polynucleotide sequences to endogenous sequences are described in U.S. Pat. No. 5,641,670 and PCT W09629411, which disclosures are herein incorporated by reference in their entireties. Such recombinant host cell producing Prospan polypeptides may be used to produce Prospan polypeptides.

Protein of SEO ID NO:34 (Internal Designation Clone 109010_{—}105-041-4-0-A5-F)

The cDNA of Clone 109010_{—}105-041-4-0-A5-F (SEQ ID NO:33) encodes Prospanyl of SEQ ID NO:34, comprising the amino-acid sequence: MWVPVVFLTLSVTWIGAAPLILSRIVGGWECEKHSQPWQVLVASRGRAVCGGVLVHPQWVLTA AHCIRNKSVILLGRHSLFHPEDTGQAR. Accordingly, it will be appreciated that all characteristics and uses of the polypeptides of SEQ ID NO:34 described throughout the present application also pertain to the polypeptides encoded by the nucleic acids comprising the human cDNA in Clone 109010_{—}105-041-4-0-A5-F. In addition, it will be appreciated that all characteristics and uses of the polynucleotides of SEQ ID NOs:## described throughout the present application also pertain to the nucleic acids comprising the human cDNA in Clone 109010_{—}105-0414-0-A5-F. A preferred embodiment of the invention is directed toward the human polynucleotide and polypeptide compositions of SEQ ID NO:34, SEQ ID NO:33, and Clone 109010_ 105-041-4-0-A5-F. Preferred Prospanyl polypeptide fragments for uses in the methods described below include the Prospanyl polypeptide comprising the amino sequence of: PLILSRIVGGWECEKHSQPWQVLVASRGRAVCGGVLVHPQWVLTAAHCIRNKSVILLGRHSLFH PEDTGQAR. Most preferred Prospan polypeptide fragments for uses in the methods described below include the Prospan polypeptide comprising the amino-acid sequence of: IVGGWECEKHSQPWQVLVASRGRAVCGGVLVHPQWVLTAAHCIRNKSVILLGRHSLFHPEDTG QAR. Also preferred are polypeptide fragments having a biological activity as described herein and the polynucleotides encoding the fragments.

The protein of the invention, Prospanyl, is a NOVEL, secreted, splice variant of the prostate specific antigen (PSA, EMBL accession number NP_{—}001639). The first two exons are identical in the Prospanyl mRNA and in the PSA mRNA. The third and fourth exons of the Prospanyl mRNA result from an intra-exonic splice of the third exon of the PSA mRNA. The fifth and sixth exons of the Prospanyl mRNA correspond to the fourth and fifth exons of the PSA mRNA. Prospanyl displays a signal peptide (MWVPVVFLTLSVTWIGAAPLILS), a propeptide that is cleaved in mature form (PLILSR) and a trypsin domain spanning an histidine active site

(RIVGGWECEKHSQPWQVLVASRGRAVCGGVLVHPQWVLTAAHCIRNKSVILLGRHSLFHPEDT
GQ).

Prospanyl is a new member of the kallimein family. It is secreted by normal, hyperplastic and cancerous prostatic epithelia. Prospanyl is upregulated by androgens, and the expression level of Prospanyl correlates positively with the malignancy grade of a prostate tumor. Prospanyl is found in semen, and at lower concentrations in blood and serum. Prospan is useful for early detection, staging, prognosis and monitoring of prostate cancer and other cancers such as breast cancer.

An embodiment of the invention is directed to a composition comprising a Prospanyl polypeptide sequence of SEQ ID NO:34, or having at least 70%, 80%, 90%, 95%, 96%, 97%, 98%, 99%, or more identity to SEQ ID NO:34.

A further embodiment of the invention is directed to a composition comprising a Prospanyl polypeptide fragment having protease activity or protease inhibitor binding activity.

As used herein, a “Prospanyl polypeptide” refers either to a Prospanyl polypeptide sequence of SEQ ID NO:34 or at least 70%, 80%; 90%, 95%, 96%, 97%, 98%, 99%, or more identical to SEQ ID NO:34.

A further embodiment of the invention is directed to a composition comprising a Prospanyl polypeptide fragment comprising one or both of the amino-acids shown at positions 66 and 67 of SEQ ID NO:34.

As used herein, a “Prospanyl polypeptide” refers either to a Prospanyl polypeptide sequence of SEQ ID NO:34 or at least 70%, 80%; 90%, 95%, 96%, 97%, 98%, 99%, or more identical to SEQ ID NO:34, or to a fragment thereof comprising one or both of the amino-acids shown at positions 66 and 67 of SEQ ID NO:34.

An embodiment of the invention is directed to a composition comprising a polynucleotide sequence of SEQ ID NO:33 encoding a Prospanyl polypeptide.

A further embodiment of the invention is directed to a composition comprising a polynucleotide sequence encoding a Prospanyl polypeptide.

As further used herein, a “Prospanyl polynucleotide” refers either to a Prospanyl polynucleotide sequence of SEQ ID NO:33 or having at least 70%, 80%, 90%, 95%, or more identity to SEQ ID NO:33, to a nucleotide sequence encoding a Prospanyl polypeptide.

A further embodiment of the invention is directed to an antibody recognizing a Prospanyl polypeptide sequence of SEQ ID NO:34 or a Prospanyl polypeptide fragment. Preferably, one or both of the 2 carboxyl-terminal amino-acids of a Prospanyl polypeptide sequence of SEQ ID NO:34 are necessary for binding of said antibody to said Prospanyl polypeptide. Preferred epitopes recognized by said antibody comprise the LFHPEDTGQA amino-acid sequence. Most preferably, the antibody binds to Prospanyl but not to PSA. As used herein, an “anti-Prospanyl antibody” refers to an antibody or antigen-binding fragment thereof that specifically binds to a Prospanyl polypeptide.

An embodiment of the present invention relates to a method of binding an anti-Prospanyl antibody to a Prospanyl polypeptide comprising the step of: contacting a Prospanyl polypeptide with said antibody under conditions that allow binding to occur. Such conditions are well known to those skilled in the art. Such methods are useful for detecting Prospanyl polypeptides as further described herein.

An embodiment of the present invention is directed to a method of detecting Prospanyl polypeptides in a biological sample, said method comprising the steps of: i) contacting a biological sample with an anti-Prospanyl antibody; and ii) detecting the antigen-antibody complex formed. The anti-Prospanyl antibody may be monoclonal or polyclonal. In addition, the anti-Prospanyl antibody may be primarily or secondarily labeled by any detectable compound (e.g., radioactive, fluorescent, luminescent, or enzymatic) common in the art. Detecting Prospanyl polypeptides is for example useful for detecting Prospanyl expression in a biological sample, e.g. for diagnostic purposes, as further described below.

An embodiment of the present invention is directed to methods of detecting Prospanyl expression in a biological sample, said methods comprising the steps of: i) providing a biological sample from an individual; ii) detecting the level of Prospanyl expression in the sample; and iii) comparing the level of Prospanyl expression in said sample to that of a control sample. The level of Prospanyl expression in the sample can be assessed using any method, such as by detecting the level of Prospanyl mRNA or Prospanyl polypeptides in the sample. For example, well-known techniques such as, e.g., western blot, immunochemical techniques and cytochemical techniques may be used to detect Prospanyl expression. Preferably, anti-Prospanyl antibodies are used to detect the level of Prospanyl expression. Also preferably, polynucleotide probes comprising a polynucleotide sequence of SEQ ID NO:33 or part thereof, or a polynucleotide sequence complementary to SEQ ID NO:33, are used to detect the level of Prospanyl expression by, e.g., northern blot or RTPCR techniques. Preferred biological samples include those obtained from blood, serum, urine or seminal fluid.

Detecting Prospanyl expression levels can serve for detection of a cancer, e.g., a prostate cancer. In such a method, a higher level of Prospanyl expression in said biological sample in comparison to the level in a control sample representative of a level in a healthy, disease-free individual is indicative of prostate cancer and/or predisposition to prostate cancer. Furthermore, the expression level of Prospanyl correlates positively with malignancy of the cancer and with stage of the cancer. Alternatively, the ratio between Prospanyl expression level and PSA expression level, or the total amount of PSA and Prospanyl expression level can be determined and be indicative of prostate cancer, predisposition to prostate cancer, malignancy of the prostate cancer or its stage. It is also possible to establish differential expression of Prospanyl and PSA in various tissues. Detecting Prospanyl expression levels for detecting a prostate cancer can for example be performed as described in U.S. Pat. No. 5,935,818 or PCr application WO/0049158, which disclosures are incorporated herein by reference in their entireties. Alternatively, detecting Prospanyl expression levels can also serve in methods for detecting ovary or breast cancers, wherein a higher level of Prospanyl expression in said biological sample in comparison to the level in a control sample representative of a level in a healthy, disease-free individual is indicative of a cancer. The methods for the diagnosis, treatment and/or monitoring of the progression or remission of breast cancer that are described in U.S. Pat. No. 6,235,486, which disclosure is incorporated herein by reference in its entirety, can for example be performed with Prospanyl instead of hK2.

In one aspect of this embodiment, the level of Prospanyl expression in a biological sample is determined using an anti-Prospanyl antibody. Such a method comprises the steps of: i) providing a biological sample from an individual; ii) contacting said biological sample with a Prospan polynucleotide probe under conditions that allow hybridization of nucleic acid sequences, thereby enabling formation of hybridization complexes between said Prospan polynucleotide probe and any endogenous Prospan polynucleotides present in said sample; and iii) comparing the level of antigen-antibody complex that is detected in said sample to that of a control sample. Such methods comprise immunoassays such as those described in, e.g., U.S. Pat. No. 5,614,372 and Liedtke et al. (Clin Chem 30:649-52 (1984)), which disclosures are incorporated herein by reference in their entireties.

In another aspect of this embodiment, the level of Prospanyl expression in a biological sample is determined using a Prospanyl polynucleotide. One such method comprises the steps of: i) providing a biological sample from an individual; ii) providing a probe comprising a Prospanyl polynucleofide or the complement thereof, that is detectable by any technique common in the art; iii) contacting a biological sample with said probe under conditions that allow hybridization of nucleic acid sequences, thereby enabling formation of hybridization complexes; iv) detecting hybridization complexes; and v) comparing the level of hybridization complexes that is detected in said sample to that of a control sample. Another such method comprises the steps of: i) providing a biological sample from an individual; ii) providing primers that specifically amplify a Prospanyl polynucleotide; iii) performing a nested polymerase chain reaction (PCR) on said biological sample of the subject using said primers; iv) detecting amplification products; and v) comparing the level of amplification products that is detected in said sample to that of a control sample.

Another embodiment of the present invention is directed to a diagnostic kit for detecting in vitro the presence of a Prospanyl polypeptide. Such kit comprises: i) an anti-Prospanyl antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Prospanyl antibodies is directed to the diagnosis of prostate cancer or breast cancer. Optionally, said diagnostic kit comprises a negative control sample representative of a level from a healthy, disease-free individual. Optionally, said diagnostic kit comprises a positive control sample representative of a level from an individual suffering from a given disorder, such as cancer, in particular prostate cancer.. Optionally, said diagnostic kit comprises other antibodies such as, e.g., anti-PSA antibodies.

An additional embodiment relates to an array of polynucleotides probes comprising a Prospanyl polynucleotide. Preferably, said Prospanyl polynucleotide is part of an array of probes each present at a known location on a solid support. Preferably, said array comprises other polynucleotides that are markers for detection, staging, prognosis and monitoring of prostate cancer. These microarrays can for example be used to diagnose prostate cancer, or to perform epidemiological studies in families predisposed to prostate cancer. Other markers that may be part of said array include but are not limited to apolipoprotein E alleles, PG1 alleles, Bap28 alleles, fibronectin alleles, PurH alleles and PCTA-1 alleles.

Another embodiment relates to a method of producing a Prospanyl polypeptide comprising the steps of: i) culturing a cell comprising a polynucleotide encoding a Prospan polypeptide under conditions conducive to the expression of said polypeptide, and ii) purifying the produced polypeptide. The purification of the polypeptide can be done following any technique well-known to those skilled in the art. Preferably, an anti-Prospanyl antibody may be bound to a chromatographic support to form an affinity chromatography column. Preferably, the cell expressing a Prospanyl polypeptide is a recombinant host cell as described below. Producing purified Prospanyl polypeptides is useful to produce anti-Prospanyl antibodies as described in the present specification.

A preferred aspect of the invention is a host cell recombinant for polynucleotides encoding a Prospanyl polypeptide. An embodiment is directed to a method of constructing a host cell recombinant for polynucleotides encoding a Prospanyl polypeptide comprising the steps of: i) constructing a recombinant vector that comprises a nucleic acid sequence encoding a Prospanyl polypeptide, operably linked to a promoter, that allows expression of said Prospanyl polypeptide under given physiological conditions, and ii) introducing said recombinant vector into a cell. In preferred embodiments, said cell is an Escherichia coli cell or into a human cell. An additional preferred aspect is a host cell recombinant for polynucleotides that, when present in a cell, cause an alteration in Prospanyl expression. A host cell recombinant for polynucleotides modifying Prospanyl expression may be constructed by a method comprising the step of: i) providing a cell comprising the Prospanyl gene; and ii) introducing a recombinant vector comprising polynucleotides into said cell, wherein the presence of said polynucleotides in said cell increases or decreases Prospanyl expression compared to Prospanyl expression in said cell before said recombinant vector is introduced. Preferably, said polynucleotides are inserted into or replace all or part of the 5′ regulatory region of the Prospanyl gene. Further preferably, said polynucleotides are located within 500 base pairs of the Prospanyl coding region. Said polynucleotides preferably comprise a promoter sequence. Techniques known in the art for introducing polynucleotide sequences to endogenous sequences are described in U.S. Pat. No. 5,641,670 and PCT WO962941 1, which disclosures are herein incorporated by reference in their entireties. Such recombinant host cell producing Prospanyl polypeptides may be used to produce Prospanyl polypeptides.

Protein of SEQ ID NO:36 (Internal designation Clone 584047_{—}181-11-4-0-G9-F)

The cDNA of Clone 584047_{—}181-11-40-G9-F (SEQ ID NO:35) Encodes Firp of SEQ ID NO:36, comprising the amino-acid sequence: MAKVFSFILVTTALIMGREISALEDCAQEQMRLRAQVRLLETRVKQQQVKIKQLLQENEVQFLD KGDENTVVDLGSKRQYADCSEIFNDGYKLSGFYKEKPLQSPAEFSVYCDMSDGGGWTVIQRRSD GSENFNRGWKDYENGFGNFVQKHGEYWLGNKNLHFLTTQDYTLKIDLADFEKNSRYAQYKN FKVGDEKNFYELNIGEYSGTAGDSLAGNHPEVQWWASHQRMKFSTWDRDHDNYEGNCAEED QSGWWFNRCHSANLNGVYYSGPYTAKTDNGWYTGLVFSEICGYEN. Accordingly, it will be appreciated that all characteristics and uses of the polypeptides of SEQ ID NO:36 described throughout the present application also pertain to the polypeptides encoded by the nucleic acids comprising the human cDNA in Clone 584047_{—}181-11-4-0-G9-F. In addition, it will be appreciated that all characteristics and uses of the polynucleotides of SEQ ID NO:35 described throughout the present application also pertain to the nucleic acids comprising the human cDNA in Clone 584047_{—}181-11-4-0-G9-F. A preferred embodiment of the invention is directed toward the human polynucleotide and polypeptide compositions of SEQ ID NO:36, SEQ ID NO:35, and Clone 584047_{—}181-11-4-0-G9-F. Preferred Firp polypeptide fragments for uses in the methods described below include the Firp polypeptide comprising the amino sequence of: LEDCAQEQMRLRAQVRLLETRVKQQQVKIKQLLQENEVQFLDKGDENTVVDLGSKRQYADCSE IFNDGYKLSGFYKIKPLQSPAEFSVYCDMSDGGGWTVIQRRSDGSENFNRGWKDYENGFGNFVQ KHGEYWLGNKNLHFLTTQEDYTLKIDLADFEKNSRYAQYKNFKVGDEKNFYELNIGEYSGTAG DSLAGNFHPEVQWWASHQRMKFSTWDRDHDNYEGNCAEEDQSGWWFNRCHSANLNGVYYS GPYTAKTDNGIVWYTWHGLVFSEICGYEN. Also preferred are polypeptide fragments having a biological activity as described herein and the polynucleotides encoding the fragments.

The protein of the invention, Firp, is a NOVEL, secreted, splice variant of the fibrinogen-related protein 1 (HFREP1, Genbank accession number NP_{—}004458). This NOVEL splice variant displays 9 exons. The Firp polypeptide displays a signal peptide (MAKVFSFILVTTALIMGREISA) and a fibrinogen C-terminal globular domain with the four conserved cysteines that are involved in disulfide bonds (cysteine residues at positions 61, 90, 226 and 239 of SEQ ID NO:36) and with a FIBRIN_AG_C_DOMAIN signature (WWFNRCHSANLNG). The resulting mature Firp polypeptide is 279 amino-acids long. The amino-acids shown at positions 1 to 268 of SEQ ID NO:36 are identical to the 268 amino-terminal amino-acids of the mature HFREP1 polypeptide, and the 11 carboxyl-terminal amino-acids of Firp are unique to this splice variant.

Firp is specifically expressed in liver, and is mainly synthesized by hepatic parenchymal cells. It is a secreted protein that is present in liver extracellular matrix and in sermun. It is expressed at high levels in embryos and in liver cancers. Firp has mitotic activity and stimulates proliferation of liver cells. Firp is also necessary for differentiation of hepatocyte precursors and plays a major role both during embryonic development of liver and in liver regeneration in adults. Furthermore, Firp overexpression contributes to development of liver cancers.

An embodiment of the invention is directed to a composition comprising a Firp polypeptide sequence of SEQ ID NO:36, or having at least 70%, 80%, 90%, 95%, 96%, 97%, 98%, 99%, or more identity to SEQ ID NO:36.

A further embodiment of the invention is directed to a composition comprising a Firp polypeptide fragment stimulating proliferation or differentiation of liver cells.

As used herein, a “Firp polypeptide” refers to a Firp polypeptide sequence of SEQ ID NO:36 or at least 70%, 80%; 90%, 95%, 96%, 97%, 98%, 99%, or more identical to SEQ ID NO:36, or to a Firp polypeptide fragment comprising at least one of the amino-acids shown at positions 269 to 279 of SEQ ID NO:36, or to a Firp polypeptide fragment stimulating proliferation or differentiation of liver cells.

An. embodiment of the invention is directed to a composition comprising a polynucleotide sequence of SEQ ID NO:35 encoding a Firp polypeptide.

A further embodiment of the invention is directed to a composition comprising a polynucleotide sequence encoding a Firp polypeptide.

As furthe used herein, a “Firp polynucleotide” refers either to a Firp polynucleotide sequence of SEQ ID NO:35 or having at least 70%, 80%, 90%, 95%, or more identity to SEQ ID NO:35, to a nucleotide sequence encoding a Firp polypeptide, or to a sequence complementary to any of these sequences.

A further embodiment of the invention is directed to an antibody that recognizes a Firp polypeptide. Preferably, the antibody recognizes one or more amino-acids located within the 11 carboxyl-terminal amino-acids of Firp, wherein said one or more amino-acids is required for binding of the antibody to a Firp polypeptide. Most preferably, the antibody specifically binds to Firp but not to HFREP1. As used herein, an “anti-Firp antibody” refers to an antibody or antigen-binding fragment thereof that specifically binds to a Firp polypeptide.

An embodiment of the present invention relates to a method of binding an anti-Firp antibody to a Firp polypeptide comprising the step of: contacting a Firp polypeptide with said antibody under conditions that allow binding to occur. Such conditions are well known to those skilled in the art. Such methods are useful for detecting Firp polypeptides as further described herein.

An embodiment of the present invention is directed to a method of detecting Firp polypeptides in a biological sample, said method comprising the steps of: i) contacting a biological sample with an anti-Firp antibody; and ii) detecting the antigen-antibody complex formed. The anti-Firp antibody may be monoclonal or polyclonal. In addition, the anti-Firp antibody may be primarily or secondarily labeled by any detectable compound (e.g., radioactive, fluorescent, luminescent, or enzymatic) common in the art. Such a method may be applied to, e.g., diagnosis of liver cancer as further described below.

An embodiment of the present invention is directed to methods of detecting Firp expression in a biological sample, said methods comprising the steps of: i) providing a biological sample from an individual; ii) detecting the level of Firp expression in the sample; and iii) comparing the level of Firp expression in said sample to that of a control sample. In one such embodiment, said biological sample is obtained from an individual suspected of suffering from liver cancer, and the method is used to diagnose liver cancer. A higher level of Firp expression in said biological sample in comparison to the level in a control sample representative of a level in a normal individual is indicative of the presence of liver cancer. As used herein, the term “liver cancer” includes malignant hepatomas and hepatocellular carcinomas. Preferred biological sample is obtained from serum. The level of Firp expression in the sample can be assessed using any method, such as by detecting the level of Firp mRNA or Firp polypeptides in the sample. Any of a number of well-known techniques such as, e.g., western blot, immunochemical techniques and cytochemical techniques may be used to detect Firp expression. Preferably, anti-Firp antibodies are used to detect the level of Firp expression. Detecting Firp expression may be performed as described in E.P. Pat. No. 0,339,097, which disclosure is incorporated by reference in its entirety, by substituting an antibody binding to the collagenase inhibitor for an anti-Firp antibody. Also preferably, polynucleotide probes comprising a polynucleotide sequence of SEQ ID NO:35 or part thereof, or a polynucleotide sequence complementary to SEQ ID NO:35, are used to detect the level of Firp expression by, e.g., northern blot or RTPCR techniques.

Another embodiment of the present invention is directed to a diagnostic kit for quantifying in vitro the presence of a Firp polypeptide. Such kit comprises: i) an anti-Firp antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Firp antibodies is used for diagnosis of a liver cancer. Optionally, said diagnostic kit comprises a negative control sample representative of the level from a healthy, e.g. cancer free individual. Optionally, said diagnostic kit comprises a positive control sample representative of the level from an individual suffering from a liver cancer.

Another embodiment relates to a method of producing a Firp polypeptide comprising the steps of: i) culturing a cell comprising a polynucleotide encoding a Firp polypeptide under conditions conducive to the expression of said polypeptide, and ii) purifying the produced polypeptide. The purification of the polypeptide can be carried out following any technique well-known to those skilled in the art. Preferably, an anti-Firp antibody may be bound to a chromatographic support to form an affinity chromatography column. Alternatively, the Firp polypeptide may be fused to a heterologous immunogenic peptide, and the fusion protein is purified using an antibody specific to the heterologous peptide. Preferably, the cell expressing a Firp polypeptide is a recombinant host cell as described below. Producing Firp polypeptides may be useful in methods and compositions as further described herein.

A preferred aspect of the invention is a host cell recombinant for polynucleotides encoding a Firp polypeptide, operably linked to a promoter. An embodiment is directed to a method of constructing a host cell recombinant for polynucleotides encoding a Firp polypeptide comprising the steps of: i) constructing a recombinant vector that comprises a nucleic acid sequence encoding a Firp polypeptide, operably linked to a promoter, and ii) introducing said recombinant vector into a cell. In preferred embodiments, the cell is an Escherichia coli cell or a human cell. An additional preferred aspect is a host cell recombinant for polynucleotides that, when present in a cell, cause an alteration in Firp expression. A host cell recombinant for polynucleotides capable of modifying Firp expression may be constructed by a method comprising the step of: i) providing a cell comprising the Firp gene; and ii) introducing a recombinant vector comprising Firp expression-altering polynucleotides into said cell, wherein the presence of said polynucleotides in said cell increases or decreases Firp expression compared to the level of Firp expression in said cell before said recombinant vector is introduced. Preferably, said polynucleotides are inserted into or replace all or part of the 5′ regulatory region of the Firp gene. Further preferably, said polynucleotides are located within 500 base pairs of the Firp coding region. Said polynucleotides preferably comprise a promoter sequence. Techniques known in the art for introducing polynucleotide sequences to endogenous sequences are described in U.S. Pat. No. 5,641,670 and PCT WO962941 1, which disclosures are herein incorporated by reference in their entireties. Such recombinant host cell producing Firp polypeptides may be used to produce Firp polypeptides.

A preferred embodiment relates to methods of screening test substances for the ability to modulate Firp expression comprising the steps of: i) contacting a cell with a test substance; and ii) comparing Firp expression in the cell after exposure to the test substance to that of an unexposed control cell, wherein an observed difference in Firp expression between the exposed cell and the unexposed control cell indicates that the test substance modulates Firp expression. Firp expression may be determined by methods common to the art including but not limited to methods of quantifying Firp polynucleotides (e.g., detection of Firp mRNA by northern blot or RTPCR) or to methods of quantifying Firp polypeptides (e.g., detection of Firp polypeptides by western blot or immunochemistry). In one embodiment, the test substance modifies the expression of Firp in a specific cell type but not in others. Preferably, the test substance modifies the expression of Firp specifically in liver cells. Most preferably, the test substance modifies the expression of Firp specifically in hepatic parenchymal cells.

A further embodiment of the present invention is directed to methods of screening test substances for the ability to modulate Firp activity comprising the steps of: i) contacting a cell with a test substance, and ii) comparing Firp activity after exposure to the test substance to that of an unexposed control cell, wherein an observed difference in Firp activity between the exposed and unexposed cells indicates that the test substance modulates Firp activity. Said cells may express Firp polypeptides or not. If said cells do not express Firp polypeptides, said cells are contacted with a given amount of Firp polypeptides in addition to being contacted or not with the test substance. Preferred cells for use in such methods include liver cells. Most preferred cells are hepatocytes. Firp activity can be monitored, e.g., by measuring liver cell proliferation. Measurement of liver cell proliferation can be performed by a variety of techniques common to the art, including measurement of DNA synthesis as described in U.S. Pat. No. 6,248,725, which disclosure is herein incorporated by reference in its entirety.

Test substances that decrease Firp expression or activity are defined as Firp antagonists. Test substances that increase Firp expression or activity are defined as Firp agonists. As used herein, the term “Firp modulator” refers to Firp agonists and Firp antagonists. Test substances that modulate the expression or activity of Firp include, but are not limited to, chemical compounds (e.g. small-molecule inhibitors or activators), oligonucleotides, antisense polynucleotides, polypeptides, ribozymes, dominant negative forms of Firp, and anti-Firp antibodies. These substances may be made and used according to methods well known in the art. Firp modulators identified using the methods above are encompassed by the present invention, as are methods of using the modulators, e.g. to alter the activity or expression of Firp in a cell, tissue, or individual.

An embodiment of the present invention relates to methods of using Firp polypeptides or Firp agonists to stimulate liver cell proliferation and/or differentiation. As used herein, the term “liver cell” includes mature hepatocytes and hepatocyte precursors. Said liver cell may come from any mammal, including mice, rats, rabbits, apes and humans. Such a method comprises the step of: contacting a liver cell with an effective amount of a Firp polypeptide or a Firp agonist. As used herein, an “effective amount of a Firp polypeptide or a Firp agonist” refers to an amount of Firp polypeptide or Firp agonist that is sufficient to stimulate liver cell proliferation and/or differentiation.

In one such embodiment, said method is performed in vitro for culturing, expanding and/or differentiating liver cells. Hepatocytes may for example be used for liver transplantation, artificial livers, and for toxicology and pharmacology studies. A wide variety of techniques for culturing hepatocytes and hepatocyte precursors are known by those skilled in the art, and said Firp polypeptide or Firp agonist may be added to any known culture medium. A Firp polypeptides or Firp agonists may be added to, e.g., the Ham's P10, Han's F12, and RPM 1640 media that are described in U.S. Pat. No. 5,576,207, which disclosure is herein incorporated by reference in its entirety. Said Firp polypeptide or Firp agonist can be added at concentrations ranging, e.g., from 1 to 1000 micrograms per milliliter, more preferably from 10 to 100 micrograms per milliliter. In a preferred aspect, said method of culturing and expanding liver cells is directed to expand hepatic precursor cells and comprises the step of: culturing liver cells which contain at least a population of hepatic precursor cells in the presence of: (i) a serum-free culture medium that comprises Firp polypeptides and/or Firp agonists; (ii) an extracellular matrix; and (iii) liver stromal cells; wherein the hepatic precursor cells contained within the cell culture are expanded. This method may for example be performed as described in U.S. Pat. No. 5,576,207, which disclosure is incorporated by reference in its entirety. In another preferred aspect, said method of culturing and expanding liver cells is directed to enhance the long-term surviving culture of hepatocytes. This method may for example be performed as described in U.S. Pat. No. 5,030,105, which disclosure is incorporated by reference in its entirety. Non-therapeutic uses of hepatocytes and hepatocyte precursors include but are not limited to studies of mechanisms involved in liver injury and repair, studies of inflammatory and infectious diseases of the liver, studies of pathogenetic mechanisms, studies of mechanisms of liver cell transformation and etiology of liver cancer. Therapeutic uses of said hepatocytes and hepatocyte precursors include but are not limited to liver transplantation for patients suffering from liver failure, hepatitis or cirrhosis, assays for chemotherapy (e.g., for liver cancers), production of vaccines for viruses that grow in the liver, and assessment of acute and chronic hepatotoxicity of drugs or of chemicals and environmental pollutants. Liver failure, hepatitis or cirrhosis may be caused by, e.g., alcoholism, infections (e.g., hepatic necrosis due to Epstein-Barr virus infection), hepatotoxic pollutants (e.g., hepatitis following halothane anesthesia) or congenital liver diseases (e.g., Giant cell hepatitis, Wilson's disease, glycogen storage diseases, urea cycle enzyme defects, and Creigler-Najir disease). The hepatocytes and hepatocyte precursors cells may also be employed as part of an “artificial liver;” i.e., the hepatocytes and hepatocyte precursors may be placed in a container or apparatus in which the hepatocytes and hepatocyte precursors generate a liver lineage and function as a liver outside of the body, and wherein the container or apparatus is connected to the circulatory system of an individual.

In another such embodiment, said method of using Firp polypeptides or Firp agonists to stimulate liver cell proliferation and/or differentiation is performed in vivo. Said method comprises the step of: administering a physiologically acceptable carrier and an effective amount of a Firp polypeptide or a Firp agonist to an individual. As used in the context of this embodiment, an “effective amount of a Firp polypeptide or a Firp agonist refers to an amount of Firp polypeptide or Firp agonist that is sufficient to stimulate liver cell proliferation and/or differentiation. Said Firp polypeptide or Firp agonist may be administered in any effective amount. In general, however, effective amounts of Firp polypeptides will range from about 10 micrograms of Firp per kg of body weight per day (F/kgd) to about 100 milligrams F/kg/d. Preferably, such a method for stimulating liver cell proliferation and/or differentiation is directed to treat liver failure, hepatitis or cirrhosis due to, e.g., alcoholism, infections, hepatotoxic pollutants or congenital liver diseases. Also preferably, such a method is directed to promote liver regeneration consecutive to liver injury or surgery. For example, this method may be performed as described in U.S. Pat. No. 6,248,725 and U.S. Pat. No. 5,814,605, which disclosures are incorporated by reference herein, by replacing the Firp polypeptide for the keratinocyte growth factor polypeptide.

Another embodiment of the present invention relates to methods of using Firp antagonists to reduce or abolish liver cell proliferation. Such a method comprises the step of: delivering a physiologically acceptable carrier and an effective amount of a Firp antagonist to a liver or an individual. As used in the context of this embodiment, an “effective amount of a Firp antagonist” refers to an amount of Firp antagonist that is sufficient to reduce or abolish proliferation of liver cells. Preferably, a composition comprising a Firp antagonist is administered to an individual suffering from a liver cancer, such as, e.g., an hepatocellular carcinoma or a hepatoma, for treating or reducing in severity said cancer. In one embodiment, said Firp antagonist is an anti-Firp antibody. In another method, the Firp antagonist is fused to a targeting molecule specific for the tumor to be treated. Said targeting molecules specific for the tumor to be treated include but are not limited to ligands and antibody that recognizes a receptor or an antigen specifically expressed on tumor cells. A large number of tumor-associated antigens have been described in the scientific literature. For example, antigens and techniques described by Wikstrand et al. (Cancer Metastasis Rev 18:451-64 (1999)), which disclosure is herein incorporated by reference in its entirety, may be used. Alternatively, the Firp antagonist may be linked to a ligand that recognizes the vasculature of a tumor. A preferred method for targeting the Firp antagonist to the vasculature of solid tumors is described in U.S. Pat. No. 6,004,554, which disclosure is incorporated by reference in its entirety.

Effective dosages of a Firp polypeptide or a Firp modulator can be readily determined by one of ordinary skill in the art through, e.g., routine trials establishing dose response curves. The effective dosage of a Firp polypeptide or a Firp modulator can for example be determined by monitoring liver cell proliferation as described in U.S. Pat. No. 6,248,725, which disclosure is herein incorporated by reference in its entirety. Alternatively, the growth inhibitory effect of Firp antagonists on tumoral liver cells may be monitored by employing the subrenal capsule assay or the subcutaneous tumor assay described in U.S. Pat. No. 6,083,915, which disclosure is herein incorporated by reference in its entirety. The frequency of administration, and the administered amount of a composition comprising a physiologically acceptable carrier and a Firp polypeptide or a Firp modulator will be in accordance with the particular disease being treated, its severity, the nature of the Firp polypeptide or Firp modulator employed, the overall condition of the individual and the judgement of the treating physician. As used herein, the term “physiologically acceptable carrier” refers to a composition that is compatible with the individual and the Firp polypeptide or Firp modulator employed, and that is approved by a regulatory agency of the Federal or a state government or listed in the U.S. Pharmacopeia or other generally recognized pharmacopeia for use in animals, and more particularly in humans. Said physiologically acceptable carriers include but are not limited to solutions and suspensions such as biological buffers (e.g., phosphate buffered saline, saline and Dulbecco's Media), starches, sugars, microcrystalline cellulose, diluents, granulating agents, lubricants, binders and disintegrating agents.

The compositions comprising a physiologically acceptable carrier and the polypeptides or modulators of the present invention may be administered in any suitable manner. Preferred routes of administration include both targeted delivery by perfusion of the liver (for example by asanguineous perfusion) and oral administration. Examples of compositions that are suitable for oral administration include pills, tablets, capsules, and liquids. When the composition is administered orally to the subject, it is particularly preferred that the peptide be coated with a substance capable of protecting it from degradation in the subject's stomach for a sufficiently long period of time. Alternatively, the composition can be prepared in a suitable form, such as a liquid, for administration into the subject via a parenteral route, such as intravenous or subcutaneous administration. Other routes of administration include transdermal (e.g., topical), transmucosal (e.g., nasal, buccal or pulmonary) and intramuscular administration.

Still another embodiment relates to methods of constructing transgenic animals (e.g., mice) using recombinant molecules comprising a nucleic acid sequence encoding a Firp polypeptide or a nucleic acid sequence that encodes a cDNA that is complementary to Firp polynucleotides. Methods of constructing transgenic animals are well-known to those skilled in the art. For example, a transgenic mouse may be constructed using the method for in vivo liver cell retroviral transduction described in U.S. Patent 6,248,725, which disclosure is herein incorporated by reference in its entirety. Such transgenic animals may be useful for, e.g., obtaining animal models being predisposed to invasion and spread of liver cancers, obtaining animal models for diseases such as liver failure, hepatitis and cirrhosis, or testing the usefulness of chemical compounds in the treatment of such diseases.

Protein of SEO ID NO:38 (Internal Designation Clone 140354_{—}105-115-4-0-F5-F)

The cDNA of Clone 140354_{—}105-1154-0-F5-F (SEQ ID NO:37) encodes Sapro of SEQ ID NO:38, comprising the amino-acid sequence: MKFLVFAELALMVSMIGADSSEEYGYGPYQPVPEQPLYPQPYQPQYQQYTF. Accordingly, it will be appreciated that all characteristics and uses of the polypeptides of SEQ ID NO:38 described throughout the present application also pertain to the polypeptides encoded by the nucleic acids comprising the human cDNA in Clone 140354_{—}105-1154-0-F5-F. In addition, it will be appreciated that all characteristics and uses of the polynucleotides of SEQ ID NO:37 described throughout the present application also pertain to the nucleic acids comprising the human cDNA in Clone 140354 105-1154-0-F5-F. A preferred embodiment of the invention is directed toward the human polynucleotide and polypeptide compositions of SEQ ID NO:38, SEQ ID NO:37, and Clone 140354_{—}105-1154-0-FS-F. Preferred Sapro polypeptide fragments for uses in the methods described below include the Sapro polypeptide comprising the amino sequence of: DSSEEYGYGPYQPVPEQPLYPQPYQPQYQQYTF. Also preferred are polypeptide fragments having a biological activity as described herein and the polynucleotides encoding the fragments.

The protein of the invention, Sapro, is a NOVEL splice variant of Statherin. (EMBL accession number AAC02781). Sapro cDNA lacks the fourth exon of Statherin cDNA. Thus Sapro lacks a span of 10 amino-acids compared to Statherin. The amino-acids shown at positions 1 to 5 of SEQ ID NO:38 are identical to the 5 amino-terminal amino-acids of a mature Statherin polypeptide, and the amino-acids shown at positions 6 to 33 of SEQ ID NO:38 are identical to the 27 carboxyl-terminal amino-acids of a mature Statherin polypeptide. Sapro displays a signal peptide (MKFLVFAFILALMVSMIGA), and a negatively charged pentapeptide sequence (DSSEE) that is responsible for binding to hydroxyapatite surfaces.

Sapro is a multifunctional salivary protein that is a component of the acquired enamel pellicle. It binds to hydroxyapatite and inhibits spontaneous precipitation of calcium phosphate salts from supersaturated saliva. By so doing, it favors remineralization of the tooth surface and prevents crystal growth and formation of dysfunctional mineral deposits. Sapro also serves as a lubricant for the tooth surface, protecting it from various physical forces such as mastication and dental bruxing. Furthermore, the carboxyl-terminal extremity of Sapro inhibits the growth of anaerobic bacteria. In addition, Sapro binds to cariogenic bacteria thus indirectly promoting caries formation by maintaining a reservoir of bound cariogenic bacteria in the oral cavity.

An embodiment of the invention is directed to a composition comprising a Sapro polypeptide sequence of SEQ ID NO:38, or having at least 70%, 80%, 90%, 95%, 96%, 97%, 98%, 99%, or more identity to SEQ ID NO:38.

A further embodiment of the invention is directed to a composition comprising a Sapro polypeptide fragment comprising the contiguous amino-acids shown as positions 5 and 6 of SEQ ID NO:38.

A further embodiment of the invention is directed to a composition comprising a Sapro polypeptide fragment binding to hydroxyapatite, binding to cariogenic bacteria, inhibiting precipitation of calcium phosphate salts, inhibiting bacterial growth, or acting as a lubricant for the tooth surface.

As used herein, a “Sapro polypeptide” refers either to a Sapro polypeptide sequence of SEQ ID NO:38 or at least 70%, 80%; 90%, 95%, 96%, 97%, 98%, 99% or more identical to SEQ ID NO:38, or to a Sapro polypeptide fragment comprising the contiguous amino-acids shown as positions 5 and 6 of SEQ ID NO:38, or to a Sapro polypeptide fragment binding to hydroxyapatite, binding to cariogenic bacteria, inhibiting precipitation of calcium phosphate salts, inhibiting bacterial growth, or acting as a lubricant for the tooth surface.

An embodiment of the invention is directed to a composition comprising a polynucleotide sequence of SEQ ID NO:37 encoding a Sapro polypeptide.

A further embodiment of the invention is directed to a composition comprising a polynucleotide sequence encoding a Sapro polypeptide.

As further used herein, a “Sapro polynucleotide” refers either to a Sapro polynucleotide sequence of SEQ ID NO:37 or having at least 70%, 80%, 90%, 95%, or more identity to SEQ ID NO:37, or to a nucleotide sequence encoding a Sapro polypeptide, or to a sequence complementary to any of these sequences.

A further embodiment of the invention is directed to an antibody that recognizes a Sapro polypeptide. Preferably, such an anti-Sapro antibody recognizes the contiguous shown as positions 5 and 6 of SEQ ID) NO:38, and these contiguous amino-acids are necessary for binding. Preferably, the antibody specifically binds to Sapro but not to Statherin. Preferred epitopes that are recognized by anti-Sapro antibodies include but are not limited to the DSSEBYGYGPYQPVPE epitope. As used herein, an “anti-Sapro antibody” refers to an antibody or antigen-binding fragment thereof that specifically binds to a Sapro polypeptide.

An embodiment of the present invention relates to a method of binding an anti-Sapro antibody to a Sapro polypeptide comprising the step of: contacting a Sapro polypeptide with said antibody under conditions that allow binding to occur. Such conditions are well known to those skilled in the art. Such methods are useful for detecting Sapro polypeptides as further described herein.

An embodiment of the present invention is directed to a method of detecting Sapro polypeptides in a biological sample, said method comprising the steps of: i) contacting a biological sample with an anti-Sapro antibody; and ii) detecting the antigen-antibody complex formed. The anti-Sapro antibody may be monoclonal or polyclonal. In addition, the anti-Sapro antibody may be primarily or secondarily labeled by any detectable compound (e.g., radioactive, fluorescent, luminescent, or enzymatic) common in the art. Such a method may be applied to, e.g., diagnosis of salivary and lacrimal gland dysfunction (sicca symptom).

An embodiment of the present invention is directed to methods of detecting Sapro expression in a biological sample, said methods comprising the steps of: i) providing a biological sample from an individual; ii) detecting the level of Sapro expression in the sample; and iii) comparing the level of Sapro expression in said sample to that of a control sample. Such a method is useful for, e.g., diagnosing sicca symptoms. A lower level of Sapro expression in said biological sample in comparison to the level in a control sample representative of a level in a healthy, disease free individual is indicative of the presence sicca symptoms. Preferred biological sample is obtained from salivary secretion. Such a method may for example be used to distinguish between sicca and non-sicca patients suffering from rheumatoid arthritis, for detecting sicca symptoms in patients undergoing a radioiodine treatment, or for detecting sicca symptoms in patients suffering from Sjogren's syndrome or primary biliary cirrhosis. Alternatively, detecting Sapro expression in a biological sample may be useful to diagnose various periodontal diseases and for an overall evaluation of the effects of a given drug (e.g., a central nervous system stimulant) on salivary secretion. The level of Sapro expression in the sample can be assessed using any method, such as by detecting the level of Sapro mRNA or Sapro polypeptides in the sample. Any of a number of well-known techniques such as, e.g., western blot, immunochemical techniques and cytochemical techniques may be used to detect Sapro expression. Preferably, anti-Sapro antibodies are used to detect the level of Sapro expression. Also preferably, polynucleotide probes comprising a polynucleotide sequence of SEQ ID NO:37 or part thereof, or a polynucleotide sequence complementary to SEQ ID NO:37, are used to detect the level of Sapro expression by, e.g., northern blot or RTPCR techniques. Detecting Sapro expression in a biological sample can for example be performed as described in Jensen et al. (Oral Dis. 3:254-61 (1997)) or in Nordgarden et al. (J Dent Res. 77:1817-22 (1998)), which disclosures are incorporated herein by reference in their entireties.

Another embodiment of the present invention is directed to a diagnostic kit for quantifying in vitro the presence of a Sapro polypeptide. Such kit comprises: i) an anti-Sapro antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Sapro antibodies is used for diagnosing sicca symptoms. Optionally, said diagnostic kit comprises a negative control sample representative of the level from a healthy, disease free individual. Optionally, said diagnostic kit comprises a positive control sample representative of the level from an individual suffering from sicca symptoms. Optionally, said kit comprises other antibodies useful for diagnosis of sicca symptoms.

Another embodiment relates to a method of producing a Sapro polypeptide comprising the steps of: i) culturing a cell comprising a polynucleotide encoding a Sapro polypeptide under conditions conducive to the expression of said polypeptide, and ii) purifying the produced polypeptide. The purification of the polypeptide can be carried out following any technique well-known to those skilled in the art. Preferably, an anti-Sapro antibody may be bound to a chromatographic support to form an affinity chromatography column. Alternatively, the Sapro polypeptide may be fused to a heterologous immunogenic peptide, and the fusion protein is purified using an antibody specific to the heterologous peptide. Preferably, the cell expressing a Sapro polypeptide is a recombinant host cell as described below. Producing Sapro polypeptides may be useful in methods and compositions as further described herein.

A preferred aspect of the invention is a host cell recombinant for polynucleotides encoding a Sapro polypeptide, operably linked to a promoter. An embodiment is directed to a method of constructing a host cell recombinant for polynucleotides encoding a Sapro polypeptide comprising the steps of: i) constructing a recombinant vector that comprises a nucleic acid sequence encoding a Sapro polypeptide, operably linked to a promoter, and ii) introducing said recombinant vector into a cell. In preferred embodiments, the cell is an Escherichia coli cell or a human cell. Such host cells are useful for, e.g., producing Sapro polypeptides. Sapro polypeptides may for example be produced from Escherichia coli using a yeast intern fusion construct as described in Gilbert et al. (Protein Expr Purif. 16:243-50 (1999)), which disclosure is incorporated herein by reference in its entirety.

An additional preferred aspect is a host cell recombinant for polynucleotides that, when present in a cell, cause an alteration in Sapro expression. A host cell recombinant for polynucleotides capable of modifying Sapro expression may be constructed by a method comprising the step of: i) providing a cell comprising the Sapro gene; and ii) introducing a recombinant vector comprising Sapro expression-altering polynucleotides into said cell, wherein the presence of said polynucleotides in said cell increases or decreases Sapro expression compared to the level of Sapro expression in said cell before said recombinant vector is introduced. Preferably, said polynucleotides are inserted into or replace all or part of the 5′ regulatory region of the Sapro gene. Further preferably, said polynucleotides are located within 500 base pairs of the Sapro coding region. Said polynucleotides preferably comprise a promoter sequence. Techniques known in the art for introducing polynucleotide sequences to endogenous sequences are described in U.S. Pat. No. 5,641,670 and PCT WO9629411, which disclosures are incorporated herein by reference in their entireties.

An embodiment relates to artificial saliva comprising a Sapro polypeptide. Artificial saliva can be used to, e.g., combat microbial mediated oral diseases, hydrodipsia or occlusal disharmony in individuals with normal salivary flow as well as those suffering from xerostomia or sialadenitis. Artificial saliva can also be used in testing, calibration, and standardization of devices and methods for collecting and analyzing oral fluids. Artificial saliva compositions in which a Sapro polypeptide may be added are described, e.g., in U.S. Pat. No. 4,879,281 and in U.S. Pat. No. 5,695,929, which disclosures are incorporated herein by reference in their entireties. The Sapro polypeptide may be present in artificial saliva at a level of from about 0.001% to about 20%.

A preferred embodiment relates to methods of screening test substances for the ability to modulate Sapro expression comprising the steps of: i) contacting a cell with a test substance; and ii) comparing Sapro expression in the cell after exposure to the test substance to that of an unexposed control cell, wherein an observed difference in Sapro expression between the exposed cell and the unexposed control cell indicates that the test substance modulates Sapro expression. Sapro expression may be determined by methods common to the art including but not limited to methods of quantifying Sapro polynucleotides (e.g., detection of Sapro mRNA by northern blot or RTPCR) or to methods of quantiling Sapro polypeptides (e.g., detection of Sapro polypeptides by western blot or immunochemistry). In one embodiment, the test substance modifies the expression of Sapro in a specific cell type or tissue but not in others. Preferred tissues include parotid and submandibular glands. Modulators identified using such methods are also encompassed by the present invention, as are methods of using the modulators, e.g., to alter the expression of Sapro in a cell, tissue, or individual.

A further embodiment of the present invention is directed to methods of screening test substances for the ability to modulate Sapro activity comprising the steps of: i) contacting a Sapro polypeptide with a test substance, and ii) comparing Sapro activity after exposure to the test substance to that of an unexposed Sapro polypeptide, wherein an observed difference in Sapro activity between the exposed and unexposed polypeptides indicates that the test substance modulates Sapro activity. Alternatively, said Sapro polypeptide is present in a cell, and an observed difference in Sapro activity between the exposed and unexposed cells indicates that the test substance modulates Sapro activity. Sapro activity can be determined by any of a number of techniques common to the art. Sapro's activity can be monitored, e.g., by studying its binding to hydroxyapatite, its binding to cariogenic bacteria, its lubricating properties, calcium phosphate precipitation, or bacterial growth. Sapro's adsorption at hydroxyapatite surfaces can be monitored, e.g., as described in Johnsson et al. (Arch Oral Biol. 36:631-6 (1991)). Sapro's capacity of binding to cariogenic bacteria can be monitored, e.g., as described in Li et al. (Infect Immun. 69:7224-33 (2001)). The effect of Sapro on calcium phosphate precipitation can be monitored, e.g., as described in Schwartz et al. (Calcif Tissue Int. 50:511-7 (1992)). The effect of Sapro on bacterial growth can be monitored, e.g., as described in Kochanska et al. (Acta Microbiol Pol. 49:243-51 (2000)). The lubricating properties of Sapro can be monitored, e.g., as described in Douglas et al. (Biochem Biophys Res Commun. 180:91-7 (1991)). The disclosures of all the articles mentioned in this embodiment are incorporated herein by reference in their entireties.

Test substances that decrease Sapro expression or activity are defined as Sapro antagonists. Test substances that increase Sapro expression or activity are defined as Sapro agonists. Sapro agonists and Sapro antagonists are defined as Sapro modulators. Test substances that modulate the expression or activity of Sapro include, but are not limited to, chemical compounds (e.g. small-molecule inhibitors or activators), oligonucleotides, antisense polynucleotides, polypeptides, ribozymes, dominant negative forms of Sapro, and anti-Sapro antibodies. These substances may be made and used according to methods well known in the art.

An embodiment of the present invention is directed to an oral composition comprising a physiologically acceptable carrier and a Sapro polypeptide, a Sapro agonist or a Sapro antagonist. Such compositions include toothpastes, mouthrinses, liquid dentifrices, lozenges, chewing gums or other vehicle suitable for use in the oral cavity. Such compositions also include drinks, cakes, wafers and snack food. Preferably, the Sapro polypeptide, the Sapro agonist or the Sapro antagonist is present in the composition at a level of from about 0.001% to about 20%. Toothpastes and mouthrinses are the preferred compositions. In addition to containing Sapro polypeptides, Sapro agonists or Sapro antagonists, such toothpastes and moutbrinses may contain any other compound, e.g., known silica dental abrasives, soluble fluoride ion sources, emulsifying agents, toothpaste binders, humectants, antiplaque agents, anticalculus agents or mixture thereof. Such an oral composition is useful in a wide variety of methods including but not limited to those described below.

A preferred embodiment of the invention relates to methods of using Sapro antagonists for inhibiting binding of bacteria to Sapro polypeptides. Such methods find use in inhibiting the development of dental plaque by blocking bacteria/pellicle binding sites. One such method comprises the step of: contacting an enamel surface or a saliva-coated hydroxyapatite surface with an anti-Sapro antibody. Such a method may be performed in vitro, for example as described in Johansson et al. (Oral Microbiol Immunol 15:112-8 (2000)), which disclosure is incorporated herein by reference in its entirety, or in vivo as further described in this embodiment. Another methods of using Sapro antagonists for inhibiting binding of bacteria to Sapro polypeptides comprises the step of: administering an effective amount of a physiologically acceptable carrier and a Sapro antagonist to an individual. As used in the context of this embodiment, an “effective amount of a Sapro antagonist” refers to an amount of Sapro antagonist that is sufficient to inhibit or reduce binding of bacteria to Sapro polypeptides. Preferably, said Sapro antagonist decreases Sapro activity. Also preferably, said physiologically acceptable carrier and said Sapro antagonist are part of the oral composition described above, and said composition is applied in the mouth of an individual susceptible to caries formation. Alternatively, said Sapro antagonist decreases Sapro expression and is administered systemically.

A preferred embodiment of the invention is a method of using Sapro to bind bacteria. This method comprises the step of: contacting a Sapro polypeptide with a bacteria-containing biological sample under conditions that allow Sapro binding. Preferred bacteria are bacteria present in the oral cavity. Such bacteria include but are not limited to actinomyces spp. (e.g., A. viscosus, A. naeslundii), Peptostreptococcus spp., Fusobaterium spp. (e.g., F. necrogenes, F. necrophoruin), Prevotella spp. (e.g., P. oralis), Bacteroides ssp. (e.g., B. forsythus, B. ureolyticus), Candida ssp. (e.g., C. albicans) and Porphyromonas spp. (e.g., P. gingivalis). This method may for example be applied to in vitro uses as further described below.

A preferred embodiment of the invention is a method of purifying bacteria present in the oral cavity. This method comprises the steps of: i) contacting a Sapro polypeptide that is immobilized on a support with a solution or sample comprising bacteria under conditions that allow binding; ii) removing contaminants from said support; and iii) eluting the bound bacteria off of said support. Preferably, the Sapro peptide is immobilized on a solid or semi-solid matrix to facilitate washing of sample to remove contaminants. Such a method is very useful for isolating the bacteria colonizing the tooth surface for the diagnosis and/or management of periodontal diseases such as, e.g., periodontitis and gingivitis.

Still another preferred embodiment of the invention is a method of using a Sapro polypeptide or a Sapro agonist to bind a hydroxyapatite surface or an enamel surface comprising the step of: contacting a hydroxyapatite surface or an enamel surface with a Sapro polypeptide or a Sapro agonist. In one aspect, this method is performed in vivo and comprises the step of: administering an effective amount of a physiologically acceptable carrier and a Sapro polypeptide or a Sapro agonist to an individual. As further used herein, “a Sapro composition” refers to any composition comprising a Sapro polypeptide or a Sapro agonist. As used in the context of this embodiment, an “effective amount of a Sapro polypeptide or a Sapro agonist refers to an amount of Sapro agonist that is sufficient for favoring remineralization of the tooth surface, preventing dysfunctional mineral deposits or acting as a lubricant. Such a method can for example be used for preventing and/or repairing dental weaknesses such as, e.g., dental caries, exposed roots and dentin sensitivity, for treating demineralized tooth enamel or for coating a dental implant covered with hydroxyapatite. Preferred Sapro compositions for use in this method are mouthrinses. Other preferred Sapro compositions are solutions, sprays or creams that are applied to teeth. The Sapro composition may be applied to teeth in any method for mineralizing tooth enamel, separately or in conjunction with other active compounds. The Sapro composition may also be applied to teeth in any anticariogenic method. The Sapro composition may contain any other compound that is effective for mineralizing tooth enamel. Methods in which Sapro compositions may be used and compositions to which Sapro polypeptides or Sapro agonists may be added are for example described in, e.g., U.S. Pat. No. 5,037,639 and U.S. Pat. No. 4,080,440, which disclosures are incorporated herein by reference in their entireties.

Effective dosages of a Sapro polypeptide or a Sapro modulator can be readily determined by one of ordinary skill in the art through, e.g., routine trials establishing dose response curves. For example, effective dosages of a Sapro antagonist for preventing carious degradation of teeth can be determined using the experimental procedure employed in U.S. Pat. No. 4,405,600, which disclosure is incorporated herein by reference in its entirety. Effective dosages of a Sapro polypeptide for remineralization of teeth can for example be evaluated through the in vivo and in vitro tests that are described in U.S. Pat. No. 6,214,321, which disclosure is incorporated herein by reference in its entirety. As used herein, the term “physiologically acceptable carrier” refers to a composition that is compatible with the individual and the Sapro polypeptide or Sapro agonist employed. Said physiologically acceptable carriers include but are not limited to solutions and suspensions such as biological buffers (e.g., phosphate buffered saline, saline and Dulbecco's Media), starches, sugars, microcrystalline cellulose, diluents, granulating agents, lubricants, binders and disintegrating agents. Preferred compositions comprising a physiologically acceptable carrier and the polypeptides or modulators of the present invention are oral compositions such as toothpastes and mouthrinses, but compositions comprising Sapro polypeptides or Sapro may be administered in any other suitable manner such as, for example, intravenous, parental, topical, oral, or local administration, or any combination thereof.

Still another embodiment relates to methods of constructing transgenic animals (e.g., mice) using recombinant molecules comprising a nucleic acid sequence encoding a Sapro polypeptide, or a nucleic acid sequence that encodes a cDNA that is complementary to Sapro polynucleotides. Methods of constructing transgenic animals are well-known to those skilled in the art. For example, a method for delivering a protein by genetically altering secretory gland cells in a mammal is described in U.S. Pat. No. 6,004,944, which disclosure is incorporated herein by reference in its entirety. Transgenic animals that display an altered Sapro expression may be useful for, e.g., obtaining animal models being predisposed to caries formation or dental weakness, obtaining animal models for sicca symtoms, or testing the usefulness of chemical compounds in the treatment of such symptoms.

Protein of SEO ID NO:40 (Internal Designation Clone 485499_{—}174-12-3-0-H7-F)

The cDNA of Clone 485499_{—}174-12-3-0-H7-F (SEQ ID NO:39) encodes Saprip of SEQ ID NO:40, comprising the amino-acid sequence: MLLILLSVALLALSSAQNLNEDVSQEESPSLIAGNPQGPSPQGGNKPQGPPPPPGKPQGPPPQGGN KPQGPPPPGKPQGPPPQGDKSRSPRSPPGKPQGPPPQGGKPQGPPPQGGNKPQGPPPPGKPQGPPA QGGSKSQSARSPPGKPQGPPQQEGNNPQGPPPPAGGNPQQPQAPPAGQPQGPPRPPQGGRPSRPH QLQPPQSSRIQ. Accordingly, it will be appreciated that all characteristics and uses of the polypeptides of SEQ ID NO:40 described throughout the present application also pertain to the polypeptides encoded by the nucleic acids comprising the human cDNA in Clone 485499_{—}174-12-3-0-H7-F. In addition, it will be appreciated that all characteristics and uses of the polynucleotides of SEQ ID NO:39 described throughout the present application also pertain to the nucleic acids comprising the human cDNA in Clone 485499_{—}174-12-3-0-H7-F. A preferred embodiment of the invention is directed toward the human polynucleotide and polypeptide compositions of SEQ ID NO:40, SEQ ID NO:39, and Clone 485499_{—}174-12-3-0-H7-F. Preferred Saprip polypeptide fragments for uses in the methods described below include the Saprip polypeptide comprising the amino sequence of: QNLNEDVSQEESPSLIAGNPQGPSPQGGNKPQGPPPPPGKPQGPPPQGGNKPQGPPPPGKPQGPPP QGDKSRSPRSPPGKPQGPPPQGGKPQGPPPQGGNKPQGPPPPGKPQGPPAQGGSKSQSARSPPGK PQGPPQQEGNNPQGPPPPAGGNPQQPQAPPAGQPQGPPRPPQGGRPSRPHQLQPPQSSRIQ. Also preferred Saprip polypeptide fragments for uses in the methods described below include the Saprip polypeptide fragments obtained by post-transcriptional processing as further described in detail. Also preferred are polypeptide fragments having a biological activity as described herein and the polynucleotides encoding the fragments.

The protein of the invention, Saprip, is a NOVEL, secreted, splice variant of the cP3 salivary proline-rich protein (PRP-1, EMBL accession number K03204). Exons 1 and 2 are identical in the Saprip mRNA and in the cP3 mRNA. The third exon of the Saprip mRNA is shorter than the third exon of the cP3 mRNA. The fourth exon of the Saprip mRNA is unique to the Saprip splice variant. Saprip displays a signal peptide (MLLILLSVALLALSSA) and the resulting mature protein is 192 amino acids long.

A wide variety of proline-rich proteins form the major protein components of human saliva. Six loci control the synthesis of saliva proline-rich proteins, and each locus encodes different proteins that are generated by alternative splicing and/or alternative post-translational processing. Saprip is a NOVEL splice variant encoded by the PRPB gene, and is a member of the basic family of saliva proline-rich proteins. Basic saliva proline-rich proteins all display a conserved amino-terminal region of 17 amino-acids, a differing number of B 1, B2 and B3 repeats and a carboxyl-terminal region (as described in detail in Maeda et al., J Biol Chem. 260:11123-30 (1985)). In cP3 for example, the unit “B₁-B₂-B₃” occurs four times contiguously. Saprip displays the following repeats: B₁-B₂-B₃-B₃-B₁. The two B₃ regions are separated by a linker region of 31 amino acids that is highly rich in proline (42%). Furthermore, Saprip displays the conserved amino-terminal region of 17 amino acids and a carboxyl-terminal region of 41 amino acids (GNPQQPQAPPAGQPQGPPRPPQGGRPSRPHQLOPPQSSRIQ). The twelve last carboxyl-terminal amino acids of this carboxyl-terminal region are unique to the Saprip splice variant.

Furthermore, the mature Saprip polypeptide can sometimes undergo proteolytic processing. One of these processings consists of cleavage by proprotein convertases at a R-S-[ASP]-R-S sequence (see, e.g., Chan et al., Eur J Biochem 268:3423-31 (2001)). Saprip displays one such sequence, and a proteolytic cleavage can occur at position 75 of SEQ ID NO:40. When Saprip is cleaved, the processing gives rise to two polypeptides, one of them being a NOVEL salivary proline-rich protein

(SPPGKPQGPPPQGGKPQGPPPQGGNPQGPPPPGKPQGPPAQGGSKSQSARSPPGKPQGPPQQEG
NNPQGPPPPAGGNPQQPQAPPAGQPQGPPRPPQGGRPSRPHQLQPPQSSRIQ).

The PRPB gene being a genetically polymorphic locus, Saprip can display some minor differences depending on the individual it has been isolated from These differences include but are not limited to conservative amino-acids changes, such as substitution of a non-polar residue for another non-polar residue or a charged residue for a similarly charged residue. These changes include those recognized by those of skill in the art as those that do not substantially alter the tertiary structure of the protein. Preferred polymorphic differences include but are not limited to: (i) replacement of asparagine at position 4 of SEQ ID NO:40 by serine; (ii) replacement of alanine at position 116 of SEQ ID NO:40 by proline; (iii) replacement of glutamine at position 123 of SEQ ID NO:40 by arginine; (iv) replacement of serine at position 127 of SEQ ID NO:40 by alanine; and (v) deletion of arginine at position 126 of SEQ ID NO:40.

Saprip is a multifunctional salivary protein that is a component of the acquired enamel pellicle. Saprip acts as a defense against dietary tannins by forming complexes with them and thereby preventing their interaction with other biological compounds and absorption from the intestinal canal. Saprip also binds to hydroxyapatite and serves as a lubricant for the tooth surface, protecting it from various physical forces such as mastication and dental bruxing. Furthermore, Saprip binds to cariogenic bacteria, thus indirectly promoting caries formation by maintaining a reservoir of bound cariogenic bacteria in the oral cavity. In addition, Saprip displays antiviral properties.

An embodiment of the invention is directed to a composition comprising a Saprip polypeptide sequence of SEQ ID NO:40, or having at least 70%, 80%, 90%, 95%, 96%, 97%, 98%, 99%, or more identity to SEQ ID NO:40.

A further embodiment of the invention is directed to a composition comprising a Saprip polypeptide fragment comprising the contiguous amino-acids shown as positions 89 and 90 of SEQ ID NO:40, or comprising at least one of the amino-acids at positions 181 to 192 of SEQ ID NO:40.

A further embodiment of the invention is directed to a composition comprising a Saprip polypeptide fragment binding to hydroxyapatite, binding to cariogenic bacteria, binding to tannins, reducing infectivity of viruses, or acting as a lubricant for the tooth surface.

As further used herein, a “Saprip polypeptide” refers either to a Saprip polypeptide sequence of SEQ ID NO:40 or at least 70%, 80%; 90%, 95%, 96%, 97%, 98%, 99% or more identical to SEQ ID NO:40, or to a Saprip polypeptide fragment comprising the contiguous amino-acids shown as positions 89 and 90 of SEQ ID NO:40, or to a Saprip polypeptide fragment comprising at least one of the amino-acids at positions 181 to 192 of SEQ ID NO:40, or to a Saprip polypeptide fragment binding to hydroxyapatite, binding to cariogenic bacteria, binding to tannins, reducing infectivity of viruses, or acting as a lubricant for the tooth surface.

An embodiment of the invention is directed to a composition comprising a polynucleotide sequence of SEQ ID NO:39 encoding a Saprip polypeptide.

A further embodiment of the invention is directed to a composition comprising a polynucleotide sequence encoding a Saprip polypeptide.

As further used herein, a “Saprip polynucleotide” refers either to a Saprip polynucleotide sequence of SEQ ID NO:39 or having at least 70%, 80%; 90%, 95%, 96%, 97%, 98%, 99% or more identity to SEQ ID NO:39, or to a nucleotide sequence encoding a Saprip polypeptide, or to a sequence complementary to any of these sequences.

A further embodiment of the invention is directed to an antibody that recognizes a Saprip polypeptide. Preferably, such an anti-Saprip antibody recognizes one or more of the amino acids at positions 181 to 192 of SEQ ID NO:40, and said one or more amino acids are necessary for binding. Preferably, the antibody specifically binds to Saprip but not to PRP-1. As used herein, an “anti-Saprip antibody” refers to an antibody or antigen-binding fragment thereof that specifically binds to a Saprip polypeptide.

An embodiment of the present invention relates to a method of binding an anti-Saprip antibody to a Saprip polypeptide comprising the step of: contacting a Saprip polypeptide with said antibody under conditions that allow binding to occur. Such conditions are well known to those skilled in the art. Such methods are useful for detecting Saprip polypeptides as further described herein.

An embodiment of the present invention is directed to a method of detecting Saprip polypeptides in a biological sample, said method comprising the steps of: i) contacting a biological sample with an anti-Saprip antibody; and ii) detecting the antigen-antibody complex formed. The anti-Saprip antibody may be monoclonal or polyclonal. In addition, the anti-Saprip antibody may be primarily or secondarily labeled by any detectable compound (e.g., radioactive, fluorescent, luminescent, or enzymatic) common in the art. Alternatively, detecting Saprip polypeptides in a biological sample may be performed by High-performance Liquid Chromatography using the method described in, e.g., Shintani et al. (Hum Hered.40:89-98 (1990)). Detecting Saprip polypeptides may be applied to, e.g., diagnosis of salivary and lacrimal gland dysfunction (sicca symptom) or detection of Saprip polymorphism as detailed below.

An embodiment of the present invention relates to methods of using a Saprip polynucleotide for DNA genotyping. Such a method comprises the steps of: i) providing a nucleotide test sample from an individual; and ii) detecting the presence of the Saprip genomic sequence. The presence of Saprip genomic sequence is preferably detected using standard molecular biological techniques (e.g., Southern blotting) and employing a Saprip nucleotide or fragment thereof as a probe. Alternatively, the method comprises the steps of i) providing a nucleotide test sample from an individual; and ii) detecting the presence of a mutation in the Saprip genomic sequence. Such mutations include deletions, translocations, inversions, and punctual mutations. As used herein, the term “mutation” comprises any polymorphic difference between the detected polynucleotide and SEQ ID NO:39. These mutations are preferably detected using standard molecular biological techniques (e.g., sequencing or PCR) and employing a Saprip nucleotide or fragment thereof as a probe or primer. Preferably, the nucleotide test sample in these methods contains DNA. Genotyping of an individual can be performed by any technique well-known to those skilled in the art such as, e.g., sequencing, microsequencing or hybridization. Such methods can be accomplished as described in U.S. Pat. No. 5,935,783 and U.S. Pat. No. 6,303,294, which disclosures are incorporated by reference in their entireties. Said Saprip polynucleotide may be used alone or in combination with other polynucleotidic sequences encoding, e.g., other salivary proteins. An additional aspect of this embodiment relates to an array of oligonucleotides probes comprising a Saprip polynucleotide. Preferably, said array of oligonucleotides probes are directed to detect polymorphism of salivary proteins.

Another embodiment of the present invention relates to methods of using a Saprip polynucleotide for detecting the presence of a Saprip mRNA. Such a method comprises the steps of: i) providing a nucleotide test sample from an individual; and ii) detecting the presence of the Saprip mRNA. The presence of Saprip mRNA is preferably detected using standard molecular biological techniques (e.g., northern blot, RTPCR) and employing a Saprip nucleotide or fragment thereof as a probe. Alternatively, the method comprises the steps of i) providing a nucleotide test sample from an individual; and ii) detecting the presence of a mutation in the Saprip mRNA.

Detecting Saprip polypeptides and/or detecting the presence of a mutation in the Saprip genomic sequence or in the Saprip mRNA may be useful in the field of forensic sciences or for performing population genetic studies.

An embodiment of the present invention is directed to methods of detecting Saprip expression in a biological sample, said methods comprising the steps of: i) providing a biological sample from an individual; ii) detecting the level of Saprip expression in the sample; and iii) comparing the level of Saprip expression in said sample to that of a control sample. Such a method is useful for, e.g., diagnosing sicca symptoms. A lower level of Saprip expression in said biological sample in comparison to the level in a control sample representative of a level in a healthy, disease free individual is indicative of the presence of sicca symptoms. Preferred biological sample is obtained from salivary secretion. Such a method may for example be used to distinguish between sicca and non-sicca patients suffering from rheumatoid arthritis, for detecting sicca symptoms in patients undergoing a radioiodine treatment, or for detecting sicca symptoms in patients suffering from Sjogren's syndrome or primary biliary cirrhosis. Alternatively, detecting Saprip expression in a biological sample may be useful to diagnose various periodontal diseases and for an overall evaluation of the effects of a given drug (e.g., a central nervous system stimulant) on salivary secretion. The level of Saprip expression in the sample can be assessed using any method, such as by detecting the level of Saprip mRNA or Saprip polypeptides in the sample. Any of a number of well-known techniques such as, e.g., western blot, immunochemical techniques and cytochemical techniques may be used to detect Saprip expression. Preferably, anti-Saprip antibodies are used to detect the level of Saprip expression. Also preferably, polynucleotide probes comprising a polynucleotide sequence of SEQ ID NO:39 or part thereof, or a polynucleotide sequence complementary to SEQ ID NO:39, are used to detect the level of Saprip expression by, e.g., northern blot or RTPCR techniques. Detecting Saprip expression in a biological sample can for example be performed as described in Jensen et al. (Oral Dis. 3:254-61 (1997)) or in Nordgarden et al. (J Dent Res. 77:1817-22 (1998)), which disclosures are incorporated herein by reference in their entireties.

Another embodiment of the present invention is directed to a diagnostic kit for quantifying in vitro the presence of a Saprip polypeptide. Such kit comprises: i) an anti-Saprip antibody; and optionally, ii) a reagent allowing the detection of the antigen-antibody complexes formed. Preferably, said antibody is detectably labeled as described above. Alternatively, the optional reagent may provide a detectable signal. The optional reagent may either bind to said antibody or react with the label on said antibody. Preferably, the kit comprising anti-Saprip antibodies is used for diagnosing sicca symptoms. Optionally, said diagnostic kit comprises a negative control sample representative of the level from a healthy, disease free individual. Optionally, said diagnostic kit comprises a positive control sample representative of the level from an individual suffering from sicca symptoms. Optionally, said kit comprises other antibodies useful for diagnosis of sicca symptoms.

Another embodiment relates to a method of producing a Saprip polypeptide comprising the steps of: i) culturing a cell comprising a polynucleotide encoding a Saprip polypeptide under conditions conducive to the expression of said polypeptide, and ii) purifying the produced polypeptide. The purification of the polypeptide can be carried out following any technique well-known to those skilled in the art. Preferably, an anti-Saprip antibody may be bound to a chromatographic support to form an affinity chromatography column. Alternatively, the Saprip polypeptide may be fused to a heterologous immunogenic peptide, and the fusion protein is purified using an antibody specific to the heterologous peptide. Preferably, the cell expressing a Saprip polypeptide is a recombinant host cell as described below. Producing Saprip polypeptides may be useful in methods and compositions as further described herein.

A preferred aspect of the invention is a host cell recombinant for polynucleotides encoding a Saprip polypeptide, operably linked to a promoter. An embodiment is directed to a method of constructing a host cell recombinant for polynucleotides encoding a Saprip polypeptide comprising the steps of: i) constructing a recombinant vector that comprises a nucleic acid sequence encoding a Saprip polypeptide, operably linked to a promoter, and ii) introducing said recombinant vector into a cell. In preferred embodiments, the cell is an Escherichia coli cell or a human cell. Such host cells are useful for, e.g., producing Saprip polypeptides.

An additional preferred aspect is a host cell recombinant for polynucleotides that, when present in a cell, cause an alteration in Saprip expression. A host cell recombinant for polynucleotides capable of modifying Saprip expression may be constructed by a method comprising the step of: i) providing a cell comprising the Saprip gene; and ii) introducing a recombinant vector comprising Saprip expression-altering polynucleotides into said cell, wherein the presence of said polynucleotides in said cell increases or decreases Saprip expression compared to the level of Saprip expression in said cell before said recombinant vector is introduced. Preferably, said polynucleotides are inserted into or replace all or part of the 5′ regulatory region of the Saprip gene. Further preferably, said polynucleotides are located within 500 base pairs of the Saprip coding region. Said polynucleotides preferably comprise a promoter sequence. Techniques known in the art for introducing polynucleotide sequences to endogenous sequences are described in U.S. Pat. No. 5,641,670 and PCT WO962941 1, which disclosures are incorporated herein by reference in their entireties.

An embodiment relates to artificial saliva comprising a Saprip polypeptide. Artificial saliva can be used to, e.g., combat microbial mediated oral diseases, hydrodipsia or occlusal disharmony in individuals with normal salivary flow as well as those suffering from xerostomia or sialadenitis. Artificial saliva can also be used in testing, calibration, and standardization of devices and methods for collecting and analyzing oral fluids. Artificial saliva compositions in which a Saprip polypeptide may be added are described, e.g., in U.S. Pat. No. 4,879,281 and in U.S. Pat. No. 5,695,929, which disclosures are incorporated herein by reference in their entireties. The Saprip polypeptide may be present in artificial saliva at a level of from about 0.001% to about 70%.

A preferred embodiment relates to methods of screening test substances for the ability to modulate Saprip expression comprising the steps of: i) contacting a cell with a test substance; and ii) comparing Saprip expression in the cell after exposure to the test substance to that of an unexposed control cell, wherein an observed difference in Saprip expression between the exposed cell and the unexposed control cell indicates that the test substance modulates Saprip expression. Saprip expression may be determined by methods common to the art including but not limited to methods of quantifying Saprip polynucleotides (e.g., detection of Saprip mRNA by northern blot or RTPCR) or to methods of quantifying Saprip polypeptides (e.g., detection of Saprip polypeptides by western blot or immunochemistry). In one embodiment, the test substance modifies the expression of Saprip in a specific cell type or tissue but not in others. Preferred tissues include parotid and submandibular glands. Modulators identified using such methods are also encompassed by the present invention, as are methods of using the modulators, e.g., to alter the expression of Saprip in a cell, tissue, or individual.

A further embodiment of the present invention is directed to methods of screening test substances for the ability to modulate Saprip activity comprising the steps of: i) contacting a Saprip polypeptide with a test substance, and ii) comparing Saprip activity after exposure to the test substance to that of an unexposed Saprip polypeptide, wherein an observed difference in Saprip activity between the exposed and unexposed polypeptides indicates that the test substance modulates Saprp activity. Alternatively, said Saprip polypeptide is present in a cell, and an observed difference in Saprip activity between the exposed and unexposed cells indicates that the test substance modulates Saprip activity. Saprip activity can be determined by any of a number of techniques common to the art. Saprip's activity can be monitored, e.g., by studying its binding to hydroxyapatite, its binding to cariogenic bacteria, its lubricating properties, its binding to tannins, or inhibiting effect on replication of viruses. Saprip's adsorption at hydroxyapatite surfaces can be monitored, e.g., as described in Moreno et a. (Calcif Tissue Int 36:48-59 (1984)). Saprip's capacity of binding to cariogenic bacteria can be monitored, e.g., as described in Gibbons et al. (Infect Immun. 56:2990-3 (1988)). Saprip's capacity of binding to tannins can be monitored, e.g., as described in Lu et al. (Arch Oral Biol 43:717-28 (1998)). Saprip's antiviral properties can be monitored, e.g., as described in Orzechowska et al. (Acta Virol 42:75-8 (1998)). The lubricating properties of Saprip can be monitored, e.g., as described in Hatton et al. (Biochem J 230:817-20 (1985)). The disclosures of all the articles mentioned in this embodiment are incorporated herein by reference in their entireties.

Test substances that decrease Saprip expression or activity are defined as Saprip antagonists. Test substances that increase Saprip expression or activity are defined as Saprip agonists. Saprip agonists and Saprip antagonists are defined as Saprip modulators. Test substances that modulate the expression or activity of Saprip include, but are not limited to, chemical compounds (e.g. small-molecule inhibitors or activators), oligonucleotides, antisense polynucleotides, polypeptides, ribozymes, dominant negative forms of Saprip, and anti-Saprip antibodies. More specifically, test substances that modulate the expression or activity of Saprip include, but are not limited to, calcium, 12-o-tetradecanoulphorbol-13-actetate, retinoids, cytokines such as IL-1 and IL-3, and polyphenolic compounds such as, e.g., isoproterenol, propanolol and atenolol. These substances may be made and used according to methods well known in the art.

An embodiment of the present invention is directed to an oral composition comprising a physiologically acceptable carrier and a Saprip polypeptide, a Saprip agonist or a Saprip antagonist Such compositions include toothpastes, mouthrinses, liquid dentifrices, lozenges, chewing gums or other vehicle suitable for use in the oral cavity. Such suitable forms are disclosed in U.S. Pat. No. 4,083,955, which disclosure is incorporated herein in its entirety by reference. Such compositions also include drinks and food. Preferably, the Saprip polypeptide, the Saprip agonist or the Saprip antagonist is present in the composition at a level of from about 0.001% to about 70%. Toothpastes, mouthrinses and food or drinks comprising tannins are the preferred compositions. In addition to comprising Saprip polypeptides, Saprip agonists or Saprip antagonists, toothpastes and mouthrinses may contain any other compound, e.g., known dental abrasives, soluble fluoride ion sources, emulsifying agents, toothpaste binders, humectants, antiplaque agents, anticalculus agents, flavoring agents or mixture thereof. Such oral compositions comprising Saprip polypeptides are useful in a wide variety of methods including but not limited to those described below.

In a preferred aspect, the composition comprising Saprip polypeptides and/or Saprip modulators is a toothpaste that comprises a dental abrasive. Such an abrasive polishing material contemplated for use in the toothpaste compositions of the present invention can be any material, which does not excessively abrade dentin. These include, for example, silicas, calcium carbonate, dicalcium orthophosphate dihydrate, calcium pyrophosphate, tricalcium phosphate, calcium polymetaphosphate, insoluble sodium polymetaphosphate, hydrated alumina, and resinous abrasive materials such as particulate condensation products of urea and formaldehyde, and others such as disclosed by Cooley et al. in U.S. Pat. No. 3,070,510, incorporated herein by reference in its entirety. Mixtures of abrasives may also be used. Silica dental abrasives are preferred for use herein. Optionally, flavoring and/or sweetening agents can be added to toothpaste compositions. Suitable flavoring agents include oil of wintergreen, oil of peppermint, oil of spearmint, oil of sassafras, and oil of clove. Sweetening agents, which can be used, include aspartame, acesulfame, saccharin, dextrose, levulose and sodium cyclamate. Flavoring and sweetening agents are generally used in toothpastes at levels of from about 0.005% to about 2% by weight. Optionally, toothpaste compositions can contain emulsifying agents. Suitable emulsifying agents are those, which are reasonably stable and foam throughout a wide pH range, including non-soap anionic, nonionic, cationic, zwittefionic and amphoteric organic synthetic surfactants. Water is also present in the toothpastes of this invention. Optionally, a thickening material that provides a desirable consistency can be added to the toothpaste. Preferred thickening agents are carboxyvinyl polymers, carrageenan, hydroxyethyl cellulose and water-soluble salts of cellulose ethers such as sodium carboxymethyl cellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents in an amount from 0.2% to 5.0% by weight of the total composition can be used. Optionally, a humectant material can be added in the toothpaste to keep it from hardening. Suitable humectants include glycerin, sorbitol, and other edible polyhydric alcohols at a level of from about 15% to about 70%.

Another preferred aspect of the present embodiment is a mouthwash composition. Mouthwashes generally comprise from about 20:1 to about 2:1 of a water/ethyl alcohol solution. The mouthwash may also be alcohol free. Preferably, other ingredients such as flavor, sweeteners, and humectants agents such as those mentioned above for dentifrices can be added. Generally, on a weight basis the mouthwashes of the invention comprise 0% to 60% (preferably 5% to 20%) ethyl alcohol, 0% to 20% (preferably 5% to 20%) of a humectant, 0% to 2% (preferably 0.01% to 1.0%) emulsifying agents, 0% to 0.5% (preferably 0.005% to 0.06%) sweetening agent such as saccharin or natural sweeteners such as stevroside 0% to 0.3% (preferably 0.03% to 0.3%) flavoring agent, and the balance water.

In still another preferred aspect, the composition comprising Saprip polypeptides and/or Saprip modulators is any food, forage or drink comprising dietary tannins. Preferred such food, forages and drinks include, but are not limited to, those comprising sorghum, beans such as faba-bean, cassava leaves, cowpeas, tea, peanut skins, Guajillo, Lotus pedunculatus or various tropical seeds such as, e.g., Prosopis africana and Lonchocarpus sericeus. Preferably, said forages are directed to feed ruminants.

A preferred embodiment of the invention is a method of using a Saprip polypeptide to bind tannins. This method comprises the step of: contacting a Saprip polypeptide with a tannin-comprising sample under conditions that allow Saprip binding. In one aspect, said method is performed in vivo and comprises the step of: administering a tannin-comprising sample in combination with a composition comprising a physiologically acceptable carrier and an effective amount of a Saprip polypeptide or a Saprip agonist to an individual. Preferred tamin-comprising samples are food, drinks and boverages comprising dietary tannins such as those listed above. In another aspect, said method is performed in vivo and comprises the step of: administering a composition comprising tannins and an effective amount of a Saprip polypeptide or a Saprip agonist to an individual. Preferred such compositions are food, drinks and boverages such as those listed above. As used in the context of this embodiment,-an “effective amount of a Saprip polypeptide or a Saprip agonist” refers to an amount of Saprip polypeptide or Saprip agonist that is sufficient for binding to at least 1%, 5%, 10%, 20%, 30%, 40%, 60%, 70%, 80%, 90% or more of the tannins present in said tannin-comprising sample or composition. Such a method can for example be used for preventing the antinutritional effects of dietary tannins.

Another preferred embodiment of the invention is a method of using a Saprip polypeptide or a Saprip agonist to inhibit the infectivity of viruses comprising the step of: contacting a virus with a Saprip polypeptide or a Saprip agonist. Such a method may be performed in vitro as described in Gu et al. (Oral Microbiol Immunol 10:54-9 (1995)). In a preferred aspect, said method is performed in vivo and comprises the step of: administering a composition comprising an effective amount of a Saprip polypeptide or a Saprip agonist to an individual infected by a virus. Said viruses include but are not limited to herpes viruses (e.g., herpes simplex virus 1, herpesvirus papio LMP2A), immunodeficiency viruses (e.g., IV-1), leukemia viruses (e.g., bovine leukemia virus gp30, murine leukemia virus), Epstein-Barr viruses (e.g., Epstein-Barr viruses LMP2A) and African horsesickness virus VP7. As used in the context of this embodiment, an “effective amount of a Saprip polypeptide or a Saprip agonist“refers to an amount of Saprip polypeptide or Saprip agonist that is sufficient for reducing virus infectivity.

Still another preferred embodiment of the invention is a method of using Saprip to bind bacteria. This method comprises the step of: contacting a Saprip polypeptide with a bacteria-containing biological sample under conditions that allow Saprip binding. Preferred bacteria are bacteria present in the oral cavity. Such bacteria include but are not limited to Candida ssp. (e.g., C. albicans), Actinomyces ssp. (e.g., A. viscosus) and Porphyromonas spp. (e.g., P. gingivalis). This method may for example be applied to in vitro and in vivo uses as further described below.

A preferred embodiment of the invention is a method of purifying bacteria present in the oral cavity. This method comprises the steps of: i) contacting a Saprip polypeptide that is immobilized on a support with a solution or sample comprising bacteria under conditions that allow binding; ii) removing contaminants from said support; and iii) eluting the bound bacteria off of said support. Preferably, the Saprip peptide is immobilized on a solid or semi-solid matrix to facilitate washing of sample to remove contaminants. Such a method is very useful for isolating the bacteria colonizing the tooth surface for the diagnosis and/or management of periodontal diseases such as, e.g., periodontitis and gingivitis.

A preferred embodiment of the invention relates to methods of using Saprip antagonists for inhibiting binding of bacteria to Saprip polypeptides. Such methods find use in inhibiting the development of dental plaque by blocking bacteria/pellicle binding sites. One such method comprises the step of: contacting an enamel surface or a saliva-coated hydroxyapatite surface with an anti-Saprip antibody. Such a method may be performed in vitro or in vivo. Another such method comprises the step of: administering an effective amount of a physiologically acceptable carrier and a Saprip antagonist to an individual. As used in the context of this embodiment, an “effective amount of a Saprip antagonist” refers to an amount of Saprip antagonist that is sufficient to inhibit or reduce binding of bacteria to Saprip polypeptides. Preferably, said Saprip antagonist decreases Saprip activity. Also preferably, said Saprip antagonist is a dominant negative form of Saprip. As used in the context of this embodiment, a dominant negative form of Saprip is a microorganism adhesive-inhibiting fragment of Saprip. Methods for obtaining and using such dominant negative forms of Saprip are described in, e.g., U.S. Pat. No. 5,013,542, which disclosure is incorporated herein by reference in its entirety. Most preferably, said physiologically acceptable carrier and said Saprip antagonist are part of the oral composition described above, and said composition is applied in the mouth of an individual susceptible to caries formation. Alternatively, said Saprip antagonist decreases Saprip expression and is administered systemically.

Still another preferred embodiment of the invention is a method of using a Saprip polypeptide or a Saprip agonist to bind a hydroxyapatite surface or an enamel surface comprising the step of: contacting a hydroxyapatite surface or an enamel surface with a Saprip polypeptide or a Saprip agonist. In one aspect, this method is performed in vivo and comprises the step of: administering an effective amount of a physiologically acceptable carrier and a Saprip polypeptide or a Saprip agonist to an individual. As further used in this embodiment, “a Saprip composition” refers to any composition comprising a physiologically acceptable carrier and a Saprip polypeptide or a Saprip agonist As used in the context of this embodiment, an “effective amount of a Saprip polypeptide or a Saprip agonist” refers to an amount of Saprip agonist that is sufficient for acting as a lubricant. Such a method can for example be used for preventing and/or repairing dental weaknesses. Preferred Saprip compositions for use in this method are mouthrinses. Other preferred Saprip compositions are solutions, sprays or creams that are applied to teeth. The Saprip composition may for example be applied to teeth in any anticariogenic method, separately or in conjunction with other active compounds. The Saprip composition may contain any other compound that is effective for mineralizing tooth enamel, preventing carie formation or strengthening teeth. Methods in which Saprip compositions may be used and compositions to which Saprip polypeptides or Saprip agonists may be added are for example described in, e.g., U.S. Pat. No. 5,037,639 and U.S. Pat. No. 4,080,440, which disclosures are incorporated herein by reference in their entireties.

Effective dosages of a Saprip polypeptide or a Saprip modulator can be readily determined by one of ordinary skill in the art through, e.g., routine trials establishing dose response curves. For example, effective dosages of a Saprip antagonist for preventing carious degradation of teeth can be determined using the experimental procedure employed in U.S. Pat. No. 4,405,600, which disclosure is incorporated herein by reference in its entirety. Effective dosages of a Saprip antagonist for reducing virus infectivity can be determined using, e.g., the experimental procedure employed in U.S. Pat. No. 6,239,099, which disclosure is incorporated herein by reference in its entirety. Effective dosages of a Saprip antagonist for preventing the antinutritional effects of dietary tannins can be determined using, e.g., the experimental procedure employed in U.S. Pat. No. 5,565,225, which disclosure is incorporated herein by reference in its entirety.

As used herein, the term “physiologically acceptable carrier” refers to a composition that is compatible with the individual and the Saprip polypeptide or Saprip modulator employed. Said physiologically acceptable carriers include but are not limited to solutions and suspensions such as biological buffers (e.g., phosphate buffered saline, saline and Dulbecco's Media), starches, sugars, microcrystaline cellulose, diluents, granulating agents, lubricants, binders and disintegrating agents. Preferred compositions comprising a physiologically acceptable carrier and the polypeptides or modulators of the present invention are oral compositions such as toothpastes and mouthrinses, but compositions comprising Saprip polypeptides or Saprip may be administered in any other suitable manner such as, for example, intravenous, parental, topical, oral, or local administration, or any combination thereof.

Still another embodiment relates to methods of constructing transgenic animals (e.g., mice) using recombinant molecules comprising a nucleic acid sequence encoding a Saprip polypeptide, or a nucleic acid sequence that encodes a cDNA that is complementary to Saprip polynucleotides. Methods of constructing transgenic animals are well-known to those skilled in the art. For example, a method for delivering a protein by genetically altering secretory gland cells in a mammal is described in U.S. Pat. No. 6,004,944, which disclosure is incorporated herein by reference in its entirety. Transgenic animals that display an altered Saprip expression may be useful for, e.g., obtaining animal models being predisposed to caries formation or dental weakness, obtaining animal models for sicca symptoms, or testing the usefulness of chemical compounds in the treatment of such symptoms.

### Uses of Antibodies

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of antigen-bearing substances, including the polypeptides of the present invention, in biological samples (See, e.g., Harlow et al., 1988). The antibodies may also be used in therapeutic compositions for killing cells expressing the protein or reducing the levels of the protein in the body.

The invention further relates to antibodies that act as agonists or antagonists of the polypeptides of the present invention. For example, the present invention includes antibodies that disrupt the receptor/ligand interactions with the polypeptides of the invention either partially or fully. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; US Pat. No. 5,811,097; Deng et al., (1998) Blood. 92(6):1981-1988; Chen et al., (1998), Cancer Res. 58(16):3668-3678; Harrop et al., (1998) Blood. 161(4): 1786-1794; Zhu, et al. (1998), Cancer Res. 58(15):3209-3214; Yoon, et al. (1998), J. Immunol. 160(7):3170-3179; Prat et al., (1998), J. Cell. Sci. 111(Pt2):237-247; Pitard et al., (1997), J. Immunol. Methods. 205(2): 177-190; Liautard et al., (1997), Cytokine. 9(4):233-241; Carlson et al. (1997), J. Immunol. Chem. 272(17):11295-11301; Taryman, et al., (1995), Neuron. 14(4):755-762; Muller et al., (1998), Structure. 6(9):1153-1167; Bartunek et al., (1996), Cytokine. 8(1):14-20.

As discussed above, antibodies of the polypeptides of the invention can, in turn, be utilized to generate anti-idiotypic antibodies that “mimic” polypeptides of the invention using techniques well known to those skilled in the art (see, e.g. Greenspan and Bona (1989), FASEB J. 7(5):437-444 and Nissinoff, (1991), J. Immunol. 147(8): 2429-2438). For example, antibodies which bind to and competitively inhibit polypeptide multimerization or binding of a polypeptide of the invention to Ligand can be used to generate anti-idiotypes that “mimic” the polypeptide multimerization or binding domain and, as a consequence, bind to and neutralize polypeptide or its ligand. Such neutralization anti-idiotypic antibodies can be used to bind a polypeptide of the invention or to bind its ligandslreceptors, and thereby block its biological activity.

Immunoaffinity Chromatography

Antibodies prepared as described herein are coupled to a support. Preferably, the antibodies are monoclonal antibodies, but polyclonal antibodies may also be used. The support may be any of those typically employed in irmnunoaffinity chromatography, and the antibodies may be coupled to the support using any standard reagent. After coupling the antibody to the support, the support is contacted with a sample which contains a target polypeptide whose isolation, purification or enrichment is desired.

Thereafter, the support is washed with an appropriate wash solution, and the specifically bound target polypeptide is eluted from the support using the high pH or low pH elution solutions typically employed in immunoaffinity chromatography.

### Expression of NOVEL Gene Products

Evaluation of Expression Levels and Patterns of NOVEL Polypeptide-Encoding mRNAs

The spatial and temporal expression patterns of NOVEL polypeptide-encoding mRNAs, as well as their expression levels, may be determined using any suitable method.

In one embodiment, expression levels and patterns of NOVEL polypeptide-encoding mRNAs is analyzed by solution hybridization with probes (see, e.g., WO 97/05277). Briefly, an RNA complementary to the mRNA of interest is labeled and derivatized with a capturable moiety, e.g., biotin. After hybridization in solution with mRNA isolated from cells or tissues of interest, unhybridized probe is removed by digestion, and the remaining, hybridized RNA is captured, e.g., on a microtitration plate, and quantified.

In another embodiment, the NOVEL polypeptide-encoding cDNAs, or fragments thereof, may also be tagged with nucleotide sequences for the serial analysis of gene expression (SAGE) (see, e.g., UK Patent Application No. 2 305 241 A).

Quantitative analysis of NOVEL gene expression may also be performed using arrays. For example, quantitative analysis of gene expression may be performed with NOVEL polynucleotides, or fragments thereof in a complementary DNA microarray as described by Schena et al. (1995) Science 270:467470 and Schena et al. (1996), PNAS, 93(20):10614-10619, Pietu et al., (1996) Genome Research 6:492-503) or using any other microarray technology. Briefly, NOVEL polypeptide-encoding cDNAs or fragments thereof are arrayed onto slides, and the arrays are hybridized with probes derived from mRNA of cells or tissues of interest. Following washing, the arrays are scanned using a fluorescence laser scanning device fitted with a custom filter set. Accurate differential expression measurements are obtained by taking the average of the ratios of two independent hybridizations.

Alternatively, expression analysis of NOVEL genes can be done through high density nucleotide arrays as described by Lockhart et al., (1996) Nature Biotechnology 14: 1675-1680 and Sosnowski, et al., (1997) PNAS 94:1119-1123. Oligonucleotides of 15-50 nucleotides corresponding to sequences of a NOVEL polynucleotide or fragments thereof are synthesized directly on the chip or synthesized and then addressed to the chip. Labeled cDNA probes are synthesized from the appropriate mRNA population and then randomly fragmented to an average size of 50 to 100 nucleotides. The probes are then hybridized to the chip. After washing, the label is detected and quantified. Comparative analysis of the intensity of the signal originating from cDNA probes on the same target oligonucleotide in different cDNA samples indicates a differential expression of the NovEL polypeptide-encoding mRNA.

Uses of NOVEL Gene Expression Data

Once the expression levels and patterns of a NOVEL polypeptide-encoding mRNA has been determined, this information may be used to design NOVEL gene specific markers for detection, identification, screening and diagnostic purposes as well as to design DNA constructs with an expression pattern similar to a NOVEL gene expression pattern.

Detection of NOVEL Polypeptide Expression and/or Biological Activity

The invention further relates to methods of detection of NOVEL polypeptide expression and/or biological activity in a biological sample using the polynucleotide and polypeptide sequences described herein. Such methods can be used, for example, as a screen for normal or abnormal NOVEL polypeptide expression and/or biological activity and, thus, can be used diagnostically.

Detection of NOVEL Polypeptides

The invention further relates to methods of detection of NOVEL polypeptides in a cell or sample. In certain embodiments, the presence of polypeptides in a cell or sample is detected indirectly, by detecting the presence of mRNA encoding the polypeptide. For example, a labeled polynucleotide probe can be used in a method to detect a NOVEL polypeptide-encoding mRNA, wherein the presence of the mRNA is indicative of the expression of the NOVEL polypeptide-encoding gene.

Consequently, the invention comprises methods for detecting the presence of a polynucleotide of the invention in a sample, the method comprising bringing into contact said sample and a nucleic acid probe or a plurality of nucleic acid probes which hybridize to the polynucleotide, and detecting the hybrid complex formed between said probe or said plurality of probes and said polynucleotide. In certain embodiments, the probe or probes are labeled or are immobilized on a substrate.

In another embodiment, the polynucleotide is detected using an amplification reaction, wherein the sample is contacted with amplification reaction reagents, an amplification reaction is performed to synthesize amplification products containing said region of said selected nucleotide sequence; and the amplification products are detected. In a preferred embodiment, when the polynucleotide to be amplified is a RNA molecule, preliminary reverse transcription and synthesis of a second cDNA strand are first performed in order to provide a DNA template to be amplified.

Alternatively, a method of detecting NOVEL polypeptide expression in a test sample can be accomplished using any product which binds to a NOVEL polypeptide of the present invention or portion thereof. Such products may be antibodies, binding fragments of antibodies, polypeptides able to bind specifically to NOVEL polypeptides or fragments thereof, including NOVEL polypeptide agonists and antagonists. Detection of specific binding to the antibody indicates the presence of a NOVEL polypeptide in the sample (e.g., ELISA).

Consequently, the invention is also directed to a method for detecting specifically the presence of a NOVEL polypeptide in a biological sample, said method comprising bringing into contact the biological sample with a product able to bind to a polypeptide of the invention or fragments thereof; allowing the product to bind to the polypeptide to form a complex; and detecting the complex. In a preferred embodiment, the product is an antibody, e.g., an antibody that is immobilized on a substrate.

The present invention also relates to kits that can be used in the detection of NOvEL polypeptide-encoding gene expression products, e.g. containing a compound that specifically binds a NOVEL polypeptide (e.g. binding proteins, antibodies or binding fragments thereof (e.g. F(ab′)2 fragments) or a NOVEL polypeptide-encoding mRNA (e.g. a complementary probe or primer), disposed within a container means. The kit can further comprise ancillary reagents, including buffers and the like.

Detection of NOVEL Polypeptide Biological Activity

The invention further includes methods of detecting specifically a NOVEL polypeptide biological activity, and to identify compounds capable of modulating the activity of a NOVEL polypeptide. Assessing the NOVEL polypeptide biological activity may be performed by the detection of a change in any cellular property associated with the NOVEL polypeptide, using a variety of techniques, including those described herein. To identify modulators of the polypeptides, a control is preferably used. For example, a control sample includes all of the same reagents but lacks the compound or agent being assessed; it is treated in the same manner as the test sample.

The present invention also relates to kits that can be used in the detection of NOVEL polypeptide biological activity, e.g., including substrates for NOVEL polypeptides, NOVEL-binding compounds, antibodies to NOVEL polypeptides, etc., disposed within a container means. The kit can further comprise ancillary reagents, including buffers and the like.

Identification of a Specific Context of NOVEL Polypeptide-Encoding Gene Expression

When the expression pattern of a NOVEL polypeptide-encoding mRNA shows that a NOVEL polypeptide-encoding gene is specifically expressed in a given context, probes and primers specific for this gene as well as antibodies binding to the NOVEL polypeptide-encoding polynucleotide may then be used as markers for the specific context. Examples of specific contexts are: specific expression in a given tissue/cell or tissue/cell type, expression at a given stage of development of a process such as embryo development or disease development, expression in response to a particular compound or drug, or specific expression in a given organeile. Such primers, probes, and antibodies are useful commercially to identify tissues/cells/organelles of unknown origin, for example, forensic samples, differentiated tumor tissue that has metastasized to foreign bodily sites, or to differentiate different tissue types in a tissue cross-section.

Determination of tissue/cell/organelle identity is based on methods that detect the presence or absence of the mRNA (or corresponding cDNA or protein) in a tissue/cell sample using methods well known in the art (e.g., hybridization, PCR based methods, immunoassays, immunochemistry, ElISA). Therefore, the invention encompasses uses of the polynucleotides, polypeptides, and antibodies of the invention as tissue markers.

Identification of Tissue Types or Cell Species by Means of Labeled Tissue Specific Antibodies

Identification of specific tissues is accomplished by the visualization of tissue specific antigens by means of antibody preparations which are conjugated, directly (e.g., green fluorescent protein) or indirectly to a detectable marker. Selected labeled antibody species bind to their specific antigen binding partner in tissue sections, cell suspensions, or in extracts of soluble proteins from a tissue sample to provide a pattern for qualitative or semi-qualitative interpretation.

A. Immunohistochernical Techniques

Purified, high-titer antibodies, prepared as described above, are conjugated to a detectable marker, as described, for example, by Fudenberg, (1980) Chap. 26 in: Basic 503 Clinical Immunology, 3rd Ed. Lange, Los Altos, California or Rose et al., (1980) Chap. 12 in: Methods in Immunodiagnosis, 2d Ed. John Wiley 503 Sons, New York.

Antibodies can be labeled using any suitable method, e.g. fluorescent labels, enzymes (e.g. horseradish peroxidase), ferritin (for detection using EM), or radiolabeling. In one embodiment, cryostat sections of the unknown tissue and known control are mounted and each slide covered with different dilutions of the antibody preparation. Following incubation, excess fluid is blotted away, and the marker detected. The antigen found in the tissues by the above procedure can be quantified by measuring the intensity of color or fluorescence on the tissue section, and calibrating that signal using appropriate standards.

B. Identification of Tissue Specific Soluble Proteins

The visualization of tissue specific proteins and identification of unknown tissues from that procedure is carried out using the labeled antibody reagents and detection strategy as described for immunohistochemistry; however the sample is prepared according to an electrophoretic technique to distribute the proteins extracted from the tissue in an orderly array on the basis of molecular weight for detection, e.g. by western blotting. See, e.g., Davis et al., Basic Methods in Molecular Biology, ed., Elsevier Press, N.Y. (1986), Section 19-3.

Screening and Diagnosis of Abnormal NOVEL Polypeptide Expression and/or Biological Activity

Moreover, antibodies and/or primers or probes specific for NOVEL polypeptide expression may also be used to identify abnormal NOVEL polypeptide expression and/or biological activity, and subsequently to screen and/or diagnose disorders associated with abnormal NOVEL polypeptide expression. For example, a particular disease may result from lack of expression, over-expression, or underexpression of a NOVEL polypeptide-encoding mRNA. By comparing mRNA expression patterns and quantities in samples taken from healthy individuals with those from individuals suffering from a particular disorder, genes responsible for this disorder may be identified.

Screening for Specific Disorders

The present invention also relates to methods and uses of NOVEL polypeptides for identifying individuals having elevated or reduced levels of NOVEL polypeptides, which individuals are likely to benefit from therapies to suppress or enhance NOVEL polypeptide-encoding gene expression, respectively. One example of such methods and uses comprises detecting the presence in a biological sample of a NOVEL polypeptide-encoding gene product (mRNA or protein); comparing the amount of the NOVEL polypeptide-encoding gene product present in said sample with that of a control sample; and determining whether the sample has a reduced or elevated level of NOVEL gene expression compared to the control sample.

A biological sample from a subject affected by, or at risk of developing, any disease or condition associated with a NOVEL polypeptide can be screened for the presence of increased or decreased levels of NOVEL gene product, relative to a normal population (standard or control), with an increased or decreased level of the NOVEL polypeptide relative to the normal population being indicative of predisposition to or a present indication of the disease or condition, or any sympton associated with the disease or condition. Such individuals would be candidates for therapies, e.g., treatment with pharmaceutical compositions comprising the NOVEL polypeptide, a polynucleotide encoding the NOVEL polypeptide, or any other compound that affects the expression or activity of the NOVEL polypeptide. Generally, the identification of elevated levels of the NOVEL polypeptide in a patient would be indicative of an individual that would benefit from treatment with agents that suppress NOVEL polypeptide expression or activity, and the identification of low levels of the NOVEL polypeptide in a patient would be indicative of an individual that would benefit from agents that induce NOVEL expression or activity.

Biological samples suitable for use in this method include any biological fluids, including, but not limited to, blood, saliva, milk, and urine, as well as tissue samples such as biopsies. Cell cultures or cell extracts derived, for example, from tissue biopsies can also be used. In preferred embodiments, the biological sample is taken from animals presenting any symptom associated with any disease or condition associated with a NOVEL gene product. In accordance with this method, the presence in the sample of altered (e.g. increased or decreased) levels of the NOVEL product indicates that the subject is predisposed to the disease or condition.

The diagnostic methodologies described herein are applicable to both humans and non-hurnan mammals.

Detection of NOVEL Gene Mutations

The invention also encompasses methods and uses of NOVEL polynucleotides to detect mutations in NOVEL polynucleotides of the invention. When the mutation was proven to be associated with a disease, the detection of such mutations may be used for screening and diagnosis purposes.

In one embodiment, an oligonucleotide probe matrix may advantageously be used to detect mutations occurring in NOVEL genes and preferably in their regulatory regions. For this particular purpose, probes are specifically designed to have a nucleotide sequence allowing their hybridization to the genes that carry known mutations (either by deletion, insertion or substitution of one or several nucleotides). By known mutations, it is meant, mutations on the NOVEL genes that have been identified according, for example to the technique used by Huang et al., (1996) Cancer Res 56(5): 1137-1141 or Samson et al., (1996) Nature, 382(6593):722-725.

Another technique that is used to detect mutations in NOVEL genes is the use of a high-density DNA array. Each oligonucleotide probe constituting a unit element of the high density DNA array is designed to match a specific subsequence of a NOVEL genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the NOVEL gene. In one such embodiment, a 4L tiled array is used (see, e.g., Chee et al., (1996) Science. 274:610-614).

Construction of DNA Constructs with a NOVEL Gene Expression Pattern

In addition, characterization of the spatial and temporal expression patterns and expression levels of NOVEL polypeptide-encoding mRNAs is also useful for constructing expression vectors capable of producing a desired level of gene product in a desired spatial or temporal manner, as discussed below.

DNA Constructs That Direct Temporal And Spatial NOVEL gene Expression In Recombinant Cell Hosts And In Transgenic Animals.

In order to study the physiological and phenotypic consequences of a lack of synthesis of a NOVEL polypeptide, both at the cellular level and at the multi-cellular organism level, the invention also encompasses DNA constructs and recombinant vectors enabling a conditional expression of a specific allele of a NOVEL polypeptide-encoding genomic sequence or cDNA, including alleles that include substitutions, deletions, or additions of one or more bases within the sequence.

In one embodiment, a DNA construct is used that is based on the tetracycline resistance operon tet from E. coli transposon TnlO for controlling the NOVEL gene expression, such as described by Gossen et al., (1992) PNAS 89:5547-5551; Gossen et al., (1995) Science 268:1766-1769; and Furth P.A. et al. (1994) PNAS 91:9302-9306.

In another embodiment, a DNA construct is used that comprises, from 5′-end to 3′-end: a first nucleotide sequence that is found in the NOVEL polypeptide-encoding genomic sequence; a nucleotide sequence comprising a positive selection marker (e.g. neo); and a second nucleotide sequence that is found in the NOVEL polypeptide-encoding genomic sequence, and is located downstream of the first NOVEL nucleotide sequence. In a preferred embodiment, the construct also comprises a negative selection marker (e.g. thymidine kinase, hygromycine beta, hprt, Diphtheria toxin A fragment) located upstream of the first NOVEL nucleotide sequence or downstream from the second NOVEL nucleotide sequence (see, e.g., Thomas et al. (1986), Cell. 44:419-428; Te Riele et al. (1990), Nature. 348:649-651; Van der Lugt et al. (1991), Gene 105:263-267; Reid et al., (1990) PNAS 87:4299-4303; Nada et al., (1993) Cell 73:1125-1135; Yagi, T., et al. (1990), PNAS 87:9918-992; Thomas et al. (1986; 1987), Mansour et al.(1988) and Koller et al., (1992) Annu. Rev. Immunol. 10:705-730).

In another embodiment, vectors are used that involve the use of the Cre-loxP system (Hoess et aL, (1986) Nucleic Acids Res. 14:2287-2300). The Cre-loxP system used in combination with a homologous recombination technique has been described by Gu H. et al., (1993) Cell 73:1155-1164 and Gu H. et al., (1994) Science 265:103-106. Briefly, a nucleotide sequence of interest to be inserted in a targeted location of the genome harbors at least two loxP sites in the same orientation and located at the respective ends of a nucleotide sequence to be excised from the recombinant genome. The excision event requires the presence of the recombinase (Cre) enzyme within the nucleus of the recombinant cell host, which may be supplied in any of a number of ways, e.g., by incubating the recombinant cell hosts in a culture medium containing this enzyme, by injecting the Cre enzyme directly into the desired cell, by lipofection of the enzyme into the cells, or by otherwise transfecting the cell with a Cre coding sequence under the control of an appropriate promoter (see, e.g., Baubonis et al (1993) Nucleic Acids Res. 21(9):2025-9); Araki et al., (1995) PNAS 92(1):1604; Gu et al. (1993); Sauer et al., (1988) PNAS 85:5166-5170; Gu et al. (1994); Zou, et al, (1994) Curr. Biol. 4:1099-1103 ; Anton and Graham, (1995), J. Virol., 69: 46004606; and Kanegae et al., (1995) Nucl. Acids Res. 23:3816-3821).

The DNA constructs described above may be used to introduce a desired nucleotide sequence of the invention, preferably a NOVEL genomic sequence or a NOVEL CDNA sequence, and most preferably an altered copy of a NOVEL genomic or cDNA sequence, within a predetermined location of the targeted genome, leading either to the generation of an altered copy of a targeted gene (knock-out homologous recombination) or to the replacement of a copy of the targeted gene by another copy sufficiently homologous to allow an homologous recombination event to occur (knock-in homologous recombination).

Modifting NOVEL Polypoptide Expression and/or Biological Activity

Modifying endogenous NOVEL expression and/or biological activity is expressly contemplated by the present invention.

Screening for Compounds that Modulate NOVEL Expression and/or Biological Activity

The present invention further relates to compounds able to modulate NOVEL expression and/or biological activity and methods to use these compounds. Such compounds may interact with the regulatory sequences of NOVEL genes or they may interact with NOVEL polypeptides directly or indirectly.

Compounds Interacting With NOVEL Regulatory Sequences

TLe preseut invention also concerns a method for screening substances or molecules that are able to interact with the regulatory sequences of a NOVEL gene, such as for example promoter or enhancer sequences in untranscribed regions of the genomic DNA, as determined using any techniques known to those skilled in the art, or such as regulatory sequences located in untranslated regions of NOVEL mRNA.

Sequences within untranscribed or untranslated regions of polynucleotides.of the invention may be identified by comparison to databases containing known regulatory sequence such as transcription start sites, transcription factor binding sites, promoter sequences, enhancer sequences, 5′UTR and 3′UTR elements (Pesole et al., (2000) Nucleic Acids Res, 28(1): 193-196; http://igs-server.cnrs-mrs.fr/˜gauthere/index.html). Alternatively, the regulatory sequences of interest may be identified through conventional mutagenesis or deletion analyses of reporter plasmids.

Following the identification of potential NOVEL regulatory sequences, proteins which interact with these regulatory sequences may be identified as described below.

Any of a number of methods can be used to identify molecules capable of interacting with the regulatory sequence of a NOVEL gene, such as gel retardation assays (see, e.g., Fried and Crothers, (1981) Nucleic Acids Res. 9:6505-6525; Garner and Revzin, (1981) Nucleic Acids Res 9:3047-3060 ; and Dent and Latchman (1993) The DNA mobility shift assay. In: Transcription Factors: A Practical Approach (Latchman DS, ed.) pp: 1-26, Oxford: IRL Press). Nucleic acids encoding proteins which are able to interact with the promoter sequence of a NOVEL gene may also be identified by using a one-hybrid system (e.g., the Matchmaker One-Hybrid System kit from Clontech (Catalog Ref. no. K1603-1)).

Ligands Interacting with NOVEL Polypeptides

For the purpose of the present invention, a ligand means a molecule, such as a protein, a peptide, an antibody or any synthetic chemical compound capable of binding to a NOVEL protein or one of its fragments or variants or to modulate the expression of the polynucleotide coding for NOVEL or a fragment or variant thereof.

In one embodiment, a biological sample or a defined molecule (e.g. a molecule generated through combinatorial chemistry) to be tested as a putative ligand of a NOVEL protein is brought into contact with the purified NOVEL protein, in order to determine if a complex is formed between this protein and a component of the sample or the defined molecule. The interaction between such molecules and the protein can be assessed in any way, e.g., using microdialysis coupled to HPLC, using affinity capillary electrophoresis, etc. (see, e.g., Wang, et al. (1997), Chromatographia, 44: 205-208; Bush et al., (1997), J. Chromatogr., 777: 311-328).

Any type of compound can be tested, using any method, for interaction with a NOVEL polypeptide or polynucleotide in the present methods, including, but not limited to, peptides, drugs, fatty acids, lipoproteins, or small molecules, and may be obtained from any source. For example, the molecule to be tested is labeled with a detectable label, such as a fluorescent, radioactive, or enzymatic tag and placed in contact with immobilized NOVEL protein, or a fragment thereof under conditions which permit specific binding to occur. After removal of non-specifically bound molecules, bound molecules are detected using appropriate means.

A. Candidate Ligands Obtained from Random Peptide Libraries

In a particular embodiment of the screening method, the putative ligand is the expression product of a DNA insert contained in a phage vector, e.g. from a random peptide phage library comprising peptides of from 8 to 20 amino acids in length (Parrnley and Smith (1988) Gene 73:305-318; Oldenburg et al. (1992) PNAS 89:5393-5397; Valadon et al. (1996) J. Mol. Biol., 261:11-22; Lucas (1994) In: Development and Clinical Uses of Haempophilus b Conjugate; Westerink (1995) PNAS 92:40214025; Felici (1991) J. MoL Biol., 222:301-310).

Once the ligand library in recombinant phages has been constructed, the phage population is brought into contact with the immobilized NOVEL protein, and, following washing, the phages that bind specifically to the NOVEL protein are either eluted by a buffer (acid pH) or immunoprecipitated using an antibody specific to the NOVEL protein. The isolated phage is subsequently amplified by an over-infection of bacteria (for example E. coli). The selection step may be repeated several times, preferably 24 times, in order to select the more specific recombinant phage clones.

B. Candidate Ligands Obtained by Competition Experiments

Alternatively, peptides, drugs or small molecules which bind to polypeptides of the present invention may be identified in competition experiments. In one such assay, a NOVEL protein, or a fragment thereof, is immobilized to a surface, such as a plastic plate. Increasing amounts of the peptides, drugs or small molecules are placed in contact with the immobilized NOVEL protein, or a fragment thereof, in the presence of a detectable labeled known NOVEL protein ligand. For example, the NOVEL ligand may be detectably labeled with a fluorescent, radioactive, or enzymatic tag. The ability of the test molecule to bind the NOVEL protein, or a fragment thereof, is determined by measuring the amount of detectably labeled known ligand bound in the presence of the test molecule. A decrease in the amount of known ligand bound to the NOVEL protein, or a fragment thereof, when the test molecule is present indicated that the test molecule is able to bind to the NOVEL protein, or a fragment thereof.

C. Candidate Ligands Obtained by Affinity Chromatography

Proteins or other molecules interacting with a polypeptide of the present invention can also be found using affinity columns which contain the NOVEL protein, or a fragment thereof. The NOVEL protein, or a fragment thereof, may be attached to the column using conventional techniques including chemical coupling to a suitable column matrix (e.g. agarose, Affi Gel®, etc.). In some embodiments of this method, the affinity column contains chimeric proteins in which the NOVEL protein, or a fragment thereof, is fused to glutathion S transferase (GST). A mixture of cellular proteins or pool of expressed proteins as described above is applied to the affinity column. Proteins or other molecules interacting with the NOVEL protein, or a fragment thereof, attached to the column can then be isolated and analyzed, e.g., on 2-D electrophoresis gel as described in Ramunsen et al., (1997), Electrophoresis, 18: 588-598. Alternatively, the proteins retained on the affinity column can be purified by electrophoresis-based methods and sequenced. The same method can be used to isolate antibodies, to screen phage display products, or to screen phage display human antibodies.

D. Candidate Ligands Obtained by Optical Biosensor Methods

Proteins interacting with a polypeptide of the present invention, can also be screened by using an Optical Biosensor (see, e.g., Edwards and Leatherbarrow (1997) Anal. Bioch. 246:1-6; Szabo et al., (1995) Curr Opin Struct Biol 5, 699-705. This technique permits the detection of interactions between molecules in real time, without the need of labeled molecules. This technique is based on the surface plasmon resonance (SPR) phenomenon. In one embodiment, a NOVEL polypeptide, or fragment thereof, is attached to a surface (such as a carboxymethyl dextran matrix) comprising one side of a cell through which flows the candidate molecule to be assayed. A light beam is directed towards the side of the surface that does not contain the sample to be tested and is reflected by said surface. The binding of a candidate ligand molecules cause a change in the refraction index on the surface, which change is detected as a change in the SPR signal. This technique may also be performed by immobilizing eukaryotic or prokaryotic cells or lipid vesicles exhibiting an endogenous or a recombinantly expressed NOVEL protein at their surface.

E. Candidate Ligands Obtained Through a Two-Hybrid Screening Assay

The yeast two-hybrid system is designed to study protein-protein interactions in vivo (Fields and Song, 1989; U.S. Pat. Nos. 5,667,973 and 5,283,173), and relies upon the fusion of a bait protein to the DNA binding domain of the yeast Gal4 protein. The general procedure of library screening by the two-hybrid assay may be performed as described by Harper et al., (1993), Cell 75:805-816; Cho et al., (1998) PNAS 95(7):3752-3757; Fromont-Racine et al., (1997) Nature Genetics 16(3):277-282. In typical embodiments, the bait protein comprises a polypeptide of the present invention, and the “prey” comprises a human CDNA library constructed such that the human cDNA insert is fused to a nucleotide sequence in the vector that encodes the transcriptional domain of the GALA protein. In another embodiment, interaction between the NOVEL polypeptide or a fragment or variant thereof with cellular proteins is assessed using the Matchmaker Two Hybrid System 2 (Catalog No. K1604-1, Clontech).

Compounds Modulating NOVEL Biological Activity

Another method of screening for compounds that modulate NOVEL expression and/or biological activity is by measuring the effects of test compounds on specific biological activity, e.g. a NOVEL biological activity in a host cell or in an in vitro assay. A NOVEL biological activity can include any of the activities, functions, or properties described herein for any NOVEL polypeptide or polynucleotide. In one embodiment, a nucleic acid construct encoding a NOVEL polypeptide is introduced into a host cell, and the host cell is maintained under conditions appropriate for expression of the encoded NOVEL polypeptide, whereby the nucleic acid is expressed. The host cell is then contacted with a test agent, wherein a detection of a change in any NOVEL polypeptide-associated property in the presence of the agent indicates that the agent alters NOVEL biological activity. In a particular embodiment, the invention relates to a method of identifying an agent which is an activator of NOVEL biological activity, wherein detection of an increase of any NOVEL polypeptide-associated property in the presence of the agent indicates that the agent activates NOVEL biological activity. In another particular embodiment, the invention relates to a method of identifying an agent which is an inhibitor of NOVEL biological activity, wherein detection of a decrease of any NOVEL polypeptide-associated property in the presence of the agent indicates that the agent inhibits NOVEL biological activity. In another particular embodiment, a high throughput screen is used to identify agents that activate (enhance) or inhibit NOVEL biological activity (see, e.g., WO 98/45438).

Methods of Screening for Compounds Modulating NOVEL Gene Expression and/or Activity

The present invention also relates to methods of screening compounds for their ability to modulate (e.g. increase or inhibit) the activity or expression of NOVEL polypeptides and polynucleotides. More specifically, the present invention relates to methods of testing compounds for their ability either to increase or to decrease expression or activity of NOVEL polypeptides and polynucleotides. The assays are performed in vitro or in vivo.

In Vitro Methods

In vitro, cells expressing NOVEL polypeptides, or capable of expressing NOVEL polypeptides, are incubated in the presence and absence of the test compound. By determining the level of NOVEL expression in the presence of the test compound or the level of NOVEL biological activity in the presence of the test compound, compounds can be identified that suppress or enhance NOVEL expression or activity. Alternatively, constructs comprising a NOVEL regulatory sequence operably linked to a reporter gene (e.g. luciferase, chloramphenicol acetyl transferase, LacZ, green fluorescent protein, etc.) can be introduced into host cells and the effect of the test compounds on expression of the reporter gene detected. Consequently, the present invention encompasses a method for screening molecules that modulate the expression of a NOVEL gene, said screening method comprising cultivating a prokaryotic or a eukaryotic cell that has been transfected with a nucleotide sequence encoding either a NOVEL polypeptide, placed under the control of its own promoter, or a detectable polypeptide, placed under the control of a NOVEL 5′ regulatory region; bringing into contact the cultivated cell with a molecule to be tested; and quantifying the expression of the NOVEL or detectable polypeptide in the presence of the molecule. The method can also be performed using fragments, variants, or derivatives of any of the NOVEL polypeptides or 5′ regulatory regions.

The quantification of the expression of a NOVEL polypeptide may be realized either at the mRNA level (using for example Northen blots, RT-PCR, preferably quantitative RT-PCR with primers and probes specific for the NOVEL mRNA of interest) or at the protein level (using polyclonal or monoclonal antibodies in immunoassays such as ELISA or RIA assays, Western blots, or immunochemistry).

In a further embodiment, the NOVEL 5′ regulatory region includes the 5′UTR region of a NOVEL polynucleotide sequence, and/or a promoter sequence which is endogenous or exogenous with respect to the NOVEL 5′UTR sequence.

The invention further relates to a method for the production of a pharmaceutical composition comprising identifying a molecule that modulates the expression of a NOVEL gene using any of the herein-described methods, and combining the identified molecule with a physiologically acceptable carrier.

Kits for the screening candidate substances for the ability to modulate the expression of a NOVEL gene. Preferably, such kits comprise a recombinant vector comprising a NOVEL 5′ regulatory region or a regulatory active fragment or a variant thereof, operably linked to a polynucleotide encoding a detectable protein or a NOVEL protein or a fragment or a variant thereof.

Another object of the present invention comprises methods and kits for the screening of candidate Isubstances that interact with a NOVEL polypeptide, fragments or variants thereof. By their capacity to bind covalently or non-covalently to a NOVEL protein, fragments or variants thereof, these substances or molecules may be advantageously used both in vitro and in vivo.

In vitro, said interacting molecules may be used as detection means in order to identify the presence of a NOVEL protein in a sample, preferably a biological sample.

In one embodiment, a method is provided for the screening of a candidate substance, the method comprising providing a NOVEL protein; bringing into contact the protein with the candidate substance; and determining whether a complex is formed between the polypeptide or fragment and the candidate substance.

The invention further relates to a method for the production of a pharmaceutical composition comprising identifying a substance that interacts with a NOVEL polypeptide using any of the herein-described methods, fragments or variants thereof and furthermore mixing the identified substance with a physiologically acceptable carrier.

The invention further concerns a kit for the screening of a candidate substance interacting with the NOVEL polypeptide, wherein said kit comprises a NOVEL polypeptide, and optionally means to detect a complex formed between the polypeptide and the candidate substance. In one embodiment, the detection means comprises a monoclonal or polyclonal antibody binding to said NOVEL protein or fragment or variant thereof.

In vivo methods

Compounds that suppress or enhance NOVEL gene expression can also be identified using in vivo screens. In a typical assay, a test compound is administered (e.g. intravenously, intraperitoneally, intramuscularly, orally, or otherwise) to an animal, at a variety of dose levels, and the effect of the compound on NOVEL gene expression is determined by comparing the levels of the mRNA or protein encoded by the gene in tissues known to express the gene of interest, e.g., using Northern blots, immunoassays, PCR, etc.. Suitable test animals include, but are not limited to, rodents (e.g., mice and rats), primates, and rabbits. Humanized mice can also be used, that is mice in which the endogenous mouse protein is ablated (knocked out) and the homologous human protein introduced using standard transgenic approaches. Such mice thus express only the human form of a protein. Humanized mice expressing only the human NOVEL polypeptide can be used to study in vivo responses to potential agents regulating NOVEL protein or mRNA levels. Such transgenic animals are useful for dissecting the biochemical and physiological steps of disease, and for development of therapies for disease intervention (see, e.g., Loring, et al, 1996).

In addition, the detection of any change in any NOVEL gene-associated behavior or characteristic of an animal following the administration of a compound can also be used as an indication that the compound modulates the expression or activity of the gene. Uses for compounds modulating NOVEL ex pression andlor biological activity Using in vivo (or in vitro) systems, it may be possible to identify compounds that exert a tissue specific effect, for example, that increase NOVEL expression or activity in one or more particular tissues of interest, such as the adrenal gland, bone marrow, brain, cerebellum, colon, fetal brain, fetal kidney, fetal liver, heart, hypertrophic prostate, kidney, liver, lung, lymph ganglia, lymphocytes, muscle, ovary, pancreas, pituitary gland, placenta, prostate, salivary gland, spinal cord, spleen, stomach, intestine, substantia nigra, testis, thyroid, umbilical cord, or uterus. Screening procedures such as those described above are also useful for identifying agents for their potential use in pharmacological intervention strategies. Agents that enhance NOVEL gene expression or stimulate its activity may thus be used to induce any phenotype associated with a NOVEL gene, or to treat disorders resulting from a deficiency of a NOVEL polypeptide activity or expression. Compounds that suppress NOVEL polypeptide expression or inhibit its activity can be used to treat any disease or condition associated with increased or deleterious NOVEL polypeptide activity or expression.

Also encompassed by the present invention is an agent which interacts with a NOVEL gene or polypeptide directly or indirectly, and inhibits or enhances NOVEL polypeptide expression and/or function. In one embodiment, the agent is an inhibitor which interferes with a NOVEL polypeptide directly (e.g., by binding the NOVEL polypeptide) or indirectly (e.g., by blocking the ability of the NOVEL polypeptide to have a NOVEL biological activity). In a particular embodiment, an inhibitor of a NOVEL protein is an antibody specific for the NOVEL protein or a functional portion of the NOVEL protein. Alternatively, the inhibitor can be an agent other than an antibody (e.g., small organic molecule, protein or peptide) which binds the NOVEL polypeptide and blocks its activity. For example, the inhibitor can be an agent which mimics the NOVEL polypeptide structurally, but lacks its function (e.g. a dominant negative form of the protein). Alternatively, it can be an agent which binds to or interacts with a molecule which the NOVEL polypeptide normally binds to or interacts with, thus blocking the NOVEL polypeptide from doing so and preventing it from exerting the effects it would normally exert.

In another embodiment, the agent is an enhancer (activator) of a NOVEL polypeptide which increases the activity of the NOVEL polypeptide (increases the effect of a given amount or level of NOVEL polypeptide), increases the length of time it is effective (by preventing its degradation or otherwise prolonging the time during which it is active) or both either directly or indirectly. For example, NOVEL polynucleotides and polypeptides can be used to identify drugs which increase or decrease the ability of NOVEL polypeptides to induce NOVEL biological activity, which drugs are useful for the treatment or prevention of any disease or condition associated with a NOVEL biological activity.

Thus the present invention relates to a method of inhibiting (partially or completely) a NOVEL biological activity in a mammal (e.g., a human), the method comprising administering to the mammal an effective amount of an inhibitor of a NOVEL polypeptide or polynucleotide. The invention also relates to a method of enhancing a NOVEL biological activity in a mrnamal, the method comprising administering to the mammal an effective amount of an enhancer of a NOVEL polypeptide or polynucleotide. Inhibiting NOVEL gene expression NOVEL gene expression can be inhibited, e.g., for therapeutic applications, in any of a large number of ways, including by using an antisense tool or a triple helix tool that inhibits the expression of the corresponding NOVEL gene.

Antisense Aproach In antisense approaches, DNA and/or RNA sequences complementary to an mRNA are hybridized to the mRNA intracellularly, thereby blocking the expression of the protein encoded by the mRNA. Preferred methods for using antisense polynucleotide according to the present invention are the procedures described by Sczakiel et al., (1995) Trends Microbiol. 3(6):213-217; Green et al., (1986) Ann. Rev. Biochem 55:569-597 ; Izant and Weintraub, (1984) Cell 36(4): 1007-15; Liu et al. (1994) 4528-4262 ; Eckner et al., (1991) EMBO J. 10:3513-3522).

Preferably, the antisense tools are chosen among the polynucleotides (15-200 bp long) that are complementary to NOVEL mRNA, more preferably to the 5′end (e.g. the translation initiation codon ATG) or to a splicing donor or acceptor site of the NOVEL rnRNA. In another embodiment, a combination of different antisense polynucleotides complementary to different parts of the desired targeted gene are used. The antisense molecules can be prepared in any way, including by reversing the orientation of a coding region of a NOVEL gene in a cell, or by synthesizing an oligonucleotide in vitro and administering it to the cell.

Any antisense sequence complementary to any portion of any of the herein described polynucleotides can be used, and can involve any number of modifications to the backbone, linkages, or bases, a large number of which are known in the art Suitable modifications, other forms of antisense molecules, and the preparation thereof, are taught, inter alia, in The Concise Encyclopedia Of Polymer Science And Engineering, pages 858-859, Kroschwitz, J. I., ed. John Wiley & Sons, 1990; Englisch et al., Angewandte Chemie, International Edition (1991), 30, 613; Sanghvi, et al., eds., (1993) Antisense Research and Applications, CRC Press, Boca Raton; U.S. Pat. No. 6,242,590; WO94/23026, WO 96/31523; WO 92/18522; European Patent Application No. 0 572 287 A2; WO 92/19732; Letsinger et al., PNAS (1989) 86: 6553-6556; Manoharan et al., Bioorg. Med. Chem Let. (1994) 4:1053-1060; Manoharan et al., Ann. N.Y. Acad. Sci. (1992) 660:306-309; Manoharan et al., Bioorg. Med. Chem Let. (1993) 3:2765-2770; Oberhauser et al., Nucl. Acids Res. (1992) 20:533-538; Saison-Behmoaras et al., EMBO J.(1991)10:1111-2118; Kabanovetal., FEBS Lett. (1990)259:327-330; Svinarchuketal., Biochimie (1993) 75:49-54; Manoharan et al., Tetrahedron Lett. (1995) 36, 3651-3654; Shea et al., Nucl. Acids Res. (1990) 18:3777-3783; Manoharan et al., Nucleosides & Nucleotides (1995) 14: 969-973; Manoharan et al., Tetrahedron Lett. (1995) 36:3651-3654; Mishra et al., BiochimL Biophys. Acta (1995) 1264:229-237; Crooke et al., J. Phamacol. Exp. Ther. (1996) 277:923-937; U.S. Pat. No. 6,242,590.

Further included in the present invention is a method of high throughput screening of antisense nucleic acids and modified versions thereof for binding to targeted NOVEL polynucleotide sequences or fragments thereof (see, e.g., U.S. Pat. No. 6,022,691).

The appropriate level of antisense nucleic acids required to inhibit gene expression may be determined using in vitro expression analysis. Typically, antisense molecules are introduced onto cell samples at a number of different concentrations preferably between 1×10⁻¹⁰M to 1×10⁻⁴M. Once the rninimum concentration that can adequately control gene expression is identified, the optimized dose is translated into a dosage suitable for use in vivo or ex vivo. For example, an inhibiting concentration in culture of 1×10⁻⁷ translates into a dose of approximately 0.6 mg/kg bodyweight.

An alternative to the antisense technology that is used according to the present invention comprises using ribozymes that will bind to a target sequence via their complementary polynucleotide tail and that will cleave the corresponding RNA by hydrolyzing its target site (see, e.g., Rossi et al., (1991) Phannacol. Ther. 50:245-254 and Sczakiel et al. (1995)).

Triple Helix Approach

The NOVEL genomic DNA may also be used to inhibit the expression of the NOVEL gene based on intracellular triple helix formation (see, e.g., Griffin et al., (1989) Science 245:967-971). To carry out gene therapy strategies using the triple helix approach, the sequences of the NOVEL genomic DNA are typically first scanned to identify 10-mer to 20-mer homopyrimidine or homopurine stretches which could be used in triple-helix based strategies for inhibiting NOVEL gene expression. Following identification of candidate homopyrimidine or homopurine stretches, their efficiency in inhibiting NOVEL gene expression is assessed by introducing varying amounts of oligonucleotides containing the candidate sequences into tissue culture cells which express the NOVEL gene. The oligonucleotides can be introduced into the cells using a variety of methods known to those skilled in the art, and treated cells are monitored for altered cell function or reduced NOVEL gene expression.

The oligonucleotides which are effective in inhibiting gene expression in tissue culture cells may then be introduced in vivo using the techniques and at a dosage calculated based on the in vitro results, as described in the section entitled “Antisense Approach”.

Treating NOVEL Gene-Related Disorders

The present invention further relates to methods, uses of NOVEL polypeptides and polynucleotides, and uses of modulators of NOVEL polypeptides and polynucleotides, for treating diseases/disorders associated with NOVEL genes by increasing or decreasing NOVEL gene activity and/or expression. These methodologies can be effected using compounds selected using screening protocols such as those described herein and/or by using the gene therapy and antisense approaches described in the art and herein. Gene therapy can be used to effect targeted expression of NOVEL genes in any tissue, e.g. a tissue associated with the disease or condition to be treated. The NOVEL coding sequence can be cloned into an appropriate expression vector and targeted to a particular cell type(s) to achieve efficient, high level expression. Introduction of the NOVEL coding sequence into target cells can be achieved, for example, using particle mediated DNA delivery, (Haynes et al., (1996) J Biotechnol. 44(1-3):3742 and Maurer et al., (1999) Mol Membr Biol. 16(1):12940), direct injection of naked DNA (Levy et al., (1996) Gene Ther. 3(3):201-21; and Felgner (1996) Hum Gene Ther. 7(15):1791-3), or viral vector mediated transport (Smith et al., (1996) Antiviral Res. 32(2):99-115, Stone et al., (2000) J Endocrinol. 164(2):103-18; Wu and Ataai (2000), Curr Opin Biotechnol. 1 1(2):205-8). Tissue specific effects can be achieved, for example, in the case of virus mediated transport by using viral vectors that are tissue specific, or by the use of promoters that are tissue specific. For instance, any tissue-specific promoter may be used to achieve specific expression, for example albumin promoters (liver specific; Pinkert et al., 1987 Genes Dev 1:268-277), lymphoid specific promoters (Calame et al., 1988 Adv. Immunol. 43:235-275), promoters of T-cell receptors (Winoto et al., 1989 EMBO J. 8:729-733) and immunoglobulins (Baneji et al., 1983 Cell 33:729-740; Queen and Baltimore 1983 Cell 33:741-748), neuron-specific promoters (e.g. the neurofilament promoter, Byrne et al., 1989 PNAS 86:5473-5477), pancreas-specific promoters (Edlunch et al., 1985 Science 230:912-916) or mammary gland-specific promoters (milk whey promoter, U.S. Pat. No. 4,873,316 and European Application Publication No. 264, 166). Developmentally-regulated promoters can also be used, such as the murine homeobox promoters (Kessel et al., 1990 Science 249:374-379) or the alpha-fetoprotein promoter (Campes et al., 1989 Genes Dev. 3:537-546).

Combinatorial approaches can also be used to ensure that the NOVEL coding sequence is activated in the target tissue (Butt and Karathanasis (1995) Gene Expr. 4(6):319-36; Miller and Whelan, (1997) Hum Gene Ther. 8(7):803-15). Antisense oligonucleotides complementary to NOVEL mRNA can be used to selectively diminish or ablate the expression of the protein (Wagner, et al. (1996), Nat Biotechnol. 14(7):8404)), for example, at sites of inflammation. More specifically, antisense constructs or antisense oligonucleotides can be used to inhibit the production of NOVEL in high expressing cells, e.g., by transfecting target cells with an expression vector comprising a NOVEL gene sequence, or portion thereof, in an antisense orientation relative to the direction of transcription, or by introducing antisense oligonucleotides directly into target cells. The therapeutic methodologies described herein are applicable to both human and non-human mammals.

Pharmaceutical and Physiologically Acceptable Compositions

The present invention also relates to pharmaceutical or physiologically acceptable compositions comprising, as active agent, the polypeptides, nucleic acids or antibodies of the invention. The invention also relates to compositions comprising, as active agent, compounds selected using the above-described screening protocols. Such compositions include the active agent in combination with a pharmaceutical or physiologically acceptable carriers such as a physiologically acceptable salt, ester, or salt of such esters. In the case of naked DNA, the “carrier” may be gold particles. The amount of active agent in the composition can vary with the agent, the patient and the effect sought. Likewise, the dosing regimen can vary depending on the composition and the disease/disorder to be treated.

Therefore, the invention related to methods for the production of pharmaceutical composition comprising a method for selecting an active agent, compound, substance or molecule using any of the screening method described herein and furthermore mixing the identified active agent, compound, substance or molecule with a physiologically acceptable carrier.

The term “physiologically acceptable salts” refers to physiologically and pharmaceutically acceptable salts of the compounds of the invention: i.e., salts that retain the desired biological activity of the parent compound and do not impart undesired toxicological effects thereto. Physiologically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucae, and procaine (see, for example, Berge et al., “Pharmaceutical Salts,” J. of Pharma Sci. (1977) 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a physiologically acceptable salt of an acid form of one of the components of the compositions of the invention. These include organic or inorganic acid salts of the amines. Preferred acid salts are the hydrochlorides, acetates, salicylates, nitrates and phosphates. Other suitable physiologically acceptable salts are well known to those skilled in the art and include basic salts of a variety of inorganic and organic acids, such as, hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Physiologically acceptable salts of compounds may also be prepared with a physiologically acceptable cation. Suitable physiologically acceptable cations are well known to those skilled in the art and include alkaline, alkaline earth, ammonium and quatemary ammonium cations. Carbonates or hydrogen carbonates are also possible. For oligonucleotides, preferred examples of physiologically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, faric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The pharmaceutical compositions utilized in this invention may be administered by any number of routes including, but not limited to: parenteral, intracranial, intraorbital, intracapsular, intraspinal, intracistemal, intrapulmonary, oral, intravenous, intramuscular, intraarterial, intramedullary, intrathecal, intraventricular, transdermal, subcutaneous, intraperitoneal, intranasal, enteral, topical, sublingual, rectal, intradermal, intravascular. In addition to the active ingredients, these pharmaceutical compositions may contain suitable physiologically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack PublishingCo. Easton, Pa).

Pharmaceutical compositions for oral administration can be formulated using physiologically acceptable carriers well known in the art in dosages suitable for oral administration. Such carriers enable the pharmaceutical compositions to be formulated as powders, tablets, pills, dragees, capsules, liquids, gels, syrups, slurries, suspensions, and the like, for ingestion by the patient. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Formulations suitable for pulmonary or respiratory delivery include dry powders, liquid solutions or suspensions suitable for nebulization, and propellant formulations suitable for use in metered dose inhalers (MDI's). In typical embodiments, dry powder formulations will have a particle size within a preferred range for deposition within the alveolar region of the lung, typically from 0.5 μm to 5pm. Respirable powders of pharmaceutical compositions within the preferred size range can be produced by a variety of conventional techniques, such as jet-milling, spray-rying, solvent precipitation, and the like. Dry powders can then be administered to the patient in conventional dry powder inhalers (DPI's) that use the patient's inspiratory breath through the device to disperse the powder or in air-assisted devices that use an external power source to disperse the powder into an aerosol cloud (see, e.g., U.S. Pat. No. 5,458,135).

Liquid formulations for use in nebulizer systems preferably employ slightly acidic buffers (pH 4-6) such as acetate, ascorbate, and citrate, at concentrations of 5 mM to 50 mM. These buffers can act as antioxidants. Physiologically acceptable components to enhance or maintain chemical stability include: antioxidants, chelating agents, protease inhibitors, isotonic modifiers, inert gases, and the like. A preferred type of nebulizer suitable for delivering such liquid formulations is described in U.S. Pat. No. 5,458,135.

For use in MDI's, the pharmaceutical composition will typically be processed into respirable particles as described for the dry powder formulations, and the particles then suspended in a suitable aerosol propellant (such as a CFC or HFC), typically being coated with a surfactant to enhance their dispersion. Such aerosol propellant formulations may further include a lower alcohol, such as ethanol (up to 30% by weight) and other additives to maintain or enhance chemical stability and physiological acceptability (see, e.g., U.S. Pat. No. 6,080,721).

For topical or nasal administration, penetrants appropriate to the particular barrier to be permeated are used in the formulation. Such penetrants are generally known in the art.

Pharmaceutical formulations suitable for parenteral administration may be formulated in aqueous solutions, preferably in physiologically compatible buffers such as Hanks solution, Ringer's solution, or physiologically buffered saline. Aqueous injection suspensions may contain substances which increase the viscosity of the suspension, such as sodium carboxymethylcellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

In one embodiment, the preparation is a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of a NOVEL polypeptide, such labeling would include, e.g., amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compound, a therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

A therapeutically effective dose refers to that amount of active ingredient, for example a NOVEL polypeptide or fragments thereof, antibodies specific to NOVEL polypeptides, agonists, antagonists or inhibitors of NOVEL polypeptides, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Other factors that may be considered when evaluating the proper dosage include the chemical nature of the compound destined for delivery, the biological responses associated with the compound (both intended and coincidental) and anticipated contraindications. Additionally, the mode of delivery, the duration and frequency of administration (e.g. n doses per hours, n doses per day, n doses per week, cumulative dosage per day, cumulative dosage per week), the biologically effective dose delivered to target site, often indicated by plasma level concentrations, and the rate or efficiency of compound clearance from the body may be considered. Long-acting pharmaceutical compositions may be administered, e.g., every 3 to 4 days, every week, or once every two weeks depending on half-life and clearance rate of the particular formulation.

Normal dosage amounts may vary depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. In general, for a 75kg individual the normal dosage range are as follows: for a small molecule compound an effective does is usually between 0.3-50 mg/kg; for recombinant polypeptides an effective dose is usually between 0.25-7.5 mg/kg; for compounds used for mediating humoral immune responses (e.g., polyvalent pneumococcal vaccine, Rh (D) immune globulin, Hepatitis B vaccine, anti-CD20 antigen) the effective dose is usually between 0.0015-1.5 mg/kg; for hormone supplemental compounds (e.g. estradiol, norethindrone) the effective dose is usually between 0.0005-0.5 mg/kg depending upon delivery system utilized (e.g. transdermal, oral, topical).

Transdermal delivery systems (e.g. estradiol transdermal system, transdermal scopolamine system, transfermal nicotine patch) must be calibrated for nominal delivery dosages based upon efficiency of percutaneous delivery for the individual and specific compounds, surface area (cm²) of transdermal system contact, quantity and form of compound integrated into transdermal delivery system and anatomical location of positioned transdermal system. The effective dosage range of compounds admistered in this manner is usually between 0.005-0.5 mg/kg

Uses of NOVEL Sequences: Computer-Related Embodiments

It will be appreciated by those skilled in the art that the nucleic acid codes of the invention and polypeptide codes of the invention can be stored, recorded, and manipulated on any medium which can be read and accessed by a computer. As used herein, the words “recorded” and “stored” refer to a process for storing information on a computer medium A skilled artisan can readily adopt any of the presently known methods for recording information on a computer readable medium to generate manufactures comprising one or more of the nucleic acid codes of the invention, or one or more of the polypeptide codes of the invention. Another aspect of the present invention is a computer readable medium having recorded thereon at least 1, 2, 5, 10, 15, 20, 25, 30, or 50 nucleic acid or polypepti.de codes of the invention.

Computer readable media include magnetically readable media, optically readable media, electronically readable media and magnetic/optical media. For example, the computer readable media may be a bard disk, a floppy disk, a magnetic tape, CD-ROM, Digital Versatile Disk (DVD), Random Access Memory , or Read Only Memory (ROM).

Embodiments of the present invention include systems, particularly computer systems which store and manipulate the sequence information described herein. As used herein, “a computer system“refers to the hardware components, software components, and data storage components used to analyze the nucleotide sequences of the nucleic acid codes of the invention or the amino acid sequences of the polypeptide codes of the invention. The computer system preferably includes a processor for processing, accessing and manipulating the sequence data. The processor can be any well-known type of central processing unit, such as the Pentium III from Intel Corporation, or similar processor from Sun, Motorola, Compaq or International Business Machines. Preferably, the computer system is a general purpose system that comprises the processor and one or more internal data storage components for storing data, and one or more data retrieving devices for retrieving the data stored on the data storage components. A skilled artisan can readily appreciate that any one of the currently available computer systems are suitable.

In some embodiments, the computer system may comprise a sequence comparer for comparing the above-described nucleic acid codes of the invention or the polypeptide codes of the invention stored on a computer readable medium to reference nucleotide or polypeptide sequences stored on a computer readable medium or present in a database. A “sequence comparer” refers to one or more programs which are implemented on the computer system to compare a nucleotide or polypeptide sequence with other nucleotide or polypeptide sequences and/or compounds including but not limited to peptides, peptidomimetics, and chemicals stored within the data storage means. For example, the sequence comparer may compare the nucleotide sequences of nucleic acid codes of the invention or the amino acid sequences of the polypeptide codes of the invention stored on a computer readable medium to reference sequences stored on a computer readable medium to identify homologies, motifs implicated in biological function, or structural motifs. The various sequence comparer programs identified elsewhere in this patent specification are particularly contemplated for use in this aspect of the invention. Alternatively, a nucleotide or amino acid sequence is compared with a database of sequences in order to determine the homology levels between the sequence and the sequences in the database. The database of sequences can be a private database stored within the computer system or a public database such as GENBANK, PIR OR SWISSPROT that is available, e.g., through the Internet.

Accordingly, one aspect of the present invention is a computer system comprising a processor, a data storage device having stored thereon a nucleic acid code of the invention or a polypeptide code of the invention, a data storage device having retrievably stored thereon reference nucleotide sequences or polypeptide sequences to be compared to the nucleic acid code of the invention or polypeptide code of the invention and a sequence comparer for conducting the comparison.

Alternatively, the computer program may be a computer program which compares the nucleotide sequences of the nucleic acid codes of the present invention, to reference nucleotide sequences in order to determine whether the nucleic acid code of the invention differs from a reference nucleic acid sequence at one or more positions. Optionally such a program records the length and identity of inserted, deleted or substituted nucleotides with respect to the sequence of either the reference polynucleotide or the nucleic acid code of the invention. In one embodiment, the computer program may be a program which determines whether the nucleotide sequences of the nucleic acid codes of the invention contain one or more single nucleotide polymorphisms (SNP) with respect to a reference nucleotide sequence. These single nucleotide polymorphisms may each comprise a single base substitution, insertion, or deletion. The method may also be performed by reading at least 2, 5, 10, 15, 20, 25, 30, or 50 of the nucleic acid codes of the invention and the reference nucleotide sequences through the use of the computer program and identifying differences between the nucleic acid codes and the reference nucleotide sequences with the computer program.

In other embodiments the computer based system may further comprise an identifier for identifying features within the nucleotide sequences of the nucleic acid codes of the invention or the amino acid sequences of the polypeptide codes of the invention. An “identifier” refers to one or more programs which identifies certain features within the above-described nucleotide sequences of the nucleic acid codes of the invention or the amino acid sequences of the polypeptide codes of the invention. In one embodiment, the identifier may comprise a program which identifies an open reading frame in the cDNAs codes of the invention.

In additional embodiments, the present amino acid sequences can be analyzed using any of a number of computer programs to identify structural features of the protein, such as secondary, tertiary, and quarternary structures, to identify potential binding partners, etc. The results of the molecular modeling analysis may then be used, e.g., in rational drug design techniques to identify agents which modulate the activity of the polypeptide codes of the invention.

Accordingly, another aspect of the present invention is a method of identifying a feature within the nucleic acid codes of the invention or the polypeptide codes of the invention comprising reading the nucleic acid code(s) or the polypeptide code(s) through the use of a computer program which identifies features therein and identifying features within the nucleic acid code(s) or polypeptide code(s) with the computer program The method may be performed by reading a single sequence or at least 2, 5, 10, 15, 20, 25, 30, or 50 of the nucleic acid codes of the invention or the polypeptide codes of the invention through the use of the computer program and identifying features within the nucleic acid codes or polypeptide codes with the computer program Motifs which may be detected using the above programs include leucine zippers, helix-turn-helix motifs, glycosylation sites, ubiquitination sites, alpha helices, beta sheets, signal sequences, sequences implicated in transcription regulation such as homeoboxes, acidic stretches, enzymatic active sites, substrate binding sites, and enzymatic cleavage sites.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims.

Throughout this application, various publications, kit manuals, patents and published patent applications are cited. The entire disclosures of each of these publications, patents, manuals and published patent specifications are hereby incorporated by reference into the present disclosure to more fully describe the state of the art to which this invention pertains.

TABLE I

| SEQ | Sequence | | |
|---|---|---|---|
| ID NO. | Type | Clone ID_Clone Name | Name |
| 1 | DNA | 500695767.cFS_188-227-2-0-G1-F | Nemeglya |
| 2 | Protein | 500695767.cFS_188-227-2-0-G1-F | Nemeglya |
| 3 | DNA | 1000772225_208-30-4-0-H4-F | Sica19 |
| 4 | Protein | 1000772225_208-30-4-0-H4-F | Sica19 |
| 5 | DNA | 500737075_cFS_205-46-1-0-D7-F | Plap11 |
| 6 | Protein | 500737075_cFS_205-46-1-0-D7-F | Plap11 |
| 7 | DNA | 500720716_204-33-1-0-E6-F_1 | Nupre |
| 8 | Protein | 500720716_204-33-1-0-E6-F_1 | Nupre |
| 9 | DNA | 599222_181-13-3-0-F11-F | NFHR |
| 10 | Protein | 599222_181-13-3-0-F11-F | NFHR |
| 11 | DNA | 238757_116-105-3-0-D9-F | NARF |
| 12 | Protein | 238757_116-105-3-0-D9-F | NARF |
| 13 | DNA | 106614_105-031-2-0-E2-F | SgIIb |
| 14 | Protein | 106614_105-031-2-0-E2-F | SgIIb |
| 15 | DNA | 1000770389_208-24-4-0-G7-F | Sepin |
| 16 | Protein | 1000770389_208-24-4-0-G7-F | Sepin |
| 17 | DNA | 1000888456_159-16-3-0-E4-F | ULBP2A |
| 18 | Protein | 1000888456_159-16-3-0-E4-F | ULBP2A |
| 19 | DNA | 1000848040_181-41-1-0-C1-F | TM4SF5A |
| 20 | Protein | 1000848040_181-41-1-0-C1-F | TM4SF5A |
| 21 | DNA | 500762393_205-70-1-0-B12-F | NoJAM |
| 22 | Protein | 500762393_205-70-1-0-B12-F | NoJAM |
| 23 | DNA | 1000848660_181-42-1-0-C11-F | NCCL14 |
| 24 | Protein | 1000848660_181-42-1-0-C11-F | NCCL14 |
| 25 | DNA | 500735264_205-41-2-0-A7-F | CS-5b |
| 26 | Protein | 500735264_205-41-2-0-A7-F | CS-5b |
| 27 | DNA | 500710542_205-8-4-0-F1-F | Placik |
| 28 | Protein | 500710542_205-8-4-0-F1-F | Placik |
| 29 | DNA | 500728413_204-53-2-0-B2-F | Codeac |
| 30 | Protein | 500728413_204-53-2-0-B2-F | Codeac |
| 31 | DNA | 131715_105-090-4-0-D2-F | Prospan |
| 32 | Protein | 131715_105-090-4-0-D2-F | Prospan |
| 33 | DNA | 109010_105-041-4-0-A5-F | Prospanyl |
| 34 | Protein | 109010_105-041-4-0-A5-F | Prospanyl |
| 35 | DNA | 584047_181-11-4-0-G9-F | Firp |
| 36 | Protein | 584047_181-11-4-0-G9-F | Firp |
| 37 | DNA | 140354_105-115-4-0-F5-F | Sapro |
| 38 | Protein | 140354_105-115-4-0-F5-F | Sapro |
| 39 | DNA | 485499_174-12-3-0-H7-F | Saprip |
| 40 | Protein | 485499_174-12-3-0-H7-F | Saprip |

TABLE II

| SEQ ID | | Signal | Mature | Polyadenylation | PolyA |
|---|---|---|---|---|---|
| NO: | ORF | Peptide | peptide | Signal | tail |
| 1 | [260-904] | — | — | [1241-1246] | [1248-1289] |
| 3 | [138-320] | [138-200] | [201-320] | [562-567] | [588-603] |
| 5 | [112-1341] | [112-165] | [ 166-1341] | [1528-1533] | [1548-1673] |
| 7 | [58-849] | [58-135] | [136-849] | [952-957] | [978-993] |
| 9 | [24-461] | [24-77] | [78-461] | [572-577] | [596-611] |
| 11 | [98-571] | — | — | — | [574-588] |
| 13 | [18-1403] | [18-86] | [87-1403] | [1594-1599] | [1618-1633] |
| 15 | [22-360] | [22-96] | [97-360] | [565-570] | [590-605] |
| 17 | [58-690] | [58-141] | [142-690] | [1177-1182] | [1197-1212] |
| 19 | [40-435] | [40-117] | [118-435] | [910-915] | [932-947] |
| 21 | [230-934] | [230-313] | [314-934] | [1353-1358] | [1374-1389] |
| 23 | [110-304] | [110-166] | [167-304] | [518-523] | [536-551] |
| 25 | [147-626] | [147-224] | [225-626] | [714-719] | [741-786] |
| 27 | [318-902] | [318-398] | [399-902] | [1345-1350] | [1368-1409] |
| 29 | [124-1074] | [124-225] | [226-1074] | [1158-1163] | [1180-1195] |
| 31 | [42-371] | [42-110] | [111-371] | [1709-1714] | [1730-1745] |
| 33 | [42-311] | [42-110] | [111-311] | [1372-1377] | [1394-1409] |
| 35 | [190-1092] | [190-255] | [256-1092] | — | [1138-1153] |
| 37 | [76-231] | [76-132] | [133-231] | [509-514] | [533-548] |
| 39 | [39-662] | [39-86] | [87-662] | [757-762] | [777-792] |

TABLE III

| SEQ ID | |
|---|---|
| NO: | Positions of immunogenic epitopes |
| 2 | 203 . . . 214 |
| 4 | −1 . . . 7 |
| 8 | 14 . . . 27: 56 . . . 64 |
| 10 | 3 . . . 8: 10 . . . 18: 40 . . . 62: 78 . . . 94: 100 . . . 112: 114 . . . 120 |
| 12 | 25 . . . 33: 52 . . . 60: 87 . . . 101: 110 . . . 115: 143 . . . 148 |
| 14 | −2 . . . 40: 47 . . . 64: 74 . . . 79: 83 . . . 100: 113 . . . 135: 132 . . . 144: 157 . . . 170: 182 . . . 199: 204 . . . 212: |
| | 218 . . . 231: 239 . . . 253: 255 . . . 272: 294 . . . 307: 311 . . . 317: 316 . . . 347: 354 . . . 393: 396 . . . 407: |
| | 424 . . . 438 |
| 18 | −8 . . . 5: 14 . . . 20: 36 . . . 43: 102 . . . 112: 124 . . . 131: 135 . . . 140 |
| 20 | 6 . . . 16: 54 . . . 61: 84 . . . 92 |
| 22 | −28 . . . −22: 2 . . . 8: 23 . . . 31: 32 . . . 40: 48 . . . 58: 69 . . . 82: 86 . . . 91: 126 . . . 136: 134 . . . 140: 138 . . . 143: |
| | 148 . . . 170: 177 . . . 203 |
| 24 | 1 . . . 16: 20 . . . 27: 31 . . . 44 |
| 28 | 27 . . . 43: 63 . . . 76: 80 . . . 93: 99 . . . 108: 151 . . . 161 |
| 30 | 5 . . . 12: 15 . . . 22: 38 . . . 44: 50 . . . 56: 57 . . . 77: 91 . . . 99: 115 . . . 124: 139 . . . 151: 170 . . . 192: 210 . . . 230: |
| | 243 . . . 257 |
| 32 | 7 . . . 15: 53 . . . 60: 65 . . . 72 |
| 34 | 7 . . . 15: 57 . . . 66 |
| 36 | 52 . . . 61: 63 . . . 73: 90 . . . 98: 101 . . . 123: 127 . . . 140: 156 . . . 171: 182 . . . 187: 184 . . . 189: 207 . . . 227: |
| | 226 . . . 235: 250 . . . 261 |
| 38 | −1 . . . 16: 18 . . . 31 |

TABLE IV

| SEQ | | | | |
|---|---|---|---|---|
| ID | | | | |
| NO: | Position | Designation | Accession number | Database |
| 2 | 3-191 | Myelin proteolipid protein (PLP | Myelin_PLP | PFAM |
| | | or lipophilin) | | |
| 2 | 139-158 | Myelin proteolipid protein | MYELIN_PLP_2 | PROSITE |
| | | signature 2 | | |
| 6 | 2-45 | Somatomedin B domain | Somatomedin_B | PFAM |
| 6 | 20-40 | Somatomedin B domain | SOMATOMEDIN_B | PROSITE |
| | | signature | | |
| 6 | 68-110 | Somatomedin B domain | Somatomedin_B | PFAM |
| 6 | 86-106 | Somatomedin B domain | SOMATOMEDIN_B | PROSITE |
| | | signature | | |
| 8 | 3-221 | Calcineurin-like phosphoesterase | Metallophos | PFAM |
| 8 | 3-15 | 5′-nucleotidase signature 1 | 5_NUCLEOTIDASE_1 | PROSITE |
| 10 | 7-61 | Sushi domain (SCR repeat) | sushi | PFAM |
| 10 | 65-125 | Sushi domain (SCR repeat) | sushi | PFAM |
| 12 | 16-158 | ADP-ribosylation factor family | arf | PFAM |
| 12 | 39-46 | ATP/GTP-binding site motif A | ATP_GTP_A | PROSITE |
| | | (P-loop) | | |
| 14 | 377-384 | ATP/GTP-binding site motif A | ATP_GTP_A | PROSITE |
| | | (P-loop) | | |
| 16 | −4-4 | ATP/GTP-binding site motif A | ATP_GTP_A | PROSITE |
| | | (P-loop) | | |
| 16 | 6-50 | WAP-type (Whey Acidic | wap | PFAM |
| | | Protein) ‘four disulfide core’ | | |
| 16 | 26-39 | WAP-type‘four disulfide core’ | 4_DISULFIDE_CORE | PROSITE |
| | | domain signature | | |
| 18 | 1-177 | Class I Histocompatibility | MHC_I | PFAM |
| | | antigen, domains alpha 1 and 2 | | |
| 22 | 15-83 | Immunoglobin domain | ig | PFAM |
| 22 | 120-188 | Immunoglobin domain | ig | PFAM |
| 24 | 6-46 | Small cytokines | IL8 | PFAM |
| | | (intecrine/chemokine) | | |
| 26 | −18-132 | Somatotropin hormone family | hormone | PFAM |
| 26 | 108-125 | Somatotropin, prolactin and | SOMATOTROPIN_2 | PROSITE |
| | | related hormones signature 2 | | |
| 28 | 49-99 | Kunitz/Bovine pancreatic trypsin | Kunitz_BPTI | PFAM |
| | | inhibitor domain | | |
| 28 | 49-99 | | BPTI_KUNITZ_2 | PFSCAN |
| 28 | 77-95 | Pancreatic trypsin inhibitor | BPTI_KUNITZ_1 | PROSITE |
| | | (Kunitz) family signature | | |
| 30 | 2-60 | Sushi domain (SCR repeat) | sushi | PFAM |
| 30 | 65-122 | Sushi domain (SCR repeat) | sushi | PFAM |
| 30 | 127-185 | Sushi domain (SCR repeat) | sushi | PFAM |
| 32 | 2-51 | | TRYPSIN_DOM | PFSCAN |
| 32 | 38-43 | Serine proteases, trypsin family, | TRYPSIN_HIS | PROSITE |
| | | histidine active site | | |
| 34 | 2-67 | | TRYPSIN_DOM | PFSCAN |
| 34 | 38-43 | Serine proteases, trypsin family, | TRYPSIN_HIS | PROSITE |
| | | histidine active site | | |
| 36 | 57-277 | Fibrinogen beta and gamma | fibrinogen_C | PFAM |
| | | chains, C-terminal globular | | |
| | | domain | | |
| 36 | 234-246 | Fibrinogen beta and gamma | FIBRIN_AG_C_DOMAIN | PROSITE |
| | | chains C-terminal domain | | |
| | | signature | | |
| 38 | 1-31 | Statherin | Statherin | PFAM |
| 40 | 20-186 | | PRO_RICH | PFSCAN |

1. An isolated polynucleotide, comprising a nucleic acid sequence selected from the group consisting of:
a) a polynucleotide of an odd SEQ ID NO. 1-39, or of a human cDNA of a deposited clone, encoding at least any single integer from 6 to 776 amino acids of any one even SEQ ID NO. 2-40;
b) a polynucleotide of an odd SEQ ID NO. 1-39, or of a human cDNA of a deposited clone, encoding the signal peptide sequence of any one even SEQ ID NO. 2-40;
c) a polynucleotide of an odd SEQ ID NO. 1-39, or of a human cDNA of a deposited clone, encoding a mature polypeptide sequence of any one even SEQ ID NO. 2-40;
d) a polynucleotide of an odd SEQ ID NO. 1-39, or of a human cDNA of a deposited clone, encoding a full length polypeptide sequence of any one even SEQ ID NO. 2-40;
e) a polynucleotide of an odd SEQ ID NO. 1-39, or of a human cDNA of a deposited clone, encoding a polypeptide sequence of a biologically active fragment of any one even SEQ ID NO. 2-40;
f) a polynucleotide encoding a polypeptide sequence of at least any single integer from 6 to 776 amino acids of any one even SEQ ID NO. 2-40 or of a polypeptide encoded by a human cDNA of a deposited clone;
g) a polynucleotide encoding a polypeptide sequence of a signal peptide of any one even SEQ ID NO. 2-40 or of a signal peptide encoded by a human cDNA of a deposited clone;
h) a polynucleotide encoding a polypeptide sequence of a mature polypeptide of any one even SEQ ID NO. 2-40 or of a mature polypeptide encoded by a human cDNA of a deposited clone;
i) a polynucleotide encoding a polypeptide sequence of a full length polypeptide of any one even SEQ ID NO. 2-40 or of a mature polypeptide encoded by a human cDNA of a deposited clone;
j) a polynucleotide encoding a polypeptide sequence of a biologically polypeptide of any one even SEQ ID NO. 2-40, or of a biologically polypeptide encoded by a human cDNA of a deposited clone;
k) a polynucleotide of any one of a) through j) further comprising an expression vector;
l) a host cell recombinant for a polynucleotide of a) through k) above;
m) a non-human transgenic animal comprising the host cell of k); and
n) a polynucleotide of a) through j) further comprising a physiologically acceptable carrier.

2. A polypeptide comprising a contiguous amino acid sequence selected from the group consisting of:
a) any single integer from 6 to 776 amino acids of any one even SEQ ID NO. 2-40 or of a polypeptide encoded by a human cDNA of a deposited clone;
b) a signal peptide sequence of any one even SEQ ID NO. 2-40 or encoded by a human cDNA of a deposited clone;
c) a mature polypeptide sequence of any one even SEQ ID NO. 2-40 or encoded by a human cDNA of a deposited clone;
d) a full length polypeptide sequence of any one even SEQ ID NO. 2-40 or encoded by a human cDNA of a deposited clone; and
e) a polypeptide of a) through d) further comprising a physiologically acceptable carrier.

3. A method of making a polypeptide, said method comprising the steps of:
a) providing a population of host cells comprising the polynucleotide of claim 1;
b) culturing said population of host cells under conditions conducive to the production of a polypeptide of claim 2 within said host cells; and
c) purifying said polypeptide from said population of host cells.

4. A method of making a polypeptide, said method comprising the steps of:
a) providing a population of cells comprising a polynucleotide encoding the polypeptide of claim 2, operably linked to a promoter;
b) culturing said population of cells under conditions conducive to the production of said polypeptide within said cells; and
c) purifying said polypeptide from said population of cells.

5. An antibody that specifically binds to the polypeptide of claim 2.

6. A method of binding a polypeptide to an antibody comprising contacting a polypeptide with an antibody that can specifically bind said polypeptide under conditions in which said antibody can specifically bind to said polypeptide, wherein said polypeptide comprises a contiguous amino acid sequence selected from the group consisting of:
a) any single integer from 6 to 776 amino acids of any one even SEQ ID NO. 2-40 or of a polypeptide encoded by a human cDNA of a deposited clone;
b) a signal peptide sequence of any one even SEQ ID NO. 2-40 or encoded by a human cDNA of a deposited clone,
c) a mature polypeptide sequence of any one even SEQ ID NO. 2-40 or encoded by a human cDNA of a deposited clone,
d) a full length polypeptide sequence of any one even SEQ ID NO. 2-40 or encoded by a human cDNA of a deposited clone; and
e) a polypeptide of a) through d) further comprising a physiologically acceptable carrier.

7. A method of determining whether a GENSET gene is expressed within a mammal, said method comprising the steps of:
a) providing a biological sample from said mammal;
b) contacting said biological sample with either of:
i) a polynucleotide that hybridizes under stringent conditions to the polynucleotide of claim 1; or
ii) a polypeptide that specifically binds to the polypeptide of claim 2; and
c) detecting the presence or absence of hybridization between said polynucleotide and an RNA species within said sample, or the presence or absence of binding of said polypeptide to a protein within said sample,
wherein a detection of said hybridization or of said binding indicates that said GENSET gene is expressed within said mammal.

8. The method of claim 7, wherein said polynucleotide is a primer, and wherein said hybridization is detected by detecting the presence of an amplification product comprising the sequence of said primer.

9. The method of claim 7, wherein said polypeptide is an antibody.

10. A method of determining whether a mammal has an elevated or reduced level of GENSET gene expression, said method comprising the steps of:
a) providing a biological sample from said mammal; and
b) comparing the amount of the polypeptide of claim 2, or of an RNA species encoding said polypeptide, within said biological sample with a level detected in or expected from a control sample,
wherein an increased amount of said polypeptide or said RNA species within said biological sample compared to said level detected in or expected from said control sample indicates that said mammal has an elevated level of said GENSET gene expression, and wherein a decreased amount of said polypeptide or said RNA species within said biological sample compared to said level detected in or expected from said control sample indicates that said mammal has a reduced level of said GENSET gene expression.

11. A method of identifying a candidate modulator of a GENSET polypeptide, said method comprising the steps of:
a) contacting the polypeptide of claim 2 with a test compound; and
b) determining whether said compound specifically binds to said polypeptide,
wherein a detection that said compound specifically binds to said polypeptide indicates that said compound is a candidate modulator of said GENSET polypeptide.

12. The method of claim 11, further comprising testing the biological activity of said GENSET polypeptide in the presence of said candidate modulator, wherein an alteration in the biological activity of said GENSET polypeptide in the presence of said compound in comparison to the activity in the absence of said compound indicates that the compound is a modulator of said GENSET polypeptide.

13. A method for the production of a pharmaceutical composition comprising the steps of:
a) identifying a modulator of a GENSET polypeptide using the method of claim 11; and
b) combining said modulator with a physiologically acceptable carrier.


SEQUENCE LISTING (40 sequences)

SEQ ID NO: 1 - DNA, 1289 bp, Homo sapiens
  5′UTR 1..259
  CDS 260..904
  3′UTR 905..1289
  polyA_signal 1241..1246
  polyA_site 1248..1289
acaaattaggaaatgagccaggattacaactcatcctctcaactgtcaagccctgtgctctttcagcctcatcaactgcctcttggattgaattgtttattttccaggttgttttgaatgctgtatcaaatgcctggggggcattccctatgcctctctgattgccaccatcctgctctatgcgggtgttgccctgttctgtggctgcggtcatgaagcgctttctggaactgtcaacattctgcaaacctactttgagatggcaagaactgctggagacacactggatgtttttaccatgattgacatctttaagtatgtgatctacggcatcgcagctgcgttctttgtgtatggcattttgctgatggtggaaggtttcttcacaactggggccatcaaagatctctatggggatttcaaaatcaccacttgtggcagatgtgtgagcgcttggttcattatgctgacatatcttttcatgttggcctggctgggagtcacggctttcacctcactgccagtttacatgtacttcaatctgtggaccatctgccggaacaccacattagtggagggagcaaatctctgcttggaccttcgtcagtttggaattgtgacaattggagaggaaaagaaaatttgtactgtctctgagaatttcttgaggatgtgcgaatctactgagctgaacatgaccttccacttgtttattgtggcacttgctggagctggggcagcagtcattgctatggttcactaccttatggttctgtctgccaactgggcctatgtgaaagacgcctgccggatgcagaagtatgaagacatcaagtcgaaggaagagcaagagcttcatgacatccactctactcgctccaaagagcggctcaatgcatacacataaatgcatcttcctgttctttctaccatttgaatgcattggtgtttaactaagggccatccaaccatccaacctttaaaaaacaaaacgaaagtgcttctcatcaatgatatgtaaggtgacttatgaatcacctgagtacaattctttgttgtttagcacttaaatttcccaatttattaaattgatgtaaatcagatcttttctacaagctcctatccagccttttttttgaaatttctcaaactcatttactagttctgtaaaatcaaagatactaacattgtcaaatataaaaaaaaaaaaaaaaaagaaaaaaaaaaaaaaaaaaaaaaaaaaaaagaaaaaaaaaaaaaaaaaaaaaaaamcaaaaaaaaaaaaaaaa

SEQ ID NO: 2 - protein, 215 aa, Homo sapiens
MARTAGDTLDVFTMIDIFKYVIYGIAAAFFVYGILLMVEGFFTTGAIKDLYGDFKITTCGRCVSAWFIMLTYLFMLAWLGVTAFTSLPVYMYFNLWTICRNTTLVEGANLCLDLRQFGIVTIGEEKKICTVSENFLRMCESTELNMTFHLFIVALAGAGAAVIAMVHYLMVLSANWAYVKDACRMQKYEDIKSKEEQELHDIHSTRSKERLNAYT

SEQ ID NO: 3 - DNA, 603 bp, Homo sapiens
  5′UTR 1..137
  CDS 138..320
  3′UTR 321..603
  polyA_signal 562..567
  polyA_site 588..603
attcccagcctcacatcactcacaccttgcatttcacccctgcatctcagtcgccctgcagcctcacacagatcctgcacacacccagacagctggcgctcacacattcaccgttggcctgcctctgttcaccctccatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtatgaagcgccgcagcagttaacctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaacgaaaaaaaaaaaaaaaa

SEQ ID NO: 4 - protein, 61 aa, Homo sapiens
  SIGNAL -21..-1
MALLLALSLLVLWTSPAPTLSGTNDAEDCCLSVTQKPIPGYIVRNFHYLLIKDGCRVPAVV

SEQ ID NO: 5 - DNA, 1673 bp, Homo sapiens
  5′UTR 1..111
  CDS 112..1341
  3′UTR 1342..1673
  polyA_signal 1528..1533
  polyA_site 1548..1673
gtctttaaaaggatcagcttgaggaataaggctcgtctgagagctgtgacattcatctgactctagtgaaagtccaacagccactccctttttggcctccaactgggcaccatgagggcctgcatctccctggtattggccgtgctgtgtggcctggcctgggctggaaaaatagaatcctgtgcatctcgatgtaatgagaaatttaaccgggacgctgcctgccagtgtgaccgccggtgtctctggcatgggaactgctgtgaagactatgaacatctgtgtactgaggaccacaaagagtcagagccattgccacagctggaggaagagacagaagaggccctcgccagcaacttgtactcggcacccacctcctgccagggccgctgctacgaagcctttgacaagcaccaccaatgtcactgcaatgcccgctgccaagagtttgggaactgctgcaaggattttgagagcctgtgtagtgaccacgaggtctcccacagcagtgatgccataacaaaagaggagattcagagcatctctgagaagatctacagggcagacaccaacaaagcccagaaggaagacatcgttctcaatagccaaaactgcatctccccgtcagagaccagaaaccaagttgatcgctgcccaaagccactcttcacttatgtcaatgagaagctgttctccaagcccacctatgcagccttcatcaacctcctcaacaactaccagcgggcaacaggccatggggagcacttcagtgcccaggagctggccgagcaggacgccttcctcagagagatcatgaagacagcagtcatgaaggagctctacagcttcctccatcaccagaatcgctatggctcagagcaagagtttgtcgatgacttgaagaacatgtggtttgggctctattcaagaggcaatgaagagggggactcgagtggctttgaacatgtcttctcaggtgaggtaaaaaaaggcaaggttactggcttccataactggatccgcttctacctggaggagaaggagggtctggttgactattacagtcacatctacgatgggccttgggattcttaccccgatgtgctggcaatgcagttcaactgggacggctactataaggaagtgggctctgctttcatcggcagcatccctgagtttgagtttgcactctactccctgtgcttcatcgccaggccaggcaaagtgtgccagttaagcctgggaggatatcccttagctgtccggacatatacctgggacaagtccacctatgggaatggcaagaagtacatcgccacagcctacatagtgtcttccacctaatagaacttcgagccagaaaggggcatgagggctcttgcgagactgaagtgctatcttctctggactagagagaagagggagaggactggaagggatcaccaaatctcaaagcaatgagaacattcctaaatcccaaagtgcccacatgggaaagagataaaatgtacaaattagaaaaatgtggataaacagtcaaaccttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaraaaaaaaaaaaraaaaaaaaaaaaaaaa

SEQ ID NO: 6 - protein, 410 aa, Homo sapiens
  SIGNAL -18..-1
MRACISLVLAVLCGLAWAGKIESCASRCNEKFNRDAACQCDRRCLWHGNCCEDYEHLCTEDHKESEPLPQLEEETEEALASNLYSAPTSCQGRCYEAFDKHHQCHCNARCQEFGNCCKDFESLCSDHEVSHSSDAITKEEIQSISEKIYRADTNKAQKEDIVLNSQNCISPSETRNQVDRCPKPLFTYVNEKLFSKPTYAAFINLLNNYQRATGHGEHFSAQELAEQDAFLREIMKTAVMKELYSFLHHQNRYGSEQEFVDDLKNMWFGLYSRGNEEGDSSGFEHVFSGEVKKGKVTGFHNWIRFYLEEKEGLVDYYSHIYDGPWDSYPDVLAMQFNWDGYYKEVGSAFIGSIPEFEFALYSLCFIARPGKVCQLSLGGYPLAVRTYTWDKSTYGNGKKYIATAYIVSST

SEQ ID NO: 7 - DNA, 993 bp, Homo sapiens
  5′UTR 1..57
  CDS 58..849
  3′UTR 850..993
  polyA_signal 952..957
  polyA_site 978..993
actcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctcggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaattttataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggtaattgtttcaaaaggattgcatgggccaggatgtccagataagcactgtgtctcttttgcctttgtaactgttattactctttttactgctatttaatatgtaatgtatattatatgatctataatatatatgtaatatacattaaatgggaacatgtgcaaatcttaaaaaaaaaaaaaaaa

SEQ ID NO: 8 - protein, 264 aa, Homo sapiens
  SIGNAL -26..-1
MCPRAARAPATLLLALGAVLWPAAGASELTILHTNDVHSRLEQTSEDSSKCVNASRCMGGVARLFTKVQQIRRAEPNVLLLDAGDQYQGTIWFTVYKGAEVAHFMNALRYDAMALGNHEFYNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNCFKRIAWARMSR

SEQ ID NO: 9 - DNA, 611 bp, Homo sapiens
  5′UTR 1..23
  CDS 24..461
  3′UTR 462..611
  polyA_signal 572..577
  polyA_site 596..611
atataagattggaactaccaagcatgtggctcctggtcagtgtaattctaatctcacggatatcctctgttgggggagaagtttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaagaaaaatagaatcaatggcattactattagtaaaatgcacacctttttctgaatttactattatatttgttttcaatttcatttttcaagtactgttttactcatttttattcataaataaagttttgtgttgatttgtgaaaaaaaaaaaaaaaa

SEQ ID NO: 10 - protein, 146 aa, Homo sapiens
  SIGNAL -18..-1
MWLLVSVILISRISSVGGEVSAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 11 - DNA, 588 bp, Homo sapiens
  5′UTR 1..97
  CDS 98..571
  3′UTR 572..588
  polyA_site 574..588
gctgttgctggctttttcaggagctgctcgctcgcagccagagacgctgctttttttttccgggttcggagccgttccggatgctttaggctgccggatgtctgatctccgaataactgaggcgtttctgtacatggattatctgtgttttagagcactttgctgcaagggaccaccacctgcacgaccagaatatgacctggtttgcataggcctcacaggttctggcaaaaccagtctgttgtccaaactctgcagtgaaagccccgataacgtcgtgtcgaccacaggttttagtattaaagcagtgccattccagaatgccatcttgaatgtaaaagaacttggaggggctgataacatccggaaatactggagccgctactaccaaggatctcaaggggtaatatttgtattagacagtgcctcttcagaggatgatttagaagctgctagaaatgagctgcactcagctcttcagcatccacagttatgcactttaccctttttaatattggccaatcatcaagacaagccagcagctcgctcagtacaagagaaaattcaggactgaaaaaaaaaaaaaaa

SEQ ID NO: 12 - protein, 158 aa, Homo sapiens
MSDLRITEAFLYMDYLCFRALCCKGPPPARPEYDLVCIGLTGSGKTSLLSKLCSESPDNVVSTTGFSIKAVPFQNAILNVKELGGADNIRKYWSRYYQGSQGVIFVLDSASSEDDLEAARNELHSALQHPQLCTLPFLILANHQDKPAARSVQEKIQD

SEQ ID NO: 13 - DNA, 1633 bp, Homo sapiens
  5′UTR 1..17
  CDS 18..1403
  3′UTR 1404..1633
  polyA_signal 1594..1599
  polyA_site 1618..1633
aagatttttcaagcaagatgaagtccatcatcctctttgtcctttccctgctccttatcttggagaagcaagcagctgtgatgggacaaaaaggtggatcaaaaggccaattgccaagcggatcttcccaatttccacatggacaaaagggccagcactattttggacaaaaagaccaacaacatactaaatccaaaggcagtttttctattcaacacacatatcatgtagacatcaatgatcatgactggacccgaaaaagtcagcaatatgatttgaatgccctacataaggcgacaaaatcaaaacaacacctaggtggaagtcaacaactgctcaattataaacaagaaggcagagaccatgataaatcaaaaggtcattttcacatgatagttatacatcataaaggaggccaagctcatcatgggacacaaaatccttctcaagatcaggggaatagcccatctggaaagggattatccagtcaatgttcaaacacagaaaaaaggctatgggttcatggactaagtaaagaacaagcttcagcctctggtgcacaaaaaggtagaacacaaggtggatcccaaagcagttatgttctccaaactgaagaactagtagttaacaaacaacaacgtgagactaaaaattctcatcaaaataaagggcattaccaaaatgtggttgacgtgagagaggaacattcaagtaaactacaaacttcactccatcctgcacatcaagacagactccaacatggacccaaagacatttttactacccaagatgagctcctagtatataacaagaatcaacaccagacaaaaaatctcagtcaagatcaagagcatggccggaaggcacataaaatatcatacccgtcttcacgtacagaagaaagacaacttcaccatggagaaaagagtgtacagaaagatgtatccaaaggcagcatttctatccaaactgaagagaaaatacatggcaagtctcaaaaccaggtaacaattcatagtcaagatcaagagcatggccataaggaaaataaaatatcataccaatcttcaagtacagaagaaagacatctcaactgtggagaaaagggcatccagaaaggtgtatccaaaggcagtatttcgatccaaactgaagagcaaatacatggcaagtctcaaatccagacaccaaatcctaatcaagatcaatggtctggccaaaatgcaaaaggaaagtctggtcaatctgcagatagcaaacaagacctactcagtcatgaacaaaaaggcagatacaaacaggaatccagtgagtcacataatattgtaattactgagcatgaggttgcccaagatgatcatttgacacaacaatataatgaagacagaaatccaatatctacatagccctgttgcttagcaaccacttgaaaagctggaccaatagcaaggtgtcacccgacctcagtgaagtctttgatgtttctgagaggcagactcccatgtggtcccagatccttggtccatggatgacaccaccttcccatgcttccttgcattaggctttctaaacccggagccccttcaaacttccaataaagggatcattttctgcttcaaaaaaaaaaaaaaaa

SEQ ID NO: 14 - protein, 462 aa, Homo sapiens
  SIGNAL -23..-1
MKSIILFVLSLLLILEKQAAVMGQKGGSKGQLPSGSSQFPHGQKGQHYFGQKDQQHTKSKGSFSIQHTYHVDINDHDWTRKSQQYDLNALHKATKSKQHLGGSQQLLNYKQEGRDHDKSKGHFHMIVIHHKGGQAHHGTQNPSQDQGNSPSGKGLSSQCSNTEKRLWVHGLSKEQASASGAQKGRTQGGSQSSYVLQTEELVVNKQQRETKNSHQNKGHYQNVVDVREEHSSKLQTSLHPAHQDRLQHGPKDIFTTQDELLVYNKNQHQTKNLSQDQEHGRKAHKISYPSSRTEERQLHHGEKSVQKDVSKGSISIQTEEKIHGKSQNQVTIHSQDQEHGHKENKISYQSSSTEERHLNCGEKGIQKGVSKGSISIQTEEQIHGKSQIQTPNPNQDQWSGQNAKGKSGQSADSKQDLLSHEQKGRYKQESSESHNIVITEHEVAQDDHLTQQYNEDRNPIST

SEQ ID NO: 15 - DNA, 605 bp, Homo sapiens
  5′UTR 1..21
  CDS 22..360
  3′UTR 361..605
  polyA_signal 565..570
  polyA_site 590..605
agagtcactcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcgttacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacaccaggctttcaagaagtgccagttgatcaatgaataaataaacgagcctatttctctttacaaaaaaaaaaaaaaaa

SEQ ID NO: 16 - protein, 113 aa, Homo sapiens
  SIGNAL -25..-1
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCVT

SEQ ID NO: 17 - DNA, 1212 bp, Homo sapiens
  5′UTR 1..57
  CDS 58..690
  3′UTR 691..1212
  polyA_signal 1177..1182
  polyA_site 1197..1212
tcatcttccaggctctccttccatcaagtctctcctccctagcgctctgggtccttaatggcagcagccgccgctaccaagatccttctgtgcctcccgcttctgctcctgctgtccggctggtcccgggctgggcgagccgaccctcactctctttgctatgacatcaccgtcatccctaagttcagacctggaccacggtggtgtgcggttcaaggccaggtggatgaaaagacttttcttcactatgactgtggcaacaagacagtcacacctgtcagtcccctggggaagaaactaaatgtcacaacggcctggaaagcacagaacccagtactgagagaggtggtggacatacttacagagcaactgcgtgacattcagctggagaattacacacccaaggaacccctcaccctgcaggccaggatgtcttgtgagcagaaagctgaaggacacagcagtggatcttggcagttcagtttcgatgggcagatcttcctcctctttgactcagagaagagaatgtggacaacggttcatcctggagccagaaagatgaaagaaaagtgggagaatgacaaggttgtggccatgtccttccattacttctcaatgggagactgtataggatggcttgaggacttcttgatgggcatggacagcaccctggagccaagtgcaggaggttaaagctgataccaaaaggctcctgtgagcacggtcttgatcaaactcgcccttctgtctggccagctgcccacgacctacggtgtatgtccagtggcctccagcagatcatgatgacatcatggacccaatagctcattcactgccttgattccttttgccaacaattttaccagcagttatacctaacatattatgcaattttctcttggtgctacctgatggaattcctgcacttaaagttctggctgactaaacaagatatatcattttctttcttctctttttgtttggaaaatcaagtacttctttgaatgatgatctctttcttgcaaatgatattgtcagtaaaataatcacgttagacttcagacctctggggattctttccgtgtcctgaaagagaatttttaaattatttaataagaaaaaatttatattaatgattgtttcctttagtaatttattgttctgtactgatatttaaataaagagttctatttcccaaaaaaaaaaaaaaaa

SEQ ID NO: 18 - protein, 211 aa, Homo sapiens
  SIGNAL -28..-1
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGG

SEQ ID NO: 19 - DNA, 947 bp, Homo sapiens
  5′UTR 1..39
  CDS 40..435
  3′UTR 436..947
  polyA_signal 910..915
  polyA_site 932..947
ttactttcactcaccgcctctccttcctgacacctcaccatgtgtacgggaaaatgtgcccgctgtgtggggctctccctcattaccctctgcctcgtctgcattgtggccaacgccctcctgctggtacctaatggggagacctcctggaccaacaccaaccatctcagcttgcaagtctggctcatgggcggcttcattggcgggggcctaatggtactgtgtccggggattgcagccgttcgggcagggggcaagggctgctgtggtgctgggtgctgtggaaaccgctgcaggatgctgcgctcggtcttctcctcggcgttcggggtgcttggtgccatctactgcctctcggtgtctggagctgggctccgaaatggacccagatgcttaatgaacggcgagtggggctaccacttcggagacaccgcgtaaggtttagcctgtattcgtgtcctccattctctgcctcttccctcccgcccttcccccgtggactgggacacctgggcgggactcgacttcgagggcactcgctccacgtgggctacctgggccttagcaaatcccctgggacgtattcttgggggcggggtggcgcacgcgcacgctgccttctcccacgtggcctcacccctcccacaggggagcttacttgctcaaccgcactctatgggatcggtgcgaggcgccccctcgcgtggtcccctggaatgtgacgctcttctcgctgctggtggccgcctcctgcctggagatagtactgtgtgggatccagctggtgaacgcgaccattggtgtcttctgcggcgattgcaggaaaaaacaggacacacctcactgaggctccactgaccgccgggttacacctgctccttcctggacgctcactcccttgctcgctagaataaactgctttgcgctctcgaaaaaaaaaaaaaaaa

SEQ ID NO: 20 - protein, 132 aa, Homo sapiens
  SIGNAL -26..-1
MCTGKCARCVGLSLITLCLVCIVANALLLVPNGETSWTNTNHLSLQVWLMGGFIGGGLMVLCPGIAAVRAGGKGCCGAGCCGNRCRMLRSVFSSAFGVLGAIYCLSVSGAGLRNGPRCLMNGEWGYHFGDTA

SEQ ID NO: 21 - DNA, 1389 bp, Homo sapiens
  5′UTR 1..229
  CDS 230..934
  3′UTR 935..1389
  polyA_signal 1353..1358
  polyA_site 1374..1389
agaacagacccccatccctgggctggaggacccgcctcttggcagccagctgagaaggcgccccggggagggggaaactgacatcccatctagagccgtccctcctcttcctcccctcccgactctctgctcctttcccgccccagaagttcaagggcccccggcctcctgcgctcctgccgccgggaccctcgacctcctcagagcagccggctgccgccccgggaagatggcgaggaggagccgccaccgcctcctcctgctgctgctgcgctacctggtggtcgccctgggctatcataaggcctatgggttttctgccccaaaagaccaacaagtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcaggtgtcctgggaaacgaatgcaaatgatctcaacataagtggcatcatagcagccgtagtagttgtggccttagtgatttccgtttgtggccttggtgtatgctatgctcagaggaaaggctacttttcaaaagaaacctccttccagaagagtaattcttcatctaaagccacgacaatgagtgaaaatgatttcaagcacacaaaatcctttataatttaaagactccactttagagatacaccaaagccaccgttgttacacaagttattaaactattataaaactctgctttgtccgacatttgcaaagaggtacacgaggaaatggaattggtatttcattttaattttcatgactactaactcacctgaacttgctattttaaacaaatagttctgtcgacacctaaaatataatctggcttcttgtgtctggactaagttaaaagaattaaaatactttgtaatgacaaaaaaaaaaaaaaaa

SEQ ID NO: 22 - protein, 235 aa, Homo sapiens
  SIGNAL -28..-1
MARRSRHRLLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQMIST

SEQ ID NO: 23 - DNA, 551 bp, Homo sapiens
  5′UTR 1..109
  CDS 110..304
  3′UTR 305..551
  polyA_signal 518..523
  polyA_site 536..551
attttaaaaaatatttggcctcaatgatcatttctcaacaattcctcaccgcaggagcctctgaagctcccaccaggccagctctcctcccacaacagcttcccacagcatgaagatctccgtggctgccattcccttcttcctcctcatcaccatcgccctagggaccaagactgaatcctcatcacggggaccttaccacccctcagagtgctgcttcacctacactacctacaagatcccgcgtcagcggattatggattactatgagaccaacagccagtgctccaagcccggaattgtgtagcttcatcaccaaaaggggccattccgtctgtaccaaccccagtgacaagtgggtccaggactatatcaaggacatgaaggagaactgagtgacccagaaggggtggcgaaggcacagctcagagacataaagagaagatgccaaggccccctcctccacccaccgctaactctcagccccagtcaccctcttggagcttccctgctttgaattaaagaccactcatgcaaaaaaaaaaaaaaaa

SEQ ID NO: 24 - protein, 65 aa, Homo sapiens
  SIGNAL -19..-1
MKISVAAIPFFLLITIALGTKTESSSRGPYHPSECCFTYTTYKIPRQRIMDYYETNSQCSKPGIV

SEQ ID NO: 25 - DNA, 786 bp, Homo sapiens
  5′UTR 1..146
  CDS 147..626
  3′UTR 627..786
  polyA_signal 714..719
  polyA_site 741..786
aataaatgtgcacagaaacaggtgggggcaacagcgagagagaaggggccagggtataaaaagggcccacaagagaccagctccagcatcccaaggcccgactccccgcaccactcagggtcctgtggacagctcacctagcggcaatggctgcaggctcccggacgtccctgctcctggcttttgccctgctctgcctgccctggcttcaagaggctgactctattccgacatcctccaacatggaggaaacgcagcagaaatccaacttagagctgctccacatctccctgctgctcatcgagtcgcggctggagcccgtgcggttcctcaggagtaccttcaccaacaacctggtgtatgacacctcggacagcgatgactatcacctcctaaaggacctagaggaaggcatccaaatgctgatggggaggctggaagacggcagccacctgactgggcagaccctcaagcagacctacagcaagtttgacacaaactcgcacaaccatgacgcactgctcaagaactacgggctgctccactgcttcaggaaggacatggacaaggtcgagacattcctgcgcatggtgcagtgccgctctgtggagggcagctgtggcttctaggggcccgcgtggcatcctgtgacccctccccagtgcctctcctggccctgaaggtgccactccagtgcccaccagccttgtcttaataaaattaagttgtattgtttcatcaaaaaaaaaaaaaaaaaaaaaaaaaaaaagaaaaaaaaaaaaaaaa

SEQ ID NO: 26 - protein, 160 aa, Homo sapiens
  SIGNAL -26..-1
MAAGSRTSLLLAFALLCLPWLQEADSIPTSSNMEETQQKSNLELLHISLLLIESRLEPVRFLRSTFTNNLVYDTSDSDDYHLLKDLEEGIQMLMGRLEDGSHLTGQTLKQTYSKFDTNSHNHDALLKNYGLLHCFRKDMDKVETFLRMVQCRSVEGSCGF

SEQ ID NO: 27 - DNA, 1409 bp, Homo sapiens
  5′UTR 1..317
  CDS 318..902
  3′UTR 903..1409
  polyA_signal 1345..1350
  polyA_site 1368..1409
attggctctggcgacctccgcgcgttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggcttcccgcacctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccgagaacgcagcatccacgagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctgtttctctgggaggtaggacggctgcttcctggtctggcagggatgggtttgctttggaaatcctctaggaggctcctcctcgcatggcctgcagtctggcagcagccccgagttgtttcctcgctgatcgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaatttcagcatgtgctttcaaaaaaaaaaaaaaaaaaaaaaaaagaaaaaaaaaaaaaaaa

SEQ ID NO: 28 - protein, 195 aa, Homo sapiens
  SIGNAL -27..-1
MAQLCGLRRSRAFLALLGSLLLSGVLAADRERSIHENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 29 - DNA, 1195 bp, Homo sapiens
  5′UTR 1..123
  CDS 124..1074
  3′UTR 1075..1195
  polyA_signal 1158..1163
  polyA_site 1180..1195
actcatttaactggtattgcggagccacgaggcttctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatttgccttaagggcagtcaatggtcagatattgaagagttctgcaatcagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcaaaaaaaaaaaaaaaa

SEQ ID NO: 30 - protein, 317 aa, Homo sapiens
  SIGNAL -34..-1
MTVARPSVPAALPLLGELPRLLLLVLLCLPAVWGDCGLPPDVPNAQPALEGRTSFPEDTVITYKCEESFVKIPGEKDSVICLKGSQWSDIEEFCNQKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 31 - DNA, 1745 bp, Homo sapiens
  5′UTR 1..41
  CDS 42..371
  3′UTR 372..1745
  polyA_signal 1709..1714
  polyA_site 1730..1745
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctggacagaagcaggacagggcctggctctgccgctgccagaggctgccgctggcctccctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacacaatcgtggccaacccctgagcacccctatcaactccctattgtagtaaacttggaaccttggaaatgaccaggccaagactcaggcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagcggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgcaaaaaaaaaaaaaaaa

SEQ ID NO: 32 - protein, 110 aa, Homo sapiens
  SIGNAL -23..-1
MWVPVVFLTLSVTWIGAAPLILSRIVGGWECEKHSQPWQVLVASRGRAVCGGVLVHPQWVLTAAHCIRNKSVILLDRSRTGPGSAAARGCRWPPYGIRLQGGRAAGMWRE

SEQ ID NO: 33 - DNA, 1409 bp, Homo sapiens
  5′UTR 1..41
  CDS 42..311
  3′UTR 312..1409
  polyA_signal 1372..1377
  polyA_site 1394..1409
agccccaagcttaccacctgcacccggagagctgtgtcaccatgtgggtcccggttgtcttcctcaccctgtccgtgacgtggattggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcgctcaatttctctgaggacacagataggatggggtgtctgtgttatttgtgggatacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtgaaaaaaaaaaaaaaaa

SEQ ID NO: 34 - protein, 90 aa, Homo sapiens
  SIGNAL -23..-1
MWVPVVFLTLSVTWIGAAPLILSRIVGGWECEKHSQPWQVLVASRGRAVCGGVLVHPQWVLTAAHCIRNKSVILLGRHSLFHPEDTGQAR

SEQ ID NO: 35 - DNA, 1153 bp, Homo sapiens
  5′UTR 1..189
  CDS 190..1092
  3′UTR 1093..1153
  polyA_site 1138..1153
atggatggaacctgagctgggtctctgactcacttctgactttagggtgaagatggagggaatctgataaagacatcttataaattcaacagacacaaaagaatttgatctcccataagcaactgtgaaattacaataacagatcctgggaagttctacaattctaattcagttttttcaagggggaacatggcaaaggtgttcagtttcatccttgttaccaccgctctgataatgggcagggaaatttcggcgctcgaggactgtgcccaggagcagatgcggctcagagcccaggtgcgcctgcttgagacccgggtcaaacagcaacaggtcaagatcaagcagcttttgcaggagaatgaagtccagttccttgataaaggagatgagaatactgtcgttgatcttggaagcaagaggcagtatgcagattgttcagagattttcaatgatgggtataagctcagtggattttacaaaatcaaacctctccagagcccagcagaattttctgtttattgtgacatgtccgatggaggaggatggactgtaattcagagacgatctgatggcagtgaaaactttaacagaggatggaaagactatgaaaatggctttggaaattttgtccaaaaacatggtgaatattggctgggcaataaaaatcttcacttcttgaccactcaagaagactacactttaaaaatcgaccttgcagattttgaaaaaaatagccgttatgcacaatataagaatttcaaagttggagatgaaaagaatttctacgagttgaatattggggaatattctggaacagctggagattcccttgcggggaattttcatcctgaggtgcagtggtgggctagtcaccaaagaatgaaattcagcacgtgggacagagatcatgacaactatgaagggaactgcgcagaagaagatcagtctggctggtggtttaacaggtgtcactctgcaaacctgaatggtgtatactacagcggcccctacacggctaaaacagacaatgggattgtctggtacacctggcatgggttggtattctctgaaatctgtggttatgaaaattaggccaaatgattttattccaaatgtaatttaattgctgctaagaaaaaaaaaaaaaaaa

SEQ ID NO: 36 - protein, 301 aa, Homo sapiens
  SIGNAL -22..-1
MAKVFSFILVTTALIMGREISALEDCAQEQMRLRAQVRLLETRVKQQQVKIKQLLQENEVQFLDKGDENTVVDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGLVFSEICGYEN

SEQ ID NO: 37 - DNA, 548 bp, Homo sapiens
  5′UTR 1..75
  CDS 76..231
  3′UTR 232..548
  polyA_signal 509..514
  polyA_site 533..548
atcatctcttgaagcttcacttcaacttcactacttctgtagtctcatcttgagtaaaagagaacccagccaactatgaagttccttgtctttgccttcatcttggctctcatggtttccatgattggagctgattcatctgaagagtatgggtatggcccttatcagccagttccagaacaaccactatacccacaaccataccaaccacaataccaacaatataccttttaatatcatcagtaactgcaggacatgattattgaggcttgattggcaaatacgacttctacatccatattctcatctttcataccatatcacactactaccactttttgaagaatcatcaaagagcaatgcaaatgaaaaacactataattactgtatactctttgtttcaggatacttgccttttcaattgtcacttgatgatataattgcaatttaaactgttaagctgtgttcagtactgtttctgaataatagaaatcacttctctaaaagcaataaatttcaagcacattttttcaaaaaaaaaaaaaaaa

SEQ ID NO: 38 - protein, 52 aa, Homo sapiens
  SIGNAL -19..-1
MKFLVFAFILALMVSMIGADSSEEYGYGPYQPVPEQPLYPQPYQPQYQQYTF

SEQ ID NO: 39 - DNA, 792 bp, Homo sapiens
  5′UTR 1..38
  CDS 39..662
  3′UTR 663..792
  polyA_signal 757..762
  polyA_site 777..792
agcacagagttgggagtgactccagagcctcctgcaagatgctgttgattctgctgtcagtggccttgctggccctgagctcagctcagaacttaaatgaagatgtcagccaggaagaatctccctccctaatagcaggaaatccacaaggaccatccccacaaggaggcaacaagccccaaggccccccacctcctccaggaaagccacaaggaccacccccacaaggaggcaacaaacctcaaggtcccccacctccaggaaagccacaaggaccacccccacaaggggacaagtcccgaagtccccgatctcctccaggaaaaccacaaggaccacccccacaaggaggaaagccacaaggaccacccccacaaggaggcaacaaacctcagggtcccccacctccaggaaagccacaaggaccacccgcacaaggaggcagcaagtcccaaagtgcccgatctcctccaggaaagccacaaggaccaccccaacaagaaggcaacaatcctcaaggtcccccacctccagcaggaggcaatccccagcagcctcaggcacctcctgctggacagccccagggaccaccacgccctcctcaagggggcagaccttccagacctcaccagttacagcctccccagtcatctaggattcaatgacaggaagtgaataagaagatgagagtgattcaaatgattcaaattccatgacattggaaaaaggtcatcatagctctaacttcaatataccaataaaataatcagcttgcgaaaaaaaaaaaaaaaa

SEQ ID NO: 40 - protein, 208 aa, Homo sapiens
  SIGNAL -16..-1
MLLILLSVALLALSSAQNLNEDVSQEESPSLIAGNPQGPSPQGGNKPQGPPPPPGKPQGPPPQGGNKPQGPPPPGKPQGPPPQGDKSRSPRSPPGKPQGPPPQGGKPQGPPPQGGNKPQGPPPPGKPQGPPAQGGSKSQSARSPPGKPQGPPQQEGNNPQGPPPPAGGNPQQPQAPPAGQPQGPPRPPQGGRPSRPHQLQPPQSSRIQ